(19) United States
(12) Patent Application Publication
(10) Pub. No.: US 2025/0109118 A1
(43) Pub. Date: Apr. 3, 2025

(54) HETEROCYCLIC COMPOUNDS AS SHP2 INHIBITORS, COMPOSITIONS COMPRISING THE HETEROCYCLIC COMPOUND, AND METHODS OF USE THEREOF

(71) Applicant: JS INNOMED HOLDINGS LTD., Grand Cayman (KY)

(72) Inventors: Qun LI, Sunnyvale, CA (US); Jintao ZHANG, Naperville, IL (US); Shanzhong JIAN, Shanghai (CN); Ao LI, Shanghai (CN); Xia YUAN, Shanghai (CN); Wen XU, Shanghai (CN)

(21) Appl. No.: 18/725,113

(22) PCT Filed: Dec. 28, 2021
(86) PCT No.: PCT/CN2021/141980

§ 371 Date: Jun. 27, 2024

(51) Int. Cl. C07D 401/04; A61K 31/497; A61K 31/519; A61P 35/00; C07D 401/14; C07D 471/10; C07D 491/107; C07D 519/00

(52) U.S. Cl. CPC C07D 401/04; A61K 31/497; A61K 31/519; A61P 35/00; C07D 401/14; C07D 471/10; C07D 491/107; C07D 519/00

(57) ABSTRACT

Disclosed herein are compounds of formula (I), and/or a stereoisomer, a stable isotope, or a pharmaceutically acceptable salt or solvate thereof; and therapeutic uses of these compounds, which are inhibitors of SHP2, potentially useful in the treatment of SHP2-associated diseases, such as SHP2-associated cancers.

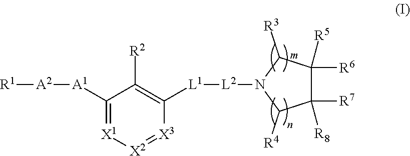

### TECHNICAL BACKGROUND

Disclosed herein are novel heterocyclic compounds that can serve as SHP2 (Srchomology-2-domain-containing protein tyrosine phosphatase 2) inhibitors. Further disclosed herein are pharmaceutical compositions, comprising at least one of such compounds, as well as methods of using at least one of such compounds in the treatment of diseases and disorders modulated by SHP2, such as cancers.

SHP2 is a non-receptor ubiquitous protein tyrosine phosphatase encoded by the PTPN11 gene in humans, with a relatively conserved structure and function. It contains a protein tyrosine phosphatase catalytic domain (PTP domain), two SH2 domains and a C-terminal tail with two tyrosine phosphorylation sites and a proline-rich motif. SHP2 catalyzes a critical control element in mammalian signal transduction: the dephosphorylation of phosphotyrosine. Upon stimulation by growth factors or cytokines, the N—SH2 domain binds to specific phosphotyrosine residues on cell surface receptors to induce a conformational change, which exposes and catalytically activates the PTP domain, resulting in SHP2 activation (Qu C K, el al. Cell Res 2000, 10, 279-88). SHP2 acts downstream of receptor tyrosine kinases but upstream of RAS (Yuan X R et. al. J Med Chem 2020, 10.1021/acs.jmedchem.0c00249). In addition, the immune check point PD-1 signals through SHP2 to suppress the activity of T cells in the tumor microenvironment (Marasco et al., Sci. Adv. 2020; 6: eaay4458).

Dysregulation of SHP2 has been implicated in a range of human pathologies, including cancer. SHP2 regulates cancer cell survival and proliferation primarily by activating the RAS-ERK signaling pathway (Matozaki T, el al. Cancer Sci 2009, 100, 1786-93). SHP2 mutations cause Noonan and LEOPARD syndromes, and mutations that increase SHP2's basal activity are the most common cause of sporadic juvenile myelomonocytic leukemia (Tartaglia, M, et al. Nat. Genet.2003, 34, 148-150). These rare diseases predispose patients to development of cancer. SHP2 activities have also been strongly implicated in oncogenesis even in instances in which the enzyme does not itself harbor mutations (Marsh-Armstrong B, et al. ACS Omega 2018, 3, 15763-15770. Recent studies have demonstrated that SHP2 is required for the growth and survival of RTK-driven (Chen Y N, et al. Nature 2016, 535, 148-52) and mutant KRAS-driven cancers (Mainardi S, et al. Nat Med 2018, 24, 961-7; Ruess D A, et al. Nat Med 2018, 24, 954-60). It has also recently shown that SHP2 inhibition triggers anti-tumor immunity and synergizes with PD-1 blockade (Zhao, MX, el al. Acta Pharmaceutica Sinica B, 2019, 9, 304-315). Therefore, SHP2 has emerged as an attractive target for the treatment of various diseases mediated by SHP2.

### SUMMARY OF THE INVENTION

Disclosed herein are a novel series of heterocyclic compounds that can serve as inhibitors of SHP2. Further disclosed herein are pharmaceutical compositions comprising at least one of such novel compounds, methods for preparing the novel compounds, as well as methods of using at least one of such compounds in the treatment of diseases and disorders mediated by SHP2 such as cancer.

Disclosed herein are compounds of Formula I:

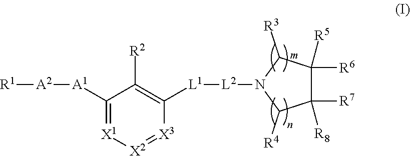

and/or stereoisomers, stable isotopes, or pharmaceutically acceptable salts or solvates thereof, wherein R¹, R², R³, R⁴, R⁵, R⁶, R⁷, R⁸, m, n, A¹, A², L¹, L², X¹, X², and X³ are defined below.

R¹ is an optionally substituted group selected from C1-C12 alkyl, C3-C12 cycloalkyl, 4-12 membered heterocyclyl containing 1-3 heteroatoms selected from N, O, and S as ring members, aryl, and heteroaryl containing 1-4 heteroatoms selected from N, O, and S as ring members; and wherein R¹ is optionally substituted with 1-4 substituents independently selected from R⁹, wherein R⁹ is selected from halo, —OH, NH₂, ═O, —CN, —OC(O)R¹⁰, —CO₂R¹⁰, —C(O)N(R^{11a}R^{11b}), —C(═NR¹²)N(R^{11a}R^{11b}), —C(O)R¹⁰, —OR¹⁰, —SR¹⁰, —S(O)₀₋₂R¹³, —S(O)(═NR¹²)R¹³, —S(O)₁₋₂N(R^{11a}R^{11b}), —N(R^{11a}R^{11b}), —N(R^{11a})C(O)R¹⁰, —N(R^{11a})C(═NR¹²)R¹³, —N(R^{11a})S(O)₁₋₂R¹³, —N(R^{11c})C(O)N(R^{11a}R^{11b}), —N(R^{11c})C(═NR¹²)N(R^{11a}R^{11b}), —N(R^{11c})S(O)₁₋₂N(R^{11a}R^{11b}), N(R^{11a})CO₂R¹³, and an optionally substituted group selected from C1-C6 alkyl, C1-C6 alkoxy, C1-C6 haloalkyl, C1-C6 haloalkoxy, C3-C6 cycloalkyl, C3-C6 cycloalkylidenyl, C3-C6 cycloalkoxy, saturated or unsaturated 4-7 membered heterocyclyl containing 1-2 heteroatoms selected from N, O, and S as ring members, aryl, and heteroaryl containing 1-4 heteroatoms selected from N, O, and S as ring members; wherein the optional substituents are 1-4 substituents independently selected from -halo, —OH, NH₂, ═O, —CN, —SO₂NH₂, C1-C6 alkyl, C1-C6 haloalkyl, C1-C6 alkoxy, C1-C6 haloalkoxy, C3-C6 cycloalkyl, C3-C6 cycloalkoxy, C1-C6 alkylsulfonyl, C3-C6 cycloalkylsulfonyl, C1-C6 alkylsulfonylamino, C3-C6 cycloalkylsulfonylamino, C1-C6 alkylaminosulfonyl, and C3-C6 cycloalkylaminosulfonyl; wherein R¹⁰, R^{11a}, R^{11b} and R^{11c} are independently selected from H and an optionally substituted group selected from C1-C6 alkyl, C3-C6 cycloalkyl, C1-C6 haloalkyl, saturated or unsaturated 4-7 membered heterocyclyl containing 1-2 heteroatoms selected from N, O, and S as ring members, aryl, heteroaryl containing 1-4 heteroatoms selected from N, O, and S as ring members; R¹² is selected from H, —CN, —OH, and an optionally substituted group selected from C1-C4 alkyl and C1-C4 alkoxy; R¹³ is an optionally substituted group selected from C1-C6 alkyl, C3-C6 cycloalkyl C1-C6 haloalkyl, saturated or unsaturated 4-7 membered heterocyclyl containing 1-2 heteroatoms selected from N, O, and S as ring members, aryl, and heteroaryl containing 1-4 heteroatoms selected from N, O, and S as ring members; wherein each of R¹⁰, R^{11a}, R^{11b}, R^{11c}, R¹² and R¹³ is optionally substituted with 1-3 groups independently selected from halo, —OH, NH₂, ═O, —CN, —SO₂NH₂, C1-C6 alkyl, C1-C6 haloalkyl, C1-C6 alkoxy, C1-C6 haloalkoxy, C3-C6 cycloalkyl, C3-C6 cycloalkoxy, C1-C6 alkylsulfonyl, C3-C6 cycloalkylsulfonyl, C1-C6 alkylsulfonylamino, C3-C6 cycloalkylsulfonylamino, C1-C6 alkylaminosulfonyl, and C3-C6 cycloalkylaminosulfonyl; wherein two substituents on the same or adjacent carbon atoms of R¹ can optionally be taken together to form a 4-7 membered ring that can be saturated or unsaturated and optionally contains 1-2 heteroatoms selected from N, O and S as ring members and can optionally be substituted with 1-2 groups independently selected from R⁹;

R² is selected from H, —CN, ethynyl, halo, —CF₃, C1-C3 alkyl, and C1-C3 alkoxy;

R³ and R⁴ are independently selected from H, ═O, OH, CN, halo, —CO₂R¹⁰, —C(O)N(R^{11a}R^{11b}), —N(R^{11a}R^{11b}), and an optionally substituted group selected from C1-C6 alkyl, C1-C6 alkyloxy, and C3-C6 cycloalkylidenyl optionally contains 1-2 heteroatoms as ring members selected from N, O and S, wherein the optional substituents are independently selected from R⁹; wherein R³ and R⁴ can optionally be taken together to form a 4-7 membered cycloalkyl or 4-7 membered heterocyclyl optionally contains 1-2 heteroatoms as ring members selected from N, O and S and can optionally be substituted with at least one group selected from R⁹;

R⁵, R⁶, R⁷ and R⁸ are independently selected from H, OH, CN, halo, —CO₂R¹⁰, —C(O)N(R^{11a}R^{11b}), —N(R^{11a}R^{11b}), and an optionally substituted group selected from C1-C6 alkyl and C1-C6 alkyloxy, wherein the optional substituents are independently selected from R⁹; or

wherein R⁵ and R⁶, or R⁷ and R⁸, can optionally be taken together to form a group selected from 3-7 membered cycloalkyl and 4-7 membered heterocyclyl containing 1-2 heteroatoms selected from N, O and S as ring members, wherein said 3-7 membered cycloalkyl or 4-7 membered heterocyclyl can optionally be fused with one or more groups selected from C3-C7 cycloalkyl, 4-10 membered heterocyclyl containing 1-3 heteroatoms selected from N, O, and S as ring members, aryl, and heteroaryl containing 1-4 heteroatoms selected from N, O, and S as ring members, and wherein any of the foregoing cyclic groups is optionally substituted with at least one group selected from R⁹;

R⁶ and R⁷ can optionally be taken together to form a 3-7 membered cycloalkyl or 4-7 membered heterocyclyl containing 1-2 heteroatoms as ring members selected from N, O and S and can optionally be substituted with at least one group selected from R⁹;

R³ and R⁷ can optionally be taken together to form a 5-7 membered cycloalkyl or 5-7 membered heterocyclyl containing 1-2 heteroatoms as ring members selected from N, O and S and can optionally be substituted with at least one group selected from R⁹;

R⁴ and R⁶ can optionally be taken together to form a 5-7 membered cycloalkyl or 5-7 membered heterocyclyl containing 1-2 heteroatoms as ring members selected from N, O and S and can optionally be substituted with at least one group selected from R⁹;

m is 0, 1, or 2;

n is 1 or 2;

A¹ is selected from —S(O)₂N(H)C(O)—, —S(O)₂N(H)C(O)N(R¹⁴)—, —C(O)N(H)S(O)₂—, —C(O)N(H)S(O)₂N(R¹⁴)—, —S(O)(═NR¹⁵)N(H)C(O)—, —S(O)(═NR¹⁵)N(H)C(O)N(R¹⁴)—, —C(O)N(H)S(O) (═NR¹⁵)—, and —C(O)N(H)S(O) (═NR¹⁵)N(R¹⁴)—; wherein R¹⁴ is selected from H and an optionally substituted group selected from C1-C6 alkyl, C3-C6 cycloalkyl, C1-C6 haloalkyl, 4-7 membered heterocyclyl containing 1-2 heteroatoms selected from N, O, and S as ring members; wherein R¹⁵ is selected from H, —CN, —OH, and an optionally substituted group selected from C1-C4 alkyl and C1-C4 alkoxy; and wherein R¹⁴ and R¹⁵ are optionally substituted with at least one group selected from R⁹;

A² is selected from a bond, —N(R¹⁴) and —O—;

X¹, X², and X³ are independently selected from —N— and —CH—,

L¹ is selected from a bond, —S—, —S(O)₁₋₂—, —O—, —C(O)—, —C(OR¹⁶)—, —C(R^{17a}R^{17b})—, and —N(R¹⁸)—; wherein R¹⁶ is selected from H and C1-C4 alkyl optionally submitted with one or more halogen; R^{17a} and R^{17b} are independently selected from H, halo and C1-C4 alkyl optionally submitted with one or more halogen; R¹⁸ is selected from H and C1-C4 alkyl optionally submitted with one or more halogen;

L² is selected from

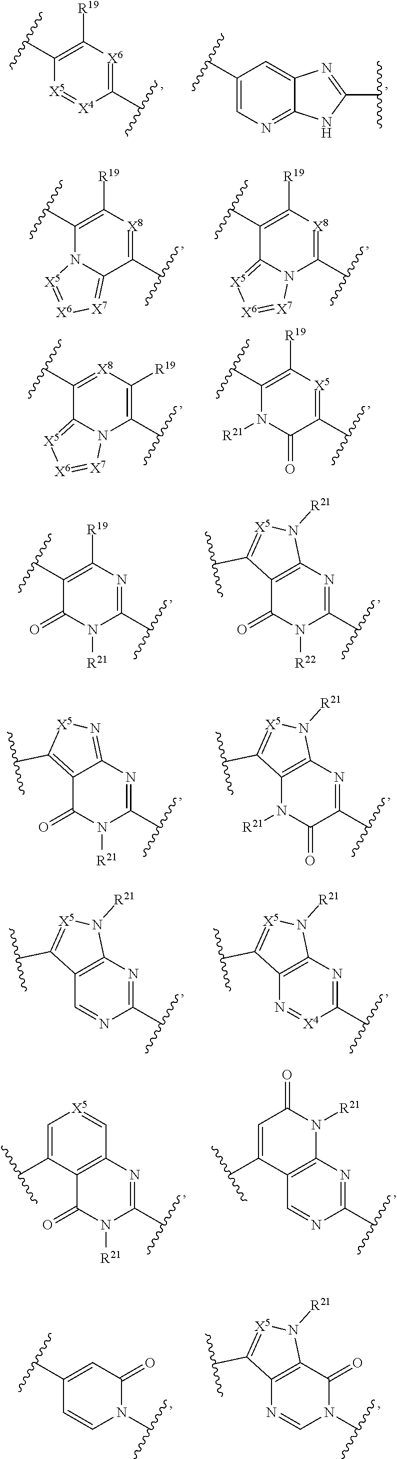

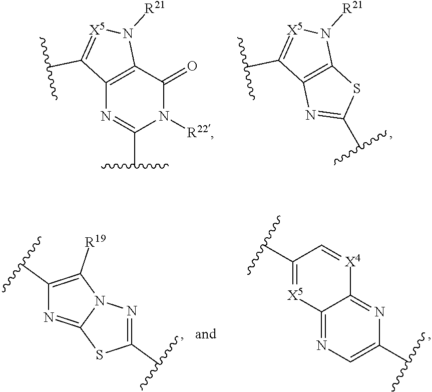

wherein R¹⁹ is selected from H, OH, CN, NH₂, halo, and an optionally substituted group selected from C1-C4 alkyl, C1-C6 haloalkyl, C3-C6 cycloalkyl, and aryl, wherein the optionally substituted groups can be substituted with at least one group selected from R⁹;

X⁴, X⁵, X⁶, X⁷, and X⁸ are independently selected from —N— or —C(R²⁰)—, wherein R²⁰ is selected from H, —CONH₂, OH, NH₂, CN, and —CH₂OH; and

R²¹ and R²² are independently selected from H, CH₃, and —CH₂OH.

Also disclosed herein are pharmaceutical compositions, comprising a compound of Formula I and/or a stereoisomer, a stable isotope, or a pharmaceutically acceptable salt or solvate thereof disclosed herein and a pharmaceutically acceptable carrier.

Further disclosed herein are methods of inhibiting the activity of SHP2 comprising contacting the protein SHP2 with an effective amount of a compound of Formula I and/or a stereoisomer, a stable isotope, or a pharmaceutically acceptable salt or solvate thereof disclosed herein.

Further disclosed herein are methods of treating a disease treatable by inhibition of SHP2 in a patient, comprising administering to the patient in recognized need of such treatment, an effective amount of a compound of Formula I and/or a stereoisomer, a stable isotope, or a pharmaceutically acceptable salt or solvate thereof disclosed herein.

Further disclosed herein are methods of treating a disease treatable by inhibition of SHP2 in a patient, comprising administering to the patient in recognized need of such treatment, an effective amount of a pharmaceutical composition comprising a compound of Formula I and/or a stereoisomer, a stable isotope, or a pharmaceutically acceptable salt or solvate thereof disclosed herein and a pharmaceutically acceptable carrier.

Further disclosed herein are methods of treating a cancer in a patient, comprising administering to the patient in recognized need of such treatment, an effective amount of a pharmaceutical composition comprising a compound of Formula I and/or a stereoisomer, a stable isotope, or a pharmaceutically acceptable salt or solvate thereof disclosed herein, and a pharmaceutically acceptable carrier. In some embodiments, the cancer is selected from juvenile myelomonocytic leukemias, neuroblastoma, melanoma, acute myeloid leukemia, breast cancer, esophageal cancer, lung cancer, colon cancer, head camcer, neuroblastoma, squamous-cell carcinoma of the head and neck, gastric carcinoma, anaplastic large-cell lymphoma and glioblastoma.

Further disclosed herein is a use of a compound of Formula I and/or a stereoisomer, a stable isotope, or a pharmaceutically acceptable salt or solvate thereof in preparation of a medication for treating a disease responsive to inhibition of SHP2, such as a cancer. In some embodiments, the cancer is selected from juvenile myelomonocytic leukemias, neuroblastoma, melanoma, acute myeloid leukemia, breast cancer, esophageal cancer, lung cancer, colon cancer, head camcer, neuroblastoma, squamous-cell carcinoma of the head and neck, gastric carcinoma, anaplastic large-cell lymphoma and glioblastoma.

Further disclosed herein are compounds of Formula I and the subgenera of Formula I disclosed herein, as well as pharmaceutically acceptable salts or solvates of these compounds, and all stereoisomers (including diastereoisomers and enantiomers, and isotopically enriched versions thereof (including deuterium substitutions). These compounds can be used to treat conditions responsive to SHP2 inhibition, such as those disclosed herein, and for use in the preparation of a medicament for treating these disorders. The pharmaceutical compositions and methods disclosed herein can also be used with or formulated with a co-therapeutic agent; for example, compounds of Formula I and sub-formula thereof can be used with or formulated with one or more agents selected from inhibitors of and non-SHP2 kinase and other therapeutic agents.

Further disclosed are methods, as well as key intermediate compounds, useful for making the compounds of Formula I as disclosed herein.

As used herein, the following words, phrases and symbols are generally intended to have the meanings as set forth below, except to the extent that the context in which they are used indicates otherwise. The following abbreviations and terms have the indicated meanings throughout.

### DETAILED DESCRIPTION

The following definitions apply unless otherwise provided or apparent from context:

A dash (“-”) that is not between two letters or symbols is used to indicate a point of attachment for a substituent. For example, —CONRaRb is attached through the carbon atom.

Unless clearly indicated otherwise, use of the terms “a”, “an” and the like refers to one or more.

The term “halogen” or “halo” as used herein refers to fluorine (F), chlorine (Cl), bromine (Br) or iodine (I). Halogen-substituted groups and moieties, such as alkyl substituted by halogen (haloalkyl) can be mono-, poly-, or per-halogenated. In some embodiments, chloro and fluoro are examples of halo substituents on alkyl or cycloalkyl groups, unless otherwise specified; fluoro, chloro, and bromo are used, for example, on aryl or heteroaryl groups, unless otherwise specified.

The term “heteroatoms” or “hetero atoms” as used herein refers to nitrogen (N) or oxygen (O) or sulfur (S) atoms, unless otherwise specified.

The term “optional” or “optionally” as used herein means that the subsequently described event or circumstance may or may not occur, and that the description includes instances where the event or circumstance occurs and instances in which it does not. For example, “alkyl optionally substituted with X” encompasses both “alkyl without substitution of X” and “alkyl substituted with X.” It will be understood by those skilled in the art, with respect to any group containing one or more substituents, that such groups are not intended to introduce any substitution or substitution patterns that are sterically impractical, synthetically non-feasible and/or inherently unstable in water at room temperature for at least long enough to be administered as a pharmaceutical agent. When multiple substituents are present, the substituents are selected independently unless otherwise indicated, so where 2 or 3 substituents are present, for example, those substituents may be the same or different.

In some embodiments, “substituted with at least one group” refers to one hydrogen on the designated atom or group being replaced with one selection from the indicated group of substituents. In some embodiments, “substituted with at least one group” refers to two hydrogens on the designated atom or group being independently replaced with two selections from the indicated group of substituents. In some embodiments, “substituted with at least one group” refers to three hydrogens on the designated atom or group being independently replaced with three selections from the indicated group of substituents. In some embodiments, “substituted with at least one group” refers to four hydrogens on the designated atom or group being independently replaced with four selections from the indicated group of substituents.

The term “alkyl” as used herein refers to a hydrocarbon group chosen from linear and branched saturated hydrocarbon groups having up to 18 carbon atoms, such as from 1 to 12, further such as from 1 to 8, even further such as from 1 to 6, carbon atoms. Representative examples of alkyl include, but not limited to, methyl, ethyl, n-propyl, iso-propyl, n-butyl, sec-butyl, iso-butyl, tert-butyl, n-pentyl, iso-pentyl, neopentyl, n-hexyl, 3-methylhexyl, 2,2-dimethylpentyl, 2,3-dimethylpentyl, n-heptyl, n-octyl, n-nonyl, n-decyl, and the like.

Unless indicated specifically, alkyl groups can be optionally substituted by one or more substituents in place of hydrogen atoms of the unsubstituted alkyl, such as one, two or three substituents, or 1-4 substituents, up to the number of hydrogens present on the unsubstituted alkyl group. Suitable substituents for alkyl groups, if not otherwise specified, may be selected from halogen, deuterium (D), CN, oxo, hydroxyl, substituted or unsubstituted C1-C4 alkoxy, substituted or unsubstituted C3-C6 cycloalkyl, substituted or unsubstituted 3-7 membered heterocycloalkyl containing 1 or 2 heteroatoms selected from N, O and S as ring members, substituted or unsubstituted aryl, substituted or unsubstituted heteroaryl containing 1 to 4 heteroatoms selected from N, O and S as ring members, amino, —NH(C1-C4 alkyl), —N(C1-C4 alkyl)₂, —S(═O)₀₋₂(C1-C4 alkyl), —S(═NR)(=O) (C1-C4 alkyl), —C(═O)(C1-C4 alkyl), —C(═NOH)(C1-C4 alkyl), —CO₂H, —CO₂(C1-C4 alkyl), —S(═O)₁₋₂NH₂, —S(═O)₁₋₂NH(C1-C4 alkyl), —S(═O)₁₋₂N(C1-C4 alkyl)₂, —CONH₂, —C(═O)NH(C1-C4 alkyl), —C(═O)N(C1-C4 alkyl)₂, —C(═NOH)NH(C1-C4 alkyl), —OC(═O)(C1-C4 alkyl), —NHC(═O)(C1-C4 alkyl), —NHC(═NOH)(C1-C4 alkyl), —NH(C═O)NH₂, —NHC(═O)O(C1-C4 alkyl), —NHC(═O)NH(C1-C4 alkyl), NHC(═NOH)NH(C1-C4 alkyl), —NHS(═O)₁₋₂(C1-C4 alkyl), —NHS(═O)₁₋₂NH₂, and —NHS(═O)₁₋₂NH(C1-C4 alkyl); wherein the substituents for substituted C1-C4 alkoxy, substituted C3-C6 cycloalkyl, substituted 3-7 membered heterocycloalkyl, substituted aryl, and substituted heteroaryl comprise up to three groups independently selected from halogen, D, —CN, C1-C4 alkyl, C1-C4 haloalkyl, oxo, hydroxy, C1-C4 alkoxy, amino, —NH(C1-C4 alkyl), and —N(C1-C4 alkyl)₂. In some embodiments, substituents for alkyl groups, unless otherwise specified, are selected, for example, from halogen, CN, oxo, hydroxy, C1-C4 alkoxy, C3-C6 cycloalkyl, phenyl, amino, —NH(C1-C4 alkyl), —N(C1-C4 alkyl)₂, C1-C4 alkylthio, C1-C4 alkylsulfonyl, —C(═O)(C1-C4 alkyl), —CO₂H, —CO₂(C1-C4 alkyl), —OC(═O)(C1-C4 alkyl), —NHC(═O)(C1-C4 alkyl), and —NHC(═O)O(C1-C4 alkyl).

The term “alkoxy” as used herein refers to a straight or branched alkyl group comprising from 1 to 18 carbon atoms attached through an oxygen bridge such as methoxy, ethoxy, propoxy, isopropoxy, n-butoxy, sec-butoxy, tert-butoxy, pentoxy, 2-pentyloxy, isopentoxy, neopentoxy, hexoxy, 2-hexoxy, 3-hexoxy, 3-methylpentoxy, and the like. Typically, alkoxy groups comprise from 1 to 6 carbon atoms, such as 1 to 4 carbon atoms, attached through the oxygen bridge.

Unless indicated specifically, alkoxy groups can be optionally substituted by one or more substituents in place of hydrogen atoms of the unsubstituted alkyl portion of the alkoxy, such as one, two or three substituents, or 1-4 substituents, up to the number of hydrogens present on the unsubstituted alkoxy group. Unless otherwise specified, suitable substituents are selected, for example, from the substituents listed above for alkyl groups, except that hydroxyl and amino are not normally present on the carbon that is directly attached to the oxygen of the substituted alkyl-O group.

The term “alkenyl” as used herein refers to a hydrocarbon group selected from linear and branched hydrocarbon groups, comprising at least one C═C double bond and from 2 to 18, such as from 2 to 6, carbon atoms. Examples of the alkenyl group may be selected from ethenyl or vinyl (—CH═CH₂), prop-1-enyl (—CH═CHCH₃), prop-2-enyl (—CH₂CH═CH₂), 2-methylprop-1-enyl, buta-1-enyl, buta-2-enyl, buta-3-enyl, buta-1,3-dienyl, 2-methylbuta-1,3-diene, hex-1-enyl, hex-2-enyl, hex-3-enyl, hex-4-enyl, and hexa-1,3-dienyl groups. The point of attachment can be on the unsaturated carbon or saturated carbon.

Unless indicated specifically, alkenyl groups can be optionally substituted by one or more substituents in place of hydrogen atoms of the unsubstituted alkenyl, such as one, two or three substituents, or 1-4 substituents, up to the number of hydrogens present on the unsubstituted alkenyl group. Unless otherwise specified, suitable substituents are selected, for example, from the substituents listed above for alkyl groups.

The term “alkynyl” as used herein refers to a hydrocarbon group selected from linear and branched hydrocarbon groups, comprising at least one —C≡C— triple bond and from 2 to 18, such as from 2 to 6, carbon atoms. Examples of the alkynyl group include ethynyl (—C≡CH), 1-propynyl (—C≡CCH₃), 2-propynyl (propargyl, —CH₂C≡CH), 1-butynyl, 2-butynyl, and 3-butynyl groups. The point of attachment can be on the unsaturated carbon or saturated carbon.

Unless indicated specifically, alkynyl groups can be optionally substituted by one or more substituents in place of hydrogen atoms of the unsubstituted alkynyl, such as one, two or three substituents, or 1-4 substituents, up to the number of hydrogens present on the unsubstituted alkynyl group. Unless otherwise specified, suitable substituents are selected, for example, from the substituents listed above for alkyl groups.

The term “alkylene” as used herein refers to a divalent alkyl group comprising from 1 to 10 carbon atoms, and two open valences to attach to other molecular components. The two molecular components attached to an alkylene can be on the same carbon atom or on different carbon atoms; thus for example propylene is a 3-carbon alkylene that can be 1,1-disubstituted, 1,2-disubstituted or 1,3-disubstituted. Unless otherwise specified, alkylene refers to moieties comprising from 1 to 6 carbon atoms, such as from 1 to 4 carbon atoms. Examples of alkylene include, but are not limited to, methylene, ethylene, n-propylene, iso-propylene, n-butylene, sec-butylene, iso-butylene, tert-butylene, n-pentylene, isopentylene, neopentylene, n-hexylene, 3-methylhexylene, 2,2-dimethylpentylene, 2,3-dimethylpentylene, n-heptylene, n-octylene, n-nonylene, n-decylene and the like. A substituted alkylene is an alkylene group containing one or more, such as one, two or three substituents; unless otherwise specified, suitable substituents are selected, for example, from the substituents listed above for alkyl groups.

Unless indicated specifically, alkylenyl groups can be optionally substituted by one or more substituents in place of hydrogen atoms of the unsubstituted alkylenyl, such as one, two or three substituents, or 1-4 substituents, up to the number of hydrogens present on the unsubstituted alkylenyl group. Unless otherwise specified, suitable substituents are selected, for example, from the substituents listed above for alkyl groups.

Similarly, the terms “alkenylene” and “alkynylene” as used herein refer to alkylene groups comprising a double bond or a triple bond, respectively; they are, for example, 2-6, such as 2-4, carbon atoms in length, and can be substituted as discussed above for alkylene groups.

The term “haloalkyl” as used herein refers to an alkyl as defined herein, which is substituted by one or more halo groups as defined herein. Unless otherwise specified, the alkyl portion of the haloalkyl comprises 1-4 carbon atoms. The haloalkyl can be monohaloalkyl, dihaloalkyl, trihaloalkyl, or polyhaloalkyl including perhaloalkyl. A monohaloalkyl can have one iodo, bromo, chloro, or fluoro within the alkyl group. Dihaloalkyl and polyhaloalkyl groups can have two or more of the same halo atoms or a combination of different halo groups within the alkyl. The polyhaloalkyl comprises, for example, up to 6, or 4, or 3, or 2 halo groups. Examples of haloalkyl include, but are not limited to, fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. A perhaloalkyl refers to an alkyl having all hydrogen atoms replaced with halo atoms, e.g., trifluoromethyl. In some embodiments, the haloalkyl groups, unless specified otherwise, include monofluoro-, difluoro- and trifluoro-substituted methyl and ethyl groups, e.g. —CF₃, —CF₂H, —CFH₂ and —CH₂CF₃.

Unless indicated specifically, haloalkyl groups can be optionally substituted by one or more substituents in place of hydrogen atoms of the unsubstituted haloalkyl, such as one, two or three substituents, or 1-4 substituents, up to the number of hydrogens present on the unsubstituted haloalkyl group. Unless otherwise specified, suitable substituents are selected, for example, from the substituents listed above for alkyl groups.

As used herein, the term “haloalkoxy” refers to haloalkyl-O—, wherein haloalkyl is defined above. Examples of haloalkoxy include, but are not limited to, fluoromethoxy, difluoromethoxy, trifluoromethoxy, trichloromethoxy, 2-chloroethoxy, 2,2,2-trifluoroethoxy, 1,1,1,3,3,3-hexafluoro-2-propoxy, and the like. In some embodiments, haloalkyloxy groups comprise 1-4 carbon atoms, and up to three halogens, e.g., monofluoro, difluoro and trifluoro substituted methoxy groups and ethoxy groups.

Unless indicated specifically, haloalkoxy group can be optionally substituted by one or more substituents in place of hydrogen atoms of the unsubstituted alkyl portion of the haloalkoxy, such as one, two or three substituents, or 1-4 substituents, up to the number of hydrogens present on the unsubstituted haloalkoxy group. Unless otherwise specified, suitable substituents are selected, for example, from the substituents listed above for alkyl groups, except that hydroxyl and amino are not normally present on the carbon that is directly attached to the oxygen of the substituted haloalkoxy (haloalkyl-0) group.

The term “cycloalkyl” as used herein refers to a hydrocarbon group selected from saturated and partially unsaturated cyclic hydrocarbon groups comprising from 3 to 20 carbon atoms, such as monocyclic and polycyclic (e.g., bicyclic and tricyclic, admantanyl and spirocycloalkly) groups. Monocycloalkyl groups are cyclic hydrocarbon groups comprising from 3 to 20 carbon atoms, such as from 3 to 8 carbon atoms. Examples of monocyclic cycloalkyl include, but are not limited to, cyclopropyl, cyclobutyl, cyclopentyl, cyclohexyl, cycloheptyl, cyclooctyl, cyclodecanyl, cyclodocecanyl, and cyclohexenyl. Bicycloalkyl groups include bridged bicycloalkyl, fused bicycloalkyl and spirocycloalkyls. Bridged bicycloalkyl groups contain a monocyclic cycloalkyl ring where two non-adjacent carbon atoms of the monocyclic ring are linked by an alkylene bridge of one to three additional carbon atoms (i.e. a bridging group of the form —(CH₂)ₙ—, wherein n is 1, 2, or 3). Examples of bridged bicycloalkyl include, but are not limited to, bicyclo[2.2.1]heptenes, bicyclo[3.1.1]heptanes, bicyclo[2.2.1]heptanes, bicyclo[2.2.2]octane, bicyclo[3.2.2]nonane, bicyclo[3.3.1]nonane, and bicycle[4.2.1]nonane. Fused bicycloalkyl contains a monocyclic cycloalkyl ring fused to either a phenyl, a monocyclic cycloalkyl, or a monocyclic heteroaryl. Examples of fused bicycloalkyl include, but are not limited to, bicyclo[4.2.0]octa-1,3,5-triene, 2,3-dihydro-1H-indene, 6,7-dihydro-5H-cyclopenta[b]pyridine, 5,6-dihydro-4H-cyclopenta[b]thiophene, and decahydronaphthalene. Spirocycloalkyl groups contain two monocyclic ring systems that share a carbon atom forming a biclyclic ring system. Examples of spirocycloalkyls include, but are not limited to, and

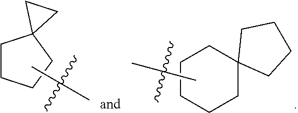

Bicyclic cycloalkyl groups comprise, for example, from 7 to 12 carbon atoms. Monocycloalkyl or bicycloalkyl groups are attached to the parent molecular moiety through any carbon atom contained within the cycloalkyl ring. Tricycloalkyl groups include bridged tricycloalkyl as used herein referring to 1) a bridged bicycloalkyl ring where two non-adjacent carbon atoms of the bridged bicycloalkyl ring are linked by an alkylene bridge of one to three additional carbon atoms (i.e. a bridging group of the form —(CH₂)ₙ—, wherein n is 1, 2, or 3), or 2) a fused bicycloalkyl ring where two unshared ring atoms on each ring are linked by an alkylene bridge of one to three additional carbon atoms (i.e. a bridging group of the form —(CH₂)ₙ—, wherein n is 1, 2, or 3), wherein “a fused bicycloalkyl ring” refers to a monocycloalkyl ring fused to a monocycloalkyl ring. Examples of bridged tricycloalkyl groups include, but are not limited to, admantanyl

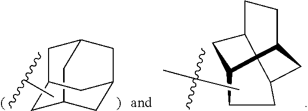

Bridged tricycloalkyl, as used herein, is appended to the parent molecular moiety through any ring atom. The ring atom disclosed herein refers to the carbon atom on the ring skeleton. The cycloalkyl may be saturated or comprise at least one double bond (i.e. partially unsaturated), but is not fully conjugated, and is not aromatic, as aromatic is defined herein. The cycloalkyl may be substituted with at least one hetero atom selected, for example, from O, S, and N.

Unless indicated specifically, cycloalkyl groups can be optionally substituted by one or more substituents in place of hydrogen atoms of the unsubstituted cycloalkyl, such as one, two or three substituents, or 1-4 substituents, up to the number of hydrogens present on the unsubstituted cycloalkyl group. In some embodiments, a substituted cycloalkyl comprises 1-4, such as 1-2, substituents. Unless otherwise specified, suitable substituents are selected, for example, from the substituents listed above for alkyl groups.

The term “cycloalkylidenyl” or “cycloalkylidene ring” as used herein refers to a divalent cycloalkane ring attached via the same carbon atom of the cycloalkane ring by removal of two hydrogen atoms from the same carbon atoms. Examples of cycloakylidenyl rings include, but are not limited to, cyclopropylidenyl, cyclobutylidenyl, cyclopentylidenyl, and cyclohexylidenyl. It can be represented in illustrative fashion by the following structure wherein n is 1, 2, 3, 4, or 5.

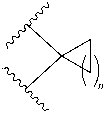

The term “heterocycloalkyl,” “heterocyclyl,” or “heterocyclic” as used herein refers to “cycloalkyl” as defined above wherein at least one ring carbon atom is replaced by a heteroatom independently selected from O, N, and S. Heterocyclyl comprises, for example, 1, 2, 3, or 4 heteroatoms, and each N, C or S can independently be oxidized in the cyclic ring system. The N atom can further be substituted to form a tertiary amine or ammonium salts. The point of attachment of a heterocyclyl can be on the heteroatom or carbon. “Heterocyclyl” herein also refers to a 5- to 7-membered saturated or partially unsaturated carbocyclic ring comprising at least one heteroatom selected, for example, from N, O, and S (heterocyclic ring) fused with 5-, 6-, and/or 7-membered cycloalkyl, heterocyclic or carbocyclic aromatic ring, provided that the point of attachment is at the heterocyclic ring when the heterocyclic ring is fused with a carbocyclic aromatic ring, and that the point of attachment can be at the cycloalkyl or heterocyclic ring when the heterocylic ring is fused with cycloalkyl. “Heterocyclyl” herein also refers to an aliphatic spirocyclic ring comprising at least one heteroatom selected, for example, from N, O, and S. The rings may be saturated or have at least one double bond (i.e. partially unsaturated). The heterocyclyl may be substituted with, for example, oxo. The point of the attachment may be carbon or heteroatom. A heterocyclyl is not a heteroaryl as defined herein.

Examples of heterocyclic groups include, but are not limited to, pyrrolidinyl, imidazolidinyl, pyrazolidinyl, piperidinyl, piperidinyl, piperazinyl, pyranyl, morpholinyl, oxiranyl, aziridinyl, thiiranyl, azetidinyl, oxetanyl, thietanyl, dithietanyl, dihydropyridinyl, tetrahydropyridinyl, thiomorpholinyl, thioxanyl, homopiperazinyl, homopiperidinyl, azepanyl, oxepanyl, thiepanyl, oxathianyl, dioxepanyl, oxathiepanyl, oxaazepanyldithiepanyl, thiazepanyl and diazepane, dithianyl, azathianyl, oxazepinyl, diazepinyl, thiazepinyl, dihydrothienyl, dihydropyranyl, dihydrofuranyl, tetrahydrofuranyl, tetrahydrothienyl, tetrahydropyranyl, tetrahydrothiopyranyl, indolinyl, dioxanyl, pyrazolinyl, dithianyl, dithiolanyl, pyrazolidinylimidazolinyl, pyrimidinonyl, 1,1-dioxo-thiomorpholinyl, 3-azabicyco[3.1.0]hexanyl, 3-azabicyclo[4.1.0]heptanyl and azabicyclo[2.2.2]hexanyl. Substituted heterocycles also include ring systems substituted with at least one oxo moiety, such as piperidinyl N-oxide, morpholinyl-N-oxide, 1-oxo-1-thiomorpholinyl, 1,1-dioxo-1-thiomorpholinyl,

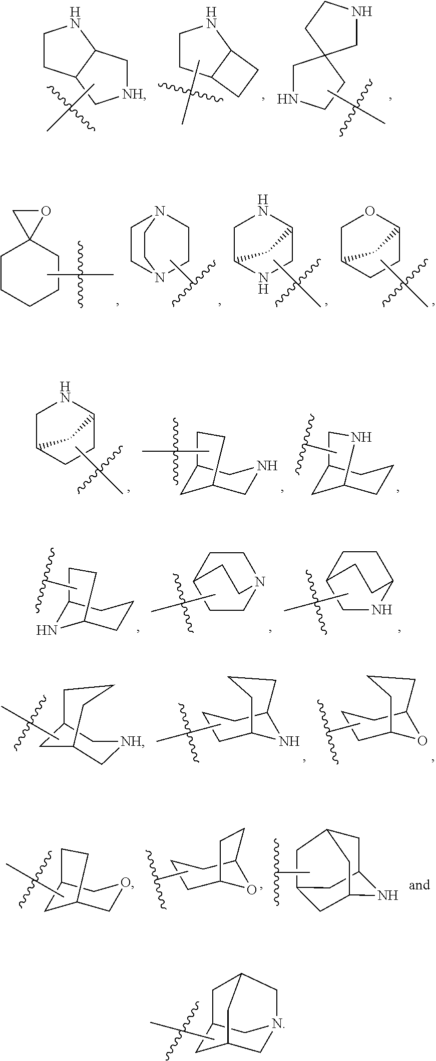

Unless indicated specifically, heterocyclyl group can be optionally substituted by one or more substituents in place of hydrogen atoms of the unsubstituted heterocyclyl, such as one, two or three substituents, or 1-4 substituents, up to the number of hydrogens present on the unsubstituted heterocyclyl group. In some embodiments, a substituted heterocycloalkyl comprises 1-4 (such as, e.g., 1-2 or 1-3) substituents. Unless otherwise specified, suitable substituents are selected, for example, from the substituents listed above for alkyl groups.

The term “aryl” as used herein refers to an aromatic hydrocarbon group comprising 5-15 carbon atoms in the ring portion. In some embodiments, aryl refers to a group selected from 5- and 6-membered carbocyclic aromatic rings, for example, phenyl; bicyclic ring systems such as 7 to 12 membered bicyclic ring systems wherein at least one ring is carbocyclic and aromatic, selected, for example, from naphthalene, indane, and 1,2,3,4-tetrahydroquinoline; and tricyclic ring systems such as 10 to 15 membered tricyclic ring systems, wherein at least one ring is carbocyclic and aromatic, for example, fluorene.

In some embodiments, the aryl group is selected from 5- and 6-membered carbocyclic aromatic rings fused to a 5- to 7-membered cycloalkyl or heterocyclic ring (as defined in “heterocyclyl” or “heterocyclic” below) optionally comprising at least one heteroatom selected, for example, from N, O, and S, provided that the point of attachment is at the carbocyclic aromatic ring when the carbocyclic aromatic ring is fused with a heterocyclic ring, and the point of attachment can be at the carbocyclic aromatic ring or at the cycloalkyl group when the carbocyclic aromatic ring is fused with a cycloalkyl group. Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene. Aryl, however, does not encompass or overlap in any way with heteroaryl, separately defined below. Hence, if one or more carbocyclic aromatic rings are fused with a heterocyclic aromatic ring (e.g., a heteroaryl as defined below), the resulting ring system is heteroaryl, not aryl, as defined herein.

Unless indicated specifically, aryl groups can be optionally substituted by one or more substituents in place of hydrogen atoms of the unsubstituted aryl, such as one, two or three substituents, or 1-4 substituents, up to the number of hydrogens present on the unsubstituted aryl group. In some embodiments, a substituted aryl group comprises 1-5 substituents. Unless otherwise specified, suitable substituents are selected, for example, from the substituents listed above for alkyl groups.

The term “heteroaryl” as used herein refers to a group selected from 5- to 7-membered aromatic, monocyclic rings comprising at least one heteroatom, for example, from 1 to 4, or, in some embodiments, from 1 to 3, heteroatoms, selected, for example, from N, O, and S, with the remaining ring atoms being carbon; 8- to 12-membered bicyclic rings comprising at least one heteroatom, for example, from 1 to 4, or, in some embodiments, from 1 to 3, or, in other embodiments, 1 or 2, heteroatoms, selected, for example, from N, O, and S, with the remaining ring atoms being carbon and wherein at least one ring is aromatic and at least one heteroatom is present in the aromatic ring, and with the point of attachment being on any ring and being on either carbon or the heteroatom; and 11- to 14-membered tricyclic rings comprising at least one heteroatom, for example, from 1 to 4, or in some embodiments, from 1 to 3, or, in other embodiments, 1 or 2, heteroatoms, selected, for example, from N, O, and S, with the remaining ring atoms being carbon and wherein at least one ring is aromatic and at least one heteroatom is present in an aromatic ring, and with the point of attachment being on any ring.

In some embodiments, the heteroaryl group includes a 5- to 7-membered heterocyclic aromatic ring fused to a 5- to 7-membered cycloalkyl ring. For such fused, bicyclic heteroaryl ring systems wherein only one of the rings comprises at least one heteroatom, the point of attachment may be at the heteroaromatic ring or at the cycloalkyl ring.

In some embodiments, the heteroaryl group includes a 5- to 7-membered heterocyclic aromatic ring fused to a 5- to 7-membered aryl ring. For such fused, bicyclic heteroaryl ring systems wherein only one of the rings comprises at least one heteroatom, the point of attachment may be at the heteroaromatic ring or at the aryl ring. Non-limiting examples include quinolinyl and quinazolinyl.

In some embodiments, the heteroaryl group includes a 5- to 7-membered heterocyclic aromatic ring fused to another 5- to 7-membered heterocyclic aromatic ring. Non-limiting examples include 1H-pyrazolo[3,4-b]pyridinyl and 1H-pyrrolo[2,3-b]pyridinyl.

When the total number of S and O atoms in the heteroaryl group exceeds 1, those heteroatoms are not adjacent to one another. In some embodiments, the total number of S and O atoms in the heteroaryl group is not more than 2. In some embodiments, the total number of S and O atoms in the aromatic heterocycle is not more than 1.

Examples of the heteroaryl group include, but are not limited to, pyridyl, cinnolinyl, pyrazinyl, pyrimidinyl, imidazolyl, imidazopyridinyl, isoxazolyl, oxazolyl, thiazolyl, isothiazolyl, thiadiazolyl, tetrazolyl, thienyl, triazinyl, benzothienyl, furyl, benzofuryl, benzoimidazolyl, indolyl, isoindolyl, indolinyl, phthalazinyl, pyrazinyl, pyridazinyl, pyrimidinyl, pyrrolyl, triazolyl, quinolinyl, isoquinolinyl, pyrazolyl, pyrrolopyridinyl (such as 1H-pyrrolo[2,3-b]pyridin-3-yl), pyrazolopyridinyl (such as 1H-pyrazolo[3,4-b]pyridin-3-yl), benzoxazolyl (such as benzo[d]oxazol-6-yl), pteridinyl, purinyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, furazanyl, benzofurazanyl, benzothiophenyl, benzothiazolyl, benzoxazolyl, quinazolinyl, quinoxalinyl, naphthyridinyl, furopyridinyl, benzothiazolyl (such as benzo[d]thiazol-6-yl), indazolyl (such as 1H-indazol-5-yl) and 5,6,7,8-tetrahydroisoquinoline.

Unless indicated specifically, heteroaryl group can be optionally substituted by one or more substituents in place of hydrogen atoms of the unsubstituted heteroaryl, such as one, two or three substituents, or 1-4 substituents, up to the number of hydrogens present on the unsubstituted heteroaryl group. In some embodiments, a substituted heteroaryl group comprises 1, 2 or 3 substituents. Unless otherwise specified, suitable substituents are selected, for example, from the substituents listed above for alkyl groups.

Compounds disclosed herein may contain an asymmetric center and may thus exist as enantiomers. Where the compounds disclosed herein possess two or more asymmetric centers, they may additionally exist as diastereomers. Enantiomers and diastereomers fall within the broader class of stereoisomers. It is well-known in the art how to prepare optically active forms, such as by resolution of materials or by asymmetric synthesis. All such possible stereoisomers as substantially pure resolved enantiomers, racemic mixtures thereof, as well as mixtures of diastereomers are intended to be included. All stereoisomers of the compounds disclosed herein and/or pharmaceutically acceptable salts thereof are intended to be included. Unless specifically mentioned otherwise, reference to one isomer applies to any of the possible isomers. Whenever the isomeric composition is unspecified, all possible isomers are included.

When the compounds disclosed herein contain olefinic double bonds, unless specified otherwise, such double bonds are meant to include both E and Z geometric isomers.

“A pharmaceutically acceptable salt” as used herein includes, but is not limited to, salts with inorganic acids, selected, for example, from hydrochlorates, phosphates, diphosphates, hydrobromates, sulfates, sulfinates, and nitrates; as well as salts with organic acids, selected, for example, from malates, maleates, fumarates, tartrates, succinates, citrates, lactates, methanesulfonates, p-toluenesulfonates, 2-hydroxyethylsulfonates, benzoates, salicylates, stearates, alkanoates such as acetate, and salts with HOOC—(CH₂)ₙ—COOH, wherein n is selected from 0 to 4. Similarly, examples of pharmaceutically acceptable cations include, but are not limited to, sodium, potassium, calcium, aluminum, lithium, and ammonium.

In addition, if a compound disclosed herein is obtained as an acid addition salt, the free base can be obtained by basifying a solution of the acid salt. Conversely, if the product is a free base, an addition salt, such as a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used without undue experimentation to prepare non-toxic pharmaceutically acceptable addition salts.

“Treating”, “treat”, “treatment” or “alleviation” as used herein refers to administering at least one compound and/or at least one stereoisomer thereof, if any, at least one stable isotope thereof, or at least one pharmaceutically acceptable salt thereof disclosed herein to a subject in recognized need thereof that has, for example, cancer.

The term “effective amount” as used herein refers to an amount of at least one compound and/or at least one stereoisomer thereof, if any, at least one stable isotope thereof, or at least one pharmaceutically acceptable salt thereof disclosed herein effective to “treat,” as defined above, a disease or disorder in a subject.

Various embodiments are disclosed herein. It will be recognized that features specified in each embodiment may be combined with other specified features to provide further embodiments of the present disclosure. The following non-limiting enumerated embodiments are representative of the present disclosure.

Embodiment 1. A compound of Formula I:

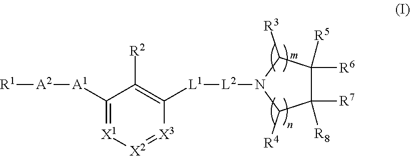

and/or stereoisomers, stable isotopes, or pharmaceutically acceptable salts or solvates thereof, wherein:
   R¹ is an optionally substituted group selected from C1-C12 alkyl, C3-C12 cycloalkyl, 4-12 membered heterocyclyl containing 1-3 heteroatoms selected from N, O, and S as ring members, aryl, and heteroaryl containing 1-4 heteroatoms selected from N, O, and S as ring members; and wherein R¹ is optionally substituted with 1-4 substituents independently selected from R⁹, wherein R⁹ is selected from halo, —OH, NH₂, ═O, —CN, —OC(O)R¹⁰, —CO₂R¹⁰, —C(O)N(R^{11a}R^{11b}), —C(═NR¹²)N(R^{11a}R^{11b}), —C(O)R¹⁰, —OR¹⁰, —SR¹⁰, —S(O)₀₋₂R¹³, —S(O)(═NR¹²)R¹³, —S(O)₁₋₂N(R^{11a}R^{11b}), —N(R^{11a}R^{11b}), —N(R^{11a})C(O)R¹⁰, —N(R^{11a})C(═NR¹²)R¹³, —N(R^{11a})S(O)₁₋₂R¹³, —N(R^{11c})C(O)N(R^{11a}R^{11b}), —N(R^{11c})C(═NR¹²)N(R^{11a}R^{11b}), —N(R^{11c})S(O)₁₋₂N(R^{11a}R^{11b}) —N(R^{11a})CO₂R¹³, and an optionally substituted group selected from C1-C6 alkyl, C1-C6 alkoxy, C1-C6 haloalkyl, C1-C6 haloalkoxy, C3-C6 cycloalkyl, C3-C6 cycloalkylidenyl, C3-C6 cycloalkoxy, saturated or unsaturated 4-7 membered heterocyclyl containing 1-2 heteroatoms selected from N, O, and S as ring members, aryl, and heteroaryl containing 1-4 heteroatoms selected from N, O, and S as ring members; wherein the optional substituents are 1-4 substituents independently selected from -halo, —OH, NH₂, ═O, —CN, —SO₂NH₂, C1-C6 alkyl, C1-C6 haloalkyl, C1-C6 alkoxy, C1-C6 haloalkoxy, C3-C6 cycloalkyl, C3-C6 cycloalkoxy, C1-C6 alkylsulfonyl, C3-C6 cycloalkylsulfonyl, C1-C6 alkylsulfonylamino, C3-C6 cycloalkylsulfonylamino, C1-C6 alkylaminosulfonyl, and C3-C6 cycloalkylaminosulfonyl; wherein R¹⁰, R^{11a}, R^{11b} and R^{11c} are independently selected from H and an optionally substituted group selected from C1-C6 alkyl, C3-C6 cycloalkyl, C1-C6 haloalkyl, saturated or unsaturated 4-7 membered heterocyclyl containing 1-2 heteroatoms selected from N, O, and S as ring members, aryl, heteroaryl containing 1-4 heteroatoms selected from N, O, and S as ring members; R¹² is selected from H, —CN, —OH, and an optionally substituted group selected from C1-C4 alkyl and C1-C4 alkoxy; R¹³ is an optionally substituted group selected from C1-C6 alkyl, C3-C6 cycloalkyl C1-C6 haloalkyl, saturated or unsaturated 4-7 membered heterocyclyl containing 1-2 heteroatoms selected from N, O, and S as ring members, aryl, heteroaryl containing 1-4 heteroatoms selected from N, O, and S as ring members; wherein each of R¹⁰, R^{11a}, R^{11b}, R^{11c}, R¹² and R¹³ is optionally substituted with 1-3 groups independently selected from halo, —OH, NH₂, ═O, —CN, —SO₂NH₂, C1-C6 alkyl, C1-C6 haloalkyl, C1-C6 alkoxy, C1-C6 haloalkoxy, C3-C6 cycloalkyl, C3-C6 cycloalkoxy, C1-C6 alkylsulfonyl, C3-C6 cycloalkylsulfonyl, C1-C6 alkylsulfonylamino, C3-C6 cycloalkylsulfonylamino, C1-C6 alkylaminosulfonyl and C3-C6 cycloalkylaminosulfonyl; wherein two substituents on the same or adjacent carbon atoms of R¹ can optionally be taken together to form a 4-7 membered ring that can be saturated or unsaturated and optionally contains 1-2 heteroatoms selected from N, O and S as ring members and can optionally be substituted with 1-2 groups independently selected from R⁹;
   R² is selected from H, —CN, ethynyl, halo, —CF₃, C1-C3 alkyl, and C1-C3alkoxy;
   R³ and R⁴ are independently selected from H, ═O, OH, CN, halo, —CO₂R¹⁰, —C(O)N(R^{11a}R^{11b}), —N(R^{11a}R^{11b}), and an optionally substituted group selected from C1-C6 alkyl, C1-C6 alkyloxy, and C3-C6 cycloalkylidenyl optionally contains 1-2 heteroatoms as ring members selected from N, O and S, wherein the optional substituents are one or more groups independently selected from R⁹; wherein R³ and R⁴ can optionally be taken together to form a 4-7 membered cycloalkyl or 4-7 membered heterocyclyl optionally contains 1-2 heteroatoms as ring members selected from N, O and S and can optionally be substituted with at least one group selected from R⁹;
   R⁵, R⁶, R⁷ and R⁸ are independently selected from H, OH, CN, halo, —CO₂R¹⁰, —C(O)N(R^{11a}R^{11b}), —N(R^{11a}R^{11b}), and an optionally substituted group selected from C1-C6 alkyl and C1-C6 alkyloxy, wherein the optional substituents are one or more groups independently selected from R⁹; or
   wherein R⁵ and R⁶, or R⁷ and R⁸, can optionally be taken together to form a group selected from 3-7 membered cycloalkyl and 4-7 membered heterocyclyl containing 1-2 heteroatoms selected from N, O and S as ring members, wherein said 3-7 membered cycloalkyl or 4-7 membered heterocyclyl can optionally be fused with one or more groups selected from C3-C7 cycloalkyl, 4-10 membered heterocyclyl containing 1-3 heteroatoms selected from N, O, and S as ring members, aryl, and heteroaryl containing 1-4 heteroatoms selected from N, O, and S as ring members, and wherein any of the foregoing cyclic groups is optionally substituted with at least one group selected from R⁹;
   m is 0, 1, or 2;
   n is 1 or 2;
   A¹ is selected from —S(O)₂N(H)C(O)—, —S(O)₂N(H)C(O)N(R¹⁴)—, —C(O)N(H)S(O)₂—, —C(O)N(H)S(O)₂N(R¹⁴)—, —S(O)(═NR¹⁵)N(H)C(O)—, —S(O)(═NR¹⁵)N(H)C(O)N(R¹⁴)—, —C(O)N(H)S(O) (═NR¹⁵)—, and —C(O)N(H)S(O) (═NR¹⁵)N(R¹⁴)—; wherein R¹⁴ is selected from H and an optionally substituted group selected from C1-C6 alkyl, C3-C6 cycloalkyl, C1-C6 haloalkyl, and 4-7 membered heterocyclyl containing 1-2 heteroatoms selected from N, O, and S as ring members; wherein R¹⁵ is selected from H, —CN, —OH, and an optionally substituted group selected from C1-C4 alkyl and C1-C4 alkoxy; and wherein R¹⁴ and R¹⁵ are optionally substituted with at least one group selected from R⁹;
   A² is selected from a bond, —N(R¹⁴)—, and —O—;
   X¹, X², and X³ are independently selected from —N— and —CH—,
   L¹ is selected from a bond, —S—, —O—, and —C(O)—,
   L² is selected from:

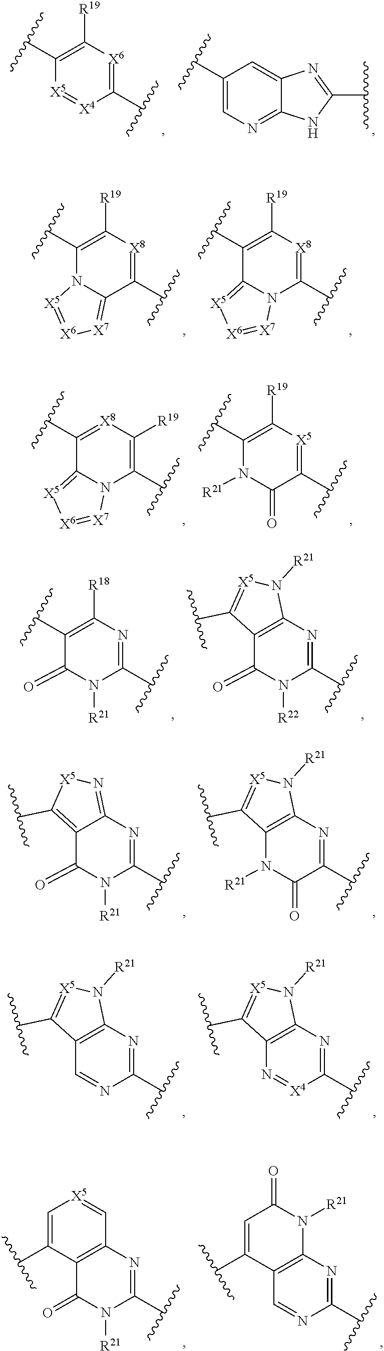

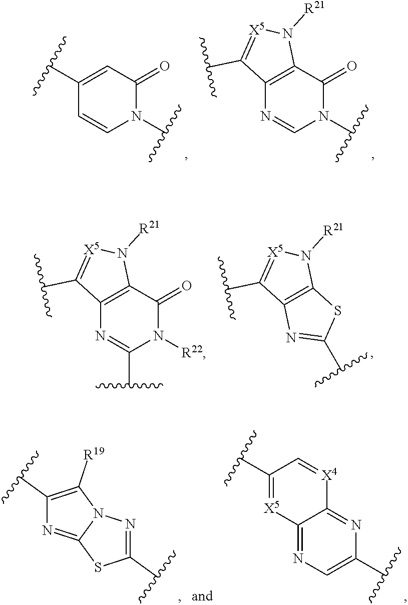

wherein R¹⁹ is selected from H, OH, CN, NH₂, halo, and an optionally substituted group selected from C1-C4 alkyl, C1-C6 haloalkyl, C3-C6 cycloalkyl, and aryl, wherein the optionally substituted groups can be substituted with at least one group selected from R⁹;
   X⁴, X⁵, X⁶, X⁷, and X⁸ are independently selected from —N— and —C(R²⁰)—, wherein R²⁰ is selected from H, —CONH₂, OH, NH₂, and CN, and —CH₂H; and R²¹ and R²² are independently selected from H, CH₃, and —CH₂H.
      Embodiment 2. The compound of Embodiment 1, wherein R¹ is selected from C1-C12 alkyl, C3-C12 cycloalkyl, 4-12 membered heterocyclyl containing 1-3 heteroatoms selected from N, O, and S as ring members, aryl, and heteroaryl containing 1-4 heteroatoms selected from N, O, and S as ring members; and wherein R¹ is optionally substituted with 1-4 substituents independently selected from R⁹.
      Embodiment 3. The compound of Embodiment 1 or Embodiment 2, wherein R¹ is selected from C1-C6 alkyl, C3-C10 cycloalkyl, 4-10 membered heterocyclyl containing 1-3 heteroatoms selected from N, O, and S as ring members, C6-C10 aryl, and 4-10 membered heteroaryl containing 1-4 heteroatoms selected from N, O, and S as ring members; and wherein R¹ is optionally substituted with 1-4 substituents independently selected from R⁹.
      Embodiment 4. The compound of any one of Embodiments 1-3, wherein R¹ is selected from phenyl, pyridyl, pyrimidinyl, pyrazinyl, pyridazinyl, cyclohexyl, cyclopentyl, cyclobutyl, cyclopropyl, methyl, and ethyl; and wherein R¹ is optionally substituted with 1-2 substituents independently selected from R⁹.
      Embodiment 5. The compound of any one of Embodiments 1-4, wherein R² is selected from H and halo.
      Embodiment 6. The compound of any one of Embodiments 1-5, wherein R² is selected from H and Cl.
      Embodiment 7. The compound of any one of Embodiments 1-6, wherein R³ is H.
      Embodiment 8. The compound of any one of Embodiments 1-7, wherein R⁴ is H.
      Embodiment 9. The compound of any one of Embodiments 1-8, wherein R⁵ and R⁶ are independently selected from H, OH, CN, halo, —N(R^{11a}R^{11b}), and C1-C6 alkyl optionally substituted with 1-3 groups independently selected from R⁹.
      Embodiment 10. The compound of any one of Embodiments 1-9, wherein R⁵ and R⁶ are independently selected from H, NH₂, and C1-C6 alkyl optionally substituted with 1-3 groups independently selected from R⁹.
      Embodiment 11. The compound of any one of Embodiments 1-10, wherein R⁵ and R⁶ are independently selected from H, NH₂, and —CH₂NH₂.
      Embodiment 12. The compound of any one of Embodiments 1-8, wherein R⁵ and R⁶ are taken together to form a group selected from 3-7 membered cycloalkyl and 4-7 membered heterocyclyl containing 1-2 heteroatoms selected from N, O and S as ring members, wherein said 3-7 membered cycloalkyl or 4-7 membered heterocyclyl are optionally substituted with 1-3 groups independently selected from R⁹.
      Embodiment 13. The compound of any one of Embodiments 1-8 and 12, wherein R⁵ and R⁶ are taken together to form a tetrahydrofuranyl optionally substituted with 1-3 groups independently selected from R⁹.
      Embodiment 14. The compound of any one of Embodiments 1-13, wherein m is 1.
      Embodiment 15. The compound of any one of Embodiments 1-13, wherein m is 2.
      Embodiment 16. The compound of any one of Embodiments 1-15, wherein n is 1.
      Embodiment 17. The compound of any one of Embodiments 1-15, wherein n is 2.
      Embodiment 18. The compound of any one of Embodiments 1-17, wherein A¹ is selected from —S(O)₂N(H)C(O)—, —S(O)₂N(H)C(O)N(R¹⁴)—, and —C(O)N(H)S(O)₂—; wherein R¹⁴ is selected from H and an optionally substituted group selected from C1-C6 alkyl, C3-C6 cycloalkyl, C1-C6 haloalkyl, and 4-7 membered heterocyclyl containing 1-2 heteroatoms selected from N, O, and S as ring members; and wherein R¹⁴ is optionally substituted with at least one group selected from R⁹;
      Embodiment 19. The compound of any one of Embodiments 1-18, wherein A¹ is selected from —S(O)₂N(H)C(O)—, —S(O)₂N(H)C(O)N(H)—, and —C(O)N(H)S(O)₂—.
      Embodiment 20. The compound of any one of Embodiments 1-19, wherein X¹ is —CH—, X² is —CH—, and X³ is —CH—.
      Embodiment 21. The compound of any one of Embodiments 1-19, wherein X¹ is N, X² is —CH—, and X³ is —CH—.
      Embodiment 22. The compound of any one of Embodiments 1-21, wherein L¹ is selected from a bond, —S—, —S(O)—, —S(O)₂—, and —O—.
      Embodiment 23. The compound of any one of Embodiments 1-22, wherein L¹ is —S—.
      Embodiment 24. The compound of any one of Embodiments 1-23, wherein L² is selected from

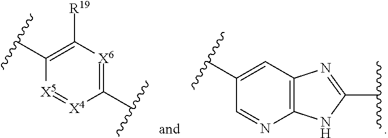

Embodiment 25. The compound of any one of Embodiments 1-24, wherein L² is selected from

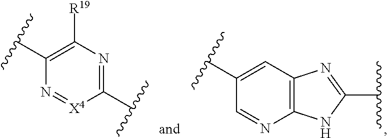

Embodiment 26. The compound of any one of Embodiments 1-25, wherein R¹⁹ is selected from H, NH₂, and C1-C4 alkyl optionally substituted with 1-3 groups independently selected from R⁹.
Embodiment 27. The compound of any one of Embodiments 1-26, wherein R¹⁹ is selected from H, NH₂, and methyl.
Embodiment 28. The compound of any one of Embodiments 1-27, wherein X⁴ is selected from —CH— and —C(CH₂OH)—.
Embodiment 29. The compound of any one of Embodiments 1-28, wherein X⁵ is —N—.
Embodiment 30. The compound of any one of Embodiments 1-29, wherein X⁶ is —N—.
Embodiment 31. The compound of any one of Embodiments 1-30, wherein R⁹ is selected from halo, NH₂, and C1-C6 alkyl.
Embodiment 32. The compound of Embodiment 1, wherein the compound is of Formula IA,

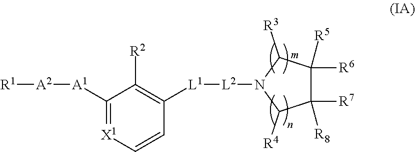

and/or a stereoisomer, a stable isotope, or a pharmaceutically acceptable salt or solvate thereof, wherein R¹, R², R³, R⁴, R⁵, R⁶, R⁷, R⁸, A¹, A², L¹, L², X¹, n, and m are as defined in Embodiment 1.
   Embodiment 33. The compound of Embodiment 1, wherein the compound is of Formula IB,

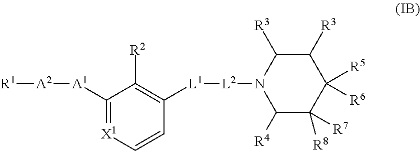

and/or a stereoisomer, a stable isotope, or a pharmaceutically acceptable salt or solvate thereof, wherein R¹, R², R³, R⁴, R⁵, R⁶, R⁷, R⁸, A¹, A², L¹, L², and X¹ are as defined in Embodiment 1.
   Embodiment 34. The compound of Embodiment 1, wherein the compound is of Formula IC,

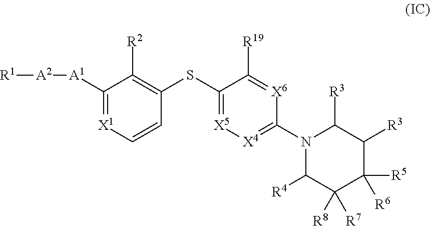

and/or a stereoisomer, a stable isotope, or a pharmaceutically acceptable salt or solvate thereof, wherein R¹, R², R³, R⁴, R⁵, R⁶, R⁷, R⁸, R¹⁹, A¹, A², X¹, X⁴, X⁵, and X⁶ are as defined in Embodiment 1.
   Embodiment 35. The compound of Embodiment 1, wherein the compound is of Formula ID,

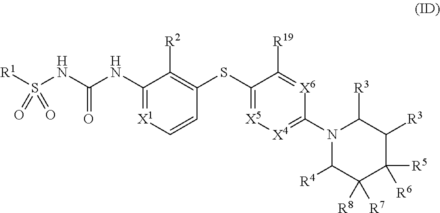

and/or a stereoisomer, a stable isotope, or a pharmaceutically acceptable salt or solvate thereof, wherein R¹, R², R³, R⁴, R⁵, R⁶, R⁷, R⁸, R¹⁹, X¹, X⁴, X⁵, and X⁶ are as defined in Embodiment 1.
   Embodiment 36. The compound of Embodiment 1, wherein the compound is of Formula IE,

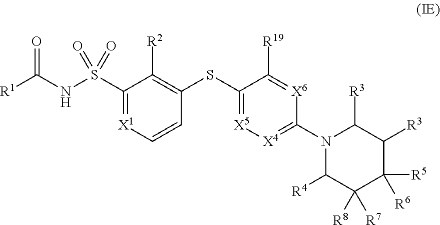

and/or a stereoisomer, a stable isotope, or a pharmaceutically acceptable salt or solvate thereof, wherein R¹, R², R³, R⁴, R⁵, R⁶, R⁷, R⁸, R¹⁹, X¹, X⁴, X⁵, and X⁶ are as defined in Embodiment 1.
   Embodiment 37. The compound of Embodiment 1, wherein the compound is of Formula IF,

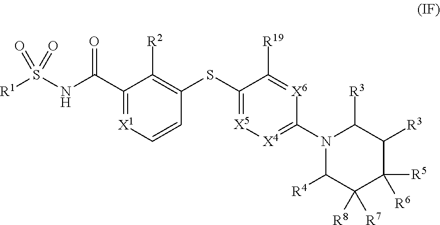

and/or a stereoisomer, a stable isotope, or a pharmaceutically acceptable salt or solvate thereof, wherein R¹, R², R³, R⁴, R⁵, R⁶, R⁷, R⁸, R¹⁹, X¹, X⁴, X⁵, and X⁶ are as defined in Embodiment 1.
   Embodiment 38. The compound of Embodiment 1, which is selected from the following compounds, and/or a stereoisomer, a stable isotope, or a pharmaceutically acceptable salt or solvate thereof:
N-((3-((5-(4-amino-4-methylpiperidin-1-yl)pyrazin-2-yl)thio)-2-chlorophenyl)carbamoyl)benzenesulfonamide,
N-((3-((5-(4-(aminomethyl)-4-methylpiperidin-1-yl)pyrazin-2-yl)thio)-2-chlorophenyl)carbamoyl)benzenesulfonamide,
3-((5-(4-amino-4-methylpiperidin-1-yl)pyrazin-2-yl)thio)-2-chloro-N-(phenylsulfonyl)benzamide,
N-((3-((3-amino-5-(4-amino-4-methylpiperidin-1-yl)pyrazin-2-yl)thio)-2-chlorophenyl)carbamoyl)benzenesulfonamide,
3-((3-amino-5-(4-(aminomethyl)-4-methylpiperidin-1-yl)pyrazin-2-yl)thio)-2-chloro-N-(phenylsulfonyl)benzamide,
N-((4-((5-((3S,4S)-4-amino-3-methyl-2-oxa-8-azaspiro[4.5]decan-8-yl)pyrazin-2-yl)thio)-3-chloropyridin-2-yl)carbamoyl)benzenesulfonamide,
N-((3-((5-(4-(aminomethyl)-4-methylpiperidin-1-yl)pyrazin-2-yl)thio)-2-chlorophenyl)sulfonyl)benzamide,
N-((3-((3-amino-5-(4-amino-4-methylpiperidin-1-yl)pyrazin-2-yl)thio)-2-chlorophenyl)sulfonyl)benzamide,
N-((4-((5-(4-(aminomethyl)-4-methylpiperidin-1-yl)-6-(hydroxymethyl)-3-methylpyrazin-2-yl)thio)-3-chloropyridin-2-yl)carbamoyl)benzenesulfonamide,
N-((3-((5-((3S,4S)-4-amino-3-methyl-2-oxa-8-azaspiro[4.5]decan-8-yl)pyrazin-2-yl)thio)-2-chlorophenyl)carbamoyl)-2-fluorobenzenesulfonamide,
N-((3-((5-((3S,4S)-4-amino-3-methyl-2-oxa-8-azaspiro[4.5]decan-8-yl)-6-(hydroxymethyl)-3-methylpyrazin-2-yl)thio)-2-chlorophenyl)carbamoyl)benzenesulfonamide,
((3S,4S)-8-(5-((2-chloro-3-((N-(phenylcarbamoyl)sulfamoyl)amino)phenyl)thio)-pyrazin-2-yl)-3-methyl-2-oxa-8-azaspiro[4.5]decan-4-yl)amine
N-((3-((5-((3S,4S)-4-amino-3-methyl-2-oxa-8-azaspiro[4.5]decan-8-yl)pyrazin-2-yl)thio)-2-chlorophenyl)carbamoyl)pyrrolidine-1-sulfonamide,
(S)—N-((4-((5-(7-amino-5,7-dihydrospiro[cyclopenta[b]pyridine-6,4′-piperidin]-1′-yl)pyrazin-2-yl)thio)-3-chloropyridin-2-yl)carbamoyl)benzenesulfonamide,
N-((4-((5-((3S,4S)-4-amino-3-methyl-2-oxa-8-azaspiro[4.5]decan-8-yl)-[1,2,4]triazolo[4,3-c]pyrimidin-8-yl)thio)-3-chloropyridin-2-yl)carbamoyl)benzenesulfonamide,
N—(N-(3-((5-((3S,4S)-4-amino-3-methyl-2-oxa-8-azaspiro[4.5]decan-8-yl)pyrazin-2-yl)thio)-2-chlorophenyl)sulfamoyl)pyrrolidine-1-carboxamide,
3-((5-((3S,4S)-4-amino-3-methyl-2-oxa-8-azaspiro[4.5]decan-8-yl)pyrazin-2-yl)thio)-2-chloro-N-(cyclopentylcarbamoyl)benzenesulfonamide,
3-((5-(4-amino-4-methylpiperidin-1-yl)pyrazin-2-yl)thio)-2-chloro-N-(cyclopropylsulfonyl)benzamide,
3-((5-(4-amino-4-methylpiperidin-1-yl)pyrazin-2-yl)thio)-2-chloro-N-(methylsulfonyl)benzamide,
3-((5-(4-amino-4-methylpiperidin-1-yl)pyrazin-2-yl)thio)-2-chloro-N-(cyclohexylsulfonyl)benzamide,
3-((5-(4-(aminomethyl)-4-methylpiperidin-1-yl)pyrazin-2-yl)thio)-2-chloro-N-(phenylsulfonyl)benzamide,
3-((5-(4-(aminomethyl)-4-methylpiperidin-1-yl)pyrazin-2-yl)thio)-2-chloro-N-(pyridin-2-ylsulfonyl)benzamide,
3-((5-(4-(aminomethyl)-4-methylpiperidin-1-yl)pyrazin-2-yl)thio)-2-chloro-N-((2-fluorophenyl)sulfonyl)benzamide
3-((5-(4-(aminomethyl)-4-methylpiperidin-1-yl)pyrazin-2-yl)thio)-2-chloro-N-(pyridin-3-ylsulfonyl)benzamide,
3-((5-(4-(aminomethyl)-4-methylpiperidin-1-yl)pyrazin-2-yl)thio)-2-chloro-N-(cyclopropylsulfonyl)benzamide,
3-((5-(4-(aminomethyl)-4-methylpiperidin-1-yl)pyrazin-2-yl)thio)-2-chloro-N-(methylsulfonyl)benzamide,
3-((5-(4-(aminomethyl)-4-methylpiperidin-1-yl)pyrazin-2-yl)thio)-2-chloro-N-(cyclohexylsulfonyl)benzamide,
3-((5-(4-(aminomethyl)-4-methylpiperidin-1-yl)pyrazin-2-yl)thio)-2-chloro-N-(pyridin-4-ylsulfonyl)benzamide,
N-((3-((5-(4-(aminomethyl)-4-methylpiperidin-1-yl)pyrazin-2-yl)thio)-2-chlorophenyl)carbamoyl)methanesulfonamide,
3-((5-(4-(aminomethyl)-4-methylpiperidin-1-yl)pyrazin-2-yl)thio)-2-chloro-N-(cyclopentylsulfonyl)benzamide,
(S)-3-((3-amino-5-(4-amino-2-oxa-8-azaspiro[4.5]decan-8-yl)pyrazin-2-yl)thio)-2-chloro-N-(phenylsulfonyl)benzamide,
N-((3-((5-(4-(aminomethyl)-4-methylpiperidin-1-yl)pyrazin-2-yl)thio)-2-chlorophenyl)carbamoyl)cyclopropanesulfonamide,
N-((3-((5-(4-amino-4-methylpiperidin-1-yl)pyrazin-2-yl)thio)-2-chlorophenyl)sulfonyl)benzamide,
3-((3-amino-5-(4-(aminomethyl)-4-methylpiperidin-1-yl)pyrazin-2-yl)thio)-2-chloro-N-((2-fluorophenyl)sulfonyl)benzamide,
3-((3-amino-5-(4-(aminomethyl)-4-methylpiperidin-1-yl)pyrazin-2-yl)thio)-2-chloro-N-(pyridin-2-ylsulfonyl)benzamide,
3-((3-amino-5-(4-(aminomethyl)-4-methylpiperidin-1-yl)pyrazin-2-yl)thio)-2-chloro-N-(cyclopropylsulfonyl)benzamide,
3-((3-amino-5-(4-(aminomethyl)-4-methylpiperidin-1-yl)pyrazin-2-yl)thio)-2-chloro-N-(methylsulfonyl)benzamide,
3-((3-amino-5-(4-(aminomethyl)-4-methylpiperidin-1-yl)pyrazin-2-yl)thio)-2-chloro-N-(pyridin-3-ylsulfonyl)benzamide,
3-((3-amino-5-(4-(aminomethyl)-4-methylpiperidin-1-yl)pyrazin-2-yl)thio)-2-chloro-N-(pyridin-4-ylsulfonyl)benzamide,
N-((3-((3-amino-5-(4-(aminomethyl)-4-methylpiperidin-1-yl)pyrazin-2-yl)thio)-2-chlorophenyl)carbamoyl)methanesulfonamide,
N-((3-((3-amino-5-(4-(aminomethyl)-4-methylpiperidin-1-yl)pyrazin-2-yl)thio)-2-chlorophenyl)carbamoyl)pyridine-4-sulfonamide,
N-((3-((3-amino-5-(4-(aminomethyl)-4-methylpiperidin-1-yl)pyrazin-2-yl)thio)-2-chlorophenyl)carbamoyl)cyclopentanesulfonamide,
N-((3-((3-amino-5-(4-(aminomethyl)-4-methylpiperidin-1-yl)pyrazin-2-yl)thio)-2-chlorophenyl)carbamoyl)cyclopropanesulfonamide,
3-((3-amino-5-(4-(aminomethyl)-4-methylpiperidin-1-yl)pyrazin-2-yl)thio)-2-chloro-N-(cyclopentylsulfonyl)benzamideN-((3-((3-amino-5-(4-(aminomethyl)-4-methylpiperidin-1-yl)pyrazin-2-yl)thio)-2-chlorophenyl)carbamoyl)pyridine-2-sulfonamide,
N-((3-((3-amino-5-(4-(aminomethyl)-4-methylpiperidin-1-yl)pyrazin-2-yl)thio)-2-chlorophenyl)carbamoyl)pyridine-3-sulfonamide,
N-((3-((3-amino-5-(4-(aminomethyl)-4-methylpiperidin-1-yl)pyrazin-2-yl)thio)-2-chlorophenyl)carbamoyl)-2-fluorobenzenesulfonamide,
N-((4-((5-(4-(aminomethyl)-4-methylpiperidin-1-yl)pyrazin-2-yl)thio)-3-chloropyridin-2-yl)carbamoyl)benzenesulfonamide,
N-((3-((5-((3S,4S)-4-amino-3-methyl-2-oxa-8-azaspiro[4.5]decan-8-yl)pyrazin-2-yl)thio)-2-chlorophenyl)carbamoyl)benzenesulfonamide,
N-((4-((5-((3S,4S)-4-amino-3-methyl-2-oxa-8-azaspiro[4.5]decan-8-yl)pyrazin-2-yl)thio)-3-chloropyridin-2-yl)carbamoyl)-3-fluorobenzenesulfonamide,
N-((4-((5-((3S,4S)-4-amino-3-methyl-2-oxa-8-azaspiro[4.5]decan-8-yl)pyrazin-2-yl)thio)-3-chloropyridin-2-yl)carbamoyl)pyridine-3-sulfonamide,
N-((4-((5-((3S,4S)-4-amino-3-methyl-2-oxa-8-azaspiro[4.5]decan-8-yl)pyrazin-2-yl)thio)-3-chloropyridin-2-yl)carbamoyl)-2-fluorobenzenesulfonamide,
N-((4-((5-((3S,4S)-4-amino-3-methyl-2-oxa-8-azaspiro[4.5]decan-8-yl)pyrazin-2-yl)thio)-3-chloropyridin-2-yl)carbamoyl)-4-fluorobenzenesulfonamide,
N-((4-((5-((3S,4S)-4-amino-3-methyl-2-oxa-8-azaspiro[4.5]decan-8-yl)pyrazin-2-yl)thio)-3-chloropyridin-2-yl)carbamoyl)-6-methoxypyridine-3-sulfonamide,
N-((4-((5-((3S,4S)-4-amino-3-methyl-2-oxa-8-azaspiro[4.5]decan-8-yl)pyrazin-2-yl)thio)-3-chloropyridin-2-yl)carbamoyl)-1-methyl-1H-pyrazole-4-sulfonamide,
N-((4-((5-((3S,4S)-4-amino-3-methyl-2-oxa-8-azaspiro[4.5]decan-8-yl)pyrazin-2-yl)thio)-3-fluoropyridin-2-yl)carbamoyl)benzenesulfonamide,
N-((4-((5-((3S,4S)-4-amino-3-methyl-2-oxa-8-azaspiro[4.5]decan-8-yl)pyrazin-2-yl)thio)-3-chloropyridin-2-yl)carbamoyl)cyclopropanesulfonamide,
N-((4-((5-((3S,4S)-4-amino-3-methyl-2-oxa-8-azaspiro[4.5]decan-8-yl)pyrazin-2-yl)thio)-3-chloropyridin-2-yl)carbamoyl)-5-fluoropyridine-3-sulfonamide,
N-((4-((5-((3S,4S)-4-amino-3-methyl-2-oxa-8-azaspiro[4.5]decan-8-yl)-6-(hydroxymethyl)-3-methylpyrazin-2-yl)thio)-3-chloropyridin-2-yl)carbamoyl)benzenesulfonamide,
N-((5-((5-((3S,4S)-4-amino-3-methyl-2-oxa-8-azaspiro[4.5]decan-8-yl)pyrazin-2-yl)thio)-4-chloropyridin-3-yl)carbamoyl)benzenesulfonamide,
N-((3-((5-((3S,4S)-4-amino-3-methyl-2-oxa-8-azaspiro[4.5]decan-8-yl)pyrazin-2-yl)thio)-2-chlorophenyl)carbamoyl)pyridine-3-sulfonamide,
N-((3-((5-((3S,4S)-4-amino-3-methyl-2-oxa-8-azaspiro[4.5]decan-8-yl)pyrazin-2-yl)thio)-2-chlorophenyl)carbamoyl)-3-fluorobenzenesulfonamide,
N-((3-((5-((3S,4S)-4-amino-3-methyl-2-oxa-8-azaspiro[4.5]decan-8-yl)pyrazin-2-yl)thio)-2-chlorophenyl)carbamoyl)pyridine-2-sulfonamide,
N-((3-((5-((3S,4S)-4-amino-3-methyl-2-oxa-8-azaspiro[4.5]decan-8-yl)pyrazin-2-yl)thio)-2-chlorophenyl)carbamoyl)-6-methoxypyridine-3-sulfonamide,
N-((3-((5-((3S,4S)-4-amino-3-methyl-2-oxa-8-azaspiro[4.5]decan-8-yl)pyrazin-2-yl)thio)-2-chlorophenyl)carbamoyl)-3,5-dimethylisoxazole-4-sulfonamide,
N-((3-((5-((3S,4S)-4-amino-3-methyl-2-oxa-8-azaspiro[4.5]decan-8-yl)pyrazin-2-yl)thio)-2-chlorophenyl)carbamoyl)cyclopropanesulfonamide,
(S)—N-((3-((5-(7-amino-5,7-dihydrospiro[cyclopenta[c]pyridine-6,4′-piperidin]-1′-yl)pyrazin-2-yl)thio)-2-chlorophenyl)carbamoyl)benzenesulfonamide,
N-((3-((5-((3S,4S)-4-amino-3-methyl-2-oxa-8-azaspiro[4.5]decan-8-yl)pyrazin-2-yl)thio)-2-chlorophenyl)carbamoyl)tetrahydro-2H-pyran-4-sulfonamide,
N-((3-((5-((3S,4S)-4-amino-3-methyl-2-oxa-8-azaspiro[4.5]decan-8-yl)pyrazin-2-yl)thio)-2-chlorophenyl)carbamoyl)cyclohexanesulfonamide,
N-((3-((5-((3S,4S)-4-amino-3-methyl-2-oxa-8-azaspiro[4.5]decan-8-yl)pyrazin-2-yl)thio)-2-chlorophenyl)carbamoyl)-1-methylpiperidine-4-sulfonamide,
4-((5-((3S,4S)-4-amino-3-methyl-2-oxa-8-azaspiro[4.5]decan-8-yl)pyrazin-2-yl)thio)-3-chloro-N-(phenylsulfonyl)picolinamide,
N-((3-((5-((3S,4S)-4-amino-3-methyl-2-oxa-8-azaspiro[4.5]decan-8-yl)pyrazin-2-yl)thio)-2-chlorophenyl)carbamoyl)pyridine-4-sulfonamide,
N-((3-((5-((3S,4S)-4-amino-3-methyl-2-oxa-8-azaspiro[4.5]decan-8-yl)pyrazin-2-yl)thio)-2-chlorophenyl)carbamoyl)cyclopentanesulfonamide,
N-((3-((5-((3S,4S)-4-amino-3-methyl-2-oxa-8-azaspiro[4.5]decan-8-yl)pyrazin-2-yl)thio)-2-chlorophenyl)carbamoyl)morpholine-4-sulfonamide,
N-((3-((5-((3S,4S)-4-amino-3-methyl-2-oxa-8-azaspiro[4.5]decan-8-yl)pyrazin-2-yl)thio)-2-chlorophenyl)carbamoyl)piperidine-1-sulfonamide,
N-((4-((5-((3S,4S)-4-amino-3-methyl-2-oxa-8-azaspiro[4.5]decan-8-yl)pyrazin-2-yl)thio)-3-chloropyridin-2-yl)carbamoyl)-3,5-dimethylisoxazole-4-sulfonamide,
N-((4-((5-((3S,4S)-4-amino-3-methyl-2-oxa-8-azaspiro[4.5]decan-8-yl)pyrazin-2-yl)thio)-3-chloropyridin-2-yl)carbamoyl)methanesulfonamide,
N-((3-((5-((3S,4S)-4-amino-3-methyl-2-oxa-8-azaspiro[4.5]decan-8-yl)pyrazin-2-yl)thio)-2-chlorophenyl)carbamoyl)-4-fluorobenzenesulfonamide,
N-((3-((5-((3S,4S)-4-amino-3-methyl-2-oxa-8-azaspiro[4.5]decan-8-yl)pyrazin-2-yl)thio)-2-chlorophenyl)carbamoyl)-1-methyl-1H-pyrazole-4-sulfonamide,
N—(N-(3-((5-((3S,4S)-4-amino-3-methyl-2-oxa-8-azaspiro[4.5]decan-8-yl)pyrazin-2-yl)thio)-2-chlorophenyl)sulfamoyl)benzamide,
N-((3-((5-((3S,4S)-4-amino-3-methyl-2-oxa-8-azaspiro[4.5]decan-8-yl)pyrazin-2-yl)thio)-2-chlorophenyl)carbamoyl)-3-hydroxypyrrolidine-1-sulfonamide,
N-((3-((5-((3S,4S)-4-amino-3-methyl-2-oxa-8-azaspiro[4.5]decan-8-yl)pyrazin-2-yl)thio)-2-chlorophenyl)carbamoyl)cyclobutanesulfonamide,
N-((3-((5-((3S,4S)-4-amino-3-methyl-2-oxa-8-azaspiro[4.5]decan-8-yl)pyrazin-2-yl)thio)-2-chlorophenyl)carbamoyl)tetrahydrofuran-3-sulfonamide, and
3-amino-N-((3-((5-((3S,4S)-4-amino-3-methyl-2-oxa-8-azaspiro[4.5]decan-8-yl)pyrazin-2-yl)thio)-2-chlorophenyl)carbamoyl)pyrrolidine-1-sulfonamide.
   Embodiment 39. A pharmaceutical composition comprising a compound of any one of Embodiments 1-38, and/or a stereoisomer, a stable isotope, or a pharmaceutically acceptable salt or solvate thereof, admixed with at least one pharmaceutically acceptable carrier.
   Embodiment 40. A method to treat a disease in a patient in need thereof whose disease is a SHP2-associated disease, comprising administering to the subject in need of such treatment a therapeutically effective amount of a compound of any one of Embodiments 1-38 and/or a stereoisomer, a stable isotope, or a pharmaceutically acceptable salt or solvate thereof, or a pharmaceutical composition of Embodiment 39.
   Embodiment 41. The method of Embodiment 40, wherein the method comprises determining if the disease in the patient is a SHP2-associated disease, and administering to a subject in need of such treatment a therapeutically effective SHP2 inhibiting amount of a compound of any one of Embodiments 1-38, and/or a stereoisomer, a stable isotope, or a pharmaceutically acceptable salt or solvate thereof, or a pharmaceutical composition or a pharmaceutical composition of Embodiment 39.
   Embodiment 42. The method of Embodiment 40 or Embodiment 41, wherein the SHP2-associated disease or disorder is mediated by the activity of SHP2.
   Embodiment 43. The method of Embodiment 42, wherein the SHP2-associated disease or disorder is selected from Noonan Syndrome, Leopard Syndrome, juvenile myelomonocytic leukemias, neuroblastoma, melanoma, acute myeloid leukemia, breast cancer, esophageal cancer, lung cancer, colon cancer, head camcer, neuroblastoma, squamous-cell carcinoma of the head and neck, gastric carcinoma, anaplastic large-cell lymphoma and glioblastoma.
   Embodiment 44. The method of any of one of Embodiment 40-43, wherein the compound of any one of Embodiments 1-38, and/or a stereoisomer, a stable isotope, or a pharmaceutically acceptable salt or solvate thereof, or a pharmaceutical composition of Embodiment 39, is orally administered.
   Embodiment 45. A use of a compound of any one of Embodiments 1-38, and/or a stereoisomer, a stable isotope, or a pharmaceutically acceptable salt or solvate thereof, or a pharmaceutical composition according to Embodiment 39 as a medicament, in the manufacture of a medicament, or in medicine for treatment of a SHP2-associated diease or disorder.
   Embodiment 46. The use of Embodiment 45 wherein the SHP2-associated disease or disorder is mediated by the activity of SHP2.
   Embodiment 47. The use of Embodiment 46, wherein the SHP2-associated disease or disorder is selected from Noonan Syndrome, Leopard Syndrome, juvenile myelomonocytic leukemias, neuroblastoma, melanoma, acute myeloid leukemia, breast cancer, esophageal cancer, lung cancer, colon cancer, head camcer, neuroblastoma, squamous-cell carcinoma of the head and neck, gastric carcinoma, anaplastic large-cell lymphoma and glioblastoma.
   Embodiment 48. The use of any of one of Embodiments 45-47, wherein the medicament is formulated for oral administration.
   Embodiment 49. A compound of any one of Embodiments 1-38, and/or a stereoisomer, a stable isotope, or a pharmaceutically acceptable salt or solvate thereof, or a pharmaceutical composition of Embodiment 39 for use in treating a SHP2-associated disease or disorder.
   Embodiment 50. The compound or pharmaceutical composition of Embodiment 49, wherein the SHP2-associated disease is a SHP2-associated cancer.
   Embodiment 51. The compound or pharmaceutical composition of Embodiment 49, wherein the SHP2-associated disease or disorder is a SHP2-associated cancer, and the use comprises determining if the cancer in a patient is a SHP2-associated cancer, and administering to the patient in need of such treatment a therapeutically effective amount of the compound or pharmaceutical composition.
   Embodiment 52. The compound or pharmaceutical composition of any of one of Embodiments 49-51, wherein the SHP2-associated cancer is selected from juvenile myelomonocytic leukemias, neuroblastoma, melanoma, acute myeloid leukemia, breast cancer, esophageal cancer, lung cancer, colon cancer, head camcer, neuroblastoma, squamous-cell carcinoma of the head and neck, gastric carcinoma, anaplastic large-cell lymphoma and glioblastoma.
   Embodiment 53. A method of inhibiting SHP2 activity in vitro or in vivo for a SHP2-associated cancer cell with a compound of any one of Embodiments 1-38, and/or a stereoisomer, a stable isotope, or a pharmaceutically acceptable salt or solvate thereof.

In some embodiments, the compound of Formula I (such as Formulae IA, IB, IC, ID, IE, and IF), has the chiral configuration shown in excess over its enantiomer, so the compound is optically active. For example, such compounds disclosed herein are substantially free of the opposite enantiomer, i.e., at least 95% of the compound has the chirality shown above.

Also disclosed herein is a pharmaceutical composition comprising a compound of Formula I (such as Formulae IA, IB, IC, ID, IE, and IF), and/or a stereoisomer, a stable isotope, or a pharmaceutically acceptable salt or solate thereof, and a pharmaceutically acceptable carrier.

Further disclosed herein is a method of inhibiting the activity of SHP2 comprising contacting the protein SHP2 with an effective amount of a compound of Formula I (such as Formulae IA, IB, IC, ID, IE, and IF), and/or a stereoisomer, a stable isotope, or a pharmaceutically acceptable salt or solvate thereof disclosed herein.

Further disclosed herein is a method of treating a disease treatable by inhibition of SHP2 in a patient, comprising administering to the patient in recognized need of such treatment, an effective amount of a compound of Formula I (such as Formulae IA, IB, IC, ID, IE, and IF), and/or a stereoisomer, a stable isotope, or a pharmaceutically acceptable salt or solvate thereof disclosed herein.

Further disclosed herein is a method of treating a disease treatable by inhibition of SHP2 in a patient, comprising administering to the patient in recognized need of such treatment, an effective amount of a pharmaceutical composition comprising a compound of Formula I (such as Formulae IA, IB, IC, ID, IE, and IF), and/or a stereoisomer, a stable isotope, or a pharmaceutically acceptable salt or solvate thereof disclosed herein and a pharmaceutically acceptable carrier.

Further disclosed herein is a method of treating a cancer in a patient, comprising administering to the patient in recognized need of such treatment, an effective amount of a pharmaceutical composition comprising a compound of Formula I (such as Formulae IA, IB, IC, ID, IE, and IF), and/or a stereoisomer, a stable isotope, or a pharmaceutically acceptable salt or solvate thereof disclosed herein and a pharmaceutically acceptable carrier. In some embodiments, the cancer is juvenile myelomonocytic leukemias, neuroblastoma, melanoma, acute myeloid leukemia, breast cancer, esophageal cancer, lung cancer, colon cancer, head camcer, neuroblastoma, squamous-cell carcinoma of the head and neck, gastric carcinoma, anaplastic large-cell lymphoma and glioblastoma.

Further disclosed herein is a use of a compound of Formula I (such as Formulae IA, IB, IC, ID, IE, and IF), and/or a stereoisomer, a stable isotope, or a pharmaceutically acceptable salt or solvate thereof in preparation of a medication for treating a disease responsive to inhibition of SHP2, such as a cancer. In some embodiments, the cancer is juvenile myelomonocytic leukemias, neuroblastoma, melanoma, acute myeloid leukemia, breast cancer, esophageal cancer, lung cancer, colon cancer, head camcer, neuroblastoma, squamous-cell carcinoma of the head and neck, gastric carcinoma, anaplastic large-cell lymphoma and glioblastoma.

The pharmaceutical composition comprising a compound of Formula I (such as Formulae IA, IB, IC, ID, IE, and IF), and/or a stereoisomer, a stable isotope, or a pharmaceutically acceptable salt or solvate thereof, and a pharmaceutically acceptable carrier, can be administered in various known manners, such as orally, topically, rectally, parenterally, by inhalation spray, or via an implanted reservoir, although the most suitable route in any given case will depend on the particular host, and nature and severity of the conditions for which the active ingredient is being administered. The term “parenteral” as used herein includes subcutaneous, intracutaneous, intravenous, intramuscular, intraarticular, intraarterial, intrasynovial, intrasternal, intrathecal, intralesional and intracranial injection or infusion techniques. The compositions disclosed herein may be conveniently presented in unit dosage form and prepared by any of the methods well known in the art.

The compound of Formula I (such as Formulae IA, IB, IC, ID, IE, and IF), and/or a stereoisomer, a stable isotope, or a pharmaceutically acceptable salt or solvate thereof can be administered orally in solid dosage forms, such as capsules, tablets, troches, dragées, granules and powders, or in liquid dosage forms, such as elixirs, syrups, emulsions, dispersions, and suspensions. The compound of Formula I (such as Formulae IA, IB, IC, ID, IE, and IF), and/or a stereoisomer, a stable isotope, or a pharmaceutically acceptable salt or solvate thereof can also be administered parenterally, in sterile liquid dosage forms, such as dispersions, suspensions or solutions. Other dosages forms that can also be used to administer the compound of Formula I (such as Formulae IA, IB, IC, ID, IE, and IF), and/or a stereoisomer, a stable isotope, or a pharmaceutically acceptable salt or solvate thereof include ointment, cream, drops, transdermal patch or powder for topical administration, an ophthalmic solution or suspension formation, i.e., eye drops, for ocular administration, an aerosol spray or powder composition for inhalation or intranasal administration, or a cream, ointment, spray or suppository for rectal or vaginal administration.

Gelatin capsules containing the compound of Formula I (such as Formulae IA, IB, IC, ID, IE, and IF), and/or a stereoisomer, a stable isotope, or a pharmaceutically acceptable salt or solvate thereof and at least one powdered carrier selected, for example, from lactose, starch, cellulose derivatives, magnesium stearate, stearic acid, and the like, can also be used. Similar diluents can be used to make compressed tablets. Both tablets and capsules can be manufactured as sustained release products to provide for continuous release of medication over a period of time. Compressed tablets can be sugar coated or film coated to mask any unpleasant taste and protect the tablet from the atmosphere, or enteric coated for selective disintegration in the gastrointestinal tract.

Liquid dosage forms for oral administration can further comprise at least one agent selected from coloring and flavoring agents to increase patient acceptance.

In general, water, suitable oil, saline, aqueous dextrose (glucose), and related sugar solutions and glycols such as propylene glycol or polyethylene gycols can be examples of suitable carriers for parenteral solutions. Solutions for parenteral administration may comprise a water soluble salt of the at least one compound disclosed herein, at least one suitable stabilizing agent, and if necessary, at least one buffer substance. Antioxidizing agents such as sodium bisulfite, sodium sulfite, or ascorbic acid, either alone or combined, can be examples of suitable stabilizing agents. Citric acid and its salts and sodium EDTA can also be used as examples of suitable stabilizing agents. In addition, parenteral solutions can further comprise at least one preservative, selected, for example, from benzalkonium chloride, methyl- and propylparaben, and chlorobutanol.

A pharmaceutically acceptable carrier is, for example, selected from carriers that are compatible with active ingredients of the pharmaceutical composition (and in some embodiments, capable of stabilizing the active ingredients) and not deleterious to the subject to be treated. For example, solubilizing agents, such as cyclodextrins (which can form specific, more soluble complexes with the at least one compound and/or at least one pharmaceutically acceptable salt disclosed herein), can be utilized as pharmaceutical excipients for delivery of the active ingredients. Examples of other carriers include colloidal silicon dioxide, magnesium stearate, cellulose, sodium lauryl sulfate, and pigments such as D&C Yellow #10. Suitable pharmaceutically acceptable carriers are disclosed in Remington's Pharmaceutical Sciences, A. Osol, a standard reference text in the art.

The compound of Formula I (such as Formulae IA, IB, IC, ID, IE, and IF), and/or a stereoisomer, a stable isotope, or a pharmaceutically acceptable salt or solvate thereof can be examined for efficacy in treating cancer by in vivo assays. For example, the compound of Formula I (such as Formulae IA, IB, IC, ID, IE, and IF), and/or a stereoisomer, a stable isotope, or a pharmaceutically acceptable salt or solvate thereof can be administered to an animal (e.g., a mouse model) having cancer and its therapeutic effects can be accessed. Positive results in one or more of such tests are sufficient to increase the scientific storehouse of knowledge and hence sufficient to demonstrate practical utility of the compounds and/or salts tested. Based on the results, an appropriate dosage range and administration route for animals, such as humans, can also be determined.

For administration by inhalation, the compound of Formula I (such as Formulae IA, IB, IC, ID, IE, and IF), and/or a stereoisomer, a stable isotope, or a pharmaceutically acceptable salt or solvate thereof may be conveniently delivered in the form of an aerosol spray presentation from pressurized packs or nebulisers. The compound of Formula I (such as Formulae IA, IB, IC, ID, IE, and IF), and/or a stereoisomer, a stable isotope, or a pharmaceutically acceptable salt or solvate thereof may also be delivered as powders, which may be formulated and the powder composition may be inhaled with the aid of an insufflation powder inhaler device. One exemplary delivery system for inhalation can be a metered dose inhalation (MDI) aerosol, which may be formulated as a suspension or solution of a compound of Formula I (such as Formulae IA, IB, IC, ID, IE, and IF), and/or a stereoisomer, a stable isotope, or a pharmaceutically acceptable salt or solvate thereof in at least one suitable propellant, selected, for example, from fluorocarbons and hydrocarbons.

For ocular administration, an ophthalmic preparation may be formulated with an appropriate weight percentage of a solution or suspension of the compound of Formula I (such as Formulae IA, IB, IC, ID, IE, and IF) and/or a stereoisomer, a stable isotope, or a pharmaceutically acceptable salt or solvate thereof in an appropriate ophthalmic vehicle, such that the compound of Formula I (such as Formulae IA, IB, IC, ID, IE, and IF), and/or a stereoisomer, a stable isotope, or a pharmaceutically acceptable salt or solvate thereof is maintained in contact with the ocular surface for a sufficient time period to allow the compound to penetrate the corneal and internal regions of the eye.

Useful pharmaceutical dosage-forms for administration of the compound of Formula I (such as Formulae IA, IB, IC, ID, IE, and IF), and/or a stereoisomer, a stable isotope, or a pharmaceutically acceptable salt or solvate thereof include, but are not limited to, hard and soft gelatin capsules, tablets, parenteral injectables, and oral suspensions.

The dosage administered will be dependent on factors, such as the age, health and weight of the recipient, the extent of disease, type of concurrent treatment, if any, frequency of treatment, and the nature of the effect desired. In general, a daily dosage of the active ingredient can vary, for example, from 0.1 to 2000 milligrams per day. For example, 10-500 milligrams once or multiple times per day may be effective to obtain the desired results.

In some embodiments, the compound of Formula I (such as Formulae IA, IB, IC, ID, IE, and IF), and/or a stereoisomer, a stable isotope, or a pharmaceutically acceptable salt or solvate thereof can be present in an amount of 1, 5, 10, 15, 20, 25, 50, 75, 80, 85, 90, 95, 100, 125, 150, 200, 250, 300, 400 and 500 mg in a capsule.

In some embodiments, a large number of unit capsules can be prepared by filling standard two-piece hard gelatin capsules each with, for example, 100 milligrams of the compound of Formula I (such as Formulae IA, IB, IC, ID, IE, and IF), and/or a stereoisomer, a stable isotope, or a pharmaceutically acceptable salt or solvate thereof in powder, 150 milligrams of lactose, 50 milligrams of cellulose, and 6 milligrams magnesium stearate.

In some embodiments, a mixture of the compound of Formula I (such as Formulae IA, IB, IC, ID, IE, and IF), and/or a stereoisomer, a stable isotope, or a pharmaceutically acceptable salt or solvate thereof and a digestible oil such as soybean oil, cottonseed oil or olive oil can be prepared and injected by means of a positive displacement pump into gelatin to form soft gelatin capsules containing 75 or 100 milligrams of the active ingredient. The capsules are washed and dried.

In some embodiments, the compound of Formula I (such as Formulae IA, IB, IC, ID, IE, and IF), and/or a stereoisomer, a stable isotope, or a pharmaceutically acceptable salt or solvate thereof can be present in an amount of 1 mg to 500 mg in a tablet, for example 1, 5, 10, 15, 20, 25, 50, 75, 80, 85, 90, 95, 100, 125, 150, 200, 250, 300, 400 and 500 mg in a tablet.

In some embodiments, a large number of tablets can be prepared by conventional procedures so that the dosage unit comprises, for example, 100 milligrams of the compound of Formula I (such as Formulae IA, IB, IC, ID, IE, and IF), and/or a stereoisomer, a stable isotope, or a pharmaceutically acceptable salt or solvate thereof, 0.2 milligrams of colloidal silicon dioxide, 5 milligrams of magnesium stearate, 275 milligrams of microcrystalline cellulose, 11 milligrams of starch and 98.8 milligrams of lactose. Appropriate coatings may, for example, be applied to increase palatability or delay absorption.

In some embodiments, a parenteral composition suitable for administration by injection can be prepared by stirring 1.5% by weight of a compound of Formula I (such as Formulae IA, IB, IC, ID, IE, and IF), and/or a stereoisomer, a stable isotope, or a pharmaceutically acceptable salt or solvate thereof in 10% by volume propylene glycol. The solution is made to the expected volume with water for injection and sterilized.

In some embodiment, an aqueous suspension can be prepared for oral administration. For example, each 5 milliliters of an aqueous suspension comprising 100 milligrams of finely divided compound of Formula I (such as Formulae IA, IB, IC, ID, IE, and IF), and/or a stereoisomer, a stable isotope, or a pharmaceutically acceptable salt or solvate thereof, 100 milligrams of sodium carboxymethyl cellulose, 5 milligrams of sodium benzoate, 1.0 grams of sorbitol solution, U.S.P., and 0.025 milliliters of vanillin can be used.

The same dosage forms can generally be used when the compound of Formula I (such as Formulae IA, IB, IC, ID, IE, and IF), and/or a stereoisomer, a stable isotope, or a pharmaceutically acceptable salt or solvate thereof are administered stepwise or in conjunction with at least one other therapeutic agent. When drugs are administered in physical combination, the dosage form and administration route should be selected depending on the compatibility of the combined drugs. Thus, the term “co-administration” is understood to include the administration of at least two agents concomitantly or sequentially, or alternatively as a fixed dose combination of the at least two active components.

The compound of Formula I (such as Formulae IA, IB, IC, ID, IE, and IF), and/or a stereoisomer, a stable isotope, or a pharmaceutically acceptable salt or solvate thereof can be administered as the sole active ingredient or in combination with at least one second active ingredient, selected, for example, from other active ingredients known to be useful for treating the target disease, such as cancers including, for example, colon cancer, gastric cancer, leukemia, lymphoma, melanoma, and pancreate cancer in a patient.

As used herein, the term “optical isomer” or “stereoisomer” refers to any of the various stereo isomeric configurations which may exist for a given compound of the present disclosure and includes geometric isomers. It is understood that a substituent may be attached at a chiral center of a carbon atom. The term “chiral” refers to molecules which have the property of non-superimposability on their mirror image partner, while the term “achiral” refers to molecules which are superimposable on their mirror image partner. The present disclosure includes enantiomers, diastereomers or racemates of the compounds. “Enantiomers” are a pair of stereoisomers that are non-superimposable mirror images of each other. A 1:1 mixture of a pair of enantiomers is a “racemic” mixture. The term is used to designate a racemic mixture where appropriate. “Diastereoisomers” are stereoisomers that have at least two asymmetric atoms, but which are not mirror-images of each other. The absolute stereochemistry is specified according to the Cahn-lngold-Prelog 1R-SJ system. When a compound is a pure enantiomer, the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Certain compounds described herein contain one or more asymmetric centers or axes and may thus give rise to enantiomers, diastereomers, and other stereoisomeric forms that may be defined, in terms of absolute stereochemistry, as (R)- or (S)-.

Depending on the choice of the starting materials and synthesis procedures, the compounds can be present in the form of one of the possible isomers or as mixtures thereof, for example as pure optical isomers, or as isomer mixtures, such as racemates and diastereoisomer mixtures, depending on the number of asymmetric carbon atoms. The present disclosure includes all such possible isomers, including racemic mixtures, diasteriomeric mixtures and optically pure forms. Optically active (R)- and (S)-isomers may be prepared using chiral synthons or chiral reagents, or resolved using conventional techniques. If the compound contains a double bond, the substituent may be E or Z configuration unless specified. If the compound contains a di-substituted cycloalkyl, the cycloalkyl substituent may have a cis- or trans-configuration, unless otherwise specified.

In many cases, the compounds of the present discloure are capable of forming acid and/or base salts by virtue of the presence of amino and/or carboxyl groups or groups similar thereto. As used herein, the terms “salt” or “salts” refers to an acid addition or base addition salt of a compound of the discloure. “Salts” include in particular “pharmaceutical acceptable salts”. The term “pharmaceutically acceptable salts” refers to salts that retain the biological effectiveness and properties of the compounds of this disclosure and, which typically are not biologically or otherwise undesirable.

Pharmaceutically acceptable acid addition salts can be formed with inorganic acids and organic acids, e.g., acetate, adipate, aluminum, ascorbate, aspartate, benzoate, besylate, bromide/hydrobromide, bicarbonate/carbonate, bisulfate/sulfate, camphorsulfonate, caproate, chloride/hydrochloride, chloroprocaine, chlortheophyllonate, citrate, edetate, calcium edetate, ethandisulfonate, ethylsulfonate, ethylene diamine, fumarate, galactarate (mucate), gluceptate, gluconate, glucuronate, glutamate, glycolate, hexyl resorcinate, hippurate, hydroiodide/iodide, hydroxynapthoate (xinafoate), isethionate, lactate, lactobionate, laurylsulfate, lithium, malate, maleate, malonate, mandelate, mesylate, methylsulphate, naphthoate, napsylate, nicotinate, nitrate, octadecanoate, oleate, oxalate, palmitate, pamoate, pantothenate, phosphate/hydrogen phosphate/dihydrogen phosphate, polygalacturonate, procaine, propionate, salicylate, sebacate, stearate, subacetate, succinate, sulfate, sulfosalicylate, tannate, tartrate, bitartrate, tosylate, triphenylacetate, and trifluoroacetate salts. Lists of additional suitable salts can be found, e.g., in REMINGTON'S PHARMACEUTICAL SCIENCES, 20th ed., Mack Publishing Company, Easton, Pa., (1985); and in HANDBOOK OF PHARMACEUTICAL SALTS: PROPERTIES, SELECTION, AND USE, by Stahl and Wermuth (Wiley-VCH, Weinheim, Germany, 2002). Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, trifluoroacetic, sulfosalicylic acid, and the like.

Pharmaceutically acceptable base addition salts can be formed with inorganic or organic bases and can have inorganic or organic counterions.

Inorganic counterions for such base salts include, for example, ammonium salts and metals from columns I to XII of the periodic table. In certain embodiments, the counterion is selected from sodium, potassium, ammonium, alkylammonium having one to four C1-C4 alkyl groups, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Suitable organic amines include isopropylamine, benzathine, cholinate, diethanolamine, diethylamine, lysine, meglumine, piperazine and tromethamine.

The pharmaceutically acceptable salts of the present disclosure can be synthesized from a basic or acidic moiety, by conventional chemical methods. Generally, such salts can be prepared by reacting free acid forms of these compounds with a stoichiometric amount of the appropriate base (such as Na, Ca, Mg, or K hydroxide, carbonate, bicarbonate or the like), or by reacting free base forms of these compounds with a stoichiometric amount of the appropriate acid. Such reactions are typically carried out in water or in an organic solvent, or in a mixture of the two. Generally, use of non-aqueous media like ether, ethyl acetate, tetrahydrofuran, toluene, chloroform, dichloromethane, methanol, ethanol, isopropanol, or acetonitrile is desirable, where practicable.

Any formula given herein is intended to represent unlabeled forms (i.e., compounds wherein all atoms are present at natural isotopic abundances and not isotopically enriched) as well as isotopically enriched or labeled forms of the compounds. Isotopically enriched or labeled compounds have structures depicted by the formulas given herein except that at least one atom of the compound is replaced by an atom of the same element but having an atomic mass or mass number different from the atomic mass or the atomic mass distribution that occurs naturally. Examples of isotopes that can be incorporated into enriched or labeled compounds of the disclosure include isotopes of hydrogen, carbon, nitrogen, oxygen, phosphorous, fluorine, and chlorine, such as ²H, ³H, ¹¹C, ¹³C, ¹⁴C, ¹⁵N, ¹⁸F, ³¹P, ³²P, ³⁵S, ³⁶Cl, and ¹²⁵I respectively. The present disclosure includes various isotopically labeled compounds as defined herein, for example those in which radioactive isotopes, such as ³H and ¹⁴C, or those in which non-radioactive isotopes, such as ²H and ¹³C, are present at levels significantly above the natural abundance for these isotopes. These isotopically labeled compounds are useful in metabolic studies (with ¹⁴C), reaction kinetic studies (with, for example ²H or 3H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies. Isotopically-labeled compounds of Formula I can generally be prepared by conventional techniques known to those skilled in the art or by processes analogous to those described in the accompanying Examples and Preparations using an appropriate isotopically-labeled reagents in place of the non-labeled reagent previously employed.

Further, substitution with heavier isotopes, particularly deuterium (i.e., ²H or D) may afford certain therapeutic advantages resulting from greater metabolic stability, for example increased in vivo half-life or reduced dosage requirements or an improvement in therapeutic index. It is understood that deuterium in this context is regarded as a substituent of a compound of the Formula I if it is incorporated at substantially above the level of natural isotopic abundance. The present disclosure includes isotopically enriched versions of the compounds, e.g., deuterated versions as well as non-deuterated versions. Deuterated versions may be deuterated at a single site, or at multiple sites.

The degree of incorporation of such an isotope in an isotopically-enriched compound, particularly deuterium, may be defined by the isotopic enrichment factor. The term “isotopic enrichment factor” as used herein means the ratio between the isotopic abundance of a specified isotope in a sample, and the natural abundance of the isotope in a non-enriched sample. If a substituent in a compound of this disclosure is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

Pharmaceutically acceptable solvates in accordance with the present disclosure include those wherein the solvent of crystallization may be isotopically substituted, e.g. D₂O, d₆-acetone, d₆-DMSO, as well as solvates with non-enriched solvents.

As used herein, the term “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drug stabilizers, binders, excipients, disintegration agents, lubricants, sweetening agents, flavoring agents, dyes, and the like and combinations thereof, as would be known to those skilled in the art (see, for example, REMINGTON'S PHARMACEUTICAL SCIENCES, 18th Ed. Mack Printing Company, 1990, pp. 1289-1329). Except insofar as any conventional carrier is incompatible with the active ingredient, its use in the therapeutic or pharmaceutical compositions is contemplated.

The term “a therapeutically effective amount” of a compound of the present disclosure refers to an amount of the compound of the present disclosure that will elicit the biological or medical response of a subject, for example, reduction or inhibition of an enzyme or a protein activity, or ameliorate symptoms, alleviate conditions, slow or delay disease progression, or prevent a disease, etc. In one non-limiting embodiment, the term “therapeutically effective amount “refers to the amount of the compound of the present disclosure that, when administered to a subject, is effective to (1) at least partially alleviate, inhibit, prevent and/or ameliorate a condition, or a disorder or a disease (i) mediated by a kinase such as SHP2 or (ii) associated with activity of a kinase such as SHP2, or (iii) characterized by activity (normal or abnormal) of SHP2; or (2) reduce or inhibit the activity of SHP2 or (3) reduce or inhibit the expression of SHP2.

In another non-limiting embodiment, the term “a therapeutically effective amount” refers to the amount of the compound of the present disclosure that, when administered to a cell, or a tissue, or a non-cellular biological material, or a medium, is effective to at least partially reduce or inhibit the activity of SHP2, or at least partially reduce or inhibit the expression of SHP2.

As used herein, the term “subject” refers to an animal. Typically the animal is a mammal. A subject also refers to for example, primates (e.g., humans, male or female), cows, sheep, goats, horses, dogs, cats, rabbits, rats, mice, fish, birds and the like. In certain embodiments, the subject is a primate. In specific embodiments, the subject is a human.

As used herein, the term “inhibit”, “inhibition” or inhibiting” refers to the reduction or suppression of a given condition, activity, effect, symptom, or disorder, or disease, or a significant decrease in the baseline activity of a biological activity or process.

As used herein, the term “treat”, “treating” or “treatment” of any disease or disorder refers in one embodiment, to ameliorating the disease or disorder (i.e., slowing or arresting or reducing the development of the disease or at least one of the clinical symptoms thereof). In another embodiment, “Treat”, “treating” or “treatment” refers to alleviating or ameliorating at least one physical parameter including those which may not be discernible by the patient. In yet another embodiment, “Treat”, “treating” or “treatment” refers to modulating the disease or disorder, either physically, (e.g., stabilization of a discernible symptom), physiologically, (e.g., stabilization of a physical parameter), or both. In yet another embodiment, “Treat”, “treating” or “treatment” refers to delaying the development or progression of the disease or disorder.

As used herein, a subject is “in recognized need of” a treatment if such subject would be expected to benefit biologically, medically or in quality of life from such treatment.

As used herein, the term “a” “an” “the” and similar terms used in the context of the present disclosure (especially in the context of the claims) are to be construed to cover both the singular and plural unless otherwise indicated herein or clearly contradicted by the context.

All methods described herein can be performed in any suitable order unless otherwise indicated herein or otherwise clearly contradicted by context. The use of any and all examples, or exemplary language (e.g. “such as”) provided herein is intended merely to better illuminate the present disclosure and does not pose a limitation on the scope of the present disclosed otherwise claimed.

Any asymmetric atom (e.g., carbon or the like) of the compound(s) of the present disclosure can be present in racemic or enantiomerically enriched, for example, the (R)-, (S)- or (R,S)-configuration. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess of either the (R)- or (S)-configuration; i.e., for optically active compounds, it is often, for example, to use one enantiomer to the substantial exclusion of the other enantiomer. Substituents at atoms with carbon-carbon double bonds may, where possible, be present in cis- (Z)- or trans-(E)-form, and both are included in the present disclosure unless otherwise indicated.

Accordingly, as used herein a compound of the present disclosure can be in the form of one of the possible isomers, rotamers, atropisomers, or as a mixture thereof, for example, as substantially pure geometric (cis or trans) isomers, diastereomers, optical isomers (antipodes), racemates or mixtures thereof. “Substantially pure” or “substantially free of other isomers” as used herein means the product contains less than 5%, and, such as, less than 2%, of other isomers relative to the amount of the preferred isomer, by weight.

Any resulting mixtures of isomers can be separated on the basis of the physicochemical differences of the constituents, into the pure or substantially pure geometric or optical isomers, diastereomers, racemates, for example, by chromatography and/or fractional crystallization.

Any resulting racemates of final products or intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds of the present disclosure into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic products can also be resolved by chiral chromatography, e.g., high pressure liquid chromatography (HPLC) using a chiral adsorbent.

Furthermore, the compounds of the present disclosure, including their salts, can also be obtained in the form of their hydrates, or include other solvents used for their crystallization. The compounds of the present disclosure may inherently or by design form solvates with pharmaceutically acceptable solvents (including water); therefore, it is intended that the present disclosure embraces both solvated and unsolvated forms. The term “solvate” refers to a molecular complex of a compound of the present disclosure (including pharmaceutically acceptable salts thereof) with one or more solvent molecules. Such solvent molecules are those commonly used in the pharmaceutical art, which are known to be innocuous to the recipient, e.g., water, ethanol, and the like. The term “hydrate” refers to the complex where the solvent molecule is water.

Schemes 1-2 show general methods for preparing the compounds of the present disclosure as well as intermediates. The detailed description and syntheses are disclosed in the Examples below. Those skilled in the art will be able to find other synthetic methods or modify the methods described below using conventional chemistry for preparing suitable compounds encompassed by Formula I. So these methods are equally applicable to preparation of compounds with other emboddiments. Although specific starting materials and reagents are depicted in the Schemes and discussed below, other starting materials and reagents can be easily substituted to provide a variety of compounds and/or reaction conditions.

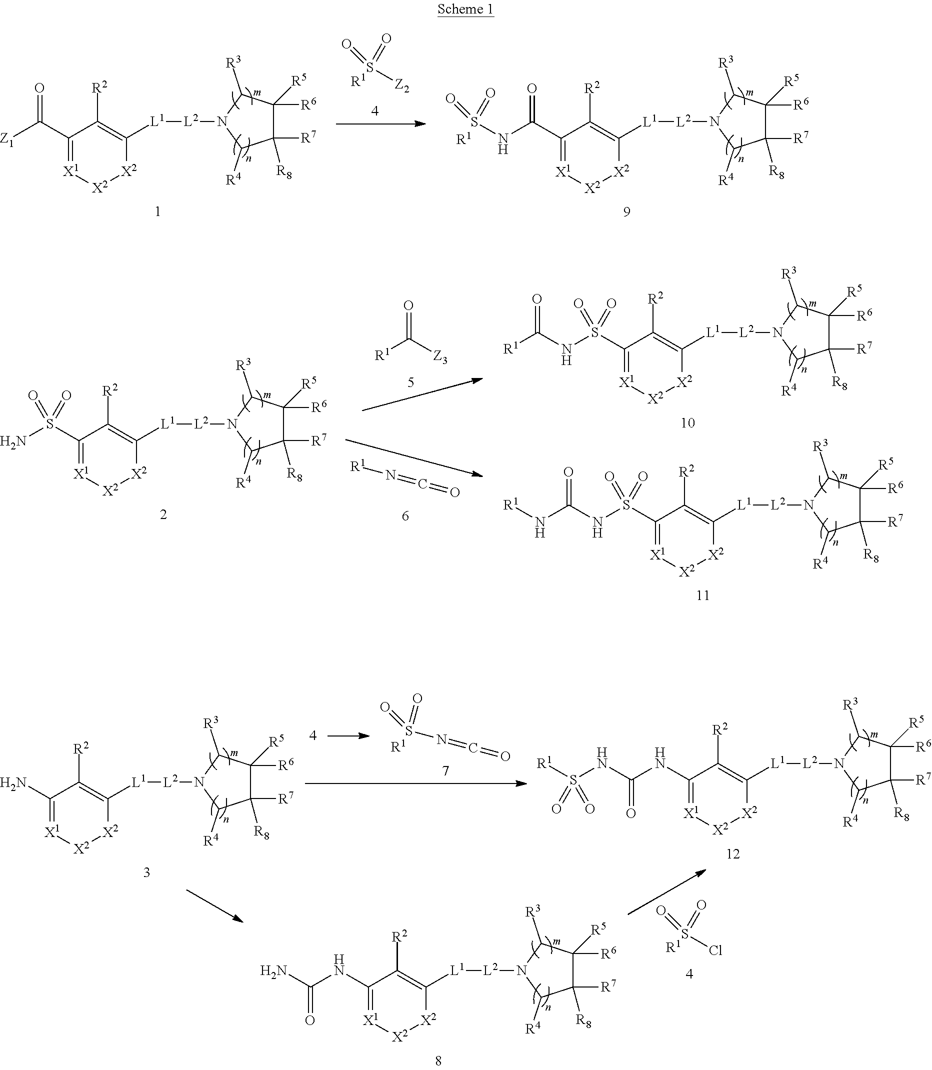

Compounds of formulas 9-12 of Formula I can be made by general synthetic method as illustrated in Scheme 1. Compounds of formula 1 (where Z₁ is OH or NH₂) can react with compounds of formula 4 (where Z₂ is NH₂ or Cl) under various conditions to give acylsulfonamides of formula 9, which are compounds of Formula I. For example, when Z₁ is OH and Z₂ is NH₂, compounds of formula 1 can react with compounds of formula 4 to give compounds of formula 9 (i.e., compounds of Formula I) using dehydrating agents such as EDCI and base such as TEA, DIPEA or DIPEA in a solvent such as DMC, acetonitrile, DMF or THF. When Z₁ is NH₂ and Z₂ is Cl, the coupling of compounds of formula 1 and compounds of formula 4 using base such as TEA, DIPEA or DMAP in a solvent such as DMC, acetonitrile, DMF or THF, to give compounds of formula 9 (i.e., compounds of Formula I). The reverse acylsulfonamides of formula 10 of Formula I can be prepared similarly between compounds of formula 2 and compounds of formula 5 (Z₃ is OH or Cl).

Sulfonylureas of formula 11 of Formula I can be made by reaction of compounds of formula 2 with isocyanates of formula 6 under basic conditions such as NaH, DBU, NaOH or K₂CO₃ in various solvent such as THF, DCM, DMF or acetone. The reverse sulfonylureas of formula 12 of Formula I can be made by coupling of compounds of formula 3 and sulfonyl isocyanates of formula 7, which can be made in situ from sulfonylchlorides of formula 4 (Z₂ is Cl) with sodium cyanate and a base such as pyridine in a solvent such as acetonitrile. Alternatively, amines of formula 3 can first converted to ureas of formula 8 under various conditions, such as reacting with sodium cyanate in acetic acid and water at room temperature; or reacting with triphosgene and TFA in toluene, followed by reacting with aqueous ammonia. The ureas of formula 8 can be converted to the sulfonylureas of formula 12 by reaction with sulfonyl chloride in an alkaline solution such as sodium hydroxide under elevated temperature.

The compounds of Formula I where L¹ is S and L² is heteroaryl group can be synthesized by general synthetic method as illustrated in Scheme 2. The heteroarenethiols of formula 15 can be made by a two step process consisting of reaction of compounds of formula 12 (Z₄ is Cl, Br, or —OTf) with 2-ethylhexyl 3-mercaptopropanoate of formula 14 under Pd catalyzed condition (such as Pd₂(dba)₃, Xantphos and DIPEA in THF or dioxane) and followed by treatment with t-BuOK in THF. The heteroarenethiols 15 can react with compound of formula 16 (Z₅ and Z₆ are independently Cl, Br, or OTf) under Pd catalyzed condition (such as Pd₂(dba)₃, Xantphos and DIPEA in THF or dioxane) to provide compounds of formula 17. Coupling of compounds of formula 17 and amines of formula 18 by nucleophilic substitution reaction or by Buchwald-Hartwig reaction to give compounds of formula 19. The compounds of formula 19 where Y is NH₂C(O)—, is HO₂C—, NH₂S(O₂)—, or NH₂(CO)NH—, obtained via functional group transformation reactions can be converted to the final compounds of formulas 20-24 of Formula I by methods described in Scheme 1.

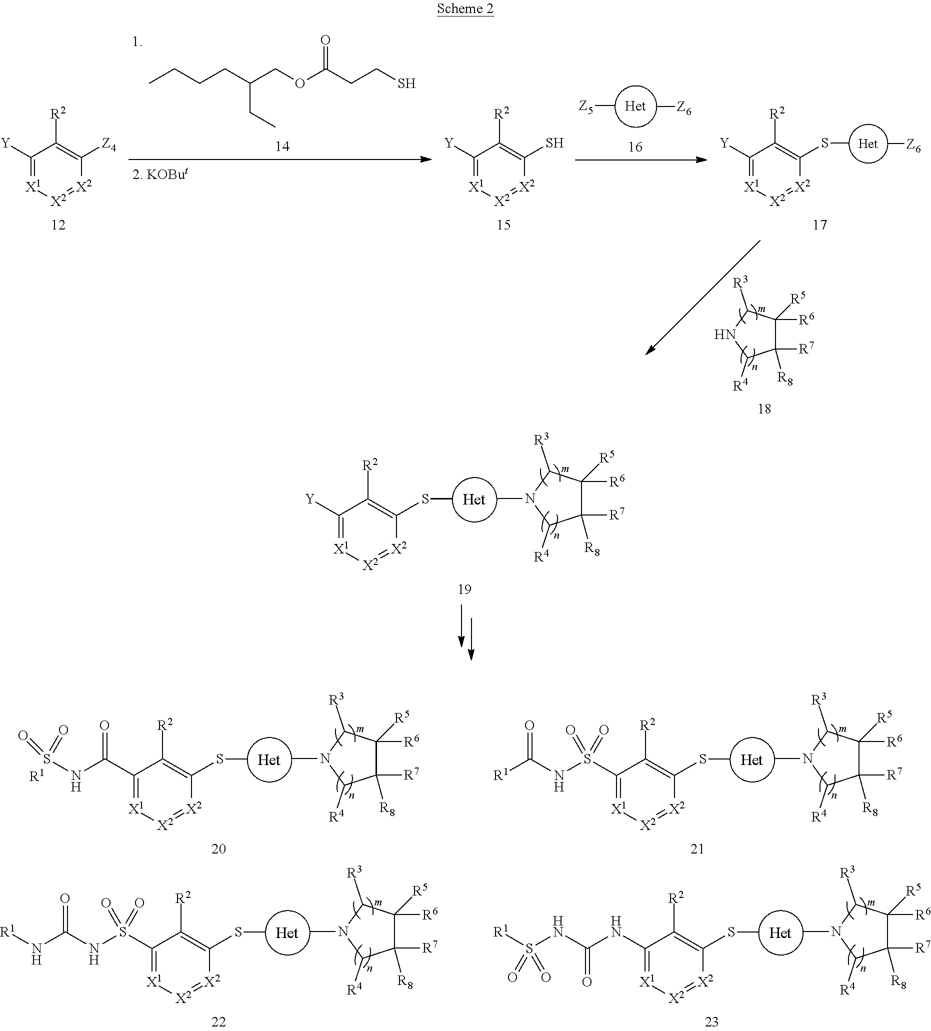

### EXAMPLES

The following examples illustrate certain embodiments of the present disclosure and how to make and use them. They are not intended to limit the scope of the invention. Those of skill in the art will readily recognize a variety of noncritical parameters and conditions which can be changed or modified to yield essentially the same results. The example compounds below were found to be inhibitors of SHP2 according to one or more of the assays described herein.

In the following examples, the abbreviations below are used:
BINAP 2,2′-Bis(diphenylphosphino)-1,1′-binaphthyl
BOC tert-Butyloxycarbonyl
B₂(Pin)₂ Bis(pinacolato)diboron
BTEAC Benzyltriethylammonium chloride
CDI Carbonyldiimidazole
dba dibenzylideneacetone
DCE 1,2-Dichloroethane
DCM Dichloromethane
DHP Dihydropyran
DIAD Diisopropyl azodicarboxylate
DIPEA di-isopropylethylamine
DMA Dimethylacetamide
DMAP 4-Dimethylaminopyridine
DMF Dimethylformamide
DMSO Dimethylsulfoxide
Dppf 1,1′Bis(diphenylphosphino)ferrocene
EDCI N-Ethyl-N′-(3-dimethylaminopropyl)carbodiimide hydrochloride
EDTA Ethylenediaminetetraacetic acid
EtOAc Ethyl acetate
EtOH Ethanol
HATU 1-[Bis(dimethylamino)methylene]-1H-1,2,3-triazolo[4,5-b]pyridinium 3-oxid hexafluorophosphate
KHMDS Potassium hexamethyldisilazane
LiHMDS Lithium hexamethyldisilazane
LG Leaving group
MeOH Methanol
MsCl Methanesulfonyl chloride
MTBE Methyl tert-butyl ether
NMM N-methylmorpholine
NMP N-methylpyrrolidinone
Pd₂dba₃ Tris(dibenzylidenacetone)palladium
Pd(dppf)Cl₂ [1,1′Bis(diphenylphosphino)ferrocene]dichloropalladium(II)
PE Petroleum ether
PG Protecting group
PPTS Pyridinium p-toluenesulfonate
Prep-TLC Preparative Thin layer chromatography
PTSA p-toluenesulfonic acid
TBAF tetra-n-butylammonium fluoride
TBDMSCl t-Butyldimethylsilyl chloride
TEA Triethylamine
TES Triethylsilyl
TFA Trifluoacetic acid
Tf Triflyl
Tf₂O Trifluoromethanesulfonic anhydride
TLC Thin layer chromatography
THF Tetrahydrofuran
THP tetrahydropyran
TMS Trimethylsilyl
TosMIC Toluenesulfonylmethyl isocyanide
Xantphos 4,5-Bis(diphenylphosphino)-9,9-dimethylxanthene
XPhos 2-Dicyclohexylphosphino-2′,4′,6′-triisopropylbiphenyl

### Example 1 (Method 1)

### Preparation of N-((3-((5-(4-amino-4-methylpiperidin-1-yl)pyrazin-2-yl)thio)-2-chlorophenyl)carbamoyl)benzenesulfonamide

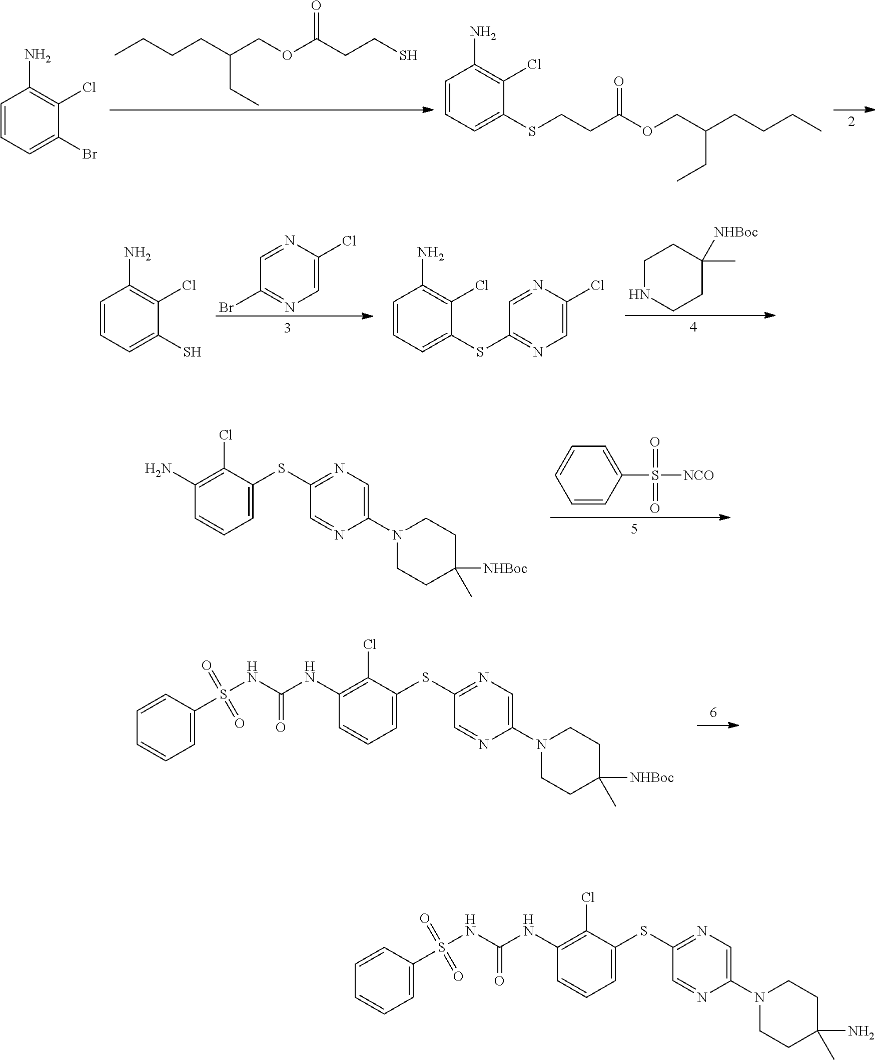

### Step 1. 2-ethylhexyl 3-((3-amino-2-chlorophenyl)thio)propanoate

A solution of 3-bromo-2-chloroaniline (1.0 g, 4.84 mmol), 2-ethylhexyl 3-mercaptopropanoate (1.16 g, 5.33 mmol), Pd₂(dba)₃ (221.6 mg, 0.242 mmol), Xantphos (280 mg, 0.484 mmol), and DIPEA (1.25 g, 9.68 mmol) in dry dioxane (10 mL) was charged with N₂ and stirred at 110′° C. for 8 hours when LC/MS showed the reaction was completed. The mixture was filtered. The filtrate was diluted with EtOAc (300 mL), washed with H₂O (50 mL×2) and brine (50 mL), died over Na₂SO₄, filtered off and concentrated. The residue was purified by flash column chromatography on silica gel (PE:EtOAc=15:1-10:1) to give the title compound (1.6 g, yield: 100%).

### Step 2. 3-amino-2-chlorobenzenethiol

To a solution of the product of Step 1 above (1.6 g, 4.84 mmol) in THF (13.3 mL) at −78° C. under N₂ was added a solution of t-BuOK in THF (1.63 g in 14.5 mL) slowly. After the addition was completed, the mixture was stirred at −78° C. for 20 min when LC/MS showed the reaction was completed. The reaction was quenched with aqueous K₂CO₃ (2 N, 2 mL) and concentrated to dryness. The resulting residue was taken up in aqueous K₂CO₃ (2 N, 60 mL), which was extracted with MTBE (50 mL×2). The aqueous phase was acidified with 6 N HCl to pH of 4 and extracted with isopropyl alcohol/DCM (1/3, 100 mL×2). The combined extracts were washed with brine (30 mL), died over Na₂SO₄, filtered off and concentrated to give the title compound (623 mg, yield: 81%).

### Step 3. 2-chloro-3-((5-chloropyrazin-2-yl)thio)aniline

A solution of the product of Step 2 above (623 mg, 3.9 mmol), 2-bromo-5-chloropyrazine (830 mg, 4.3 mmol), Pd₂(dba)₃ (178.6 mg, 0.195 mmol), Xantphos (226 mg, 0.39 mmol), DIPEA (1.008 g, 7.8 mmol) in dioxane (6 mL) was charged with N₂ and stirred at 110° C. for 6 hours when LC/MS showed the reaction was completed. The mixture was extracted with EtOAc (100 mL), washed with H₂O (30 mL×2) and brine (30 mL×2), dried over Na₂SO₄, filtered off and concentrated. The residue was purified by a silica gel column (PE:EtOAc=10:−6:1) to give the title compound (487 mg, yield: 46%).

### Step 4. tert-butyl (1-(5-((3-amino-2-chlorophenyl)thio)pyrazin-2-yl)-4-methylpiperidin-4-yl)carbamate

A solution of the product of Step 3 above (100 mg, 0.367 mmol), tert-butyl (4-methylpiperidin-4-yl)carbamate (157 mg, 0.735 mmol) and DIPEA (237 mg, 1.835 mmol) in NMP (2 mL) was charged with N₂ and stirred at 130° C. for 10 hours when LC/MS showed the reaction was completed. Purification of the reaction mixture by the reverse phase flash column chromatography (MeOH/H₂O) gave the title compound (148 mg, yield: 90%).

### Step 5. tert-butyl (1-(5-((2-chloro-3-(3-(phenylsulfonyl)ureido)phenyl)thio)pyrazin-2-yl)-4-methylpiperidin-4-yl)carbamate

A solution of the product of Step 4 above (148 mg, 0.33 mmol), benzenesulfonyl isocyanate (60 mg, 0.33 mmol), NMM (0.04 mL, 0.33 mmol) in dry THF (5 mL) at RT was charged with N₂ and stirred at RT overnight when LC/MS showed the reaction wasn't completed. More benzenesulfonyl isocyanate (0.5 eq) was added. The mixture was stirred at RT for another 4 hours. More benzenesulfonyl isocyanate (0.5 eq) was added and the mixture was stirred at RT for another 3 hours until LC/MS showed the reaction was completed. The mixture was extracted with EtOAc (100 mL), washed with H₂O (50 mL×2) and brine (50 mL), dried over Na₂SO₄, filtered off and concentrated to give the title compound (275 mg, crude product), which was used in the next step without any further purification.

### Step 6. N-((3-((5-(4-amino-4-methylpiperidin-1-yl)pyrazin-2-yl)thio)-2-chlorophenyl)carbamoyl)benzenesulfonamide

To a solution of the product of Step 5 above (100 mg, 0.16 mmol) in DCM/MeOH (0.8 mL/0.2 mL) at RT was added 4 N HCl/dioxane (0.5 mL) at 0° C. The mixture was charged with N₂ and stirred at RT for 4 hours when LC/MS showed the reaction was completed. The mixture was concentrated and the residue was purified by reverse phase flash column chromatography (MeOH/H₂O) to give the title compound (19 mg, yield: 22%). MS (ESI) m/z: 533.2 [M+1]+; 1H NMR (400 MHz, DMSO-d6) δ 8.40 (s, 1H), 8.18 (s, 1H), 8.04 (s, 1H), 7.75 (s, 5H), 7.49 (s, 1H), 7.37 (s, 3H), 7.02 (s, 1H), 6.44 (s, 1H), 4.06 (s, 2H), 3.35 (s, 2H), 1.70 (s, 4H), 1.34 (s, 3H).

### Example 2 (Method 2)

### Preparation of N-((3-((5-(4-(aminomethyl)-4-methylpiperidin-1-yl)pyrazin-2-yl)thio)-2-chlorophenyl)carbamoyl)benzenesulfonamide

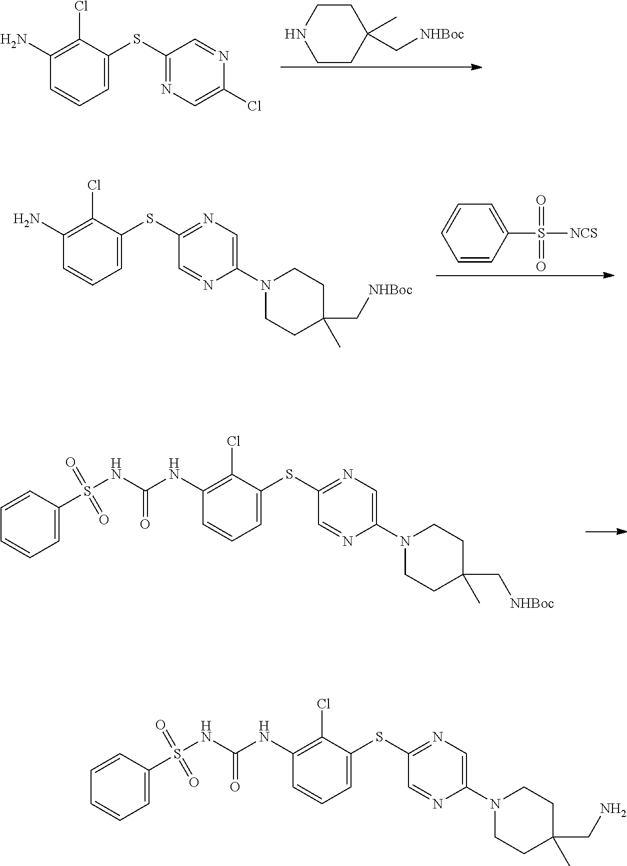

### Step 1. tert-butyl ((1-(5-((3-amino-2-chlorophenyl)thio)pyrazin-2-yl)-4-methylpiperidin-4-yl)methyl)carbamate

A solution the product of Step 3 of Example 1 (100 mg, 0.367 mmol), tert-butyl (4-methylpiperidin-4-yl)carbamate (167 mg, 0.735 mmol), DIPEA (237 mg, 1.835 mmol) in NMP (2 mL) at RT was charged with N₂ and stirred at 130° C. for 10 hours. LC/MS showed the reaction was completed. Purification of the reaction mixture by flash column chromatography (MeOH/H₂O) gave the title compound (147 mg, yield: 86%).

### Step 2. tert-butyl ((1-(5-((2-chloro-3-(3-(phenylsulfonyl)ureido)phenyl)thio)pyrazin-2-yl)-4-methylpiperidin-4-yl)methyl)carbamate

A solution of the product of Step 1 above (147 mg, 0.32 mmol), benzenesulfonyl isocyanate (58 mg, 0.32 mmol), NMM (0.035 mL, 0.32 mmol) in dry THF (5 mL) was charged with N₂ and stirred at RT overnight. More benzenesulfonyl isocyanate (0.5 eq) was added. The mixture was stirred at RT for 4 hours. More benzenesulfonyl isocyanate (0.5 eq) was added. The mixture was stirred at RT for another 3 hours when LC/MS showed the reaction was completed. The reaction mixture was extracted with EtOAc (100 mL), washed with H₂O (50 mL×2) and brine (50 mL), dried over Na₂SO₄, filtered off and concentrated to give the title compound (321 mg, crude).

### Step 3. N-((3-((5-(4-(aminomethyl)-4-methylpiperidin-1-yl)pyrazin-2-yl)thio)-2-chlorophenyl)carbamoyl)benzenesulfonamide

To a solution of the product of Step 2 above (100 mg, 0.15 mmol) in DCM/MeOH (0.8 mL/0.2 mL) was added 4 N HCl/dioxane (0.5 mL) at 0° C. under N₂. The mixture was stirred at RT for 4 hours when LC/MS showed the reaction was completed. The mixture was concentrated. The residue was purified by reverse phase flash column chromatography (MeOH/H₂O) to give the title compound (22 mg, yield: 27%). MS (ESI) m/z: 547.2 [M+1]+; 1H NMR (400 MHz, DMSO-d6) δ 8.37 (s, 1H), 8.16 (s, 1H), 8.03 (d, J=7.7 Hz, 1H), 7.75 (s, 2H), 7.51 (m, 4H), 7.37 (s, 3H), 7.01 (s, 1H), 6.40 (d, J=7.7 Hz, 1H), 3.87 (s, 2H), 3.39 (s, 2H), 2.76 (s, 2H), 1.46 m, 4H), 1.05 (s, 3H).

### Example 3 (Method 3)

### Preparation of 3-((5-(4-amino-4-methylpiperidin-1-yl)pyrazin-2-yl)thio)-2-chloro-N-(phenylsulfonyl)benzamide

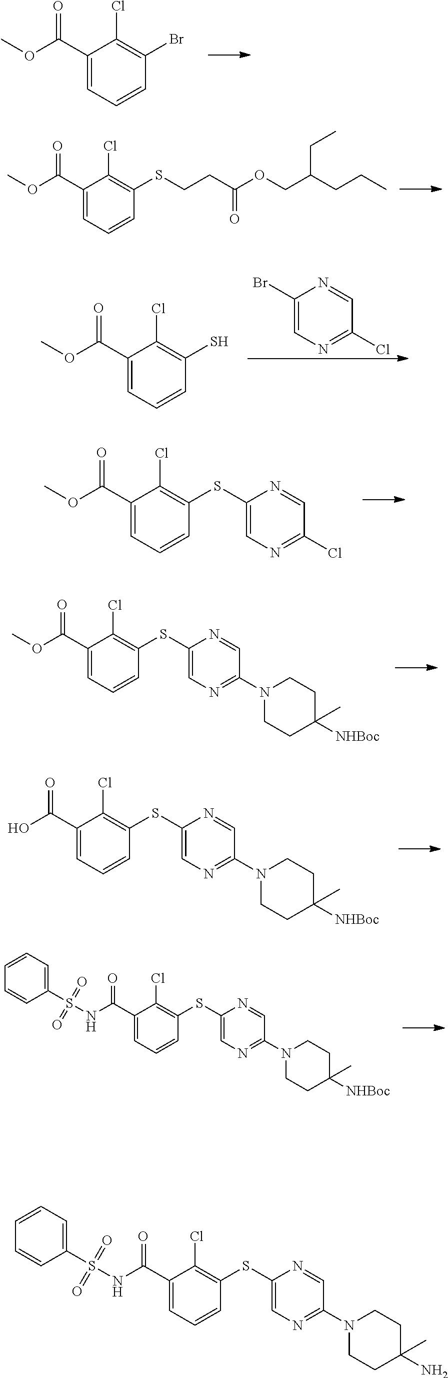

### Step 1. methyl 2-chloro-3-((3-((2-ethylpentyl)oxy)-3-oxopropyl)thio)benzoate

A solution of methyl 3-bromo-2-chlorobenzoate (2.0 g, 8.02 mmol), 2-ethylhexyl 3-mercaptopropanoate (1.93 g, 8.8 mmol), Pd₂(dba)₃ (369 mg, 0.4 mmol), Xantphos (510 mg, 0.88 mmol), and DIPEA (2.08 g, 16 mmol) in dry dioxane (20 mL) was charged with N₂ and stirred at 110° C. for 3 hours when LC/MS showed the reaction was completed. The mixture was filtered off. The filtrate was diluted with H₂O (20 mL) and extracted with EtOAc (20 mL×2). The combined organic layers were washed with H₂O (20 mL) and brine (20 mL), dried over Na₂SO₄, filtered off and concentrated. The residue was purified by flash column chromatographya on silica gel (PE:EtOAc=50:1 to 20:1) to give the title compound (1.01 g, yield: 66%).

### Step 2. methyl 2-chloro-3-mercaptobenzoate

To a solution of the product of Step 1 above (1.03 g, 2.66 mmol) in THF (10 mL) at −78° C. under N₂ was added t-BuOK/THF (598 mg/6 mL) slowly. After the addition was completed, the mixture was stirred at −78° C. for 20 min when LC/MS showed the reaction was completed. The reaction was quenched with saturated aqueous NH₄Cl (3 mL) and concentrated. The resulting residue was poured into a separatory funnel containing saturated aqueous K₂CO₃ (10 mL) and extracted with MTBE (20 mL×2). The combined extracts were washed with brine (30 mL), died over Na₂SO₄, filtered off and concentrated. The residue was purified by flash column chromatography in silica gel to give the title compound (269 mg, yield: 49%).

### Step 3. methyl 2-chloro-3-((5-chloropyrazin-2-yl)thio)benzoate

A solution of the product of Step 2 above (269 mg, 1.33 mmol), 2-bromo-5-chloropyrazine (308 mg, 1.59 mmol), Pd₂(dba)₃ (60.8 mg, 0.066 mmol), Xantphos (84.5 mg, 0.146 mmol), and DIPEA (343 mg, 2.65 mmol) in dioxane (3 mL) was charged with N₂ and stirred at 110° C. for 3 hours when LC/MS showed the reaction was completed. The mixture mixture was purified by reverse phase flash column chromatography (MeOH/H₂O) to give the title compound (149 mg, yield: 35%).

### Step 4. methyl 3-((5-(4-((tert-butoxycarbonyl)amino)-4-methylpiperidin-1-yl)pyrazin-2-yl)thio)-2-chlorobenzoate

A solution the product of Step 3 above (149 mg, 0.473 mmol), tert-butyl (4-methylpiperidin-4-yl)carbamate (202 mg, 0.946 mmol) and DIPEA (184 mg, 1.42 mmol) in NMP (2 mL) was charged with N₂ and stirred at 130° C. for 4 hours until LC/MS showed the reaction was completed. Purification of the reaction mixture by the reverse phase flash column chromatography (MeOH/H₂O) gave the title compound (90 mg, yield: 38%).

### Step 5. 3-((5-(4-((tert-butoxycarbonyl)amino)-4-methylpiperidin-1-yl)pyrazin-2-yl)thio)-2-chlorobenzoic acid

To a solution of the product of Step 4 above (90 mg, 0.183 mmol) in MeOH/H₂O (2 mL/0.4 mL) was added lithium hydroxide hydrate (15.4 mg, 0.365 mmol). The mixture was stirred at rt for 3 h, diluted with water, and concentrated to remove MeOH. The aqueous phase was acidified with aqueous citric acid (10%) to pH of 4 and extracted with DCM/MeOH (10/1, 20 mL). The extract was dried over Na₂SO₄, filtered off and concentrated to give the title compound (84 mg, yield: 94%), which was used in the next step without any further purification.

### Step 6. tert-butyl (1-(5-((2-chloro-3-((phenylsulfonyl)carbamoyl)phenyl)thio)pyrazin-2-yl)-4-methylpiperidin-4-yl)carbamate

A solution of the product of Step 5 above (84 mg, 0.175 mmol), benzenesulfonamide (27 mg, 0.175 mmol), DMAP (42 mg, 0.35 mmol), and EDCI (67 mg, 0.35 mmol) in DCM (8 mL) was stirred at rt overnight. The mixture was diluted DCM (20 mL), washed with water (5 mL) and aqueous citric acid (10%, 10 mL), dried over Na₂SO₄, filtered off and concentrated. The residue was purified by flash column chromatography on silica gel (DCM/MeOH=100/1 to 40/1) to give the title compound (77 mg, yield: 71%).

### Step 7. 3-((5-(4-amino-4-methylpiperidin-1-yl)pyrazin-2-yl)thio)-2-chloro-N-(phenylsulfonyl)benzamide

To a solution of the product of Step 6 above (77 mg, 0.125 mmol) in DCM/MeOH (4 mL/1 mL) was added 4N HCl/dioxane (1.5 mL). The mixture was stirred at rt for 2 hours. The reaction mixture was concentrated to dryness. The residue was purified by reverse phase flash column chromatography (MeOH/H₂O) to give the title compound (11.1 mg, 17%). MS (ESI) m/z: 518.3 [M+1]+; 1H NMR (400 MHz, DMSO-d6) δ 8.41 (s, 1H), 8.20 (s, 1H), 7.94 (s, 3H), 7.81 (s, 2H), 7.45 (m, 3H), 7.19-7.10 (m, 2H), 6.93-6.85 (m, 1H), 4.05 (d, J=12.8 Hz, 2H), 3.34 (m, 2H), 1.70 (m, 4H), 1.35 (s, 3H).

### Example 4 (Method 4)

### Preparation of N-((3-((3-amino-5-(4-amino-4-methylpiperidin-1-yl)pyrazin-2-yl)thio)-2-chlorophenyl)carbamoyl)benzenesulfonamide

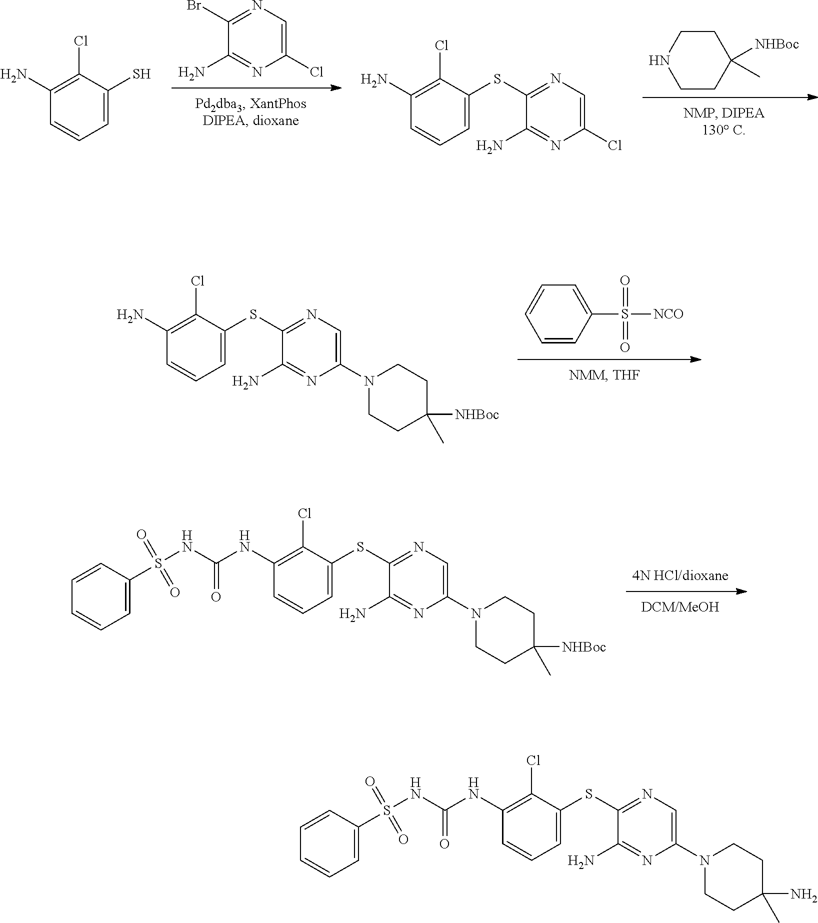

### Step 1. 3-((3-amino-2-chlorophenyl)thio)-6-chloropyrazin-2-amine

A solution of 3-amino-2-chlorobenzenethiol (200 mg, 1.25 mmol), 3-bromo-6-chloropyrazin-2-amine (287 mg, 1.37 mmol), Pd₂dba₃ (57 mg, 0.0625 mmol), Xantphos (72 mg, 0.125 mmol), DIPEA (323 mg, 2.5 mmol) in dioxane (2 mL) was charged with N₂ and stirred at 110° C. for 8 hours. LC/MS showed the reaction was completed. The mixture was extracted with EtOAc (100 mL), washed with H₂O (30 mL×2) and brine (30 mL×2), dried over Na₂SO₄, filtered off and concentrated. The residue was purified by a silica gel column (PE:EtOAc=10:1-4:1) to give the title compound (200 mg, yield: 56%).

### Step 2. tert-butyl (1-(6-amino-5-((3-amino-2-chlorophenyl)thio)pyrazin-2-yl)-4-methylpiperidin-4-yl)carbamate

A solution of the product of Step 1 above (100 mg, 0.348 mmol), tert-butyl (4-methylpiperidin-4-yl)carbamate (149 mg, 0.696 mmol) and DIPEA (135 mg, 1.044 mmol) in NMP (2 mL) was charged with N₂ and stirred at 130° C. for 10 hours. LC/MS showed the reaction was completed. Purification of the reaction mixture by the reverse phase FCC (MeOH/H₂O) gave the title compound (134 mg, yield: 83%).

### Step 3. tert-butyl (1-(6-amino-5-((2-chloro-3-(3-(phenylsulfonyl)ureido)phenyl)thio)pyrazin-2-yl)-4-methylpiperidin-4-yl)carbamate

A solution of the product of Step 2 above (134 mg, 0.288 mmol), benzenesulfonyl isocyanate (53 mg, 0.288 mmol), NMM (0.032 mL, 0.288 mmol) in dry THF (5 mL) at RT was charged with N₂ and stirred at RT overnight. LC/MS showed the reaction wasn't completed and more benzenesulfonyl isocyanate (0.5 eq) was added. The mixture was stirred at RT for 4 hours and LC/MS showed the reaction still wasn't completed. More benzenesulfonyl isocyanate (0.5 eq) was added and the mixture was stirred at RT for 3 hours. LC/MS showed the reaction was completed. The mixture was extracted with EtOAc (100 mL), washed with H₂O (50 mL×2) and brine (50 mL), dried over Na₂SO₄, filtered off and concentrated. The residue was purified by the reverse phase FCC (MeOH/H₂O) to give the title compound (111 mg, yield: 59%).

### Step 4. N-((3-((3-amino-5-(4-amino-4-methylpiperidin-1-yl)pyrazin-2-yl)thio)-2-chlorophenyl)carbamoyl)benzenesulfonamide

To a solution of the product of Step 3 above (111 mg, 0.171 mmol) in DCM/MeOH (4 mL/1 mL) at RT was added 4 N HCl/dioxane (0.5 mL) at 0° C. The mixture was charged with N₂ and stirred at RT overnight. LC/MS showed the reaction was completed. The mixture was concentrated and the residue was purified by reverse phase FCC (MeOH/H₂O) to give the title compound (21 mg, yield: 22%). MS (ESI) m/z: 548.3 [M+1]+; ¹H NMR (400 MHz, DMSO-d6) δ 7.95 (d, J=8.3 Hz, 1H), 7.75 (b, 5H), 7.61 (s, 1H), 7.47 (s, 1H), 7.38-7.33 (m, 3H), 6.95 (t, J=8.1 Hz, 1H), 6.15-6.04 (m, 3H), 4.00 (d, J=13.6 Hz, 2H), 3.25 (m, 2H), 1.67 (s, 4H), 1.34 (s, 3H).

### Example 5 (Method 5)

### Preparation of 3-((3-amino-5-(4-(aminomethyl)-4-methylpiperidin-1-yl)pyrazin-2-yl)thio)-2-chloro-N-(phenylsulfonyl)benzamide

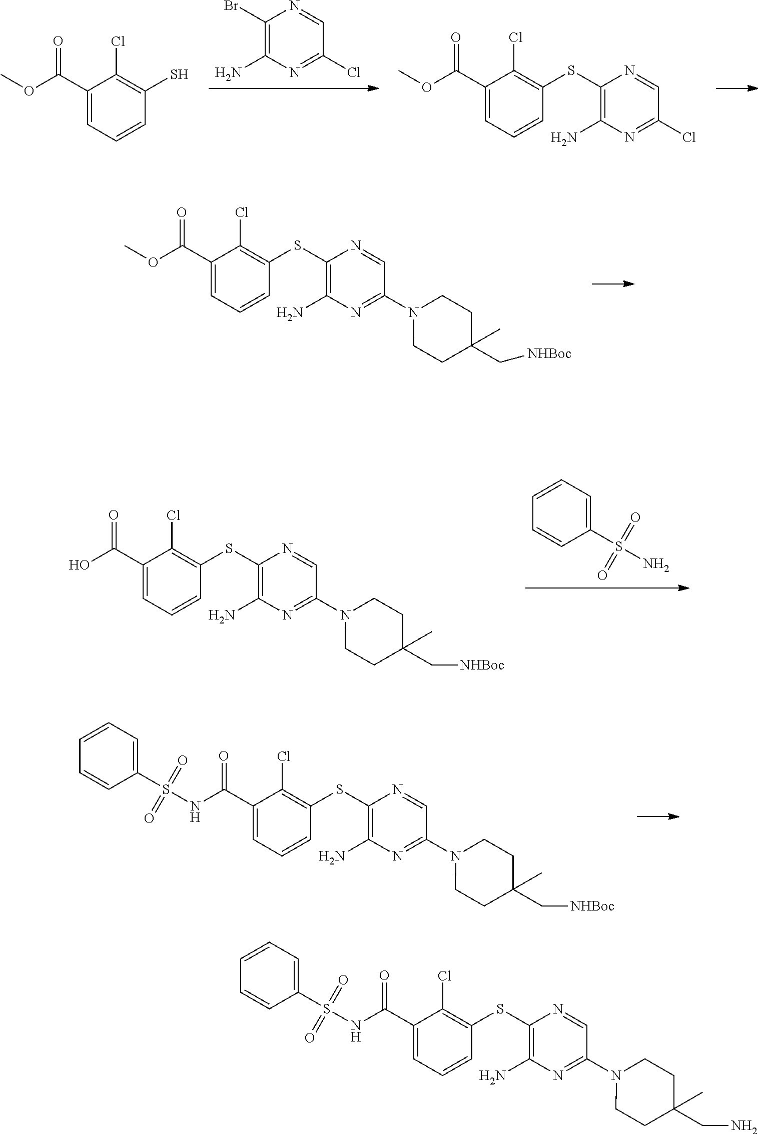

### Step 1. methyl 3-((3-amino-5-chloropyrazin-2-yl)thio)-2-chlorobenzoate

A solution of the product of Step 2 in example 3 (4.05 g, 20 mmol), 3-bromo-6-chloropyrazin-2-amine (5.0 g, 24 mmol), Pd₂dba₃ (915 mg, 1.0 mmol), Xantphos (1.272 g, 2.2 mmol), and DIPEA (5.17 g, 40 mmol) in dioxane (40 mL) was charged with N₂ and stirred at 110° C. for 3 hours when LC/MS showed the reaction was completed. The mixture was diluted with water (30 mL) and extracted with EtOAc (50 mL×3). The combined organics were dried over Na₂SO₄, filtered off and concentrated. The residue was purified by reverse phase flash column chromatography on a C18 reverse phase column (MeOH/H₂O) to give the title compound (3.8 g, yield: 54%).

### Step 2. methyl 3-((3-amino-5-(4-(((tert-butoxycarbonyl)amino)methyl)-4-methylpiperidin-1-yl)pyrazin-2-yl)thio)-2-chlorobenzoate

A solution the product of Step 1 above (3.6 g, 11 mmol), tert-butyl ((4-methylpiperidin-4-yl)methyl)carbamate (2.99 g, 13 mmol) and DIPEA (4.23 g, 33 mmol) in NMP (36 mL) was charged with N₂ and stirred at 130° C. for 4 hours until LC/MS showed the reaction was completed. Purification of the reaction mixture by the reverse phase flash column chromatography on C18 (MeOH/H₂O) gave the title compound (3.89 g, yield: 68%).

### Step 3. 3-((3-amino-5-(4-(((tert-butoxycarbonyl)amino)methyl)-4-methylpiperidin-1-yl)pyrazin-2-yl)thio)-2-chlorobenzoic acid

To a solution of the product of Step 2 above (3.89 g, 7.45 mmol) in MeOH/H₂O (50 mL/10 mL) was added lithium hydroxide hydrate (1.56 g, 37.3 mmol). The mixture was stirred overnight, diluted with water (50 mL), and concentrated to remove MeOH. The aqueous phase was acidified with aqueous citric acid (10%) to pH of 4 and extracted with DCM/MeOH (10/1, 200 mL). The extract was dried over Na₂SO₄, filtered off and concentrated to give the title compound (3.87 g, crude yield: 100%), which was used in the next step without any further purification.

### Step 4. tert-butyl ((1-(6-amino-5-((2-chloro-3-((phenylsulfonyl)carbamoyl)phenyl)thio)pyrazin-2-yl)-4-methylpiperidin-4-yl)methyl)carbamate

A solution of the product of Step 3 above (120 mg, 0.236 mmol), benzenesulfonamide (74 mg, 0.472 mmol), DMAP (57.7 mg, 0.472 mmol), and EDCI (90.6 mg, 0.472 mmol) in DCM (10 mL) was stirred at rt overnight. The mixture was diluted with water (5 mL), acidified with aqueous citric acid (10%) to pH of 4, and extracted with DCM/MeOH (10/1, 10 mL×2). The combined organics were dried over Na₂SO₄, filtered off and concentrated to give the crude title compound (180 mg, crude yield: 100%), which was used in the next step without any further purification.

### Step 5. 3-((3-amino-5-(4-(aminomethyl)-4-methylpiperidin-1-yl)pyrazin-2-yl)thio)-2-chloro-N-(phenylsulfonyl)benzamide

To a solution of the product of Step 4 above (180 mg, 0.278 mmol) in DCM/MeOH (10/1, 2 mL) was added 4N HCl/dioxane (2 mL). The mixture was stirred at rt overnight and concentrated to dryness. The residue was purified by reverse phase flash column chromatography on a C18 reverse phase column (MeOH/H₂O) to give the title compound (42 mg, yield: 28%). MS (ESI) m/z: 547.3 [M+1]+; ¹H NMR (400 MHz, dmso) δ 7.84-7.75 (m, 2H), 7.73-7.57 (m, 3H), 7.61 (s, 1H), 7.43-7.36 (m, 3H), 7.09-7.01 (m, 2H), 6.53-6.45 (m, 1H), 6.04 (s, 2H), 3.90-3.77 (m, 2H), 3.37-3.32 (m, 2H), 2.75 (s, 2H), 1.53-1.42 (m, 2H), 1.37 (d, J=13.0 Hz, 2H), 1.05-1.02 (m, 3H).

### Example 6 (Method 6)

### Preparation of N-((4-((5-((3S,4S)-4-amino-3-methyl-2-oxa-8-azaspiro[4.5]decan-8-yl)pyrazin-2-yl)thio)-3-chloropyridin-2-yl)carbamoyl)benzenesulfonamide

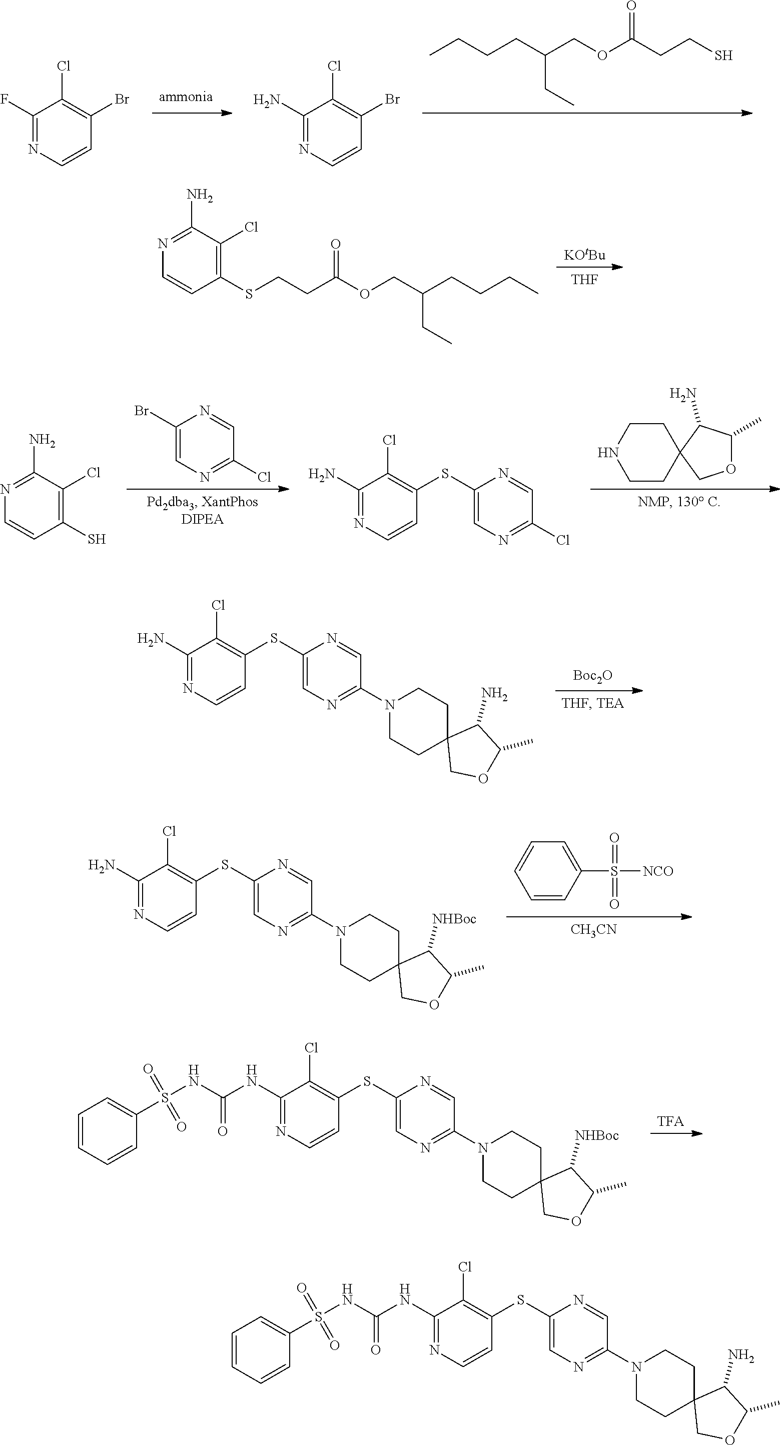

### Step 1. 4-bromo-3-chloropyridin-2-amine

A solution of 4-bromo-3-chloro-2-fluoropyridine (25 g, 118.8 mmol) in ammonia (28%, 200 mL) was stirred at 120° C. in an autoclave for 2 h. The mixture was cooled to rt and extracted with DCM (500 mL×2). The combined organic layers were washed with water (100 mL×2), dried over Na₂SO₄, filtered off and concentrated to give the product as a white solid (24 g, yield: 98%).

### Step 2. 2-ethylhexyl 3-((2-amino-3-chloropyridin-4-yl)thio)propanoate

A solution of the product of Step 1 above (24 g, 115.7 mmol), 2-ethylhexyl 3-mercaptopropanoate (27.8 g, 127.3 mmol), Pd₂dba₃ (2.65 g, 2.9 mmol), Xantphos (3.35 g, 5.79 mmol), and DIPEA (30 g, 231.4 mmol) in dioxane (240 mL) was charged with N₂ and stirred at 110° C. for 4 hours. The mixture was cooled to rt and filtered off. The filtrate was diluted with EtOAc (1000 mL), washed with H₂O (300 mL×2) and brine (300 mL), died over Na₂SO₄, filtered off and concentrated. The residue was purified by flash column chromatography on silica gel (PE/EtOAc=5/1-2/1) to give the title compound (43 g, yield: ˜100%).

### Step 3. 2-amino-3-chloropyridine-4-thiol

To a solution of the product of Step 2 above (5.0 g, 14.5 mmol) in THF (40 mL) at −78° C. under N₂ was added t-BuOK/THF (4.9 g/44 mL) slowly. After the addition was complete, the mixture was stirred at −78° C. for 30 min. The mixture was allowed to warm to rt and concentrated. The residue was purified by flash column chromatography on silica gel (DCM/MeOH=50/1 to 10/1) to give the title compound (1.7 g, yield: 74%).

### Step 4. 3-chloro-4-((5-chloropyrazin-2-yl)thio)pyridin-2-amine

A solution of the product of Step 3 above (700 mg, 4.37 mmol), 2-bromo-5-chloropyrazine (931 mg, 4.8 mmol), Pd₂dba₃ (200 mg, 0.218 mmol), Xantphos (253 mg, 0.437 mmol), DIPEA (1.13 g, 8.75 mmol) in dioxane (7 mL) was charged with N₂ and stirred at 110° C. for 6 hours. The mixture was cooled to rt and concentrated. The residue was swirled with EtOAc (100 mL) and filtered. The filtrate was concentrated and purified by reverse phase flash column chromatography on a C18 reverse phase column (MeOH/H₂O) to give the title compound (655 mg, yield: 55%).

### Step 5. (3S,4S)-8-(5-((2-amino-3-chloropyridin-4-yl)thio)pyrazin-2-yl)-3-methyl-2-oxa-8-azaspiro[4.5]decan-4-amine

A solution of the product of Step 4 above (214 mg, 0.783 mmol), (3S,4S)-3-methyl-2-oxa-8-azaspiro[4.5]decan-4-amine (200 mg, 0.735 mmol), and DIPEA (304 mg, 2.35 mmol) in NMP (2 mL) was charged with N₂ and stirred at 130° C. for 18 hours. The mixture was purified by reverse phase flash column chromatography on a C18 reverse phase column (MeOH/H₂O) to give the crude title compound (396 mg, crude yield: >100%).

### Step 6. tert-butyl ((3S,4S)-8-(5-((2-amino-3-chloropyridin-4-yl)thio)pyrazin-2-yl)-3-methyl-2-oxa-8-azaspiro[4.5]decan-4-yl)carbamate

To an ice-water cooled solution of the product of Step 5 above (396 mg, crude, ˜0.783 mmol) and TEA (238 mg, 2.35 mmol) in THF (10 mL) was added Boc₂O (205 mg, 0.94 mmol). The mixture was allowed to warm to rt, stirred for 0.5 h, and concentrated. The residue was purified by flash column chromatography on silica gel to give the title compound (205 mg, yield: 50%).

### Step 7. tert-butyl ((3S,4S)-8-(5-((3-chloro-2-(3-(phenylsulfonyl)ureido)pyridin-4-yl)thio)pyrazin-2-yl)-3-methyl-2-oxa-8-azaspiro[4.5]decan-4-yl)carbamate

To a solution of the product of Step 6 above (153 mg, 0.3 mmol) in DCM (2 mL) was added NMM (61 mg, 0.60 mmol) and benzenesulfonyl isocyanate (83 mg, 0.45 mmol) sequentially at rt. The mixture was stirred at rt for 0.5 h and concentrated. The residue was purified by prep-TLC (PE/EA=1/1) to give the title compound (123 mg, yield: 59%).

### Step 8. N-((4-((5-((3S,4S)-4-amino-3-methyl-2-oxa-8-azaspiro[4.5]decan-8-yl)pyrazin-2-yl)thio)-3-chloropyridin-2-yl)carbamoyl)benzenesulfonamide

To an ice-water cooled solution of the product of Step 7 above (123 mg, 0.178 mmol) in DCM (5 mL) was added TFA (0.5 mL). The mixture was stirred at rt for 0.5 h, neutralized with 5N NaOH to pH=9-10, and concentrated to remove DCM. The residue was purified by reverse phase flash column chromatography on a C18 reverse phase column (MeOH/H₂O) to give the title compound (20 mg, yield: 19%). MS (ESI) m/z: 590.0 [M+1]+; ¹H NMR (400 MHz, DMSO-d6) δ 8.53 (s, 1H), 8.33 (s, 1H), 7.35 (m, 6H), 6.23 (s, 1H), 4.14 (m, 3H), 3.81 (d, J=8.4 Hz, 1H), 3.62 (d, J=8.8 Hz, 1H), 3.28 (m, 3H), 1.67 (m, 4H), 1.16 (d, J=6.3 Hz, 3H).

### Example 7 (Method 7)

### Preparation of N-((3-((5-(4-amino-4-methylpiperidin-1-yl)pyrazin-2-yl)thio)-2-chlorophenyl)sulfonyl)benzamide

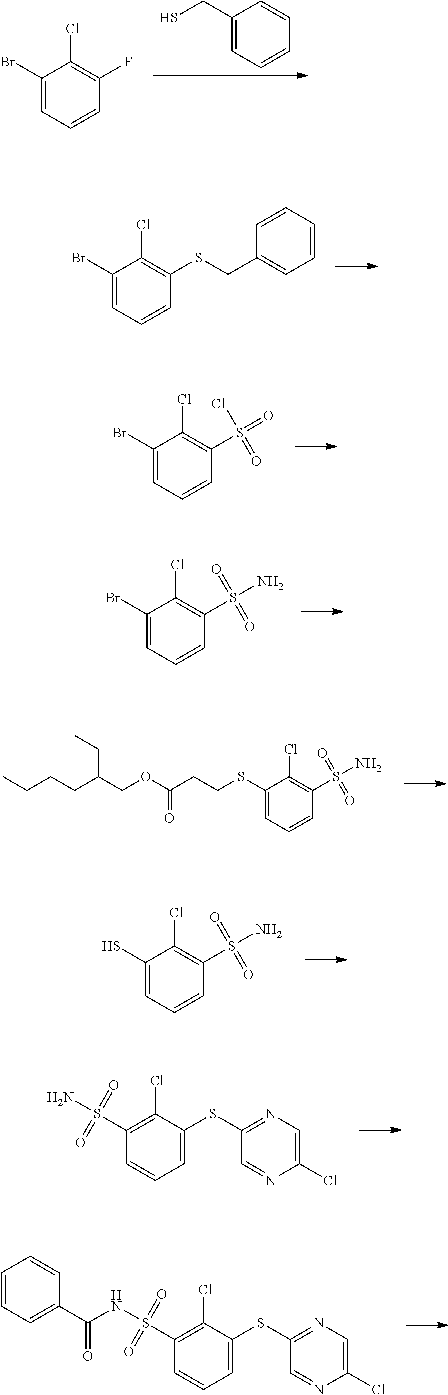

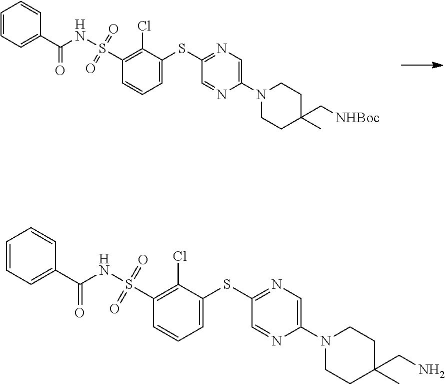 [SMILES: CC1(CNC(=O)OC(C)(C)C)CCN(c2cnc(Sc3cccc(S(=O)(=O)NC(=O)c4ccccc4)c3Cl)cn2)CC1 ; CC1(CN)CCN(c2cnc(Sc3cccc(S(=O)(=O)NC(=O)c4ccccc4)c3Cl)cn2)CC1]

### Step 1. benzyl(3-bromo-2-chlorophenyl)sulfane

To a solution of phenylmethanethiol (1.2 g, 9.55 mmol) in DMF (14 mL) was added Cs₂CO₃ (3.1 g, 9.55 mmol). The mixture was stirred at rt for 10 min and a solution of 1-bromo-2-chloro-3-fluorobenzene (2.0 g, 9.55 mmol) in DMF (2 mL) was added. The mixture was stirred at rt under N₂ overnight and then at 80° C. for 3 h. The mixture was diluted with EtOAc (200 mL), and 2N HCl (6 mL) was added. The mixture was washed with water (100 mL×2) and brine (100 mL), dried over Na₂SO₄, filtered off, and concentrated. The residue was purified by flash column chromatography on silica gel (PE) to give the title compound (2.0 g, yield: 66%).

### Step 2. 3-bromo-2-chlorobenzene-1-sulfonyl chloride

To a solution of the product of Step 1 above (2.0 g, 6.39 mmol) in AcOH/H₂O (16 mL/4 mL) was added NCS (3.4 g, 25.56 mmol) at rt. The mixture was stirred at rt for 5 h, diluted with EtOAc (100 mL), washed with water (30 mL×2) and brine (30 mL), dried over Na₂SO₄, filtered off, and concentrated to give the title compound (3.0 g, crude yield: 100%), which was used in the next step without any further purification.

### Step 3. 3-bromo-2-chlorobenzenesulfonamide

To a solution of the product of Step 2 above (3.0 g, −6.39 mmol) in acetone (100 mL) was added ammonia (28%, 170 mL) slowly at 0° C. The mixture was stirred at rt overnight and concentrated to give the crude title compound (3.5 g, crude yield: 100%), which was used in the next step without any further purification.

### Step 4. 2-ethylhexyl 3-((2-chloro-3-sulfamoylphenyl)thio)propanoate

To a solution of the product of Step 3 above (3.5 g, −6.39 mmol) in dioxane (20 mL) was added 2-ethylhexyl 3-mercaptopropanoate (1.54 g, 7.05 mmol), Pd₂dba₃ (117 mg, 0.128 mmol), XantPhos (320 mg, 0.639 mmol) and DIPEA (1.6 g, 12.78 mmol). The mixture was charged with N₂, stirred at 110° C. overnight, cooed to rt, diluted with EtOAc (200 mL), washed with water (100 mL×2) and brine (50 mL), dried over Na₂SO₄, filtered off, and concentrated. The residue was purified by flash column chromatography on silica gel (PE/EtOAc=2/1) to give the title compound (1.9 g, yield: 73%).

### Step 5. 2-chloro-3-mercaptobenzenesulfonamide

To a solution of the product of Step 4 above (1.9 g, 4.66 mmol) in THF (15 mL) at −78° C. under N₂ was added a solution of t-BuOK (1.5 g, 14 mmol) in THF (44 mL) slowly. After the addition was complete, the mixture was stirred at −78° C. for 30 min, quenched with aqueous K₂CO₃ (2 N, 1.6 mL), and extracted with MBTE (100 mL×2). The aqueous phase was acidified by 6N HCl to pH of 4 and extracted with DCM/i-PrOH (3/1, 200 mL×2). The combined organics were washed with brine (60 mL), dried over Na₂SO₄, filtered off, and concentrated to give the crude title compound (860 mg, yield: 83%), which was used in the next step without any further purification.

### Step 6. 2-chloro-3-((5-chloropyrazin-2-yl)thio)benzenesulfonamide

A solution of the product of Step 5 above (85 mg, 0.38 mmol), 2-bromo-5-chloropyrazine (220 mg, 1.14 mmol), Pd₂dba₃ (17 mg, 0.019 mmol), Xantphos (22 mg, 0.038 mmol), DIPEA (98 mg, 0.76 mmol) in dioxane (3 mL) was charged with N₂ and stirred at 110° C. for 8 hours. The mixture was cooled to rt, diluted with EtOAc (50 mL), washed with water (20 mL) and brine (20 mL), dried over Na₂SO₄, filtered off, and concentrated. The residue was purified by prep-TLC (PE/EtOAc=1/1) to give the title compound (172 mg, yield: 62%).

### Step 7 N-((2-chloro-3-((5-chloropyrazin-2-yl)thio)phenyl)sulfonyl)benzamide

A mixture of the product of Step 6 above (172 mg, 0.512 mmol), benzoic acid (68 mg, 0.563 mmol), DMAP (125 mg, 1.024 mmol), and EDCI (245 mg, 1.28 mmol) in acetonitrile (3 mL) was stirred at rt overnight. The mixture was treated with citric acid (aqueous, 10%) to pH of 4-5 and extracted with DCM/MeOH (10/1, 50 mL). The organic phase was washed with water (15 mL) and brine (15 mL), dried over Na₂SO₄, filtered off, and concentrated. The residue was purified by prep-TLC (DCM/MeOH=10/1) to give the title compound (231 mg, crude yield: 100%), which was used in the next step without any further purification.

### Step 8. tert-butyl ((1-(5-((3-(N-benzoylsulfamoyl)-2-chlorophenyl)thio)pyrazin-2-yl)-4-methylpiperidin-4-yl)methyl)carbamate

To a solution of the product of Step 7 above (60 mg, 0.136 mmol) in NMP (3 mL) was added tert-butyl ((4-methylpiperidin-4-yl)methyl)carbamate (37 mg, 0.164 mmol) and DIPEA (53 mg, 0.408 mmol). The mixture was charged with N₂, stirred at 130° C. overnight, cooled to rt, and purified by reverse phase flash column chromatography (MeOH/H₂O) to give the title compound (70 mg, yield: 81%).

### Step 9. N-((3-((5-(4-(aminomethyl)-4-methylpiperidin-1-yl)pyrazin-2-yl)thio)-2-chlorophenyl)sulfonyl)benzamide

To a solution of the product of Step 8 above (70 mg, 0.111 mmol) in DCM/MeOH (10/1, 6 mL) was added 4N HCl/dioxane (2 mL) at rt. The mixture was stirred at rt for 1 h and concentrated. The residue was purified by reverse phase flash column chromatography (MeOH/H₂O) to give the title compound (23 mg, yield: 39%). MS (ESI) m/z: 532.0 [M+1]+; ¹H NMR (400 MHz, dmso) δ 8.42 (s, 1H), 8.20 (s, 1H), 7.87 (d, J=7.2 Hz, 2H), 7.81 (d, J=7.6 Hz, 1H), 7.64 (s, 3H), 7.40-7.33 (m, 1H), 7.29 (t, J=7.2 Hz, 2H), 7.22 (t, J=7.9 Hz, 1H), 6.84 (d, J=7.7 Hz, 1H), 3.90 (m, 2H), 3.40 (m, 2H), 2.76 (s, 2H), 1.56-1.46 (m, 2H), 1.44-1.36 (m, 2H), 1.05 (s, 3H).

### Example 8 (Method 8)

### Preparation of N-((3-((3-amino-5-(4-amino-4-methylpiperidin-1-yl)pyrazin-2-yl)thio)-2-chlorophenyl)sulfonyl)benzamide

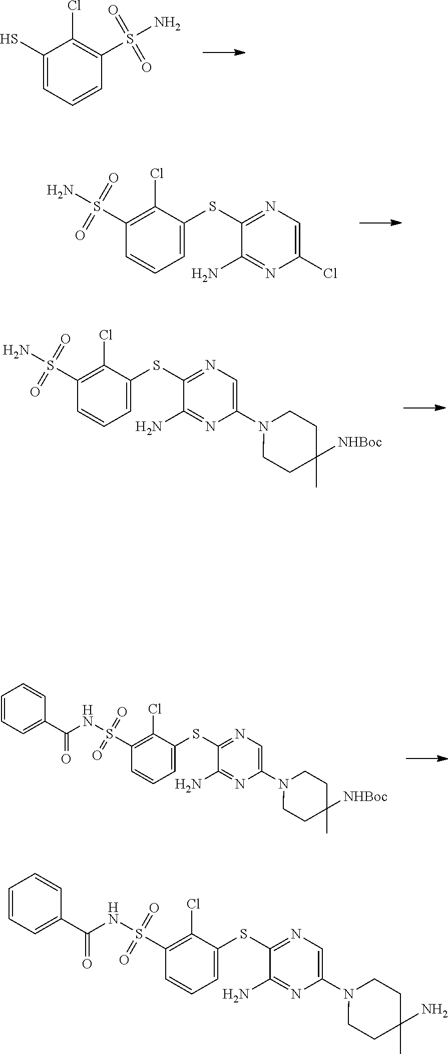 [SMILES: NS(=O)(=O)c1cccc(S)c1Cl ; Nc1nc(Cl)cnc1Sc1cccc(S(N)(=O)=O)c1Cl ; CC1(NC(=O)OC(C)(C)C)CCN(c2cnc(Sc3cccc(S(N)(=O)=O)c3Cl)c(N)n2)CC1 ; CC1(NC(=O)OC(C)(C)C)CCN(c2cnc(Sc3cccc(S(=O)(=O)NC(=O)c4ccccc4)c3Cl)c(N)n2)CC1 ; CC1(N)CCN(c2cnc(Sc3cccc(S(=O)(=O)NC(=O)c4ccccc4)c3Cl)c(N)n2)CC1]

### Step 1. 3-((3-amino-5-chloropyrazin-2-yl)thio)-2-chlorobenzenesulfonamide

A solution of the product of Step 5 in example 7 (100 mg, 0.45 mmol), 3-bromo-6-chloropyrazin-2-amine (102 mg, 0.49 mmol), Pd₂dba₃ (21 mg, 0.0225 mmol), Xantphos (29 mg, 0.05 mmol), DIPEA (116 mg, 0.90 mmol) in dioxane (3 mL) was charged with N₂ and stirred at 110° C. for 8 hours. The mixture was cooled to rt, diluted with EtOAc (100 mL), washed with water (30 mL×2) and brine (30 mL), dried over Na₂SO₄, filtered off, and concentrated. The residue was purified by flash column chromatography (DCM/MeOH=50/1 to 20/1) to give the title compound (158 mg, yield: 100%).

### Step 2. tert-butyl (1-(6-amino-5-((2-chloro-3-sulfamoylphenyl)thio)pyrazin-2-yl)-4-methylpiperidin-4-yl)carbamate

To a solution of the product of Step 1 above (158 mg, 0.45 mmol) in NMP (5 mL) was added tert-butyl (4-methylpiperidin-4-yl)carbamate (193 mg, 0.9 mmol) and DIPEA (175 mg, 1.35 mmol). The mixture was charged with N₂, stirred at 130° C. overnight, cooled to rt, diluted with EtOAc (100 mL), washed with water (30 mL×2) and brine (30 mL), dried over Na₂SO₄, filtered off, and concentrated. The residue was purified by reverse phase flash column chromatography (MeOH/H₂O) to give the title compound (140 mg, yield: 59%).

### Step 3. tert-butyl (1-(6-amino-5-((3-(N-benzoylsulfamoyl)-2-chlorophenyl)thio)pyrazin-2-yl)-4-methylpiperidin-4-yl)carbamate

A mixture of the product of Step 2 above (53 mg, 0.1 mmol), benzoic acid (12 mg, 0.1 mmol), DMAP (24 mg, 0.2 mmol), and EDCI (38 mg, 0.2 mmol) in acetonitrile (3 mL) was stirred at rt overnight. The mixture was diluted with EtOAc (50 mL), washed with water (15 mL×2) and brine (15 mL), dried over Na₂SO₄, filtered off, and concentrated. The residue was purified by prep-TLC (DCM/MeOH=15/1) to give the title compound (41 mg, yield: 65%).

### Step 4. N-((3-((3-amino-5-(4-amino-4-methylpiperidin-1-yl)pyrazin-2-yl)thio)-2-chlorophenyl)sulfonyl)benzamide

To a solution of the product of Step 3 above (41 mg, 0.065 mmol) in DCM (1 mL) was added 4N HCl/dioxane (2 mL) at rt. The mixture was stirred at rt overnight and concentrated. The residue was triturated with DCM (3 mL), filtered and dried to give the title compound (29 mg, yield: 84%). MS (ESI) m/z: 533.2 [M+1]+; ¹H NMR (400 MHz, dmso) δ 8.25 (s, 3H), 7.90 (m, 3H), 7.68-7.58 (m, 2H), 7.48 (m, 3H), 6.87 (d, J=7.9 Hz, 1H), 4.01 (m, 2H), 3.35 (m, 2H), 1.73 (m, 4H), 1.36 (s, 3H).

### Example 9 (Method 9)

### Preparation of N-((4-((5-(4-(aminomethyl)-4-methylpiperidin-1-yl)-6-(hydroxymethyl)-3-methylpyrazin-2-yl)thio)-3-chloropyridin-2-yl)carbamoyl)benzenesulfonamide

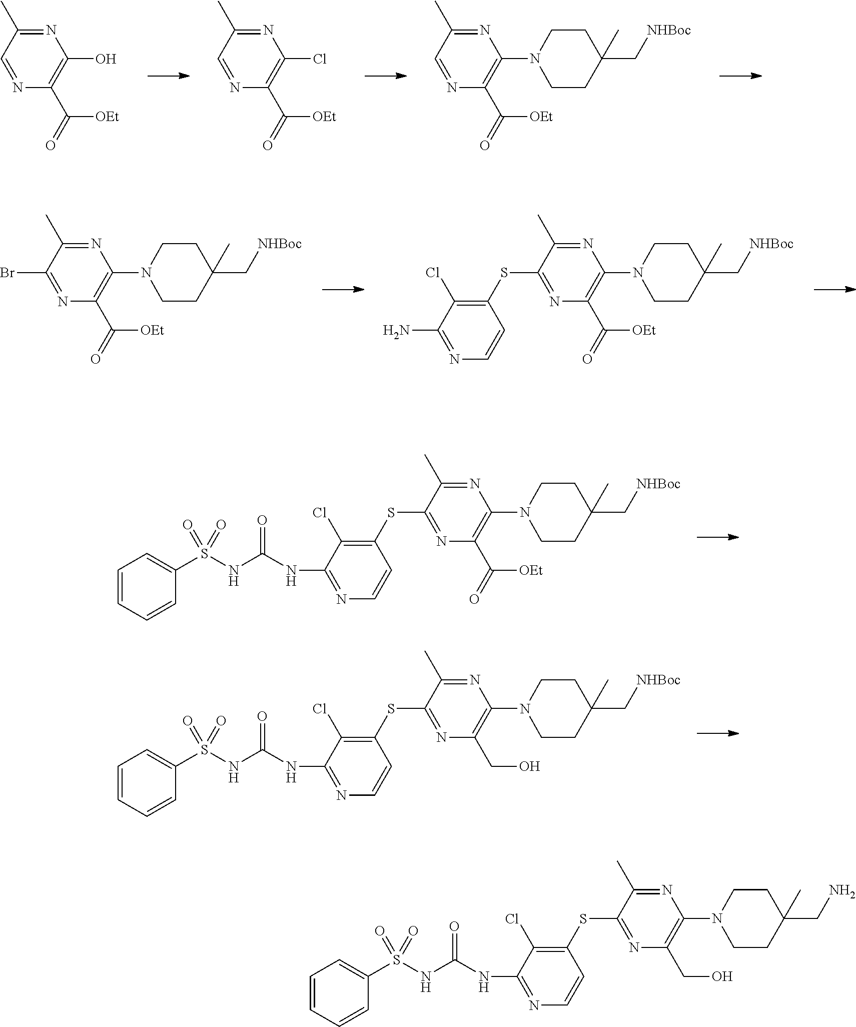

### Step 1. ethyl 3-chloro-5-methylpyrazine-2-carboxylate

A solution of ethyl 3-hydroxy-5-methylpyrazine-2-carboxylate (3.0 g, 16.4 mmol) in POCl₃ (40 mL) was stirred at 110° C. for 3 h. The mixture was cooled to rt and concentrated. The residue was added to crushed ice, neutralized to pH >8 with solid Na₂CO₃, and extracted with EtOAc (50 mL×3). The combined organics were washed with water (50 mL×3) and brine (50 mL), dried over Na₂SO₄, filtered off, and concentrated. The residue was purified by flash column chromatography (PE/EtOAc=4/1) to give the title compound (0.88 g, yield: 27%).

### Step 2. ethyl 3-(4-(((tert-butoxycarbonyl)amino)methyl)-4-methylpiperidin-1-yl)-5-methylpyrazine-2-carboxylate

A mixture of the product of Step 1 above (0.88 g, 4.39 mmol), tert-butyl ((4-methylpiperidin-4-yl)methyl)carbamate (1.2 g, 5.26 mmol), and K₂CO₃ (1.8 g, 13.17 mmol) in NMP (3 mL) was stirred at 100° C. overnight. The mixture was cooled to rt, diluted with EtOAc (100 mL), washed with water (30 mL×2) and brine (30 mL), dried over Na₂SO₄, filtered off, and concentrated. The residue was purified by flash column chromatography (PE/EtOAc=3/2) to give the title compound (1.7 g, yield: 98%).

### Step 3. ethyl 6-bromo-3-(4-(((tert-butoxycarbonyl)amino)methyl)-4-methylpiperidin-1-yl)-5-methylpyrazine-2-carboxylate

To a ice-water cooled solution of the product of Step 2 above (1.7 g, 4.33 mmol) in DCM (20 mL) was added NBS (1.2 g, 6.50 mmol). The mixture was stirred at rt for 1 h, quenched with aqueous Na₂S₂O₃, and extracted with DCM (50 mL×3). The combined organics were washed with brine (50 mL), dried over Na₂SO₄, filtered off, and concentrated. The residue was purified by flash column chromatography (PE/EtOAc=10/1 to 5/1) to give the title compound (920 mg, yield: 45%).

### Step 4. ethyl 6-((2-amino-3-chloropyridin-4-yl)thio)-3-(4-(((tert-butoxycarbonyl)amino)methyl)-4-methylpiperidin-1-yl)-5-methylpyrazine-2-carboxylate

A mixture of the product of Step 3 above (701 mg, 1.49 mmol), 2-amino-3-chloropyridine-4-thiol (262 mg, 1.64 mmol), Pd₂dba₃ (137 mg, 0.15 mmol), BINAP (174 mg, 0.3 mmol), and K₂CO₃ (415 mg, 3.0 mmol) in dioxane (10 mL) was charged with N₂ and stirred at 125° C. overnight. The mixture was cooled to rt, diluted with DCM/MeOH (10/1, 100 mL), washed with water (30 mL×2) and brine (30 mL), dried over Na₂SO₄, filtered off, and concentrated. The residue was purified by flash column chromatography (PE/EtOAc=1/1) to give the title compound (140 mg, yield: 13%).

### Step 5. ethyl 3-(4-(((tert-butoxycarbonyl)amino)methyl)-4-methylpiperidin-1-yl)-6-((3-chloro-2-(3-(phenylsulfonyl)ureido)pyridin-4-yl)thio)-5-methylpyrazine-2-carboxylate

To an ice-water cooled solution of the product of Step 4 above (110 mg, 0.2 mmol) in THF (3 mL) was added benzenesulfonyl isocyanate (55 mg, 0.3 mmol). The mixture was stirred at rt for 0.5 h, diluted with EtOAc (100 mL), washed with water (30 mL×2) and brine (30 mL), dried over Na₂SO₄, filtered off, and concentrated. The residue was purified by prep-TLC (PE/EtOAc=1/1) to give the title compound (105 mg, yield: 72%).

### Step 6. tert-butyl ((1-(5-((3-chloro-2-(3-(phenylsulfonyl)ureido)pyridin-4-yl)thio)-3-(hydroxymethyl)-6-methylpyrazin-2-yl)-4-methylpiperidin-4-yl)methyl)carbamate

To an ice-water cooled solution of the product of Step 5 above (50 mg, 0.068 mmol) in THF (1 mL) was added Super-hydride (1.0 M in THF, 0.2 mL, 0.2 mmol) dropwise. The mixture was stirred at rt for 2 h, cooled in ice-water bath, treated with MeOH (2 mL) and saturated NH₄Cl (10 mL), stirred for 0.5 h, and extracted with EtOAc (30 mL×3). The combined organics were washed with brine (30 mL), dried over Na₂SO₄, filtered off, and concentrated. The residue was purified by prep-TLC (DCM/MeOH=25/1) to give the title compound (25 mg, yield: 53%).

### Step 7. N-((4-((5-(4-(aminomethyl)-4-methylpiperidin-1-yl)-6-(hydroxymethyl)-3-methylpyrazin-2-yl)thio)-3-chloropyridin-2-yl)carbamoyl)benzenesulfonamide

To an ice-water cooled solution of the product of Step 6 above (25 mg, 0.036 mmol) in DCM (2 mL) was added TFA (0.5 mL) slowly. The mixture was stirred at rt for 1 h, diluted with DCM (2 mL), and concentrated. The residue was purified by reverse phase flash column chromatography (MeOH/H₂O) to give the title compound (8 mg, yield: 38%). MS (ESI) m/z: 592.2 [M+1]+; ¹H NMR (400 MHz, DMSO-d6) δ 7.88 (d, J=4.9 Hz, 1H), 7.76 (s, 1H), 7.86-7.62 (b, 3H), 7.37 (s, 3H), 6.18 (d, J=4.5 Hz, 1H), 5.49 (s, 1H), 4.44 (s, 2H), 3.65 (d, J=12.9 Hz, 2H), 3.35 (s, 2H), 2.77 (s, 2H), 2.39 (s, 3H), 1.58 (m, 2H), 1.47 (m, 2H), 1.05 (s, 3H).

### Example 10 (Method 10)

### Preparation of N-((3-((5-((3S,4S)-4-amino-3-methyl-2-oxa-8-azaspiro[4.5]decan-8-yl)pyrazin-2-yl)thio)-2-chlorophenyl)carbamoyl)-2-fluorobenzenesulfonamide

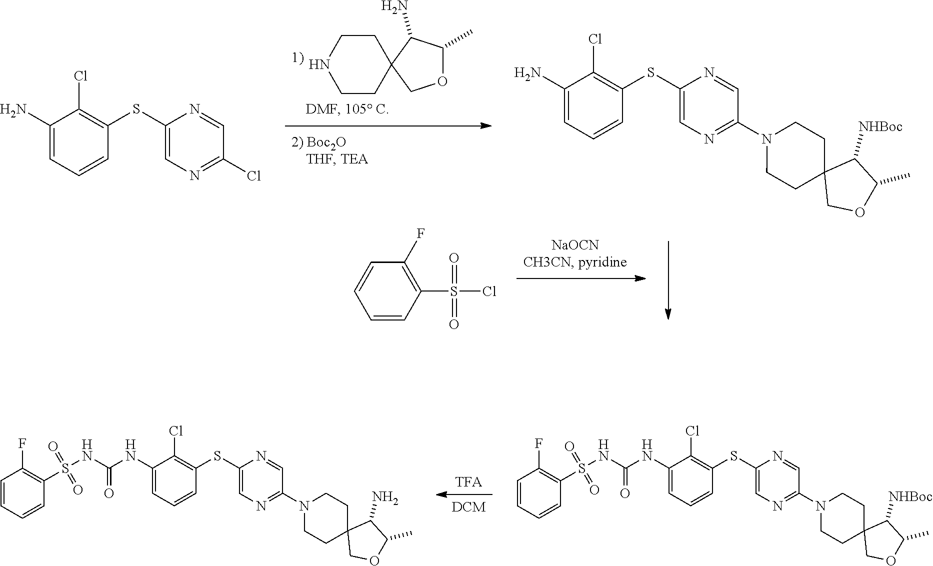

### Step 1. tert-Butyl ((3S,4S)-8-(5-((3-amino-2-chlorophenyl)thio)pyrazin-2-yl)-3-methyl-2-oxa-8-azaspiro[4.5]decan-4-yl)carbamate

A solution of the product of Step 3 in Example 1 (4.2 g, 15.4 mmol), (3S,4S)-3-methyl-2-oxa-8-azaspiro[4.5]decan-4-amine (4.88 g, 20.1 mmol), and DIPEA (9.95 g, 77.2 mmol) in DMF (42 mL) was charged with N₂ and stirred at 105° C. for 18 hours. The mixture was cooed to rt and Boc₂O (6.73 g, 308 mmol) was added. The mixture was stirred at rt for 3 h, diluted with water (100 mL), and extracted with EtOAc (200 mL×2). The combined organic layers were washed with brine (200 mL×2), dried over anhydrous Na₂SO₄, filtered off and concentrated. The residue was purified by flash column chromatography on silica gel (PE/EtOAc=15/1) to give the title compound (7.2 g, yield: 77%).

### Step 2. tert-Butyl ((3S,4S)-8-(5-((2-chloro-3-(3-((2-fluorophenyl)sulfonyl)ureido)phenyl)thio)pyrazin-2-yl)-3-methyl-2-oxa-8-azaspiro[4.5]decan-4-yl)carbamate

To a solution of 2-fluorobenzene-1-sulfonyl chloride (230 mg, 1.18 mmol) in acetonitrile (4 mL) were added NaOCN (130 mg, 2.01 mmol) and pyridine (233 mg, 2.95 mmol) sequentially at rt. The mixture was stirred at rt for 1 h treated with the product of Step 1 above (120 mg, 0.237 mmol). The mixture was stirred at rt for 1 h and concentrated. The residue dissolved in DCM/MeOH (10/1, 60 mL), washed with water (10 mL), dried over anhydrous Na₂SO₄, filtered off, and concentrated. The residue was purified by prep-TLC (DCM/MeOH=15/1) to give the title compound (50 mg, yield: 30%).

### Step 3. N-((3-((5-((3S,4S)-4-amino-3-methyl-2-oxa-8-azaspiro[4.5]decan-8-yl)pyrazin-2-yl)thio)-2-chlorophenyl)carbamoyl)-2-fluorobenzenesulfonamide

To an ice-water cooled solution of the product of Step 2 above (50 mg, 0.071 mmol) in DCM (4 mL) was added TFA (1 mL). The mixture was stirred at rt for 2 h and concentrated. The residue was purified by prep-TLC (DCM/MeOH=5/1) to give the title compound (27 mg, yield: 63%). MS (ESI) m/z: 607.2 [M+1]+; ¹H NMR (400 MHz, DMSO-d6) δ 8.42 (s, 1H), 8.19 (s, 1H), 7.97 (d, J=8.2 Hz, 1H), 7.77 (t, J=7.3 Hz, 1H), 7.59 (s, 1H), 7.44 (m, 2H), 7.18 (m, 2H), 7.02 (t, J=8.1 Hz, 1H), 6.42 (d, J=7.8 Hz, 1H), 4.29-4.07 (m, 3H), 3.91 (d, J=8.8 Hz, 1H), 3.64 (d, J=9.0 Hz, 1H), 3.30 (m, 1H), 3.08 (m, 2H), 1.77 (m, 2H), 1.62 (m, 2H), 1.22 (d, J=6.3 Hz, 3H).

### Example 11 (Method 11)

### Preparation of N-((3-((5-((3S,4S)-4-amino-3-methyl-2-oxa-8-azaspiro[4.5]decan-8-yl)-6-(hydroxymethyl)-3-methylpyrazin-2-yl)thio)-2-chlorophenyl)carbamoyl)benzenesulfonamide

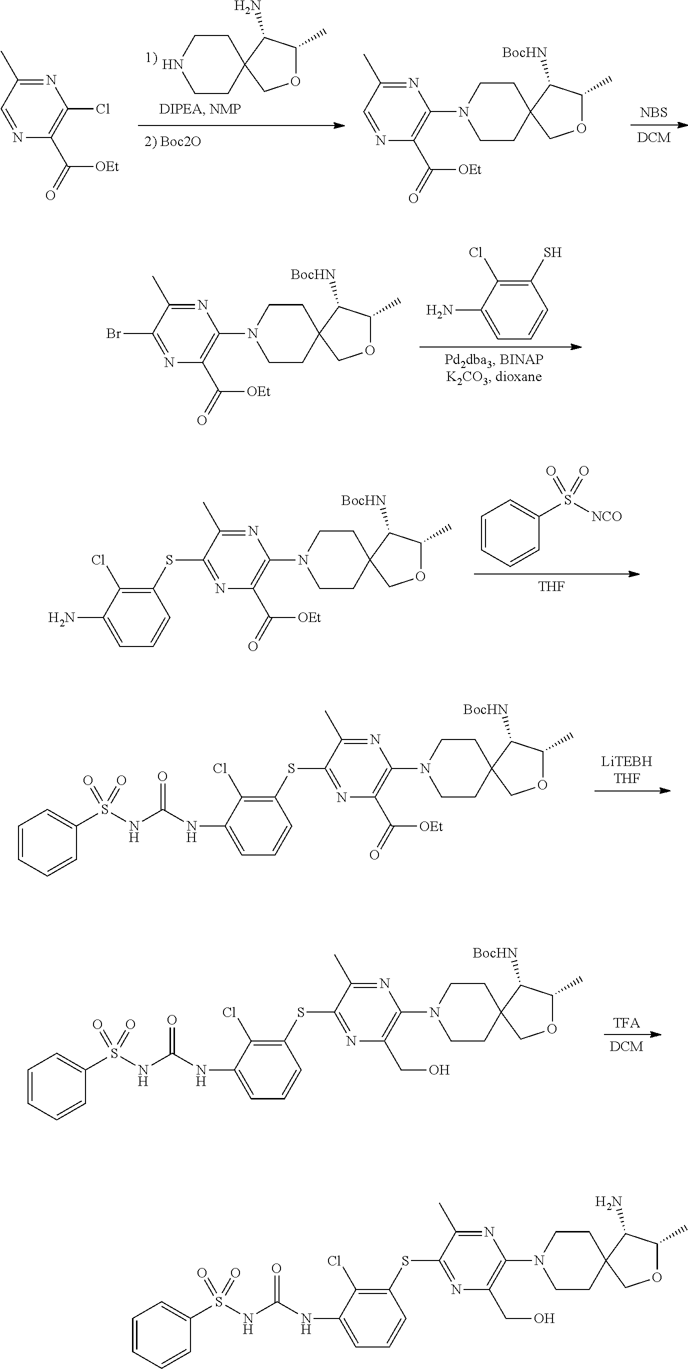

### Step 1. Ethyl 3-((3S,4S)-4-((tert-butoxycarbonyl)amino)-3-methyl-2-oxa-8-azaspiro[4.5]decan-8-yl)-5-methylpyrazine-2-carboxylate

A mixture of ethyl 3-chloro-5-methylpyrazine-2-carboxylate (1.34 g, 6.68 mmol), (3S,4S)-3-methyl-2-oxa-8-azaspiro[4.5]decan-4-amine hydrogen chloride (1.8 g, 7.35 mmol), and DIPEA (4.3 g, 33.4 mmol) in NMP (10 mL) was stirred at 100° C. overnight. The mixture was cooled to rt and (Boc)₂O (2.2 g, 10.0 mmol) was added. The mixture was stirred at RT for 2 h, diluted with EtOAc (150 mL), washed with water (50 mL×2) and brine (50 mL), dried over anhydrous Na₂SO₄, filtered off, and concentrated. The residue was purified by flash column chromatography on silica gel (PE/EtOAc=4/1-3/2) to give the title compound (2.3 g, yield: 79%).

### Step 2. Ethyl 6-bromo-3-((3S,4S)-4-((tert-butoxycarbonyl)amino)-3-methyl-2-oxa-8-azaspiro[4.5]decan-8-yl)-5-methylpyrazine-2-carboxylate

To an ice-water cooled solution of the product of Step 1 above (434 mg, 1.0 mmol) in DCM (5 mL) was added NBS (356 mg, 2.0 mmol). The mixture was stirred at rt for 2 h, quenched with aqueous Na₂S₂O₃, and extracted with DCM (50 mL×3). The combined organic layers were washed with brine (50 mL), dried over anhydrous Na₂SO₄, filtered off, and concentrated. The residue was purified by flash column chromatography on silica gel (PE/EtOAc=4/1 to 2/1) to give the title compound (602 mg, yield: 95%).

### Step 3. Ethyl 6-((3-amino-2-chlorophenyl)thio)-3-((3S,4S)-4-((tert-butoxycarbonyl)amino)-3-methyl-2-oxa-8-azaspiro[4.5]decan-8-yl)-5-methylpyrazine-2-carboxylate

A mixture of the product of Step 2 above (250 mg, 0.49 mmol), 3-amino-2-chlorobenzenethiol (93 mg, 0.59 mmol), Pd₂dba₃ (46 mg, 0.05 mmol), BINAP (58 mg, 0.1 mmol), and K₂CO₃ (138 mg, 1.0 mmol) in dioxane (5 mL) was charged with N₂ and stirred at 100° C. overnight. The mixture was cooled to rt, diluted with DCM/MeOH (10/1, 100 mL), washed with water (30 mL×2) and brine (30 mL), dried over anhydrous Na₂SO₄, filtered off, and concentrated. The residue was purified by flash column chromatography on silica gel (PE/EtOAc=4/1-3/2) to give the title compound (130 mg, yield: 45%).

### Step 4. Ethyl 3-((3S,4S)-4-((tert-butoxycarbonyl)amino)-3-methyl-2-oxa-8-azaspiro[4.5]decan-8-yl)-6-((2-chloro-3-(3-(phenylsulfonyl)ureido)phenyl)thio)-5-methylpyrazine-2-carboxylate

To an ice-water cooled solution of the product of Step 3 above (170 mg, 0.29 mmol) in THF (5 mL) was added benzenesulfonyl isocyanate (79 mg, 0.43 mmol). The mixture was stirred at rt for 10 min and concentrated. The residue was purified by prep-TLC (DCM/MeOH=20/1) to give the title compound (160 mg, yield: 72%).

### Step 5. tert-Butyl ((3S,4S)-8-(5-((2-chloro-3-(3-(phenylsulfonyl)ureido)phenyl)thio)-3-(hydroxymethyl)-6-methylpyrazin-2-yl)-3-methyl-2-oxa-8-azaspiro[4.5]decan-4-yl)carbamate

To an ice-water cooled solution of the product of Step 4 above (110 mg, 0.14 mmol) in THF (3 mL) was added Super-hydride (1.0 M in THF, 1 mL, 1 mmol) dropwise. The mixture was stirred at 45° C. for 6 h, cooled in ice-water bath, treated with MeOH (2 mL) and saturated NH₄Cl (10 mL), stirred for 0.5 h, and extracted with EtOAc (30 mL×3). The combined organic layers were washed with brine (30 mL), dried over anhydrous Na₂SO₄, filtered off, and concentrated. The residue was purified by prep-TLC (DCM/MeOH=20/1) to give the title compound (55 mg, yield: 53%).

### Step 6. N-((3-((5-((3S,4S)-4-amino-3-methyl-2-oxa-8-azaspiro[4.5]decan-8-yl)-6-(hydroxymethyl)-3-methylpyrazin-2-yl)thio)-2-chlorophenyl)carbamoyl)benzenesulfonamide

To an ice-water cooled solution of the product of Step 5 above (55 mg, 0.075 mmol) in DCM (3 mL) was added TFA (2 mL) slowly. The mixture was stirred at rt for 2 h, diluted with DCM (2 mL), and concentrated. The residue was purified by reverse phase flash column chromatography on a C18 column (MeOH/H₂O) to give the title compound (25 mg, yield: 45%). MS (ESI) m/z: 633.2 [M+1]+; ¹H NMR (400 MHz, DMSO-d6) δ 8.03 (d, J=8.3 Hz, 1H), 7.83-7.72 (m, 2H), 7.55 (s, 1H), 7.44-7.30 (m, 3H), 7.02 (t, J=8.1 Hz, 1H), 6.34 (d, J=7.7 Hz, 1H), 5.35 (t, J=5.2 Hz, 1H), 4.42 (d, J=4.8 Hz, 2H), 4.23-4.11 (m, 1H), 3.90-3.59 (m, 4H), 3.42-3.22 (m, 1H), 3.12-2.91 (m, 2H), 2.38 (s, 3H), 1.89-1.48 (m, 4H), 1.20 (d, J=8.6 Hz, 3H).

### Example 12 (Method 12)

### Preparation of ((3S,4S)-8-(5-((2-chloro-3-((N-(phenylcarbamoyl)sulfamoyl)amino)phenyl)thio)pyrazin-2-yl)-3-methyl-2-oxa-8-azaspiro[4.5]decan-4-yl)amine

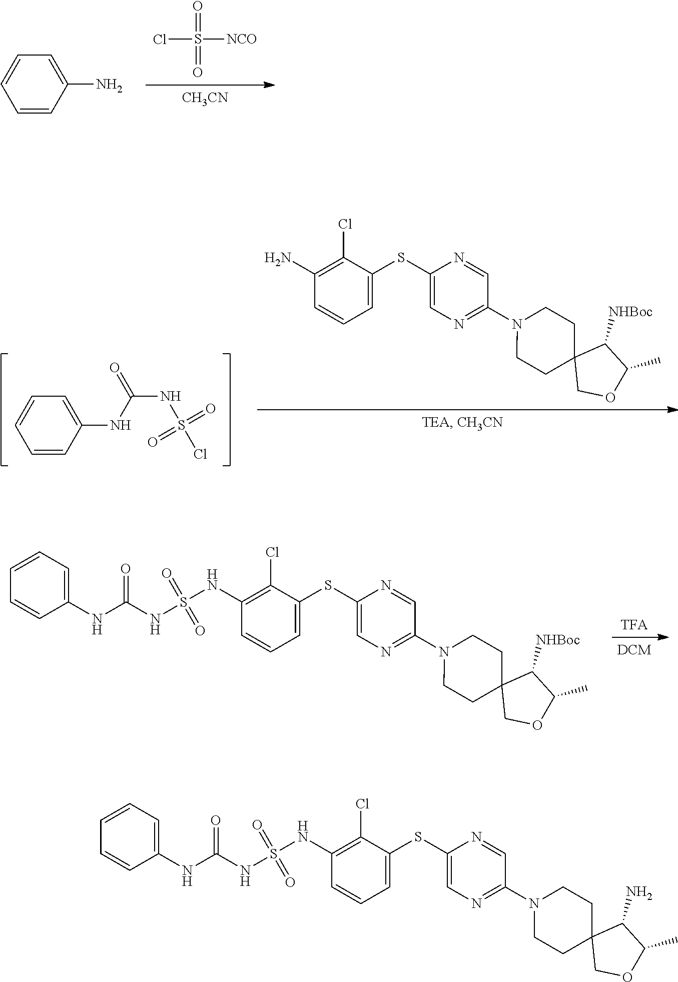

### Step 1. tert-butyl ((3S,4S)-8-(5-((2-chloro-3-((N-(phenylcarbamoyl)sulfamoyl)amino)-phenyl)thio)pyrazin-2-yl)-3-methyl-2-oxa-8-azaspiro[4.5]decan-4-yl)carbamate

A solution of chlorosulfonyl isocyanate (142 mg, 1.0 mmol) in CH₃CN (1 mL) was cooled to −20° C. and a solution of aniline (93 mg, 1.0 mmol) in CH₃CN (2 mL) was added. The mixture was stirred at −10° C. for 10 min and used directly. To an ice-water cooled solution of the product of Step 1 in Example 10 (202 mg, 0.4 mmol) and TEA (81 mg, 0.8 mmol) in CH₃CN (2 mL) was added the solution of (phenylcarbamoyl)sulfamoyl chloride obtained previously. The mixture was stirred at rt overnight, diluted with EtOAc (100 mL), washed by H₂O (30 mL×2) and brine (30 mL), dried by anhydrous Na₂SO₄, filtered off and concentrated. The reside was purified by flash column chromatography on silica gel (DCM/MeOH=40/1) to give the title compound (140 mg, yield: 50%)

### Step 2. ((3S,4S)-8-(5-((2-chloro-3-((N-(phenylcarbamoyl)sulfamoyl)amino)phenyl)thio)-pyrazin-2-yl)-3-methyl-2-oxa-8-azaspiro[4.5]decan-4-yl)amine

To an ice-water cooled solution of the product of Step 1 above (140 mg, 0.2 mmol) in DCM (3 mL) was added TFA (2 mL) slowly. The mixture was stirred at rt for 2 h and concentrated. The residue was purified by reverse phase flash column chromatography on a C18 column (MeOH/H₂O) to give the compound (55 mg, yield: 46%). MS (ESI) m/z: 604.3 [M+1]+; ¹H NMR (400 MHz, DMSO-d6) δ 8.42 (s, 1H), 8.20 (s, 1H), 7.54 (d, J=8.2 Hz, 1H), 7.45 (d, J=8.2 Hz, 2H), 7.18-6.99 (m, 3H), 6.79 (d, J=7.2 Hz, 1H), 6.46 (d, J=7.8 Hz, 1H), 4.24-4.09 (m, 3H), 3.87 (d, J=8.9 Hz, 1H), 3.67 (d, J=9.1 Hz, 1H), 3.37 (d, J=4.7 Hz, 1H), 3.19-3.03 (m, 2H), 1.66 (m, 4H), 1.18 (d, J=6.5 Hz, 3H).

### Example 13 (Method 13)

### Preparation of N-((3-((5-((3S,4S)-4-amino-3-methyl-2-oxa-8-azaspiro[4.5]decan-8-yl)pyrazin-2-yl)thio)-2-chlorophenyl)carbamoyl)pyrrolidine-1-sulfonamide

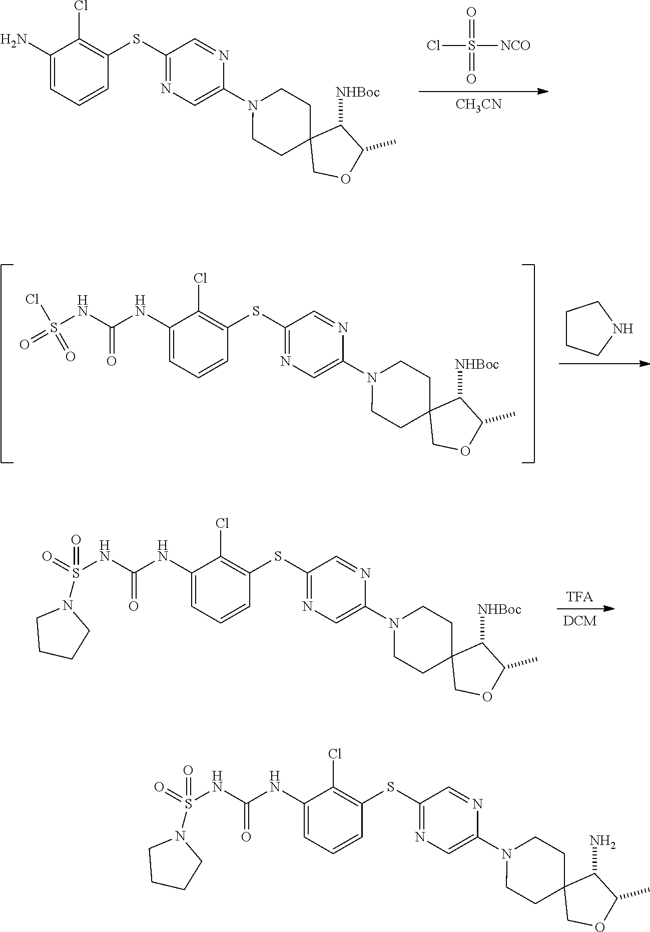

### Step 1. tert-butyl ((3S,4S)-8-(5-((2-chloro-3-(3-(pyrrolidin-1-ylsulfonyl)ureido)phenyl)thio)pyrazin-2-yl)-3-methyl-2-oxa-8-azaspiro[4.5]decan-4-yl)carbamate

To a solution of chlorosulfonyl isocyanate (73 mg, 0.52 mmol) in acetonitrile (1.0 mL) was added a solution of the product of Step 1 in Example 10 (202 mg, 0.4 mmol) in acetonitrile (2.0 mL) dropwise at −20° C. The mixture was stirred at −20° C. for 2 h before adding pyrrolidine (142 mg, 2.0 mmol) in one portion. The mixture was stirred at rt for 2 h and concentrated. The residue was purified by flash column chromatography on silica gel (PE/EtOAc=2/1) to give the title compound (49 mg, yield: 21%).

### Step 2. N-((3-((5-((3S,4S)-4-amino-3-methyl-2-oxa-8-azaspiro[4.5]decan-8-yl)pyrazin-2-yl)thio)-2-chlorophenyl)carbamoyl)pyrrolidine-1-sulfonamide

To an ice-water cooled solution the product of Step 1 above (49 mg, 0.0718 mmol) in DCM (2 mL) was added TFA (1 mL). The mixture was stirred at rt for 2 h and concentrated. The residue was purified by reverse phase flash column chromatography on a C18 column (MeOH/H₂O) to give the title compound (9 mg, yield: 23%). MS (ESI) m/z: 582.1 [M+1]+; ¹H NMR (400 MHz, DMSO-d6) δ 8.38 (s, 1H), 8.18 (s, 1H), 8.10 (s, 1H), 7.04 (t, J=8.0 Hz, 1H), 6.37 (d, J=7.6 Hz, 1H), 4.11-4.01 (m, 1H), 3.92-3.78 (m, 2H), 3.65 (d, J=8.3 Hz, 1H), 3.47 (d, J=8.1 Hz, 1H), 3.31 (m, 1H), 3.11-3.02 (m, 5H), 2.89 (d, J=4.6 Hz, 1H), 1.74-1.68 (m, 5H), 1.54-1.46 (m, 2H), 1.24-1.20 (m, 1H), 1.06 (d, J=6.3 Hz, 3H).

### Example 14

### Preparation of (S)—N-((4-((5-(7-amino-5,7-dihydrospiro[cyclopenta[b]pyridine-6,4′-piperidin]-1′-yl)pyrazin-2-yl)thio)-3-chloropyridin-2-yl)carbamoyl)benzenesulfonamide

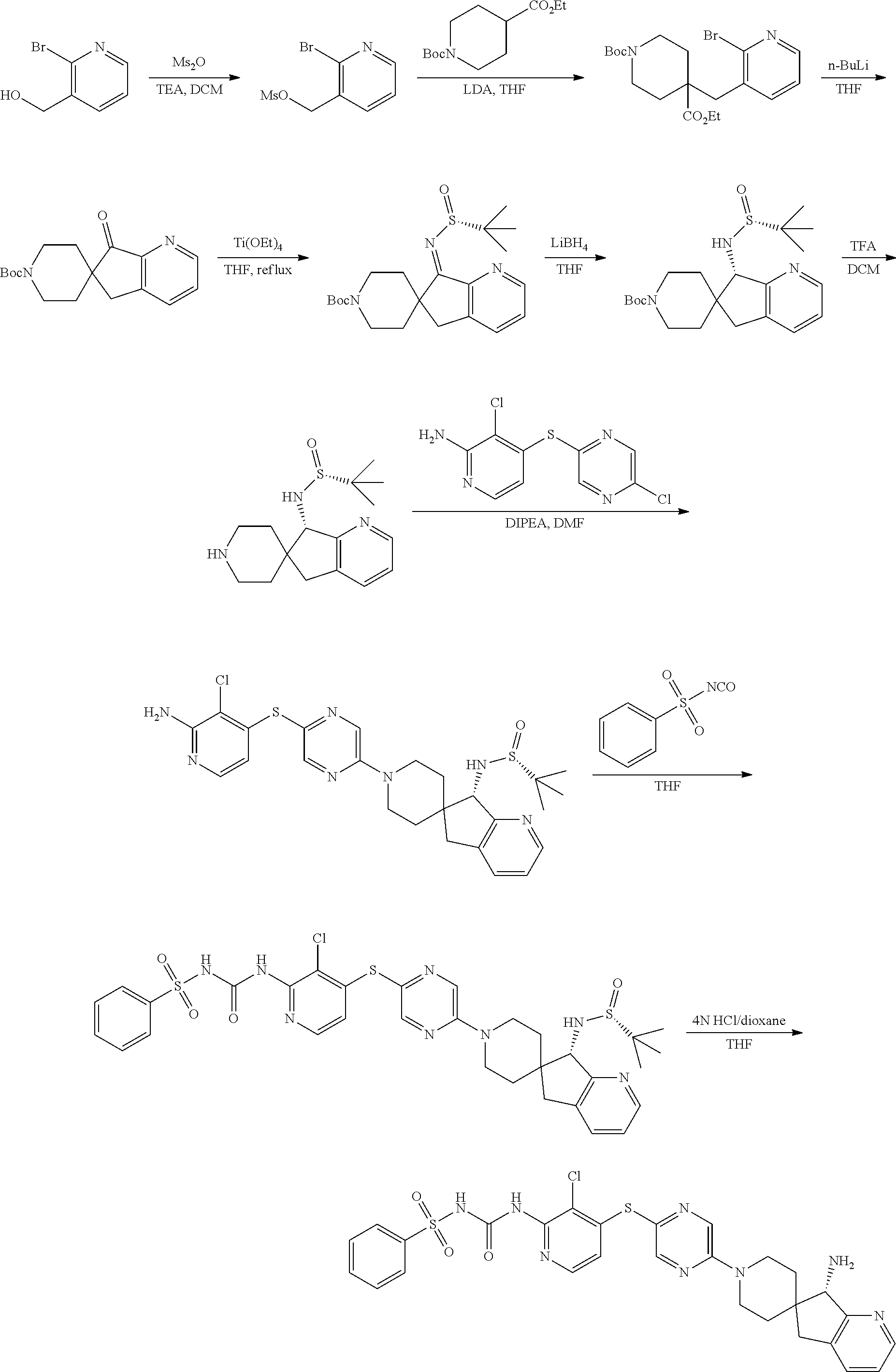

### Step 1. (2-bromopyridin-3-yl)methyl methanesulfonate

To an ice-water cooled solution of (2-bromopyridin-3-yl)methanol (4.0 g, 21.3 mmol) in DCM (60 mL) was added methanesulfonic anhydride (4.08 g, 23.4 mmol) and TEA (4.3 g, 42.6 mmol). The mixture was stirred at rt for 3 h before concentrated. The residue was diluted with ethyl acetate (200 mL), washed with water (30 mL) and brine (30 mL), dried over Na₂SO₄, filtered off, and concentrated. The residue was purified by flash column chromatography on silica gel (PE/EtOAc=4/1) to give the title compound (4.6 g, yield: 82%).

### Step 2. 1-tert-butyl 4-ethyl 4-((2-bromopyridin-3-yl)methyl)piperidine-1,4-dicarboxylate

A solution of the product of 1-tert-butyl 4-ethyl piperidine-1,4-dicarboxylate (5.34 g, 20.7 mmol) in THF (60 mL) was cooled to −78° C. and LDA (1N in THF, 24.2 mL, 24.2 mmol) was added dropwise. The reaction was stirred at −78° C. for 1 h and a solution of the product of Step 1 above in THF (30 mL) was added dropwise. The reaction was stirred at −78° C. for 2 h, quenched with sat. aqueous NH₄Cl (30 mL), extracted with EA (200 mL), washed with water (20 mL) and brine (30 mL), dried over Na₂SO₄, filtered off and concentrated. The residue was purified by flash column chromatography on silica gel (PE/EtOAc=6/1) to give the title compound (5.9 g, yield: 80%).

### Step 3. tert-butyl 7-oxo-5,7-dihydrospiro[cyclopenta[b]pyridine-6,4′-piperidine]-1′-carboxylate

To a solution of the product of Step 2 above (5.9 g, 13.8 mmol) in THF (80 mL) was added n-BuLi (8.3 mmol, 20.7 mmol, 2.5N in hexane) dropwise at −78° C. under N₂. The reaction was stirred at −78° C. for 2 h, quenched with sat. aq. NH₄Cl (20 mL), extracted with EtOAc (100 mL), washed with water (30 mL) and brine (30 mL), dried over Na₂SO₄, filtered off, and concentrated. The residue was purified by by flash column chromatography on silica gel (PE/EtOAc=2/1 to 1/1) to give the title compound (1.46 g, yield: 33%).

### Step 4. (R,Z)-tert-butyl 7-((tert-butylsulfinyl)imino)-5,7-dihydrospiro[cyclopenta[b]pyridine-6,4′-piperidine]-1′-carboxylate

To a solution of the product of Step 3 above (1.3 g, 4.3 mmol) in THF (30 mL) was added (R)-(+)-2-Methyl-2-propanesulfinamide (2.09 g, 17.2 mmol) and Ti(OEt)₄ (8 mL) successively. The reaction was refluxed overnight, cooled to rt, diluted with brine, stirred for 15 min, and filtered through celite. The filtrate was extracted with EtOAc (100 mL×2). The combined organics were washed with brine (30 mL), dried over Na₂SO₄, filtered off, and concentrated. The residue was purified by flash column chromatography on silica gel (PE/EtOAc=1/1 to DCM/MeOH=50/1) to give the title compound (1.8 g, yield: 92%).

### Step 5. (S)-tert-butyl 7-((R)-1,1-dimethylethylsulfinamido)-5,7-dihydrospiro[cyclopenta[b]pyridine-6,4′-piperidine]-1′-carboxylate

To a solution of the product of Step 4 above (1.6 g, 3.95 mmol) in dry THF (30 mL) was added LiBH₄ (3.95 mL, 7.9 mmol, 2N in THF) dropwise at 0° C. The mixture was stirred at rt for 1 h before quenching with methanol (30 mL). The mixture was concentrated and the residue was purified by flash column chromatography on silica gel (PE/EtOAc=1/1 to DCM/MeOH=50/1) to give the title compound (1.6 g, yield: 88%).

### Step 6. (R)—N—((S)-5,7-dihydrospiro[cyclopenta[b]pyridine-6,4′-piperidin]-7-yl)-2-methylpropane-2-sulfinamide

To a solution of the product of Step 5 above (1.6 g, 3.93 mmol) in DCM (20 mL) was added TFA (5 mL) at 0° C. The mixture was stirred at rt for 1 h before concentrated. The residue was treated with sat. Na₂CO₃ (30 mL) and extracted with DCM/MeOH (10/1, 100 mL×2). The combined organic layers were dried over Na₂SO₄, filtered off and concentrated to give the title compound, which was used in the next step without any further purification (1.2 g, yield: 99%).

### Step 7. (R)—N—((S)-1′-(5-((2-amino-3-chloropyridin-4-yl)thio)pyrazin-2-yl)-5,7-dihydrospiro[cyclopenta[b]pyridine-6,4′-piperidin]-7-yl)-2-methylpropane-2-sulfinamide

To a solution of the product of Step 6 above in DMF (20 mL) was added DIPEA (1.722 g, 13.35 mmol). The mixture was stirred at 100° C. overnight, cooled to rt, and extracted with EA (100 mL). The organic layer was washed with water (20 mL) and brine (30 mL), dried over Na₂SO₄, filtered off, and concentrated. The residue was purified by flash column chromatography on silica gel (PE/EtOAc=1/1 to DCM/MeOH=40/1 to 20/1) to give the title compound (546 mg, yield: 38%).

### Step 8. N-((3-chloro-4-((5-((S)-7-((R)-1,1-dimethylethylsulfinamido)-5,7-dihydrospiro[cyclopenta[b]pyridine-6,4′-piperidin]-1′-yl)pyrazin-2-yl)thio)pyridin-2-yl)carbamoyl)benzenesulfonamide

To a solution of the product of Step 7 above (109 mg, 0.2 mmol) in THF (3 mL) was added benzenesulfonyl isocyanate (55 mg, 0.3 mmol) at rt. The mixture was stirred at rt for 20 min and concentrated. The residue was purified by pre-TLC (DCM/MeOH=15/1) to give the title compound (81 mg, yield: 56%).

### Step 9. (S)—N-((4-((5-(7-amino-5,7-dihydrospiro[cyclopenta[b]pyridine-6,4′-piperidin]-1′-yl)pyrazin-2-yl)thio)-3-chloropyridin-2-yl)carbamoyl)benzenesulfonamide

To a solution of the product of Step 8 above (81 mg, 0.111 mmol) in THF (1 mL) was added 4N HCl/dioxane (1 mL) at 0° C. The mixture was stirred at rt for 1 h and concentrated. The residue was treated with sat. aq. NaHCO₃ (3 mL) and water (5 mL), extracted with DCM/isopropyl alcohol (3/1, 60 mL). The organic layer was dried over Na₂SO₄, filtered off, and concentrated. The residue was purified by pre-TLC (DCM/MeOH=6/1) to give the title compound (34 mg, yield: 49%). MS (ESI) m/z: 623.4 [M+1]+; ¹H NMR (400 MHz, DMSO-d6) δ 8.53 (s, 1H), 8.47 (d, J=4.7 Hz, 1H), 8.32 (s, 1H), 7.91 (d, J=5.7 Hz, 1H), 7.76 (m, 4H), 7.40-7.31 (m, 7H), 6.29 (d, J=5.7 Hz, 1H), 4.34 (s, 3H), 3.31-3.22 (m, 3H), 2.91 (d, J=16.5 Hz, 1H), 1.99 (m, 1H), 1.69 (m, 2H), 1.34 (d, J=13.0 Hz, 1H).

### Example 15

### Preparation of N-((4-((5-((3S,4S)-4-amino-3-methyl-2-oxa-8-azaspiro[4.5]decan-8-yl)[1,2,4]triazolo[4,3-c]pyrimidin-8-yl)thio)-3-chloropyridin-2-yl)carbamoyl)benzenesulfonamide TFA salt

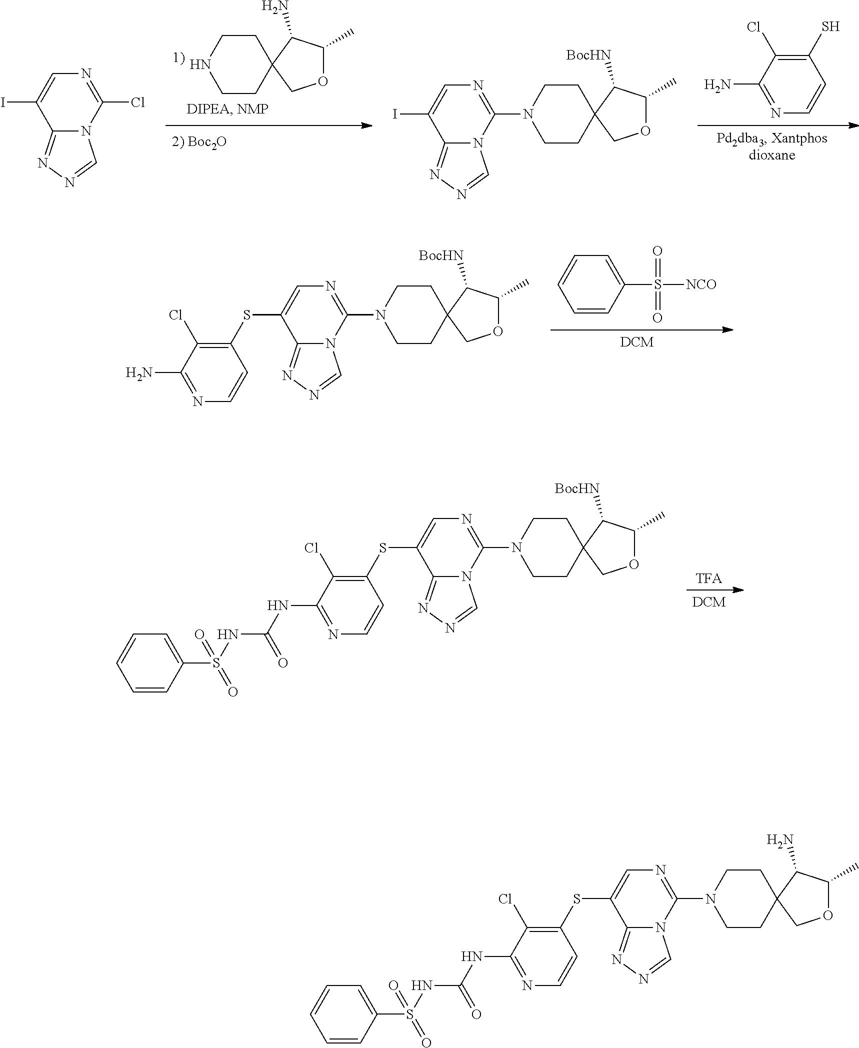

### Step 1. tert-butyl ((3S,4S)-8-(8-iodo-[1,2,4]triazolo[4,3-c]pyrimidin-5-yl)-3-methyl-2-oxa-8-azaspiro[4.5]decan-4-yl)carbamate

A mixture of 5-chloro-8-iodo-[1,2,4]triazolo[4,3-c]pyrimidine (280 mg, 1.0 mmol), (3S,4S)-3-methyl-2-oxa-8-azaspiro[4.5]decan-4-amine dihydrochloride (486 mg, 2.0 mmol), and DIPEA (507 mg, 4.0 mmol) in NMP (5 mL) was stirred at 100° C. for 5 h. The mixture was cooled to rt and Boc₂O (327 mg, 1.5 mmol) was added. The reaction mixture was stirred for 2 h at rt. The mixture was diluted with EtOAc (100 mL). The organic phase was washed with water (30 mL×2) and brine (30 mL), dried over Na₂SO₄, filtered off, and concentrated. The residue was purified by flash column chromatography on silica gel (DCM/MeOH=50/1 to 25/1) to give the title compound (370 mg, yield: 72%)

### Step 2. tert-butyl ((3S,4S)-8-(8-((2-amino-3-chloropyridin-4-yl)thio)-[1,2,4]triazolo[4,3-c]pyrimidin-5-yl)-3-methyl-2-oxa-8-azaspiro[4.5]decan-4-yl)carbamate

A mixture of the product of Step 1 above (370 mg, 0.72 mmol), 2-amino-3-chloropyridine-4-thiol (127 mg, 0.79 mmol), Pd₂dba₃ (64 mg, 0.07 mmol), Xantphos (81 mg, 0.14 mmol), and DIPEA (186 mg, 1.44 mmol) in dioxane (8 mL) was charged with N₂ and stirred at 100° C. for 3 h. The mixture was cooled to rt, diluted with H₂O (50 mL), and filtered off. The filtrate was concentrated. The residue was purified by Prep-TLC (DCM/MeOH=20/1) to give the title compound (180 mg, yield: 46%).

### Step 3. tert-butyl ((3S,4S)-8-(8-((3-chloro-2-(3-(phenylsulfonyl)ureido)pyridin-4-yl)thio)-[1,2,4]triazolo[4,3-c]pyrimidin-5-yl)-3-methyl-2-oxa-8-azaspiro[4.5]decan-4-yl)carbamate

To an ice-water cooled solution of the product of Step 2 above (180 mg, 0.33 mmol) in DCM (5 mL) was added benzenesulfonyl isocyanate (90 mg, 0.49 mmol). The mixture was stirred at rt for 1 h, diluted with Et₂O (10 mL), and filtered. The filter cake was dried to give the title compound (155 mg, yield: 65%).

### Step 4. N-((4-((5-((3S,4S)-4-amino-3-methyl-2-oxa-8-azaspiro[4.5]decan-8-yl)[1,2,4]triazolo[4,3-c]pyrimidin-8-yl)thio)-3-chloropyridin-2-yl)carbamoyl)benzenesulfonamide TFA salt

To an ice-water cooled solution of the product of Step 3 above (155 mg, 0.213 mmol) in DCM (5 mL) was added TFA (1 mL) slowly. The mixture was stirred at rt for 1 h, diluted with DCM (2 mL), and concentrated. The residue was triturated with Et₂O/DCM (5/5 mL) and filtered. The filter cake was dried to give the title compound (105 mg, yield: 66%). MS (ESI) m/z: 630.3 [M+1]+; ¹H NMR (400 MHz, CD₃OD) δ 9.32 (d, J=9.4 Hz, 1H), 8.07 (t, J=9.1 Hz, 2H), 7.98 (d, J=7.7 Hz, 1H), 7.90 (d, J=6.1 Hz, 1H), 7.69 (t, J=6.9 Hz, 1H), 7.61-7.47 (m, 3H), 4.37-4.09 (m, 3H), 4.02 (d, J=9.2 Hz, 1H), 3.92 (d, J=9.6 Hz, 1H), 3.52-3.40 (m, 3H), 2.06 (m, 3H), 1.83 (m, 1H), 1.34 (d, J=6.4 Hz, 3H).

### Example 16

### Preparation of N—(N-(3-((5-((3S,4S)-4-amino-3-methyl-2-oxa-8-azaspiro[4.5]decan-8-yl)pyrazin-2-yl)thio)-2-chlorophenyl)sulfamoyl)pyrrolidine-1-carboxamide

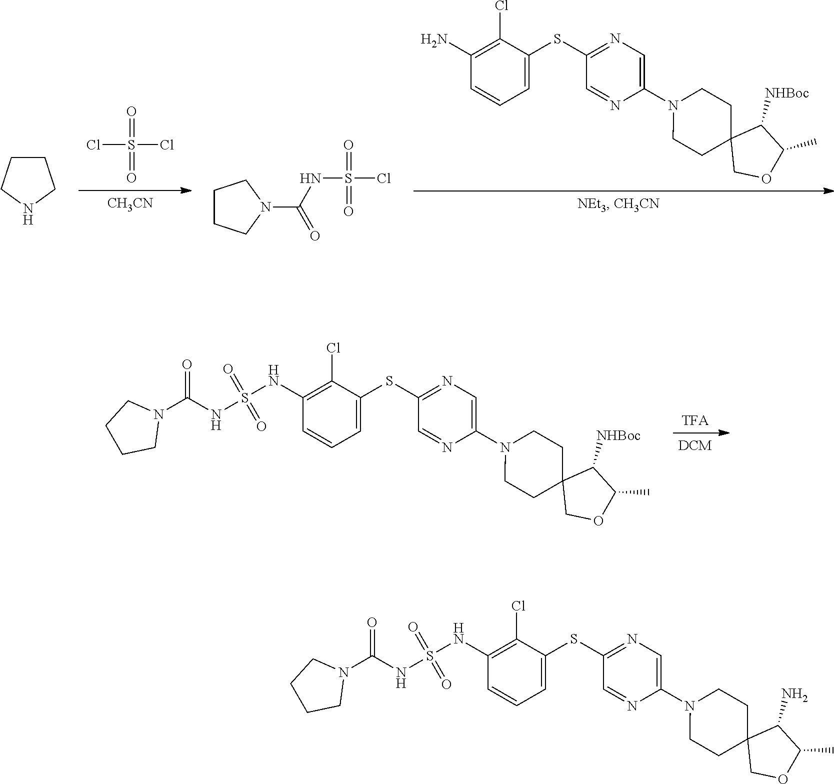

### Step 1. (pyrrolidine-1-carbonyl)sulfamoyl chloride

To a solution of sulfurisocyanatidic chloride (283 mg, 2.0 mmol) in CH₃CN (2 mL) cooled in ice-water bath was added pyrrolidine (142 mg, 2.0 mmol) drop-wise. The mixture was stirred for 2 hours in ice-water bath and was used in the next step directly.

### Step 2 N-((2-chloro-3-((5-chloropyrazin-2-yl)thio)phenyl)sulfonyl)benzamide

A solution of the product of Step 1 above (0.4 mmol) was added into the solution of the product of Step 1 in Example 10 (200 mg, 0.4 mmol) and TEA (40 mg, 0.8 mmol) in acetonitrile (2 mL) over an ice-water bath. The mixture was stirred at rt overnight, diluted with EtOAc (100 mL), washed with water (30 mL) and brine (30 mL), dried over Na₂SO₄, filtered off, and concentrated. The residue was purified by prep-TLC (DCM/MeOH/NH₄OH=10/1/0.02) to give the title compound (50 mg, crude yield: 18%).

### Step 3. N—(N-(3-((5-((3S,4S)-4-amino-3-methyl-2-oxa-8-azaspiro[4.5]decan-8-yl)pyrazin-2-yl)thio)-2-chlorophenyl)sulfamoyl)pyrrolidine-1-carboxamide

To a solution of the product of Step 2 above (50 mg, 0.073 mmol) in DCM (2 mL) was added TFA (1 mL) at rt. The mixture was stirred at rt for 1 h before concentrated. The residue was treated with sat. aq. NaHCO₃ (2 mL) and concentrated to remove the volatile. The residue was extracted with DCM/MeOH=10/1 (30 mL), dried by Na₂SO₄, filtered off, and concentrated. The residue was purified by prep-TLC (DCM/MeOH/NH₄OH=10/1/0.02) to give the title compound (18 mg, crude yield: 43%). MS (ESI) m/z: 582.2 [M+1]+; ¹H NMR (400 MHz, DMSO-d6) δ 8.40 (s, 1H), 8.18 (s, 1H), 7.51 (d, J=8.2 Hz, 1H), 7.04 (s, 1H), 6.41 (d, J=7.6 Hz, 1H), 4.12 (d, J=6.0 Hz, 3H), 3.79 (d, J=8.5 Hz, 1H), 3.59 (d, J=8.9 Hz, 1H), 3.3-3.16 (m, 7H), 1.77-1.48 (m, 8H), 1.14 (d, J=6.1 Hz, 3H).

### Example 17

### Preparation of 3-((5-((3S,4S)-4-amino-3-methyl-2-oxa-8-azaspiro[4.5]decan-8-yl)pyrazin-2-yl)thio)-2-chloro-N-(cyclopentylcarbamoyl)benzenesulfonamide

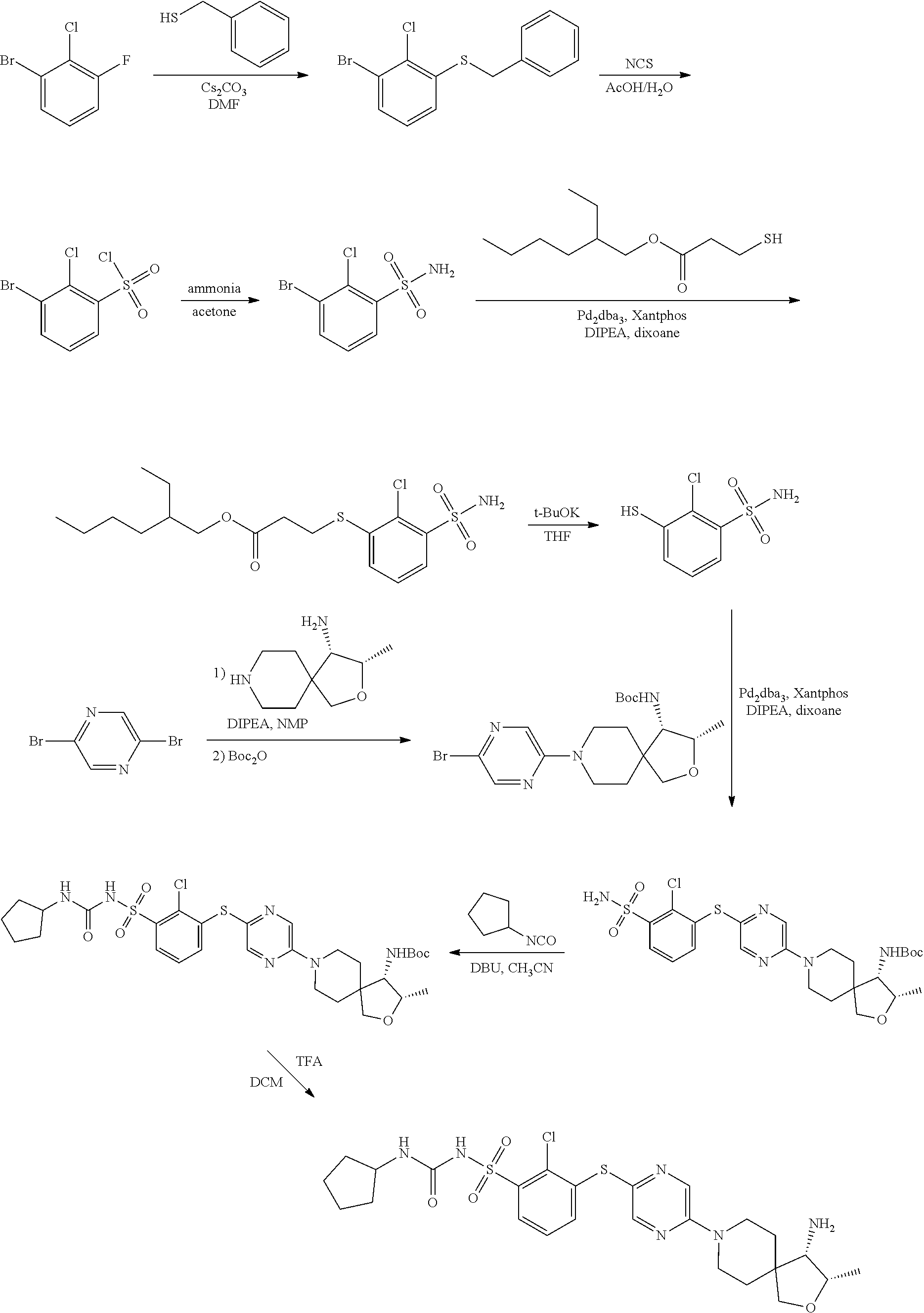

### Step 1. benzyl(3-bromo-2-chlorophenyl)sulfane

To a solution of phenylmethanethiol (6.5 g, 53 mmol) in DMF (100 mL) was added Cs₂CO₃ (20.3 g, 62 mmol). The mixture was stirred at rt for 10 min and a solution of 1-bromo-2-chloro-3-fluorobenzene (10 g, 48 mmol) in DMF (50 mL) was added. The mixture was stirred at rt under N₂ overnight and then at 75° C. overnight. The mixture was cooled to rt and diluted with EtOAc (300 mL), washed with water (100 mL×2) and brine (100 mL), dried over Na₂SO₄, filtered off, and concentrated. The residue was treated with EtOAc (20 mL), filtered and dried to give the title compound (10.2 g, yield: 68%).

### Step 2. 3-bromo-2-chlorobenzene-1-sulfonyl chloride

To a solution of the product of Step 1 above (5.0 g, 16 mmol) in AcOH/H₂O (40 mL/10 mL) was added NCS (8.5 g, 64 mmol) at rt. The mixture was stirred at rt for 2 h, diluted with EtOAc (500 mL), washed with water (100 mL×2) and brine (100 mL), dried over Na₂SO₄, filtered off, and concentrated to give the title compound (9.5 g, crude yield: 100%), which was used in the next step without any further purification.

### Step 3. 3-bromo-2-chlorobenzenesulfonamide

To a solution of the product of Step 2 above (9.5 g, crude) in acetone (100 mL) was added ammonia (28%, 200 mL) slowly at 0° C. The mixture was stirred at rt overnight before concentrated. The reside was added H₂O (20 mL), filtered and dried to give the compound (5.0 g, yield: 100%), which was used in the next step without any further purification.

### Step 4. 2-ethylhexyl 3-((2-chloro-3-sulfamoylphenyl)thio)propanoate

To a solution of the product of Step 3 above (5 g, 18 mmol) in dioxane (100 mL) was added 2-ethylhexyl 3-mercaptopropanoate (4.4 g, 20 mmol), Pd₂dba₃ (823 mg, 0.9 mmol), XantPhos (1.0 g, 1.8 mmol) and DIPEA (4.7 g, 36 mmol). The mixture was charged with N₂, stirred at 110° C. overnight, cooed to rt, diluted with EtOAc (500 mL), washed with water (100 mL×2) and brine (100 mL), dried over Na₂SO₄, filtered off, and concentrated. The residue was purified by flash column chromatography on silica gel (PE/EtOAc=5/1-2/1) to give the title compound (5.6 g, yield: 77%).

### Step 5. 2-chloro-3-mercaptobenzenesulfonamide

To a solution of the product of Step 4 above (5.6 g, 14 mmol) in THF (100 mL) at −78° C. under N₂ was added a solution of t-BuOK (4.6 g, 41 mmol) in THF (41 mL) slowly. After the addition was complete, the mixture was stirred at −78° C. for 30 min before quenching with aqueous K₂CO₃ solution (2 N, 50 mL). The mixture was extracted with MBTE (30 mL×2). The aqueous phase was acidified by 6N HCl to pH of 4, filtered off and dried to give the title compound (2.7 g, yield: 86%), as off-white solid.

### Step 6. tert-butyl ((3S,4S)-8-(5-bromopyrazin-2-yl)-3-methyl-2-oxa-8-azaspiro[4.5]decan-4-yl)carbamate

To a solution of 2,5-dibromopyrazine (357 mg, 1.5 mmol) and (3S,4S)-3-methyl-2-oxa-8-azaspiro[4.5]decan-4-amine dihydrochloride (1.95 mmol, 1.3 mmol) in NMP (10 mL) was added DIPEA (775 mg, 6.0 mmol). The mixture was stirred at 110° C. overnight, cooled to rt and Boc₂O (491 mg, 2.25 mmol) was added. The reaction mixture was stirred at rt for 2 h, diluted with EA (100 mL), washed with water (30 mL×2) and brine (30 mL), dried over Na₂SO₄, filtered off and concentrated. The residue was purified by flash column chromatography on silica gel (PE/EtOAc=4/1 to 3/2) to give the title compound (540 mg, yield: 85%).

### Step 7. tert-butyl ((3S,4S)-8-(5-((2-chloro-3-sulfamoylphenyl)thio)pyrazin-2-yl)-3-methyl-2-oxa-8-azaspiro[4.5]decan-4-yl)carbamate

A solution of the product of Step 5 above (130 mg, 0.58 mmol), the product of Step 6 above (165 mg, 0.39 mmol), Pd₂dba₃ (29 mg, 0.029 mmol), Xantphos (34 mg, 0.058 mmol), DIPEA (150 mg, 1.16 mmol) in dioxane (5 mL) was charged with N₂ and stirred at 120° C. for 3 days. The mixture was cooled to rt, diluted with EtOAc (100 mL), washed with water (30 mL) and brine (30 mL), dried over Na₂SO₄, filtered off, and concentrated. The residue was purified by prep-TLC (DCM/MEOH=15/1) to give the title compound (85 mg, yield: 38%).

### Step 8. tert-butyl ((3S,4S)-8-(5-((2-chloro-3-(N-(cyclopentylcarbamoyl)sulfamoyl)phenyl)thio)pyrazin-2-yl)-3-methyl-2-oxa-8-azaspiro[4.5]decan-4-yl)carbamate

A mixture of the product of Step 7 above (100 mg, 0.18 mmol), isocyanatocyclopentane (23 mg, 0.21 mmol) and DBU (40 mg, 0.26 mmol) in acetonitrile (3 mL) was stirred at 80° C. for 3 h. The mixture was diluted with EtOAc (100 mL), washed with water (30 mL) and brine (30 mL), dried over Na₂SO₄, filtered off, and concentrated. The residue was purified by prep-TLC (DCM/MEOH=10/1) to give the title compound (80 mg, yield: 66%).

### Step 9. 3-((5-((3S,4S)-4-amino-3-methyl-2-oxa-8-azaspiro[4.5]decan-8-yl)pyrazin-2-yl)thio)-2-chloro-N-(cyclopentylcarbamoyl)benzenesulfonamide

To a solution of the product of Step 8 above (80 mg, 0.12 mmol) in DCM (2 mL) was added TFA (1 mL) at rt. The mixture was stirred at rt for 1 h and concentrated. The residue was purified by flash column chromatography on a C18 reverse phase column (MeOH/H₂O) to give the title compound (45 mg, yield: 64%). MS (ESI) m/z: 581.2 [M+1]+; ¹H NMR (400 MHz, DMSO-d6) δ 8.48 (s, 1H), 8.29 (s, 1H), 7.84 (d, J=7.6 Hz, 1H), 7.39 (t, J=7.5 Hz, 1H), 7.11 (d, J=7.6 Hz, 1H), 4.31-4.10 (m, 2H), 3.89 (d, J=9.1 Hz, 1H), 3.69 (t, J=10.7 Hz, 2H), 3.38 (s, 2H), 3.21-3.06 (m, 2H), 1.72 (s, 6H), 1.57 (s, 2H), 1.45 (s, 2H), 1.27 (d, J=6.2 Hz, 2H), 1.21 (d, J=6.4 Hz, 3H).

Table 1 summarizes and lists examples that were prepared according to the procedures as described in Examples 1-17 as indicated below the structure of each example by using the corresponding intermediates and reagents under appropriate conditions that could be accomplished by the skilled persons.

TABLE 1

| Ex- | | | | |
|---|---|---|---|---|
| am- | | | LC/MS | |
| ple | | | [M + | |
| # | Structure | Chemical Name | H]⁺ | ¹H NMR |
| 1 | 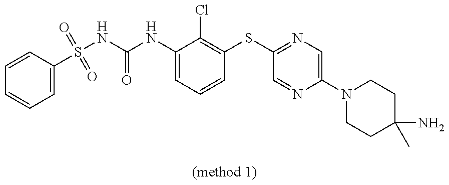 [SMILES: CC1(N)CCN(c2cnc(Sc3cccc(NC(=O)NS(=O)(=O)c4ccccc4)c3Cl)cn2)CC1] | N-((3-((5-(4-amino-4- methylpiperidin-1- yl)pyrazin-2-yl)thio)- 2-chlorophenyl)carba- moyl)benzene- sulfonamide | 533.2 | ¹H NMR (400 MHz, DMSO-d6) δ 8.40 (s, 1H), 8.18 (s, 1H), 8.04 (s, 1H), 7.75 (s, 5H), 7.49 (s, 1H), 7.37 (s, 3H), 7.02 (s, 1H), 6.44 (s, 1H), 4.06 (s, 2H), 3.35 (s, 2H), 1.70 (s, 4H), 1.34 (s, 3H). |
| 2 | 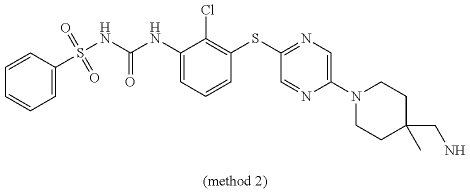 [SMILES: CC1(CN)CCN(c2cnc(Sc3cccc(NC(=O)NS(=O)(=O)c4ccccc4)c3Cl)cn2)CC1] | N-((3-((5-(4- (aminomethyl)-4- methylpiperidin-1-yl) pyrazin-2-yl)thio)-2- chlorophenyl)carba- moyl)benzene- sulfonamide | 547.2 | ¹H NMR (400 MHz, DMSO-d6) δ 8.37 (s, 1H), 8.16 (s, 1H), 8.03 (d, J = 7.7 Hz, 1H), 7.75 (s, 2H), 7.51 (m, 4H), 7.37 (s, 3H), 7.01 (s, 1H), 6.40 (d, J = 7.7 Hz, 1H), 3.87 (s, 2H), 3.39 (s, 2H), 2.76 (s, 2H), 1.46 m, 4H), 1.05 (s, 3H). |
| 3 | 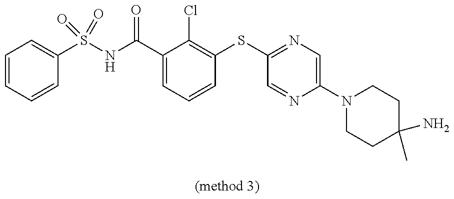 [SMILES: CC1(N)CCN(c2cnc(Sc3cccc(C(=O)NS(=O)(=O)c4ccccc4)c3Cl)cn2)CC1] | 3-((5-(4-amino-4- methylpiperidin-1- yl)pyrazin-2-yl)thio)- 2-chloro-N- (phenylsulfonyl)ben- zamide | 518.3 | ¹H NMR (400 MHz, DMSO-d6) δ 8.41 (s, 1H), 8.20 (s, 1H), 7.94 (s, 3H), 7.81 (s, 2H), 7.45 (m, 3H), 7.19-7.10 (m, 2H), 6.93-6.85 (m, 1H), 4.05 (d, J = 12.8 Hz, 2H), 3.34 (m, 2H), 1.70 (m, 4H), 1.35 (s, 3H). |
| 4 | 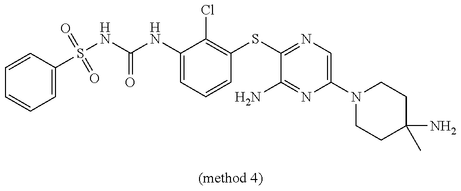 [SMILES: CC1(N)CCN(c2cnc(Sc3cccc(NC(=O)NS(=O)(=O)c4ccccc4)c3Cl)c(N)n2)CC1] | N-((3-((3-amino-5-(4- amino-4- methylpiperidin-1- yl)pyrazin-2-yl)thio)- 2-chlorophenyl)carba- moyl)benzene- sulfonamide | 548.3 | ¹H NMR (400 MHz, DMSO-d6) δ 7.95 (d, J = 8.3 Hz, 1H), 7.75 (b, 5H), 7.61 (s, 1H), 7.47 (s, 1H), 7.38-7.33 (m, 3H), 6.95 (t, J = 8.1 Hz, 1H), 6.15- 6.04 (m, 3H), 4.00 (d, J = 13.6 Hz, 2H), 3.25 (m, 2H), 1.67 (s, 4H), 1.34 (s, 3H). |
| 5 | 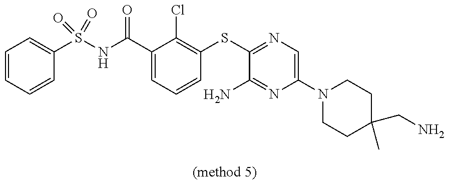 [SMILES: CC1(CN)CCN(c2cnc(Sc3cccc(C(=O)NS(=O)(=O)c4ccccc4)c3Cl)c(N)n2)CC1] | 3-((3-amino-5-(4- (aminomethyl)-4- methylpiperidin-1- yl)pyrazin-2-yl)thio)- 2-chloro-N- (phenylsulfonyl)ben- zamide | 547.3 | ¹H NMR (400 MHz, DMSO-d6) δ 7.84-7.75 (m, 2H), 7.73-7.57 (m, 3H), 7.61 (s, 1H), 7.43- 7.36 (m, 3H), 7.09-7.01 (m, 2H), 6.53-6.45 (m, 1H), 6.04 (s, 2H), 3.90- 3.77 (m, 2H), 3.37-3.32 (m, 2H), 2.75 (s, 2H), 1.53-1.42 (m, 2H), 1.37 (d, J = 13.0 Hz, 2H), |
| | | | | 1.05-1.02 (m, 3H). |
| 6 | 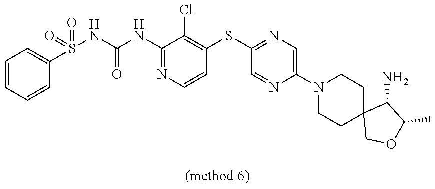 [SMILES: C[C@@H]1OCC2(CCN(c3cnc(Sc4ccnc(NC(=O)NS(=O)(=O)c5ccccc5)c4Cl)cn3)CC2)[C@@H]1N] | N-((4-((5-((3S,4S)-4- amino-3-methyl-2- oxa-8-azaspiro [4.5]decan-8-yl) pyrazin-2-yl)thio)- 3-chloropyridin-2- yl)carbamoyl)ben- zenesulfonamide | 590.0 | ¹H NMR (400 MHz, DMSO-d6) δ 8.53 (s, 1H), 8.33 (s, 1H), 7.35 (m, 6H), 6.23 (s, 1H), 4.14 (m, 3H), 3.81 (d, J = 8.4 Hz, 1H), 3.62 (d, J = 8.8 Hz, 1H), 3.28 (m, 3H), 1.67 (m, 4H), 1.16 (d, J = 6.3 Hz, 3H). |
| 7 | 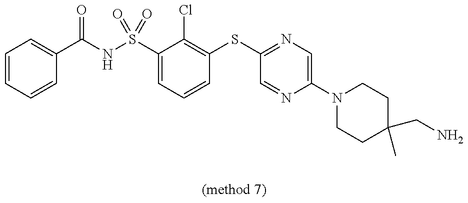 [SMILES: CC1(CN)CCN(c2cnc(Sc3cccc(S(=O)(=O)NC(=O)c4ccccc4)c3Cl)cn2)CC1] | N-((3-((5-(4- (aminomethyl)-4- methylpiperidin-1- yl)pyrazin-2-yl)thio)- 2-chlorophenyl) sulfonyl)benzamide | 532.0 | ¹H NMR (400 MHz, DMSO-d6) δ 8.42 (s, 1H), 8.20 (s, 1H), 7.87 (d, J = 7.2 Hz, 2H), 7.81 (d, J = 7.6 Hz, 1H), 7.64 (s, 3H), 7.40-7.33 (m, 1H), 7.29 (t, J = 7.2 Hz, 2H), 7.22 (t, J = 7.9 Hz, 1H), 6.84 (d, J = 7.7 Hz, 1H), 3.90 (m, 2H), 3.40 (m, 2H), 2.76 (s, 2H), 1.56- 1.46 (m, 2H), 1.44-1.36 (m, 2H), 1.05 (s, 3H). |
| 8 | 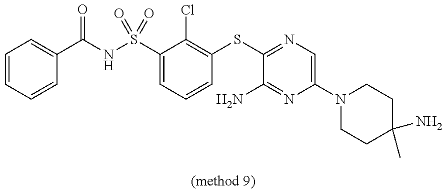 [SMILES: CC1(N)CCN(c2cnc(Sc3cccc(S(=O)(=O)NC(=O)c4ccccc4)c3Cl)c(N)n2)CC1] | N-((3-((3-amino-5-(4- amino-4- methylpiperidin-1-yl) pyrazin-2-yl)thio)-2- chlorophenyl)sulfo- nyl)benzamide | 533.2 | ¹H NMR (400 MHz, DMSO-d6) δ 8.25 (s, 3H), 7.90 (m, 3H), 7.68- 7.58 (m, 2H), 7.48 (m, 3H), 6.87 (d, J = 7.9 Hz, 1H), 4.01 (m, 2H), 3.35 (m, 2H), 1.73 (m, 4H), 1.36 (s, 3H). |
| 9 | 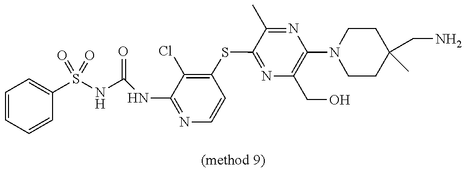 [SMILES: Cc1nc(N2CCC(C)(CN)CC2)c(CO)nc1Sc1ccnc(NC(=O)NS(=O)(=O)c2ccccc2)c1Cl] | N-((4-((5-(4- (aminomethyl)-4- methylpiperidin-1-yl)- 6-(hydroxymethyl)-3- methylpyrazin-2- yl)thio)-3- chloropyridin-2- yl)carbamoyl)ben- zenesulfonamide | 592.2 | ¹H NMR (400 MHz, DMSO-d6) δ 7.88 (d, J = 4.9 Hz, 1H), 7.76 (s, 1H), 7.86-7.62 (b, 3H), 7.37 (s, 3H), 6.18 (d, J = 4.5 Hz, 1H), 5.49 (s, 1H), 4.44 (s, 2H), 3.65 (d, J = 12.9 Hz, 2H), 3.35 (s, 2H), 2.77 (s, 2H), 2.39 (s, 3H), 1.58 (m, 2H), 1.47 (m, 2H), 1.05 (s, 3H). |
| 10 | 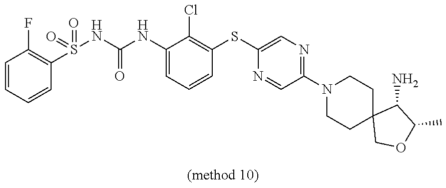 [SMILES: C[C@@H]1OCC2(CCN(c3cnc(Sc4cccc(NC(=O)NS(=O)(=O)c5ccccc5F)c4Cl)cn3)CC2)[C@@H]1N] | N-((3-((5-((3S,4S)-4- amino-3-methyl-2- oxa-8-azaspiro [4.5]decan-8-yl) pyrazin-2-yl)thio)- 2-chloro- phenyl)carbamoyl)- 2-fluoro- benzenesulfonamide | 607.2 | ¹H NMR (400 MHz, DMSO-d6) δ 8.42 (s, 1H), 8.19 (s, 1H), 7.97 (d, J = 8.2 Hz, 1H), 7.77 (t, J = 7.3 Hz, 1H), 7.59 (s, 1H), 7.44 (m, 2H), 7.18 (m, 2H), 7.02 (t, J = 8.1 Hz, 1H), 6.42 (d, J = 7.8 Hz, 1H), 4.29 - 4.07 (m, 3H), 3.91 (d, J = 8.8 Hz, 1H), 3.64 (d, J = 9.0 Hz, 1H), 3.30 (m, 1H), 3.08 |
| | | | | (m, 2H), 1.77 (m, 2H), |
| | | | | 1.62 (m, 2H), 1.22 (d, J = |
| | | | | 6.3 Hz, 3H). |
| 11 | 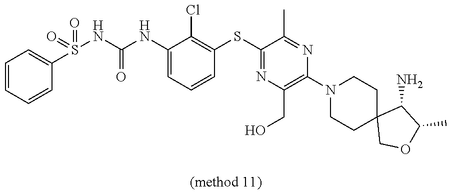 [SMILES: Cc1nc(N2CCC3(CC2)CO[C@@H](C)[C@H]3N)c(CO)nc1Sc1cccc(NC(=O)NS(=O)(=O)c2ccccc2)c1Cl] | N-((3-((5-((3S,4S)- 4-amino-3- methyl-2-oxa- 8-azaspiro[4.5]decan- 8-yl)-6- (hydroxymethyl)-3- methylpyrazin-2- yl)thio)-2-chloro- phenyl)carbamoyl) benzenesulfonamide | 633.2 | ¹H NMR (400 MHz, DMSO-d6) δ 8.03 (d, J = 8.3 Hz, 1H), 7.83-7.72 (m, 2H), 7.55 (s, 1H), 7.44-7.30 (m, 3H), 7.02 (t, J = 8.1 Hz, 1H), 6.34 (d, J = 7.7 Hz, 1H), 5.35 (t, J = 5.2 Hz, 1H), 4.42 (d, J = 4.8 Hz, 2H), 4.23- 4.11 (m, 1H), 3.90- 3.59 (m, 4H), 3.42-3.22 (m, 1H), 3.12-2.91 (m, |
| | | | | 2H), 2.38 (s, 3H), 1.89- |
| | | | | 1.48 (m, 4H), 1.20 (d, J = |
| | | | | 8.6 Hz, 3H). |
| 12 | 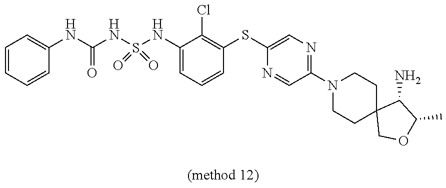 [SMILES: C[C@@H]1OCC2(CCN(c3cnc(Sc4cccc(NS(=O)(=O)NC(=O)Nc5ccccc5)c4Cl)cn3)CC2)[C@@H]1N] | ((3S,4S)-8-(5-((2- chloro-3-((N-(phenyl- carbamoyl)sulfa- moyl)amino)phenyl) thio)pyrazin-2-yl)-3- methyl-2-oxa-8- azaspiro[4.5]decan-4- yl)amine | 604.3 | ¹H NMR (400 MHz, DMSO-d6) δ 8.42 (s, 1H), 8.20 (s, 1H), 7.54 (d, J = 8.2 Hz, 1H), 7.45 (d, J = 8.2 Hz, 2H), 7.18-6.99 (m, 3H), 6.79 (d, J = 7.2 Hz, 1H), 6.46 (d, J = 7.8 Hz, 1H), 4.24-4.09 (m, 3H), 3.87 (d, J = 8.9 Hz, 1H), 3.67 (d, J = 9.1 Hz, 1H), 3.37 (d, J = 4.7 Hz, 1H), 3.19-3.03 (m, 2H), |
| | | | | 1.66 (m, 4H), 1.18 (d, J = |
| | | | | 6.5 Hz, 3H). |
| 13 | 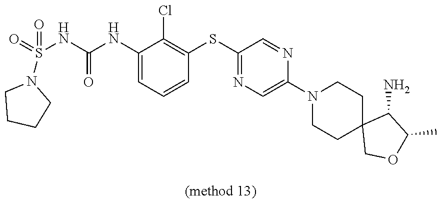 [SMILES: C[C@@H]1OCC2(CCN(c3cnc(Sc4cccc(NC(=O)NS(=O)(=O)N5CCCC5)c4Cl)cn3)CC2)[C@@H]1N] | N-((3-((5-((3S,4S)-4- amino-3-methyl-2- oxa-8-azaspiro [4.5]decan-8- yl)pyrazin-2-yl)thio)- 2-chloro- phenyl)carbamoyl) pyrrolidine-1- sulfonamide | 582.1 | ¹H NMR (400 MHz, DMSO-d6) δ 8.38 (s, 1H), 8.18 (s, 1H), 8.10 (s, 1H), 7.04 (t, J = 8.0 Hz, 1H), 6.37 (d, J = 7.6 Hz, 1H), 4.11-4.01 (m, 1H), 3.92-3.78 (m, 2H), 3.65 (d, J = 8.3 Hz, 1H), 3.47 (d, J = 8.1 Hz, 1H), 3.31 (m, 1H), 3.11-3.02 (m, 5H), 2.89 (d, J = 4.6 Hz, 1H), 1.74-1.68 (m, 5H), 1.54-1.46 (m, 2H), 1.24- |
| | | | | 1.20 (m, 1H), 1.06 (d, J = |
| | | | | 6.3 Hz, 3H). |
| 14 | 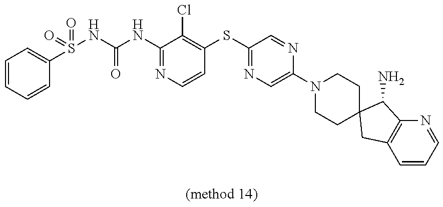 [SMILES: N[C@@H]1c2ncccc2CC12CCN(c1cnc(Sc3ccnc(NC(=O)NS(=O)(=O)c4ccccc4)c3Cl)cn1)CC2] | (S)-N-((4-((5-(7- amino-5,7-dihydro- spiro[cyclopenta [b]pyridine-6,4′- piperidin]-1′- yl)pyrazin-2-yl)thio)- 3-chloropyridin-2- yl)carbamoyl)ben- zenesulfonamide | 623.4 | ¹H NMR (400 MHz, DMSO-d6) δ 8.53 (s, 1H), 8.47 (d, J = 4.7 Hz, 1H), 8.32 (s, 1H), 7.91 (d, J = 5.7 Hz, 1H), 7.76 (m, 4H), 7.40-7.31 (m, 7H), 6.29 (d, J = 5.7 Hz, 1H), 4.34 (s, 3H), 3.31-3.22 (m, 3H), 2.91 (d, J = 16.5 Hz, 1H), 1.99 (m, 1H), 1.69 (m, 2H), 1.34 (d, J = 13.0 Hz, 1H). |
| 15 | 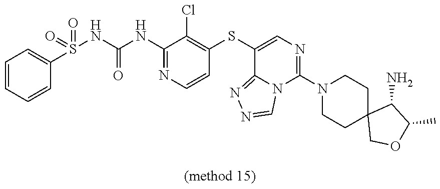 [SMILES: C[C@@H]1OCC2(CCN(c3ncc(Sc4ccnc(NC(=O)NS(=O)(=O)c5ccccc5)c4Cl)c4nncn34)CC2)[C@@H]1N] | N-((4-((5-((3S,4S)-4- amino-3-methyl-2- oxa-8-azaspiro [4.5]decan- 8-yl)-[1,2,4]tri- azolo[4,3-c] pyrimidin-8-yl)thio)- 3-chloropyridin-2- yl)carbamoyl)ben- zenesulfonamide | 630.3 | ¹H NMR (400 MHz, CD₃OD) δ 9.32 (d, J = 9.4 Hz, 1H), 8.07 (t, J = 9.1 Hz, 2H), 7.98 (d, J = 7.7 Hz, 1H), 7.90 (d, J = 6.1 Hz, 1H), 7.69 (t, J = 6.9 Hz, 1H), 7.61-7.47 (m, 3H), 4.37-4.09 (m, 3H), 4.02 (d, J = 9.2 Hz, 1H), 3.92 (d, J = 9.6 Hz, 1H), 3.52-3.40 (m, 3H), 2.06 (m, 3H), 1.83 (m, |
| | | | | 1H), 1.34 (d, J = 6.4 Hz, |
| | | | | 3H). |
| 16 | 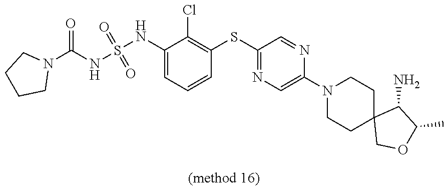 [SMILES: C[C@@H]1OCC2(CCN(c3cnc(Sc4cccc(NS(=O)(=O)NC(=O)N5CCCC5)c4Cl)cn3)CC2)[C@@H]1N] | N-(N-(3-((5-((3S,4S)- 4-amino-3-methyl-2- oxa-8- azaspiro[4.5]decan-8- yl)pyrazin-2-yl)thio)- 2-chlorophenyl)sulfa- moyl)pyrrolidine-1- carboxamide | 582.2 | ¹H NMR (400 MHz, DMSO-d6) δ 8.40 (s, 1H), 8.18 (s, 1H), 7.51 (d, J = 8.2 Hz, 1H), 7.04 (s, 1H), 6.41 (d, J = 7.6 Hz, 1H), 4.12 (d, J = 6.0 Hz, 3H), 3.79 (d, J = 8.5 Hz, 1H), 3.59 (d, J = 8.9 Hz, 1H), 3.3-3.16 (m, 7H), 1.77-1.48 (m, 8H), 1.14 (d, J = 6.1 Hz, 3H). |
| 17 | 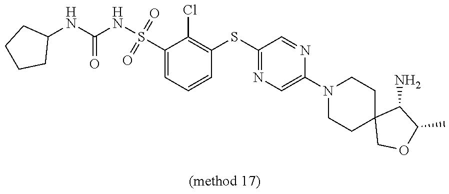 [SMILES: C[C@@H]1OCC2(CCN(c3cnc(Sc4cccc(S(=O)(=O)NC(=O)NC5CCCC5)c4Cl)cn3)CC2)[C@@H]1N] | 3-((5-((3S,4S)-4- amino-3-methyl-2- oxa-8-azaspiro[4.5] decan-8-yl)pyrazin-2- yl)thio)-2-chloro-N- (cyclopentyl- carbamoyl) benzenesulfonamide | 581.2 | ¹H NMR (400 MHz, DMSO-d6) δ 8.48 (s, 1H), 8.29 (s, 1H), 7.84 (d, J = 7.6 Hz, 1H), 7.39 (t, J = 7.5 Hz, 1H), 7.11 (d, J = 7.6 Hz, 1H), 4.31-4.10 (m, 2H), 3.89 (d, J = 9.1 Hz, 1H), 3.69 (t, J = 10.7 Hz, 2H), 3.38 (s, 2H), 3.21-3.06 (m, 2H), 1.72 (s, 6H), 1.57 (s, 2H), 1.45 (s, 2H), 1.27 (d, J = 6.2 |
| | | | | Hz, 2H), 1.21 (d, J = 6.4 |
| | | | | Hz, 3H). |
| 18 | 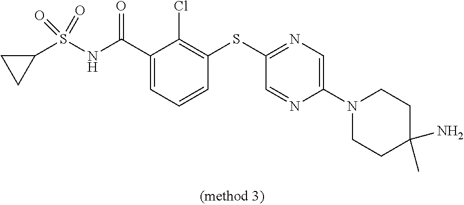 [SMILES: CC1(N)CCN(c2cnc(Sc3cccc(C(=O)NS(=O)(=O)C4CC4)c3Cl)cn2)CC1] | 3-((5-(4-amino-4- methylpiperidin-1- yl)pyrazin-2-yl)thio)- 2-chloro-N-(cyclo- propylsulfonyl)ben- zamide | 482.3 | ¹H NMR (400 MHz, DMSO-d6) δ 8.41 (s, 1H), 8.22 (s, 1H), 7.88 (s, 3H), 7.20-7.07 (m, 2H), 6.83 (d, J = 7.3 Hz, 1H), 4.01 (d, J = 13.7 Hz, 2H), 3.32 (s, 2H), 2.82 (m, 1H), 1.65 (m, 4H), 1.33 (s, 3H), 0.81 (m, 2H), 0.71 (m, 2H). |
| 19 | 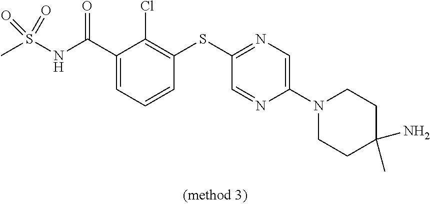 [SMILES: CC1(N)CCN(c2cnc(Sc3cccc(C(=O)NS(C)(=O)=O)c3Cl)cn2)CC1] | 3-((5-(4-amino-4- methylpiperidin-1- yl)pyrazin-2-yl)thio)- 2-chloro-N- (methylsulfonyl)ben- zamide | 456.2 | ¹H NMR (400 MHz, DMSO-d6) δ 8.42 (s, 1H), 8.19 (s, 1H), 7.96 (b, 3H), 7.23-7.10 (m, 2H), 6.88 (d, J = 7.3 Hz, 1H), 4.05 (d, J = 13.8 Hz, 2H), 3.43-3.33 (m, 2H), 2.89 (s, 3H), 1.70 (b, 4H), 1.35 (s, 3H). |
| 20 | 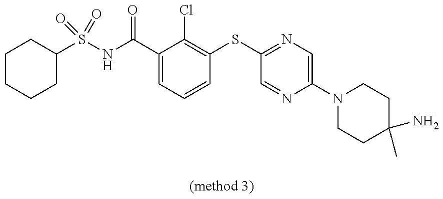 [SMILES: CC1(N)CCN(c2cnc(Sc3cccc(C(=O)NS(=O)(=O)C4CCCCC4)c3Cl)cn2)CC1] | 3-((5-(4-amino-4- methylpiperidin-1- yl)pyrazin-2-yl)thio)- 2-chloro-N-(cyclo- hexylsulfonyl)ben- zamide | 524.4 | ¹H NMR (400 MHz, DMSO-d6) δ 8.41 (s, 1H), 8.20 (s, 1H), 7.36 (d, J = 37.2 Hz, 3H), 7.13 (s, 2H), 6.85 (d, J = 7.2 Hz, 1H), 3.96 (s, 2H), 3.39 (s, 3H), 2.09-1.91 (m, 6H), 1.66 (t, J = 24.1 Hz, 8H), 1.31 (s, 3H). |
| 21 | 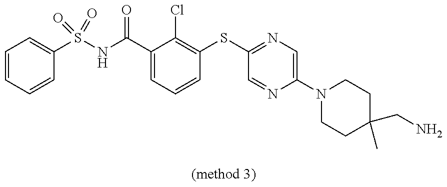 [SMILES: CC1(CN)CCN(c2cnc(Sc3cccc(C(=O)NS(=O)(=O)c4ccccc4)c3Cl)cn2)CC1] | 3-((5-(4- (aminomethyl)-4- methylpiperidin-1- yl)pyrazin-2-yl)thio)- 2-chloro-N- (phenylsulfonyl)ben- zamide | 532.3 | ¹H NMR (400 MHz, DMSO-d6) δ 8.38 (s, 1H), 8.16 (s, 1H), 7.83- 7.73 (m, 2H), 7.64 (s, 3H), 7.39 (s, 3H), 7.16- 7.04 (m, 2H), 6.80 (d, J = 7.3 Hz, 1H), 3.88 (d, J = 13.5 Hz, 2H), 3.38 (t, J = 10.0 Hz, 2H), 2.75 (s, 2H), 1.54-1.34 (m, 4H), 1.04 (s, 3H). |
| 22 | 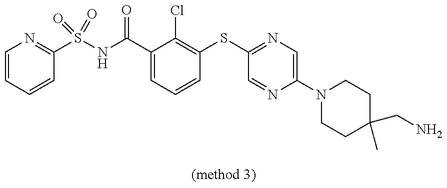 [SMILES: CC1(CN)CCN(c2cnc(Sc3cccc(C(=O)NS(=O)(=O)c4ccccn4)c3Cl)cn2)CC1] | 3-((5-(4- (aminomethyl)-4- methylpiperidin-1- yl)pyrazin-2-yl)thio)-2- chloro-N-(pyridin-2- ylsulfonyl)benzamide | 533.1 | ¹H NMR (400 MHz, DMSO-d6) δ 8.53 (d, , J = 6.6 Hz, 1H), 8.38 (s, 1H), 8.17 (s, 1H), 7.91- 7.84 (m, 2H), 7.67 (s, 3H), 7.41 (s, 1H), 7.20 (d, J = 6.6 Hz, 1H), 7.14- 7.08 (m, 1H), 6.82 (d, J = 7.6 Hz, 1H), 3.88 (m, 2H), 3.42-3.34 (m, 2H), 2.76 (s, 2H), 1.53-1.45 (m, 2H), 1.39 (m, 2H), |
| | | | | 1.04 (s, 3H). |
| 23 | 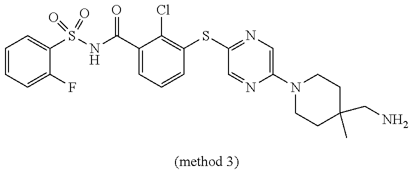 [SMILES: CC1(CN)CCN(c2cnc(Sc3cccc(C(=O)NS(=O)(=O)c4ccccc4F)c3Cl)cn2)CC1] | 3-((5-(4- (aminomethyl)-4- methylpiperidin-1- yl)pyrazin-2-yl)thio)- 2-chloro-N-((2- fluorophenyl)sulfo- nyl)benzamide | 550.4 | ¹H NMR (400 MHz, DMSO-d6) δ 8.38 (s, 1H), 8.17 (s, 1H), 7.86- 7.75 (m, 1H), 7.67 (b, 3H), 7.45 (s, 1H), 7.25- 7.06 (m, 4H), 6.83 (d, J = 7.5 Hz, 1H), 3.88 (m, 2H), 3.46- 3.34 (m, 2H), 2.75 (s, 2H), 1.58-1.44 (m, 2H), 1.41 (m, 2H), 1.04 (s, 3H). |
| 24 | 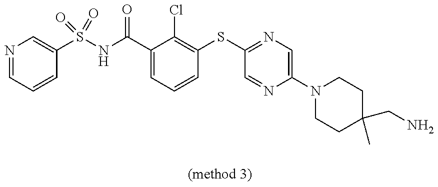 [SMILES: CC1(CN)CCN(c2cnc(Sc3cccc(C(=O)NS(=O)(=O)c4cccnc4)c3Cl)cn2)CC1] | 3-((5-(4- (aminomethyl)-4- methylpiperidin-1-yl) pyrazin-2-yl)thio)-2- chloro-N-(pyridin-3- ylsulfonyl)benzamide | 533.2 | ¹H NMR (400 MHz, DMSO-d6) δ 8.90 (s, 1H), 8.59 (d, J = 3.3 Hz, 1H), 8.38 (s, 1H), 8.16 (s, 1H), 8.12 (d, J = 7.7 Hz, 1H), 7.66 (bs, 3H), 7.51- 7.40 (m, 1H), 7.18-7.07 (m, 2H), 6.83 (d, J = 7.3 Hz, 1H), 3.88 (m, 2H), 3.43-3.35 (m, 2H), 2.76 (s, 2H), 1.59-1.45 (m, 2H), 1.41 (m, 2H), 1.04 |
| | | | | (s, 3H). |
| 25 | 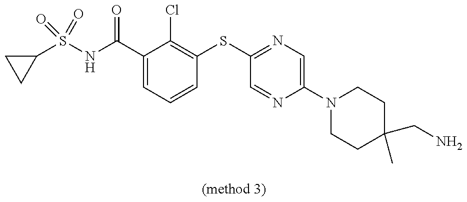 [SMILES: CC1(CN)CCN(c2cnc(Sc3cccc(C(=O)NS(=O)(=O)C4CC4)c3Cl)cn2)CC1] | 3-((5-(4- (aminomethyl)-4- methylpiperidin-1- yl)pyrazin-2-yl)thio)- 2-chloro-N-(cyclo- propylsulfonyl)ben- zamide | 496.3 | ¹H NMR (400 MHz, DMSO-d6) δ 8.41 (s, 1H), 8.18 (s, 1H), 7.85- 7.49 (m, 3H), 7.24-7.06 (m, 2H), 6.83 (d, J = 7.5 Hz, 1H), 3.92-3.83 (m, 2H), 3.44-3.35 (m, 2H), 2.84 (m, 1H), 2.75 (s, 2H), 1.49 (m, 2H), 1.42 (m, 2H), 1.04 (s, 3H), 0.80 (m, 2H), 0.74 (m, 2H). |
| 26 | 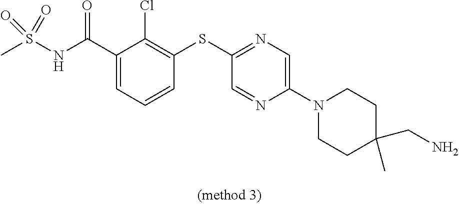 [SMILES: CC1(CN)CCN(c2cnc(Sc3cccc(C(=O)NS(C)(=O)=O)c3Cl)cn2)CC1] | 3-((5-(4- (aminomethyl)-4- methylpiperidin-1- yl)pyrazin-2-yl)thio)- 2-chloro-N- (methylsulfonyl)ben- zamide | 470.3 | ¹H NMR (400 MHz, DMSO-d6) δ 8.39 (s, 1H), 8.18 (s, 1H), 7.67 (s, 3H), 7.19-7.07 (m, 2H), 6.82 (d, J = 7.0 Hz, 1H), 3.88 (m, 2H), 3.39 (m, 2H), 2.84 (s, 3H), 2.75 (s, 2H), 1.56-1.44 (m, 2H), 1.41 (m, 2H), 1.04 (s, 3H). |
| 27 | 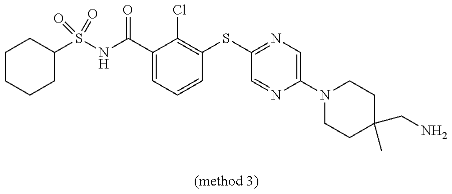 [SMILES: CC1(CN)CCN(c2cnc(Sc3cccc(C(=O)NS(=O)(=O)C4CCCCC4)c3Cl)cn2)CC1] | 3-((5-(4- (aminomethyl)-4- methylpiperidin-1- yl)pyrazin-2-yl)thio)- 2-chloro-N-(cyclo- hexylsulfonyl)ben- zamide | 538.3 | ¹H NMR (400 MHz, DMSO-d6) δ 8.38 (s, 1H), 8.18 (s, 1H), 7.89- 7.42 (m, 3H), 7.17-7.02 (m, 2H), 6.79 (d, J = 6.8 Hz, 1H), 3.87 (m, 2H), 3.44-3.34 (m, 2H), 3.27- 3.21 (m, 1H), 2.73 (s, 2H), 2.16-1.99 (m, 2H), 1.74 (m, 2H), 1.64-1.24 (m, 8H), 1.14 (m, 2H), 1.04 (s, 3H). |
| 28 | 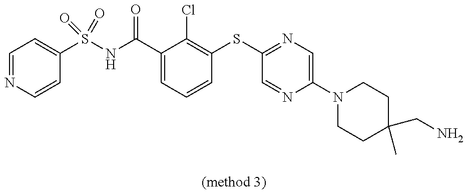 [SMILES: CC1(CN)CCN(c2cnc(Sc3cccc(C(=O)NS(=O)(=O)c4ccncc4)c3Cl)cn2)CC1] | 3-((5-(4- (aminomethyl)-4- methylpiperidin-1-yl) pyrazin-2-yl)thio)-2- chloro-N-(pyridin-4- ylsulfonyl)benzamide | 533.2 | ¹H NMR (400 MHz, DMSO-d6) δ 8.79 (d, J = 3.9 Hz, 2H), 8.39 (s, 1H), 8.20 (s, 1H), 7.82 (m, 5H), 7.28-7.14 (m, 2H), 6.90 (m, 1H), 3.89 (m, 2H), 3.42 (m, 2H), 2.76 (s, 2H), 1.51 (m, 2H), 1.41 (m, 2H), 1.05 (s, 3H). |
| 29 | 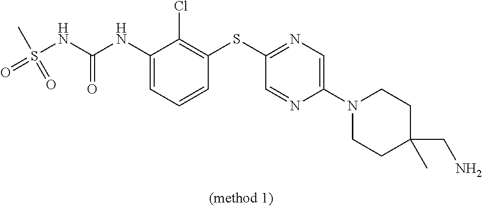 [SMILES: CC1(CN)CCN(c2cnc(Sc3cccc(NC(=O)NS(C)(=O)=O)c3Cl)cn2)CC1] | N-((3-((5-(4- (aminomethyl)-4- methylpiperidin-1- yl)pyrazin-2-yl)thio)- 2-chlorophe- nyl)carbamoyl) methanesulfonamide | 485.2 | ¹H NMR (400 MHz, DMSO-d6) δ 8.38 (s, 1H), 8.17 (s, 1H), 8.12 (d, J = 8.0 Hz, 1H), 7.50 (s, 3H), 7.15 (s, 1H), 7.05 (s, 1H), 6.41 (d, J = 7.1 Hz, 1H), 3.87 (s, 2H), 3.39 (s, 2H), 2.75 (s, 5H), 1.50 (s, 2H), 1.41 (s, 2H), 1.04 (s, 3H). |
| 30 | 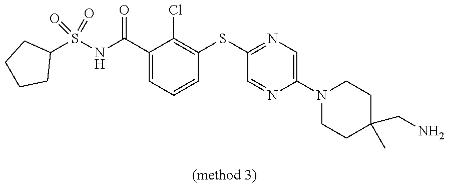 [SMILES: CC1(CN)CCN(c2cnc(Sc3cccc(C(=O)NS(=O)(=O)C4CCCC4)c3Cl)cn2)CC1] | 3-((5-(4- (aminomethyl)-4- methylpiperidin-1- yl)pyrazin-2-yl)thio)- 2-chloro-N- (cyclopentyl- sulfonyl)ben- zamide | 524.4 | ¹H NMR (400 MHz, DMSO-d6) δ 8.39 (s, 1H), 8.18 (s, 1H), 7.64 (s, 3H), 7.11 (m, 2H), 6.79 (d, J = 6.0 Hz, 1H), 3.88 (m, 3H), 3.39 (m, 2H), 2.74 (s, 2H), 1.88-1.74 (m, 4H), 1.65-1.56 (m, 2H), 1.52-1.37 (m, 6H), 1.04 (s, 3H). |
| 31 | 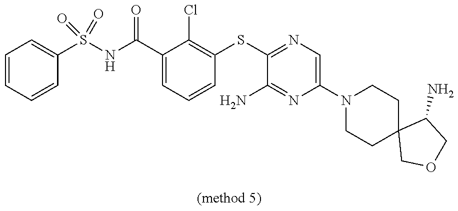 [SMILES: Nc1nc(N2CCC3(CC2)COC[C@H]3N)cnc1Sc1cccc(C(=O)NS(=O)(=O)c2ccccc2)c1Cl] | (S)-3-((3-amino-5-(4- amino-2-oxa-8- azaspiro[4.5]decan-8- yl)pyrazin-2-yl)thio)- 2-chloro-N- (phenylsulfonyl)benza- mide | 575.3 | ¹H NMR (400 MHz, DMSO-d6) δ 12.79 (s, 1H), 8.35 (s, 3H), 7.97 (d, J = 7.6 Hz, 2H), 7.75 (m, 1H), 7.70-7.62 (m, 3H), 7.37-7.05 (m, 3H), 6.70 (d, J = 7.7 Hz, 1H), 4.08 (m, 3H), 3.77 (m, 3H), 3.47 (m, 1H), 3.16 (t, J = 10.8 Hz, 1H), 3.05 (t, J = 11.0 Hz, 1H), 1.74-1.49 (m, 4H). |
| 32 | 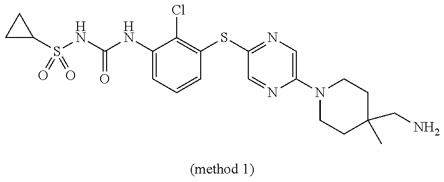 [SMILES: CC1(CN)CCN(c2cnc(Sc3cccc(NC(=O)NS(=O)(=O)C4CC4)c3Cl)cn2)CC1] | N-((3-((5-(4- (aminomethyl)-4- methylpiperidin-1- yl)pyrazin-2-yl)thio)- 2-chlorophe- nyl)carbamoyl) cyclopropanesulfona- mide | 511.3 | ¹H NMR (400 MHz, DMSO-d6) δ 8.38 (s, 1H), 8.20-8.11 (m, 2H), 7.55 (s, 3H), 7.44 (s, 1H), 7.05 (t, J = 7.9 Hz, 1H), 6.41 (d, J = 7.6 Hz, 1H), 3.88 (d, J = 12.9 Hz, 2H), 3.39 (s, 2H), 2.75 (s, 2H), 2.67 (s, 1H), 1.50 (s, 2H), 1.41 (s, 2H), 1.04 (s, 3H), 0.76 (s, 2H), 0.66 (d, J = 5.9 Hz, 2H). |
| 33 | 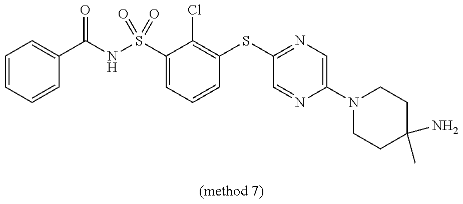 [SMILES: CC1(N)CCN(c2cnc(Sc3cccc(S(=O)(=O)NC(=O)c4ccccc4)c3Cl)cn2)CC1] | N-((3-((5-(4-amino-4- methylpiperidin-1- yl)pyrazin-2-yl)thio)- 2-chlorophenyl)sulfo- nyl)benzamide | 518.2 | ¹H NMR (400 MHz, DMSO-d6) δ 8.44 (s, 1H), 8.32 (s, 2H), 8.28 (s, 1H), 7.98 (d, J = 7.9 Hz, 1H), 7.89 (d, J = 7.7 Hz, 2H), 7.63 (t, J = 7.4 Hz, 1H), 7.48 (q, J = 7.3 Hz, 3H), 7.15 (d, J = 8.1 Hz, 1H), 4.06 (m 2H), 3.41 (m, 2H), 1.75 (m, 4H), 1.36 (s, 3H). |
| 34 | 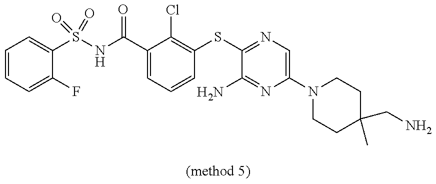 [SMILES: CC1(CN)CCN(c2cnc(Sc3cccc(C(=O)NS(=O)(=O)c4ccccc4F)c3Cl)c(N)n2)CC1] | 3-((3-amino-5-(4- (aminomethyl)-4- methylpiperidin-1- yl)pyrazin-2-yl)thio)- 2-chloro-N-((2- fluorophenyl)sulfonyl) benzamide | 565.2 | ¹H NMR (400 MHz, DMSO-d6) δ 7.79 (t, J = 7.1 Hz, 1H), 7.62 (m, 4H), 7.49-7.38 (m, 1H), 7.24-7.12 (m, 2H), 7.13- 7.02 (m, 2H), 6.50 (d, J = 7.2 Hz, 1H), 6.02 (s, 2H), 3.83 (d, J = 13.5 Hz, 2H), 3.32 (d, J = 13.3 Hz, 2H), 2.75 (s, 2H), 1.46 (m, 2H), 1.38 (m, 2H), 1.03 (s, 3H). |
| 35 | 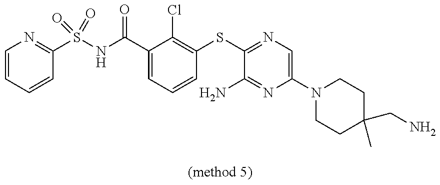 [SMILES: CC1(CN)CCN(c2cnc(Sc3cccc(C(=O)NS(=O)(=O)c4ccccn4)c3Cl)c(N)n2)CC1] | 3-((3-amino-5-(4- (aminomethyl)-4- methylpiperidin-1- yl)pyrazin-2-yl)thio)- 2-chloro-N-(pyridin- 2-ylsulfonyl)benza- mide | 548.3 | ¹H NMR (400 MHz, DMSO-d6) δ 8.52 (d, J = 3.6 Hz, 1H), 7.93-7.82 (m, 2H), 7.62 (m, 4H), 7.45-7.35 (m, 1H), 7.13 (d, J = 7.2 Hz, 1H), 7.05 (t, J = 7.6 Hz, 1H), 6.50 (d, J = 7.6 Hz, 1H), 6.02 (s, 2H), 3.83 (m, 2H), 3.38-3.30 (m, 2H), 2.75 (s, 2H), 1.47 (m, 2H), |
| | | | | 1.36 (m, 2H), 1.03 (s, |
| | | | | 3H) |
| 36 | 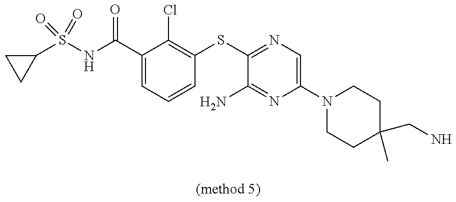 [SMILES: CC1(CN)CCN(c2cnc(Sc3cccc(C(=O)NS(=O)(=O)C4CC4)c3Cl)c(N)n2)CC1] | 3-((3-amino-5-(4- (aminomethyl)-4- methylpiperidin-1- yl)pyrazin-2-yl)thio)- 2-chloro-N-(cyclo- propylsulfonyl)ben- zamide | 511.4 | ¹H NMR (400 MHz, DMSO-d6) δ 7.63 (m, 4H), 7.14-6.97 (m, 2H), 6.49 (d, J = 6.4 Hz, 1H), 6.02 (s, 2H), 3.83 (m, 2H), 3.38-3.30 (m, 2H), 2.86-2.77 (m, 1H), 2.73 (s, 2H), 1.45 (m, 2H), 1.36 (m, 2H), 1.03 (s, 3H), 0.80 (m, 2H), 0.69 (m, 2H). |
| 37 | 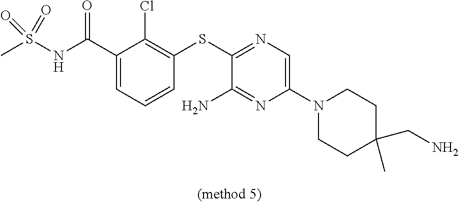 [SMILES: CC1(CN)CCN(c2cnc(Sc3cccc(C(=O)NS(C)(=O)=O)c3Cl)c(N)n2)CC1] | 3-((3-amino-5-(4- (aminomethyl)-4- methylpiperidin-1- yl)pyrazin-2-yl)thio)- 2-chloro-N- (methylsulfonyl)ben- zamide | 485.3 | ¹H NMR (400 MHz, DMSO-d6) δ 7.63 (m, 4H), 7.11-6.99 (m, 2H), 6.50 (d, J = 4.8 Hz, 1H), 6.03 (s, 2H), 3.83 (m, 2H), 3.37-3.30 (m, 2H), 2.83 (s, 3H), 2.74 (s, 2H), 1.52-1.41 (m, 2H), 1.36 (m, 2H), 1.03 (s, 3H). |
| 38 | 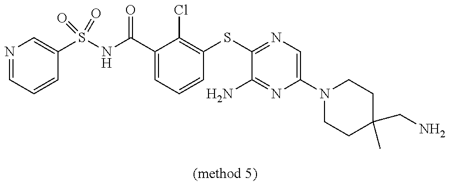 [SMILES: CC1(CN)CCN(c2cnc(Sc3cccc(C(=O)NS(=O)(=O)c4cccnc4)c3Cl)c(N)n2)CC1] | 3-((3-amino-5-(4- (aminomethyl)-4- methylpiperidin-1- yl)pyrazin-2-yl)thio)- 2-chloro-N-(pyridin- 3- ylsulfonyl)benzamide | 548.4 | ¹H NMR (400 MHz, DMSO-d6) δ 8.90 (s, 1H), 8.59 (d, J = 3.8 Hz, 1H), 8.12 (d, J = 7.8 Hz, 1H), 7.63 (m, 4H), 7.48- 7.40 (m, 1H), 7.10-7.00 (m, 2H), 6.50 (dd, J = 6.0, 2.8 Hz, 1H), 6.02 (s, 2H), 3.83 (m, 2H), 3.33 (s, 2H), 2.75 (s, 2H), 1.47 (m, 2H), 1.36 (m, 2H), 1.03 (s, 3H). |
| 39 | 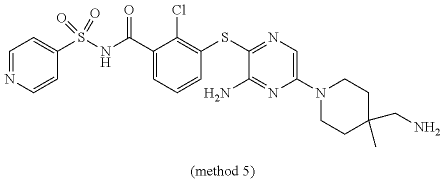 [SMILES: CC1(CN)CCN(c2cnc(Sc3cccc(C(=O)NS(=O)(=O)c4ccncc4)c3Cl)c(N)n2)CC1] | 3-((3-amino-5-(4- (aminomethyl)-4- methylpiperidin-1- yl)pyrazin-2-yl)thio)- 2- chloro-N-(pyridin-4- ylsulfonyl)benzamide | 548.4 | ¹H NMR (400 MHz, DMSO-d6) δ 8.65 (d, J = 4.6 Hz, 2H), 7.73-7.57 (m, 6H), 7.13-7.00 (m, 2H), 6.51 (d, J = 7.1 Hz, 1H), 6.03 (s, 2H), 3.83 (m, 2H), 3.40-3.31 (m, 2H), 2.75 (s, 2H), 1.46 (m, 2H), 1.37 (m, 2H), 1.04 (s, 3H). |
| 40 | 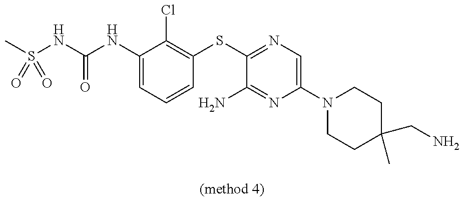 [SMILES: CC1(CN)CCN(c2cnc(Sc3cccc(NC(=O)NS(C)(=O)=O)c3Cl)c(N)n2)CC1] | N-((3-((3-amino-5-(4- (aminomethyl)-4- methylpiperidin-1- yl)pyrazin-2-yl)thio)- 2-chlorophe- nyl)carbamoyl) methanesulfonamide | 500.3 | ¹H NMR (400 MHz, DMSO-d6) δ 8.03 (d, J = 7.4 Hz, 1H), 7.73 (s, 2H), 7.61 (s, 1H), 7.21 (s, 1H), 7.01 (t, J = 8.1 Hz, 1H), 6.14 (d, J = 6.7 Hz, 1H), 6.06 (s, 2H), 3.84 (m, 2H), 3.32 (m, 2H), 2.78 (s, 3H), 2.76 (s, 2H), 1.47 (m, 2H), 1.38 (m, 2H), 1.04 (s, 3H). |
| 41 | 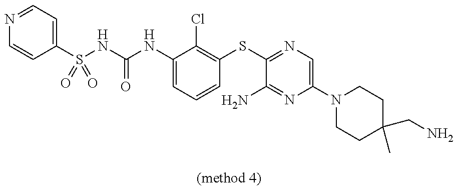 [SMILES: CC1(CN)CCN(c2cnc(Sc3cccc(NC(=O)NS(=O)(=O)c4ccncc4)c3Cl)c(N)n2)CC1] | N-((3-((3-amino-5-(4- (aminomethyl)-4- methylpiperidin-1- yl)pyrazin-2-yl)thio)- 2-chlorophe- nyl)carbamoyl) pyridine-4- sulfonamide | 563.3 | ¹H NMR (400 MHz, DMSO-d6) δ 8.61 (d, J = 6.0 Hz, 2H), 7.89-7.83 (m, 1H), 7.65 (dd, J = 4.4, 1.6 Hz, 4H), 7.60 (s, 1H), 7.37 (s, 1H), 6.97 (t, J = 8.1 Hz, 1H), 6.12 (dd, J = 7.9, 1.4 Hz, 1H), 6.04 (s, 2H), 3.84 (m, 2H), 3.26 (m, 2H), 2.76 (s, 2H), 1.47 (m, 2H), 1.39 (m, 2H), 1.04 (s, 3H). |
| 42 | 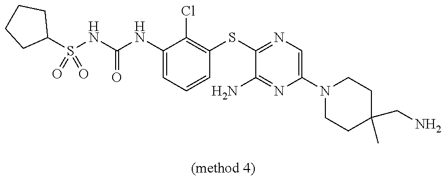 [SMILES: CC1(CN)CCN(c2cnc(Sc3cccc(NC(=O)NS(=O)(=O)C4CCCC4)c3Cl)c(N)n2)CC1] | N-((3-((3-amino-5-(4- (aminomethyl)-4- methylpiperidin-1- yl)pyrazin-2-yl)thio)- 2-chloro- phenyl)carbamoyl) cyclopentanesulfona- mide | 554.4 | ¹H NMR (400 MHz, DMSO-d6) δ 8.05 (d, J = 8.0 Hz, 1H), 7.64 (s, 2H), 7.60 (s, 1H), 7.00 (t, J = 8.2 Hz, 1H), 6.12 (d, J = 7.4 Hz, 1H), 6.06 (s, 2H), 3.84 (m, 2H), 3.68 (m, 1H), 3.35 (m, 2H), 2.74 (m, 2H), 1.86-1.79 (m, 2H), 1.74 (m, 2H), 1.60 (m, 2H), 1.47 (m, 2H), |
| | | | | 1.39 (m, 2H), 1.04 (s, |
| | | | | 3H). |
| 43 | 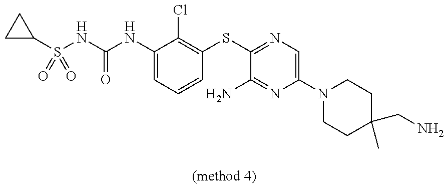 [SMILES: CC1(CN)CCN(c2cnc(Sc3cccc(NC(=O)NS(=O)(=O)C4CC4)c3Cl)c(N)n2)CC1] | N-((3-((3-amino-5-(4- (aminomethyl)-4- methylpiperidin-1- yl)pyrazin-2-yl)thio)- 2-chlorophe- nyl)carbamoyl) cyclopropanesulfona- mide | 526.3 | ¹H NMR (400 MHz, DMSO-d6) δ 8.06 (d, J = 7.7 Hz, 1H), 7.60 (d, J = 6.3 Hz, 1H), 7.45 (s, 1H), 7.01 (t, J = 8.1 Hz, 1H), 6.13 (dd, J = 7.9, 1.4 Hz, 1H), 6.06 (s, 2H), 3.84 (m, 2H), 3.34 (m, 2H), 2.75 (s, 2H), 2.71-2.61 (m, 1H), 1.47 (m, 2H), 1.39 (m, 2H), 1.04 (s, 3H), 0.78 (m, 2H), 0.67 |
| | | | | (m, 2H). |
| 44 | 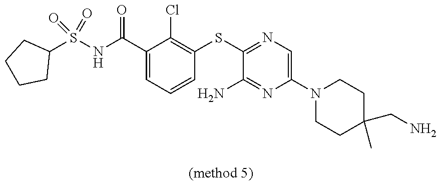 [SMILES: CC1(CN)CCN(c2cnc(Sc3cccc(C(=O)NS(=O)(=O)C4CCCC4)c3Cl)c(N)n2)CC1] | 3-((3-amino-5-(4- (aminomethyl)-4- methylpiperidin-1- yl)pyrazin-2-yl)thio)- 2-chloro-N-(cyclo- pentylsulfonyl)ben- zamide | | ¹H NMR (400 MHz, DMSO-d6) δ 7.85-7.44 (b, 4H), 7.04 (d, J = 4.3 Hz, 2H), 6.56-6.40 (m, 1H), 6.03 (s, 2H), 3.83 (m, 3H), 3.37-3.31 (m, 2H), 2.72 (s, 2H), 1.91- 1.69 (m, 4H), 1.67-1.55 (m, 2H), 1.53-1.41 (m, 4H), 1.37 (m, 2H), 1.03 (s, 3H). |
| 45 | 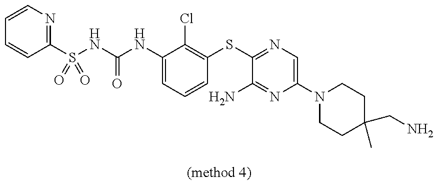 [SMILES: CC1(CN)CCN(c2cnc(Sc3cccc(NC(=O)NS(=O)(=O)c4ccccn4)c3Cl)c(N)n2)CC1] | N-((3-((3-amino-5-(4- (aminomethyl)-4- methylpiperidin-1- yl)pyrazin-2-yl)thio)- 2-chlorophe- nyl)carbamoyl) pyridine-2- sulfonamide | 563.3 | ¹H NMR (400 MHz, DMSO-d6) δ 8.51 (d, J = 4.5 Hz, 1H), 7.93-7.80 (m, 3H), 7.66 (b, 3H), 7.59 (s, 1H), 7.39 (s, 1H), 6.95 (t, J = 8.2 Hz, 1H), 6.11 (d, J = 7.7 Hz, 1H), 6.05 (s, 2H), 3.83 (m, 2H), 3.34 (m, 2H), 2.76 (s, 2H), 1.52-1.42 (m, 2H), 1.38 (m, 2H), 1.04 (s, 3H). |
| 46 | 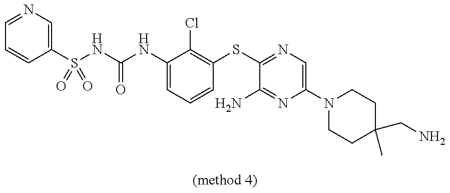 [SMILES: CC1(CN)CCN(c2cnc(Sc3cccc(NC(=O)NS(=O)(=O)c4cccnc4)c3Cl)c(N)n2)CC1] | N-((3-((3-amino-5-(4- (aminomethyl)-4- methylpiperidin-1- yl)pyrazin-2-yl)thio)- 2-chlorophe- nyl)carbamoyl) pyridine-3- sulfonamide | 563.3 | ¹H NMR (400 MHz, DMSO-d6) δ 8.87 (s, 1H), 8.55 (d, J = 4.7 Hz, 1H), 8.08 (d, J = 8.0 Hz, 1H), 7.88 (d, J = 8.3 Hz, 1H), 7.65 (b, 3H), 7.59 (s, 1H), 7.41 (dd, J = 7.8, 4.9 Hz, 1H), 7.31 (s, 1H), 6.96 (t, J = 8.1 Hz, 1H), 6.12 (d, J = 7.9 Hz, 1H), 6.04 (s, 2H), 3.84 (m, 2H), 3.31-3.25 (m, 2H), |
| | | | | 2.76 (s, 2H), 1.47 (m, |
| | | | | 2H), 1.37 (m, 2H), 1.04 |
| | | | | (s, 3H). |
| 47 | 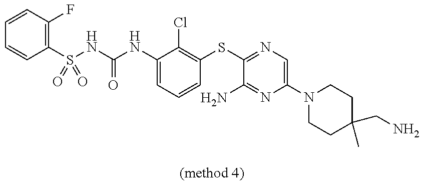 [SMILES: CC1(CN)CCN(c2cnc(Sc3cccc(NC(=O)NS(=O)(=O)c4ccccc4F)c3Cl)c(N)n2)CC1] | N-((3-((3-amino-5-(4- (aminomethyl)-4- methylpiperidin-1- yl)pyrazin-2-yl)thio)- 2- chlorophenyl)carba- moyl)-2-fluoro- benzenesulfonamide | 580.3 | ¹H NMR (400 MHz, DMSO-d6) δ 7.92 (d, J = 8.2 Hz, 1H), 7.75 (t, J = 7.5 Hz, 1H), 7.56 (m, 5H), 7.41 (d, J = 5.4 Hz, 1H), 7.20-7.10 (m, 2H), 6.95 (t, J = 8.1 Hz, 1H), 6.11 (d, J = 7.9 Hz, 1H), 6.04 (s, 2H), 3.84 (m, 2H), 3.25 (m, 2H), 2.76 (s, 2H), 1.48 (m, 2H), 1.37 (m, 2H), 1.04 (s, |
| | | | | 3H). |
| 48 | 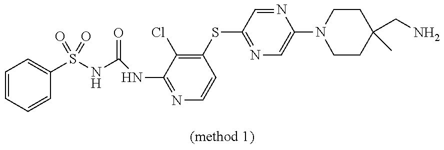 [SMILES: CC1(CN)CCN(c2cnc(Sc3ccnc(NC(=O)NS(=O)(=O)c4ccccc4)c3Cl)cn2)CC1] | N-((4-((5-(4- (aminomethyl)-4- methylpiperidin-1- yl)pyrazin-2-yl)thio)- 3-chloropyridin-2- yl)carbamoyl)ben- zenesulfonamide | 548.3 | |
| 49 | 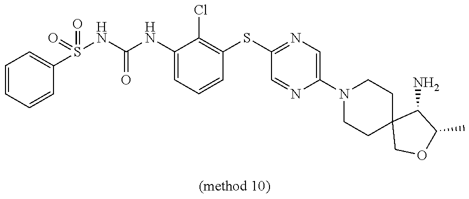 [SMILES: C[C@@H]1OCC2(CCN(c3cnc(Sc4cccc(NC(=O)NS(=O)(=O)c5ccccc5)c4Cl)cn3)CC2)[C@@H]1N] | N-((3-((5-((3S,4S)-4- amino-3-methyl-2- oxa-8-azaspiro [4.5]decan-8-yl) pyrazin-2-yl)thio)- 2-chlorophe- nyl)carbamoyl) benzenesulfonamide | 589.2 | ¹H NMR (400 MHz, DMSO-d6) δ 8.41 (s, 1H), 8.18 (s, 1H), 8.04 (s, 1H), 7.76 (s, 2H), 7.53 (s, 1H), 7.38 (s, 5H), 7.03 (s, 1H), 6.45 (s, 1H), 4.16 (m, 3H), 3.84 (m, 1H), 3.66 (m, 1H), 3.17 (m, 3H), 1.69 (m, 3H), 1.55 (m, 1H), 1.18 (s, 3H). |
| 50 | 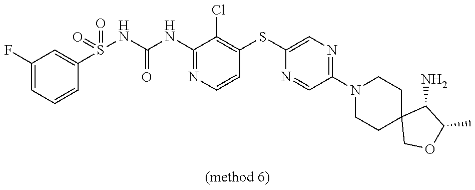 [SMILES: C[C@@H]1OCC2(CCN(c3cnc(Sc4ccnc(NC(=O)NS(=O)(=O)c5cccc(F)c5)c4Cl)cn3)CC2)[C@@H]1N] | N-((4-((5-((3S,4S)-4- amino-3-methyl-2- oxa-8-azaspiro [4.5]decan-8-yl) pyrazin-2-yl)thio)- 3-chloropyridin-2- yl)carbamoyl)-3- fluorobenzene- sulfonamide | 608.1 | ¹H NMR (400 MHz, DMSO-d6) δ 8.52 (s, 1H), 8.32 (s, 1H), 7.90 (m, 1H), 7.55 (m, 2H), 7.42 (m, 2H), 7.22 (m, 2H), 6.28 (m, 1H), 4.18 (m, 3H), 3.85 (d, J = 8.7 Hz, 1H), 3.66 (d, J = 8.8 Hz, 1H), 3.17 (m, 3H), 1.71 (m, 2H), 1.51 (m, 2H), 1.18 (d, J = 6.4 Hz, 3H). |
| 51 | 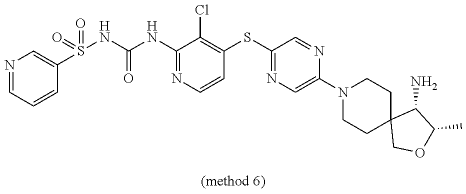 [SMILES: C[C@@H]1OCC2(CCN(c3cnc(Sc4ccnc(NC(=O)NS(=O)(=O)c5cccnc5)c4Cl)cn3)CC2)[C@@H]1N] | N-((4-((5-((3S,4S)-4- amino-3-methyl-2- oxa-8-azaspiro [4.5]decan-8-yl) pyrazin-2-yl)thio)- 3-chloropyridin-2- yl)carbamoyl)pyri- dine-3-sulfonamide | 591.1 | |
| 52 | 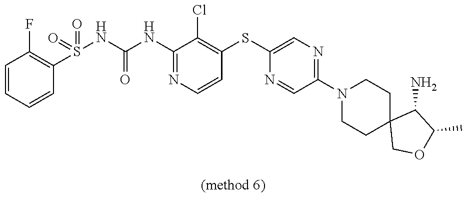 [SMILES: C[C@@H]1OCC2(CCN(c3cnc(Sc4ccnc(NC(=O)NS(=O)(=O)c5ccccc5F)c4Cl)cn3)CC2)[C@@H]1N] | N-((4-((5-((3S,4S)- 4-amino-3- methyl-2-oxa- 8-azaspiro[4.5]de- can-8-yl)pyra- zin-2-yl)thio)- 3-chloropyridin-2- yl)carbamoyl)-2- fluorobenzene- sulfonamide | 608.2 | ¹H NMR (400 MHz, DMSO-d6) δ 8.53 (s, 1H), 8.33 (s, 1H), 7.90 (s, 1H), 7.74 (s, 1H), 7.40 (s, 1H), 7.13 (s, 3H), 6.84 (s, 1H), 6.26 (s, 1H), 4.17 (m, 3H), 3.83 (d, J = 8.6 Hz, 1H), 3.64 (d, J = 8.7 Hz, 1H), 3.27-3.11 (m, 3H), 1.70 (s, 3H), 1.57 (s, 1H), 1.17 (d, J = 6.3 Hz, 3H). |
| 53 | 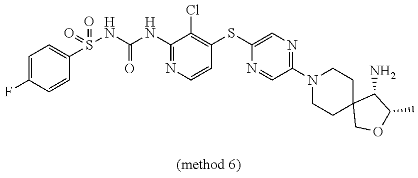 [SMILES: C[C@@H]1OCC2(CCN(c3cnc(Sc4ccnc(NC(=O)NS(=O)(=O)c5ccc(F)cc5)c4Cl)cn3)CC2)[C@@H]1N] | N-((4-((5-((3S,4S)- 4-amino-3- methyl-2-oxa-8- azaspiro[4.5]decan- 8-yl)pyrazin- 2-yl)thio)- 3-chloropyridin-2- yl)carbamoyl)-4- fluorobenzene- sulfonamide | 608.0 | ¹H NMR (400 MHz, DMSO-d6) δ 8.52 (s, 1H), 8.33 (s, 1H), 8.07 (s, 1H), 7.74 (s, 2H), 7.12 (s, 3H), 7.04-6.61 (m, 1H), 6.28 (d, J = 33.6 Hz, 1H), 4.11 (m 3H), 3.77 (d, J = 8.6 Hz, 1H), 3.58 (d, J = 8.6 Hz, 1H), 3.33 (s, 2H), 3.16 (s, 1H), 1.78-1.67 (m, 2H), 1.65 (m, 1H), |
| | | | | 1.53 (m, 1H), 1.13 (d, J = |
| | | | | 6.4 Hz, 3H). |
| 54 | 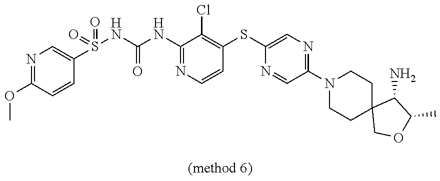 [SMILES: COc1ccc(S(=O)(=O)NC(=O)Nc2nccc(Sc3cnc(N4CCC5(CC4)CO[C@@H](C)[C@H]5N)cn3)c2Cl)cn1] | N-((4-((5-((3S,4S)- 4-amino-3- methyl-2-oxa- 8-azaspiro[4.5]decan- 8-yl)pyrazin- 2-yl)thio)- 3-chloropyridin-2- yl)carbamoyl)-6- methoxypyridine-3- sulfonamide | 621.2 | ¹H NMR (400 MHz, CDCl₃) δ 8.78 (s, 1H), 8.22-8.12 (m, 3H), 7.85 (d, J = 5.1 Hz, 1H), 6.77 (d, J = 8.6 Hz, 1H), 6.33 (d, J = 5.4 Hz, 1H), 4.17 (m, 1H), 3.99 (m, 2H), 3.94 (m, 3H), 3.80 (d, J = 8.8 Hz, 1H), 3.68 (d, J = 8.8 Hz, 1H), 3.31 (m, 2H), 2.98 (m, 1H), 1.94 |
| | | | | (d, J = 5.9 Hz, 1H), 1.83 |
| | | | | (m, 1H), 1.68 (m, 1H), |
| | | | | 1.55 (m, 1H), 1.21 (m, |
| | | | | 3H). |
| 55 | 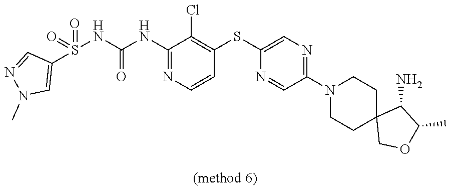 [SMILES: C[C@@H]1OCC2(CCN(c3cnc(Sc4ccnc(NC(=O)NS(=O)(=O)c5cnn(C)c5)c4Cl)cn3)CC2)[C@@H]1N] | N-((4-((5-((3S,4S)- 4-amino-3- methyl-2-oxa- 8-azaspiro[4.5]de- can-8-yl)pyra- zin-2-yl)thio)- 3-chloropyridin-2- yl)carbamoyl)-1- methyl-1H-pyrazole- 4-sulfonamide | 594.0 | ¹H NMR (400 MHz, CDCl₃) δ 8.21 (d, J = 10.0 Hz, 2H), 8.04 (s, 1H), 7.84 (d, J = 5.2 Hz, 2H), 6.31 (d, J = 5.3 Hz, 1H), 4.21-4.12 (m, 1H), 4.05-3.93 (m, 2H), 3.87 (s, 3H), 3.81 (d, J = 8.8 Hz, 1H), 3.69 (d, J = 8.8 Hz, 1H), 3.47-3.41 (m, 1H), 3.37-3.28 (m, 1H), 2.99 (d, J = 4.5 Hz, 1H), |
| | | | | 1.90-1.64 (m, 4H), 1.22 |
| | | | | (d, J = 6.2 Hz, 3H). |
| 56 | 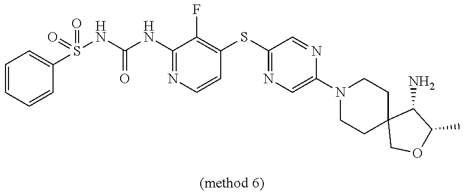 [SMILES: C[C@@H]1OCC2(CCN(c3cnc(Sc4ccnc(NC(=O)NS(=O)(=O)c5ccccc5)c4F)cn3)CC2)[C@@H]1N] | N-((4-((5-((3S,4S)- 4-amino-3- methyl-2-oxa- 8-azaspiro[4.5]decan- 8-yl)pyrazin-2- yl)thio)-3- fluoropyridin-2-yl) carbamoyl)benzene- sulfonamide | 574.3 | ¹H NMR (400 MHz, CDC1₃) δ 8.20 (s, 1H), 8.18 (s, 1H), 8.14 (s, 1H), 8.08 (d, J = 7.8 Hz, 1H), 8.03 (s, 1H), 7.84 (d, J = 7.5 Hz, 1H), 7.47 (m, 1H), 7.38 (t, J = 7.5 Hz, 1H), 7.31 (m, 1H), 4.16 (m, 1H), 3.95 (m, 2H), 3.78 (d, J = 8.7 Hz, 1H), 3.67 (d, J = 8.7 Hz, 1H), 3.32 (m, 2H), 2.95 (m, |
| | | | | 1H), 1.96 (m, 1H), 1.82 |
| | | | | (m, 1H), 1.67-1.62 (m, |
| | | | | 1H), 1.55 (m, 1H), 1.20 |
| | | | | (m, 3H). |
| 57 | 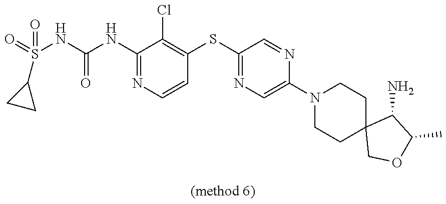 [SMILES: C[C@@H]1OCC2(CCN(c3cnc(Sc4ccnc(NC(=O)NS(=O)(=O)C5CC5)c4Cl)cn3)CC2)[C@@H]1N] | N-((4-((5-((3S,4S)- 4-amino-3- methyl-2-oxa- 8-azaspiro[4.5]decan- 8-yl)pyrazin-2- yl)thio)-3- chloropyridin-2- yl)carbamoyl)cyclo- propanesulfonamide | 554.2 | ¹H NMR (400 MHz, CDCl₃) δ 8.28 (d, J = 6.7 Hz, 2H), 7.92 (d, J = 5.5 Hz, 1H), 6.40 (d, J = 5.6 Hz, 1H), 4.27-4.22 (m, 1H), 4.09 (m, 2H), 3.87 (d, J = 8.8 Hz, 1H), 3.76 (d, J = 8.8 Hz, 1H), 3.53- 3.42 (m, 1H), 3.38 (m, 1H), 3.06 (m, 1H), 2.02 (m, 1H), 1.88 (m, 1H), 1.81 (m, 2H), 1.73 (m, 1H), 1.35 (m, 2H), 1.27 |
| | | | | (m, 3H), 1.12 (m, 2H). |
| 58 | 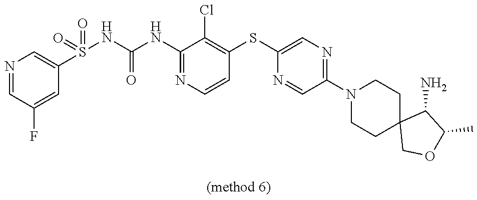 [SMILES: C[C@@H]1OCC2(CCN(c3cnc(Sc4ccnc(NC(=O)NS(=O)(=O)c5cncc(F)c5)c4Cl)cn3)CC2)[C@@H]1N] | N-((4-((5-((3S,4S)-4- amino-3-methyl-2- oxa-8-aza- spiro[4.5]decan-8- yl)pyrazin-2-yl)thio)- 3-chloropyridin-2- yl)carbamoyl)-5- fluoropyridine-3- sulfonamide | 609.1 | ¹H NMR (400 MHz, DMSO-d₆) δ 8.74 (s, 1H), 8.59 (s, 1H), 8.50 (s, 1H), 8.30 (s, 1H), 8.13 (s, 1H), 7.92 (s, 2H), 6.34-6.27 (m, 1H), 4.19-4.01 (m, 3H), 3.79 (d, J = 8.8 Hz, 1H), 3.59 (d, J = 8.8 Hz, 1H), 3.30 (m, 1H), 3.17 (d, J = 8.1 Hz, 2H), 1.79- 1.67 (m, 2H), 1.63 (m, 1H), 1.54 (m, 1H), 1.15 (d, J = 6.5 Hz, 3H). |
| 59 | 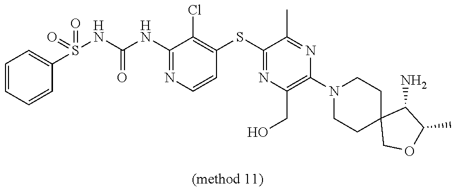 [SMILES: Cc1nc(N2CCC3(CC2)CO[C@@H](C)[C@H]3N)c(CO)nc1Sc1ccnc(NC(=O)NS(=O)(=O)c2ccccc2)c1Cl] | N-((4-((5-((3S,4S)-4- amino-3-methyl-2- oxa-8-aza- spiro[4.5]decan- 8-yl)-6- (hydroxymethyl)-3- methylpyrazin-2- yl)thio)-3- chloropyridin-2- yl)carbamoyl)ben- zenesulfonamide | 634.1 | ¹H NMR (400 MHz, DMSO-d6) δ 7.90 (d, J = 5.3 Hz, 1H), 7.81-7.69 (m, 2H), 7.38 (m, 4H), 6.21 (d, J = 5.2 Hz, 1H), 5.54 (s, 1H), 4.45 (d, J = 4.5 Hz, 2H), 4.24-4.09 (m, 1H), 3.86 (m, 3H), 3.65 (d, J = 9.1 Hz, 1H), 3.41-3.21 (m, 1H), 3.07 (m, 2H), 2.40 (s, 3H), 1.87-1.53 (m, 4H), 1.18 |
| | | | | (d, J = 6.4 Hz, 3H). |
| 60 | 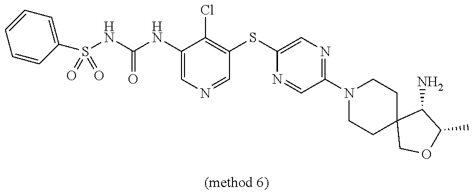 [SMILES: C[C@@H]1OCC2(CCN(c3cnc(Sc4cncc(NC(=O)NS(=O)(=O)c5ccccc5)c4Cl)cn3)CC2)[C@@H]1N] | N-((5-((5-((3S,4S)-4- amino-3-methyl-2- oxa-8-azaspiro[4.5] decan-8-yl)pyra- zin-2-yl)thio)- 4-chloropyridin-3- yl)carba- moyl)benzene- sulfonamide | 590.1 | ¹H NMR (400 MHz, DMSO-d6) δ 9.05 (s, 1H), 8.38 (s, 1H), 8.23 (s, 1H), 7.81-7.75 (m, 3H), 7.73 (s, 1H), 7.40 (m, 4H), 4.19 (m, 2H), 4.12 (m, 1H), 3.90 (d, J = 9.0 Hz, 1H), 3.64 (d, J = 9.0 Hz, 1H), 3.15 (d, J = 4.8 Hz, 1H), 3.12-3.01 (m, 2H), 1.81-1.52 (m, 4H), 1.22 (d, J = 6.5 Hz, 3H). |
| 61 | 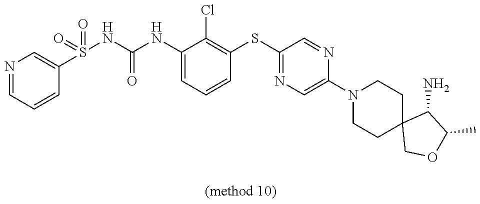 [SMILES: C[C@@H]1OCC2(CCN(c3cnc(Sc4cccc(NC(=O)NS(=O)(=O)c5cccnc5)c4Cl)cn3)CC2)[C@@H]1N] | N-((3-((5-((3S,4S)- 4-amino-3- methyl-2-oxa- 8-azaspiro[4.5]decan- 8-yl)pyrazin-2-yl) thio)-2-chlorophe- nyl)carbamoyl) pyridine-3- sulfonamide | 590.2 | ¹H NMR (400 MHz, DMSO-d6) δ 8.88 (s, 1H), 8.56 (d, J = 4.7 Hz, 1H), 8.40 (s, 1H), 8.17 (s, 1H), 8.09 (d, J = 7.8 Hz, 1H), 7.93 (s, 1H), 7.44- 7.38 (m, 2H), 7.02 (t, J = 8.1 Hz, 2H), 6.44 (d, J = 7.9 Hz, 1H), 4.24-4.08 (m, 3H), 3.86 (d, J = 9.1 Hz, 1H), 3.65 (d, J = 8.9 Hz, 1H), 3.37 (d, J = 4.9 Hz, 1H), 3.15 - 3.07 (m, |
| | | | | 2H), 1.70 (m, 3H), 1.54 |
| | | | | (m, 1H), 1.18 (d, J = 6.4 |
| | | | | Hz, 3H). |
| 62 | 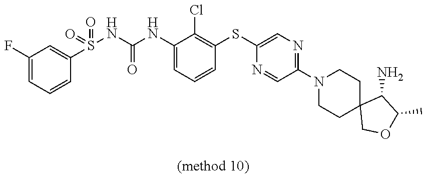 [SMILES: C[C@@H]1OCC2(CCN(c3cnc(Sc4cccc(NC(=O)NS(=O)(=O)c5cccc(F)c5)c4Cl)cn3)CC2)[C@@H]1N] | N-((3-((5-((3S,4S)-4- amino-3-methyl-2- oxa-8- azaspiro[4.5]decan- 8-yl)pyrazin-2-yl) thio)-2-chloro- phenyl)carbamoyl)- 3-fluoro- benzenesulfonamide | 607.4 | ¹H NMR (400 MHz, DMSO-d6) δ 8.40 (s, 1H), 8.18 (s, 1H), 7.96 (d, J = 8.3 Hz, 1H), 7.58 (d, J = 7.6 Hz, 1H), 7.51 (d, J = 10.2 Hz, 1H), 7.43 (m, 3H), 7.24 (d, J = 8.3 Hz, 1H), 7.03 (t, J = 8.1 Hz, 1H), 6.42 (d, J = 7.8 Hz, 1H), 4.17 (m, 3H), 3.88 |
| | | | | (d, J = 9.0 Hz, 1H), 3.64 |
| | | | | (d, J = 9.0 Hz, 1H), 3.34 |
| | | | | (d, J = 5.2 Hz, 1H), 3.09 |
| | | | | (m, 2H), 1.69 (m, 3H), |
| | | | | 1.55 (m, 1H), 1.20 (d, J = |
| | | | | 6.5 Hz, 3H). |
| 63 | 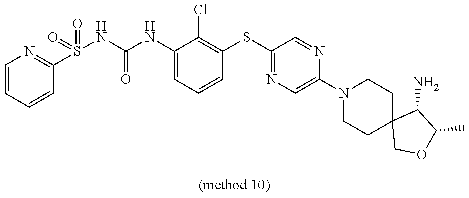 [SMILES: C[C@@H]1OCC2(CCN(c3cnc(Sc4cccc(NC(=O)NS(=O)(=O)c5ccccn5)c4Cl)cn3)CC2)[C@@H]1N] | N-((3-((5-((3S,4S)-4- amino-3-methyl-2- oxa-8-aza- spiro[4.5]decan- 8-yl)pyrazin-2-yl) thio)-2-chlorophe- nyl)carbamoyl) pyridine-2- sulfonamide | 590.3 | ¹H NMR (400 MHz, DMSO-d6) δ 8.50 (d, J = 4.7 Hz, 1H), 8.37 (s, 1H), 8.17 (s, 1H), 7.94 (d, J = 8.3 Hz, 1H), 7.86 (q, J = 7.8 Hz, 2H), 7.60 (s, 1H), 7.39 (t, J = 5.7 Hz, 1H), 6.99 (t, J = 8.1 Hz, 1H), 6.36 (d, J = 8.0 Hz, 1H), 4.10-4.01 (m, 1H), 3.93- 3.81 (m, 2H), 3.65 (d, J = 8.5 Hz, 1H), 3.47 (d, J = 8.5 Hz, 1H), 3.44-3.31 |
| | | | | (m, 2H), 2.91 (d, J = 4.9 |
| | | | | Hz, 1H), 1.78-1.68 (m, |
| | | | | 1H), 1.66-1.57 (m, 1H), |
| | | | | 1.56-1.43 (m, 2H), 1.06 |
| | | | | (d, J = 6.3 Hz, 3H). |
| 64 | 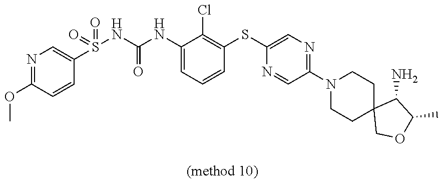 [SMILES: COc1ccc(S(=O)(=O)NC(=O)Nc2cccc(Sc3cnc(N4CCC5(CC4)CO[C@@H](C)[C@H]5N)cn3)c2Cl)cn1] | N-((3-((5-((3S,4S)-4- amino-3-methyl-2- oxa-8-aza- spiro[4.5]decan- 8-yl)pyrazin-2- yl)thio)-2-chloro- phenyl)carbamoyl)- 6-methoxypyridine- 3-sulfonamide | 620.2 | ¹H NMR (400 MHz, DMSO-d6) δ 8.61 (d, J = 2.2 Hz, 1H), 8.43 (s, 1H), 8.21 (s, 1H), 8.10 (dd, J = 8.8, 2.4 Hz, 1H), 7.82 (d, J = 8.4 Hz, 1H), 7.10 (t, J = 8.2 Hz, 1H), 6.93 (d, J = 8.7 Hz, 1H), 6.54 (d, J = 7.8 Hz, 1H), 4.19 (m, 3H), 3.94 (s, 3H), 3.85 (d, J = 9.1 Hz, 1H), 3.68 (d, |
| | | | | J = 9.1 Hz, 1H), 3.39 (d, |
| | | | | J = 8.8 Hz, 1H), 3.22-3.03 |
| | | | | (m, 2H), 1.70 (m, 4H), |
| | | | | 1.20 (d, J = 6.5 Hz, 3H). |
| 65 | 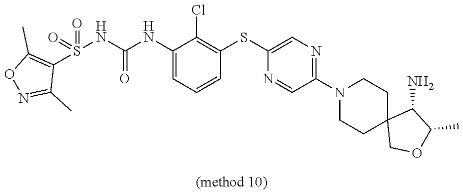 [SMILES: Cc1noc(C)c1S(=O)(=O)NC(=O)Nc1cccc(Sc2cnc(N3CCC4(CC3)CO[C@@H](C)[C@H]4N)cn2)c1Cl] | N-((3-((5-((3S,4S)-4- amino-3-methyl-2- oxa-8-aza- spiro[4.5]decan- 8-yl)pyrazin-2- yl)thio)-2-chloro- phenyl)carbamoyl)- 3,5- dimethylisoxazole-4- sulfonamide | 608.2 | ¹H NMR (400 MHz, DMSO-d6) δ 8.43 (s, 1H), 8.20 (s, 1H), 7.94 (d, J = 8.4 Hz, 1H), 7.85 (b, 2H), 7.54 (s, 1H), 7.08 (t, J = 8.1 Hz, 1H), 6.50 (d, J = 7.8 Hz, 1H), 4.26-4.13 (m, 3H), 3.87 (d, J = 9.1 Hz, 1H), 3.68 (d, J = 9.1 Hz, 1H), 3.39 (d, J = 4.4 Hz, 1H), 3.16-3.09 (m, 2H), 2.52 (s, 3H), 2.28 (s, |
| | | | | 3H), 1.70 (m, 3H), 1.55 |
| | | | | (m, 1H), 1.19 (d, J = 6.4 |
| | | | | Hz, 3H). |
| 66 | 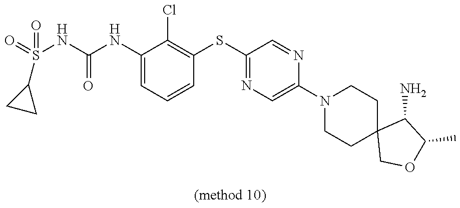 [SMILES: C[C@@H]1OCC2(CCN(c3cnc(Sc4cccc(NC(=O)NS(=O)(=O)C5CC5)c4Cl)cn3)CC2)[C@@H]1N] | N-((3-((5-((3S,4S)-4- amino-3-methyl-2- oxa-8-aza- spiro[4.5]decan- 8-yl)pyrazin-2-yl) thio)-2-chlorophe- nyl)carbamoyl) cyclopropanesulfona- mide | 553.4 | ¹H NMR (400 MHz, DMSO-d6) δ 8.41 (d, J = 6.4 Hz, 1H), 8.20 (s, 1H), 8.10 (d, J = 8.1 Hz, 1H), 7.57 (s, 1H), 7.08 (t, J = 8.2 Hz, 1H), 6.44 (d, J = 7.7 Hz, 1H), 4.26-4.02 (m, 3H), 3.87 (d, J = 8.8 Hz, 1H), 3.61 (d, J = 8.9 Hz, 1H), 3.25 (d, J = 5.3 Hz, 1H), 3.23-3.10 (m, 2H), 2.69 (m, 1H), 1.83- 1.69 (m, 2H), 1.66-1.53 |
| | | | | (m, 2H), 1.20 (d, J = 6.5 |
| | | | | Hz, 3H), 0.83-0.78 (m, |
| | | | | 2H), 0.72 (d, J = 7.9 Hz, |
| | | | | 2H). |
| 67 | 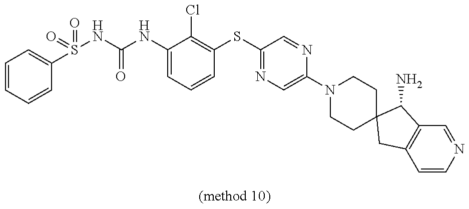 [SMILES: N[C@@H]1c2cnccc2CC12CCN(c1cnc(Sc3cccc(NC(=O)NS(=O)(=O)c4ccccc4)c3Cl)cn1)CC2] | (S)-N-((3-((5-(7- amino-5,7-dihydro- spiro[cyclopenta [c]pyridine-6,4′- piperidin]-1′- yl)pyrazin-2- yl)thio)-2-chloro- phenyl)carbamoyl) benzenesulfonamide | 622.2 | ¹H NMR (400 MHz, DMSO-d6) δ 8.52 (s, 1H), 8.41 (s, 1H), 8.39 (d, J = 4.9 Hz, 1H), 8.19 (s, 1H), 8.03 (d, J = 8.3 Hz, 1H), 7.78-7.74 (m, 2H), 7.48 (s, 1H), 7.41-7.34 (m, 4H), 7.27 (d, J = 4.8 Hz, 1H), 7.02 (t, J = 8.1 Hz, 1H), 6.39 (d, J = 7.9 Hz, 1H), 4.25-4.19 (m, 2H), 4.09 (s, 1H), 3.26- 3.15 (m, 2H), 3.11 (d, J = |
| | | | | 16.6 Hz, 1H), 2.77 (d, J = |
| | | | | 16.7 Hz, 1H), 1.75-1.67 |
| | | | | (m, 2H), 1.50 (m, 1H), |
| | | | | 1.20 (m, 1H). |
| 68 | 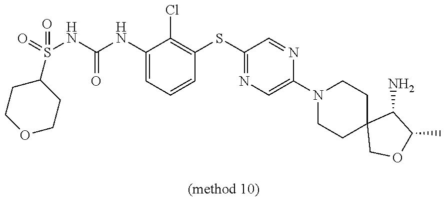 [SMILES: C[C@@H]1OCC2(CCN(c3cnc(Sc4cccc(NC(=O)NS(=O)(=O)C5CCOCC5)c4Cl)cn3)CC2)[C@@H]1N] | N-((3-((5-((3S,4S)-4- amino-3-methyl- 2-oxa-8- azaspiro[4.5]decan- 8-yl)pyrazin-2- yl)thio)-2-chloro- phenyl)carbamoyl) tetrahydro-2H- pyran-4-sulfonamide | 597.0 | ¹H NMR (400 MHz, DMSO-d6) δ 8.41 (s, 1H), 8.18 (s, 1H), 8.10 (d, J = 8.3 Hz, 1H), 7.69 (s, 1H), 7.25 (s, 1H), 7.07 (t, J = 8.1 Hz, 1H), 6.45 (d, J = 7.8 Hz, 1H), 4.20-4.04 (m, 3H), 3.91-3.79 (m, 3H), 3.63 (d, J = 9.0 Hz, 1H), 3.36 (m, 1H), 3.18 (m, 5H), 1.77 (m, 2H), 1.73-1.64 (m, 3H), 1.57 |
| | | | | (m, 3H), 1.16 (d, J = 6.4 |
| | | | | Hz, 3H). |
| 69 | 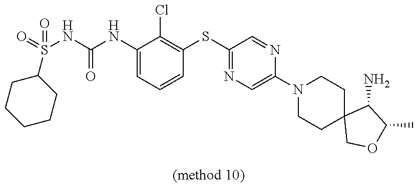 [SMILES: C[C@@H]1OCC2(CCN(c3cnc(Sc4cccc(NC(=O)NS(=O)(=O)C5CCCCC5)c4Cl)cn3)CC2)[C@@H]1N] | N-((3-((5-((3S,4S)-4- amino-3-methyl-2- oxa-8-aza- spiro[4.5]decan- 8-yl)pyrazin-2-yl) thio)-2-chloro- phenyl)carbamoyl) cyclohexanesulfona- mide | 595.1 | ¹H NMR (400 MHz, DMSO-d6) δ 8.41 (s, 1H), 8.19 (s, 1H), 8.07 (d, J = 8.2 Hz, 1H), 7.85 (s, 1H), 7.07 (t, J = 8.1 Hz, 1H), 6.46 (d, J = 7.9 Hz, 1H), 4.20-4.02 (m, 3H), 3.81 (d, J = 8.8 Hz, 1H), 3.61 (d, J = 9.0 Hz, 1H), 3.25 (d, J = 4.9 Hz, 1H), 3.12 (m, 3H), 1.96 (m, |
| | | | | 2H), 1.69 (m, 5H), 1.59- |
| | | | | 1.46 (m, 2H), 1.37-1.23 |
| | | | | (m, 2H), 1.19-1.05 (m, |
| | | | | 3H), 1.17 (d, J = 8.8 Hz, |
| | | | | 3H). |
| 70 | 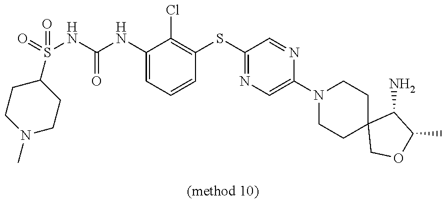 [SMILES: C[C@@H]1OCC2(CCN(c3cnc(Sc4cccc(NC(=O)NS(=O)(=O)C5CCN(C)CC5)c4Cl)cn3)CC2)[C@@H]1N] | N-((3-((5-((3S,4S)-4- amino-3-methyl-2- oxa-8-aza- spiro[4.5]decan- 8-yl)pyrazin-2-yl) thio)-2-chloro- phenyl)carbamoyl)- 1-methylpiperidine- 4-sulfonamide | 610.3 | ¹H NMR (400 MHz, DMSO-d₆) δ 8.41 (s, 1H), 8.19 (s, 1H), 8.08 (d, J = 8.4 Hz, 1H), 7.58 (s, 1H), 7.06 (t, J = 8.0 Hz, 1H), 6.41 (d, J = 8.0 Hz, 1H), 4.15 (t, J = 5.7 Hz, 3H), 3.84 (d, J = 8.8 Hz, 1H), 3.58 (d, J = 8.9 Hz, 1H), 3.15 (m, 6H), 2.97 (m, 2H), 2.27 (s, 3H), 2.03- 1.86 (m, 4H), 1.81-1.63 |
| | | | | (m, 4H), 1.18 (d, J = 6.4 |
| | | | | Hz, 3H). |
| 71 | 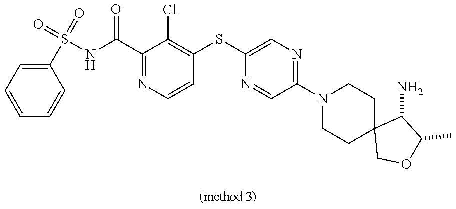 [SMILES: C[C@@H]1OCC2(CCN(c3cnc(Sc4ccnc(C(=O)NS(=O)(=O)c5ccccc5)c4Cl)cn3)CC2)[C@@H]1N] | 4-((5-((3S,4S)-4- amino-3-methyl-2- oxa-8-aza- spiro[4.5]decan- 8-yl)pyrazin-2-yl) thio)-3-chloro-N- (phenylsulfonyl)pico- linamide | 575.1 | ¹H NMR (400 MHz, CD₃OD) δ 8.25 (d, J = 13.7 Hz, 2H), 8.14-8.01 (m, 3H), 7.62 (m, 3H), 7.07 (s, 1H), 4.29 (m, 3H), 4.00 (d, J = 9.1 Hz, 1H), 3.88 (d, J = 9.0 Hz, 1H), 3.44 (m, 1H), 3.24- 3.09 (m, 2H), 1.91-1.65 (m, 4H), 1.32 (d, J = 6.3 Hz, 3H). |
| 72 | 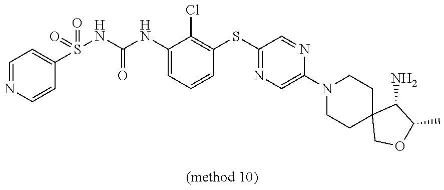 [SMILES: C[C@@H]1OCC2(CCN(c3cnc(Sc4cccc(NC(=O)NS(=O)(=O)c5ccncc5)c4Cl)cn3)CC2)[C@@H]1N] | N-((3-((5-((3S,4S)-4- amino-3-methyl-2- oxa-8-aza- spiro[4.5]decan- 8-yl)pyrazin-2-yl) thio)-2- chlorophenyl)carba- moyl)pyridine-4- sulfonamide | 590.1 | ¹H NMR (400 MHz, DMSO-d₆) δ 8.65-8.59 (m, 2H), 8.42 (s, 1H), 8.19 (s, 1H), 7.97 (d, J = 8.3 Hz, 1H), 7.68-7.63 (m, 2H), 7.40 (s, 1H), 7.04 (m, 1H), 6.46 (m, 1H), 4.19 (m, 3H), 3.86 (d, J = 9.0 Hz, 1H), 3.67 (d, J = 9.0 Hz, 1H), 3.37 (d, J = 4.9 Hz, 1H), 3.16- 3.11 (m, 2H), 1.69 (m, |
| | | | | 3H), 1.53 (m, 1H), 1.18 |
| | | | | (d, J = 6.3 Hz, 3H). |
| 73 | 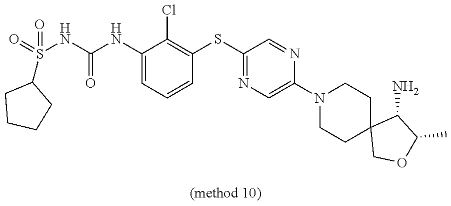 [SMILES: C[C@@H]1OCC2(CCN(c3cnc(Sc4cccc(NC(=O)NS(=O)(=O)C5CCCC5)c4Cl)cn3)CC2)[C@@H]1N] | N-((3-((5-((3S,4S)-4- amino-3-methyl-2- oxa-8-aza- spiro[4.5]decan- 8-yl)pyrazin-2-yl) thio)-2-chloro- phenyl)carbamoyl) cyclopentanesulfona- mide | 581.2 | ¹H NMR (400 MHz, DMSO-d6) δ 8.42 (s, 1H), 8.20 (s, 1H), 8.07 (d, J = 8.2 Hz, 1H), 7.88 (s, 1H), 7.33-7.19 (m, 1H), 7.10 (t, J = 8.2 Hz, 1H), 6.49 (m, 1H), 4.22-3.96 (m, 3H), 3.82 (d, J = 8.8 Hz, 1H), 3.78 (m, 1H), 3.63 (d, J = 9.0 Hz, 1H), 3.27-3.10 (m, 3H), 1.90- 1.78 (m, 4H), 1.74- |
| | | | | 1.60 (m, 5H), 1.56-1.47 |
| | | | | (m, 3H), 1.16 (d, J = 6.3 |
| | | | | Hz, 3H). |
| 74 | 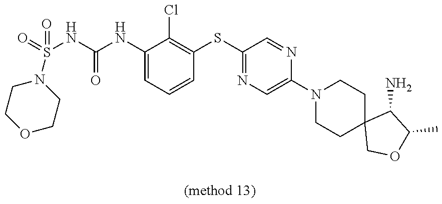 [SMILES: C[C@@H]1OCC2(CCN(c3cnc(Sc4cccc(NC(=O)NS(=O)(=O)N5CCOCC5)c4Cl)cn3)CC2)[C@@H]1N] | N-((3-((5-((3S,4S)-4- amino-3-methyl-2- oxa-8-aza- spiro[4.5]decan- 8-yl)pyrazin-2-yl) thio)-2-chloro- phenyl)carbamoyl) morpholine-4- sulfonamide | 598.3 | ¹H NMR (400 MHz, DMSO-d6) δ 8.42 (s, 1H), 8.19 (s, 1H), 8.16 (s, 1H), 8.11 (s, 1H), 7.42 (s, 2H), 7.09 (t, J = 8.1 Hz, 1H), 6.48 (d, J = 7.9 Hz, 1H), 4.21-4.03 (m, 3H), 3.84 (d, J = 8.9 Hz, 1H), 3.65 (d, J = 8.9 Hz, 1H), 3.58 (s, 4H), 3.32 (m, 1H), 3.17 (m, 2H), 3.00 (m, 4H), 1.68 m, 3H), |
| | | | | 1.54 (m, 1H), 1.18 (d, |
| | | | | J = 6.3 Hz, 3H). |
| 75 | 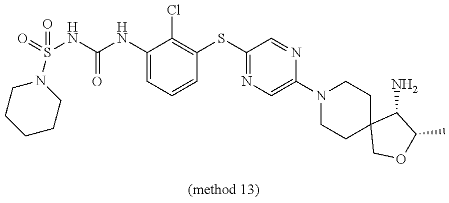 [SMILES: C[C@@H]1OCC2(CCN(c3cnc(Sc4cccc(NC(=O)NS(=O)(=O)N5CCCCC5)c4Cl)cn3)CC2)[C@@H]1N] | N-((3-((5- ((3S,4S)-4-amino- 3-methyl-2-oxa- 8-azaspiro[4.5]decan- 8-yl)pyrazin- 2-yl)thio)-2-chloro- phenyl)carbamoyl) piperidine-1- sulfonamide | 596.3 | |
| 76 | 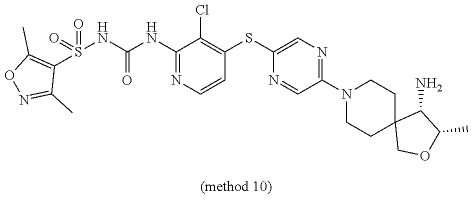 [SMILES: Cc1noc(C)c1S(=O)(=O)NC(=O)Nc1nccc(Sc2cnc(N3CCC4(CC3)CO[C@@H](C)[C@H]4N)cn2)c1Cl] | N-((4-((5-((3S,4S)- 4-amino-3-methyl- 2-oxa-8- azaspiro[4.5]decan- 8-yl)pyrazin-2-yl) thio)-3-chloro- pyridin-2- yl)carbamoyl)-3,5- dimethylisoxazole-4- sulfonamide | 609.1 | ¹H NMR (400 MHz, CDCl₃) δ 8.20 (d, J = 10.5 Hz, 2H), 7.84 (m, 1H), 6.34 (d, J = 4.9 Hz, 1H), 4.16 (m, 1H), 3.97 (m, 2H), 3.80 (d, J = 8.8 Hz, 1H), 3.68 (d, J = 8.8 Hz, 1H), 3.43-3.30 (m, 3H), 2.63 (s, 3H), 2.37 (s, 3H), 1.95 (m, 2H), 1.55 (m, 2H), 1.27 (m, 3H). |
| 77 | 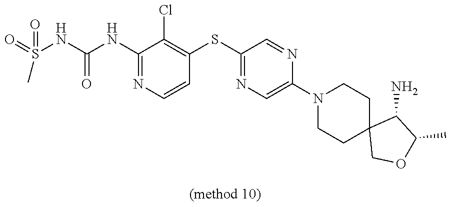 [SMILES: C[C@@H]1OCC2(CCN(c3cnc(Sc4ccnc(NC(=O)NS(C)(=O)=O)c4Cl)cn3)CC2)[C@@H]1N] | N-((4-((5-((3S,4S)- 4-amino-3-methyl- 2-oxa-8-aza- spiro[4.5]decan- 8-yl)pyrazin-2- yl)thio)-3- chloropyridin-2- yl)carba- moyl)methane- sulfonamide | 528.048 | |
| 78 | 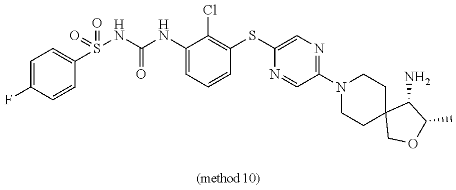 [SMILES: C[C@@H]1OCC2(CCN(c3cnc(Sc4cccc(NC(=O)NS(=O)(=O)c5ccc(F)cc5)c4Cl)cn3)CC2)[C@@H]1N] | N-((3-((5-((3S,4S)- 4-amino-3-methyl- 2-oxa-8-aza- spiro[4.5]decan- 8-yl)pyrazin-2- yl)thio)-2-chloro- phenyl)carbamoyl)- 4-fluoro- benzenesulfonamide | 607.2 | |
| 79 | 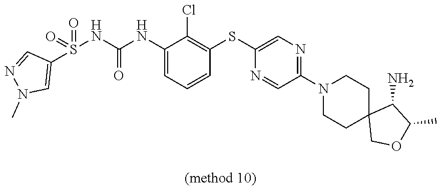 [SMILES: C[C@@H]1OCC2(CCN(c3cnc(Sc4cccc(NC(=O)NS(=O)(=O)c5cnn(C)c5)c4Cl)cn3)CC2)[C@@H]1N] | N-((3-((5-((3S,4S)- 4-amino-3-methyl- 2-oxa-8-aza- spiro[4.5]decan- 8-yl)pyrazin-2- yl)thio)-2-chloro- phenyl)carbamoyl)- 1-methyl-1H- pyrazole-4- sulfonamide | 593.2 | |
| 80 | 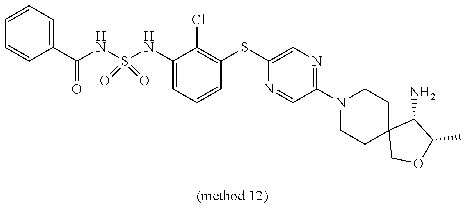 [SMILES: C[C@@H]1OCC2(CCN(c3cnc(Sc4cccc(NS(=O)(=O)NC(=O)c5ccccc5)c4Cl)cn3)CC2)[C@@H]1N] | N-(N-(3-((5-((3S,4S)- 4-amino-3-methyl-2- oxa-8- azaspiro[4.5]decan-8- yl)pyrazin-2-yl)thio)- 2-chlorophenyl)sulfa- moyl)benzamide | 589.1 | |
| 81 | 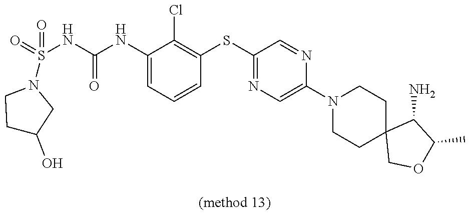 [SMILES: C[C@@H]1OCC2(CCN(c3cnc(Sc4cccc(NC(=O)NS(=O)(=O)N5CCC(O)C5)c4Cl)cn3)CC2)[C@@H]1N] | N-((3-((5-((3S,4S)- 4-amino-3-methyl- 2-oxa-8-aza- spiro[4.5]decan- 8-yl)pyrazin-2- yl)thio)-2-chloro- phenyl)carbamoyl)- 3- hydroxypyrrolidine-1- sulfonamide | 598.1 | |
| 82 | 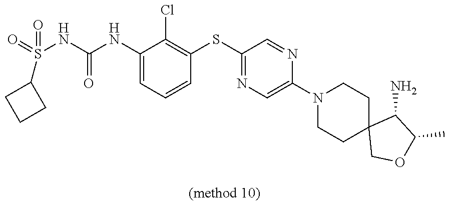 [SMILES: C[C@@H]1OCC2(CCN(c3cnc(Sc4cccc(NC(=O)NS(=O)(=O)C5CCC5)c4Cl)cn3)CC2)[C@@H]1N] | N-((3-((5-((3S,4S)- 4-amino-3-methyl- 2-oxa-8-aza- spiro[4.5]decan- 8-yl)pyrazin-2- yl)thio)-2-chloro- phenyl)carbamoyl) cyclobutanesulfona- mide | 567.1 | |
| 83 | 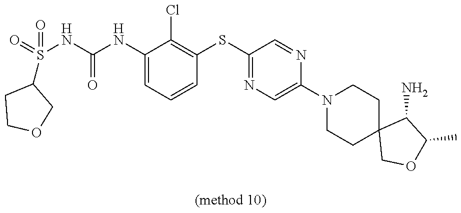 [SMILES: C[C@@H]1OCC2(CCN(c3cnc(Sc4cccc(NC(=O)NS(=O)(=O)C5CCOC5)c4Cl)cn3)CC2)[C@@H]1N] | N-((3-((5-((3S,4S)- 4-amino-3- methyl-2-oxa-8- azaspiro[4.5]decan- 8-yl)pyrazin-2- yl)thio)-2-chloro- phenyl)carbamoyl) tetrahydrofuran-3- sulfonamide | 583.3 | ¹H NMR (400 MHz, DMSO-d6) δ 8.42 (s, 1H), 8.19 (s, 1H), 8.11 (d, J = 8.4 Hz, 1H), 7.56 (s, 1H), 7.08 (t, J = 7.8 Hz, 1H), 6.48 (d, J = 7.7 Hz, 1H), 4.29-3.94 (m, 4H), 3.84 (t, J = 7.5 Hz, 3H), 3.76-3.68 (m, 1H), 3.67- 3.59 (m, 2H), 3.31 (m, 1H), 3.15 (m, 2H), 2.17- 2.06 (m, 1H), 1.99 (d, |
| | | | | J = 9.3 Hz, 1H), |
| | | | | 1.71 (d, J = 12.5 Hz, |
| | | | | 3H), 1.54 (d, J = |
| | | | | 13.3 Hz, 1H), 1.18 (d, |
| | | | | J = 6.1 Hz, 3H). |
| 84 | 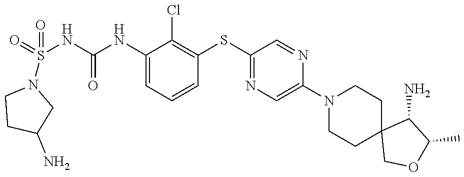 [SMILES: C[C@@H]1OCC2(CCN(c3cnc(Sc4cccc(NC(=O)NS(=O)(=O)N5CCC(N)C5)c4Cl)cn3)CC2)[C@@H]1N] | 3-amino-N-((3-((5- ((3S,4S)-4-amino-3- methyl-2-oxa-8-aza- spiro[4.5]decan-8- yl)pyrazin-2-yl)thio)- 2-chloro- phenyl)carbamoyl) pyrrolidine-1- sulfonamide | 597.3 | ¹H NMR (400 MHz, DMSO-d6) δ 8.42 (d, J = 7.0 Hz, 1H), 8.21 (s, 1H), 7.99 (s, 1H), 7.10 (t, J = 8.1 Hz, 1H), 6.49 (d, J = 8.0 Hz, 1H), 4.23-4.16 (m, 2H), 3.85 (d, J = 9.0 Hz, 1H), 3.76-3.61 (m, 4H), 3.46-3.40 (m, 2H), 3.13 (s, 2H), 2.16-2.06 (m, 1H), 1.89-1.80 (m, |
| | | | | 1H), 1.71 (m, 4H), 1.55 |
| | | | | (m, 2H), 1.18 (d, J = 6.4 |
| | | | | Hz, 3H). |

### Biochemical Assay

SHP2 is allosterically activated through binding of bis-tyro syiphorphorylated peptides to its Src Homology 2 (SH2) domains. The latter activation step leads to the release of the auto-inhibitory interface of SHP2, which in turn renders the SHP2 PTP active and available for substrate recognition and reaction catalysis. The catalytic activity of SHP2 was monitored using the surrogate substrate DiFMUP in a prompt fluorescence assay format. Specifically, the phosphatase reactions were performed at room temperature in 384-well black polystyrene plate, flat bottom, low flange, non-binding surface (Corning, Cat #3575) using a final reaction volume of 20 μL and the following assay buffer conditions: 60 mM HEPES, pH 7.2, 75 mM NaCl, 75 mM KCl, 1 mM EDTA, 0.01% Brij-35, 5 mM DTT, and 10% DMSO (final). The inhibition of SHP2 from the tested compounds (final concentrations varying from 0.003-100 μM) was monitored using an assay in which 0.5 nM of SHP2 (PTPN11/SHP2-FL, RBC produced, no cat #) was incubated with 0.35 μM of the peptide H₂NLN(pY)IDLDLV(dPEG8)LST(pY)ASINFQK-amide. After 20 minutes of incubation at room temperature, the surrogate substrate DiFMUP (6,8-difluoro-7-hydroxy-4-methylcoumarin, Invitrogen, cat #D6567, 100 μM final concentration) was added to the reaction. The fluorescence signal was monitored using a microplate reader (Envision, Perkin-Elmer) using excitation and emission wavelengths of 355 nm and 460 nm, respectively. The inhibitor dose response curves were analyzed using normalized IC₅₀ regression curve fitting with control based normalization.

The IC₅₀ values of for Compounds disclosed are listed in Table 2, A: ≤10 nM; B: >10 nM and ≤100 nM; C: >100 nM and ≤1 uM; and D: >1 μM.

### Cellular Assay

MiaPaCa-2 Cell proliferation assay in 3D culture. To evaluate cell viability, MiaPaCa-2 cells in logarithmic growth phase were seeded at optimal density and grown in as spheroids. Cells were incubated for 24 hours prior to the addition of different concentrations of the compounds. Cells were cultured with the compounds for 5 days and the cell viability was assessed using CCK8. Briefly, 2500 cells were seeded in round bottom ultra-low attachment 96 well plates (coming) in growth media supplemented with 10% FBS and incubated at 37° C. for 24 hours. The compounds were dissolved in DMSO (Sigma) to obtain 10 mM stock solution. Then the compounds were serially diluted in 3-fold to ten concentrations; 5 μL of the dilution above was transferred to 95 uL culture media to obtain the 50× compound stock solution; 4 uL of the 50× compound above was then transferred to the wells containing cells with 196 μL of the culture medium to obtain the final 1× concentration. The final concentration of DMSO was 0.1%. Spheroid cells were incubated in compounds for 5 days. Then 20 μL WST-8 solution (DOJINDO, Cell Counting KIT-8) was added to each well. The plates were further incubated at 37° C. for 1.5 hours, and then absorbance at 450 nm was measured using a microplate reader. Each compound was tested in triplicate and the IC50 value was calculated using GraphPad Prism.

The IC₅₀ values of for Compounds disclosed are listed in Table 2, A: ≤500 nM; B: >500 nM and ≤5 μM; C: >5 μM and ≤50 μM; and D: >50 μM.

TABLE 2

| | SHP2 | Cell |
|---|---|---|
| | bichemical | Proliferation |
| Example | activity* | MiaPaCa-2** |
| 1 | B | A |
| 2 | A | A |
| 3 | A | B |
| 4 | A | A |
| 5 | A | A |
| 6 | | A |
| 7 | B | B |
| 8 | B | C |
| 9 | | A |
| 10 | | A |
| 11 | | A |
| 12 | | A |
| 13 | | A |
| 14 | | B |
| 15 | | B |
| 16 | | A |
| 17 | | A |
| 18 | B | C |
| 19 | B | C |
| 20 | B | C |
| 21 | A | A |
| 22 | A | A |
| 23 | A | B |
| 24 | A | B |
| 25 | A | A |
| 26 | A | A |
| 27 | A | A |
| 28 | A | B |
| 29 | A | A |
| 30 | A | B |
| 31 | A | A |
| 32 | A | A |
| 33 | B | A |
| 34 | A | B |
| 35 | A | A |
| 36 | B | B |
| 37 | B | C |
| 38 | A | B |
| 39 | B | C |
| 40 | A | A |
| 41 | A | C |
| 42 | A | A |
| 43 | A | A |
| 44 | | A |
| 45 | | A |
| 46 | | A |
| 47 | | B |
| 48 | | A |
| 49 | | A |
| 50 | | A |
| 51 | | B |
| 52 | | A |
| 53 | | A |
| 54 | | A |
| 55 | | A |
| 56 | | A |
| 57 | | A |
| 58 | | A |
| 59 | | A |
| 60 | | B |
| 61 | | A |
| 62 | | A |
| 63 | | A |
| 64 | | A |
| 65 | | A |
| 66 | | A |
| 67 | | A |
| 68 | | A |
| 69 | | A |
| 70 | | A |
| 71 | | B |
| 72 | | A |
| 73 | | A |
| 74 | | A |
| 75 | | A |
| 76 | | A |
| 77 | | A |
| 78 | | A |
| 79 | | A |
| 80 | | A |
| 81 | | A |
| 82 | | A |
| 83 | | A |
| 84 | | A |

*The IC₅₀ values: A: ≤10 nM; B: >10 nM and ≤100 nM; C: >100 nM and ≤1 uM; and D: >1 μM.
*The IC₅₀ values: A: ≤500 nM; B: >500 nM and ≤5 μM; C: >5 μM and ≤50 μM; and D: >50 μM.

What is claimed is:

1. A compound of Formula I: 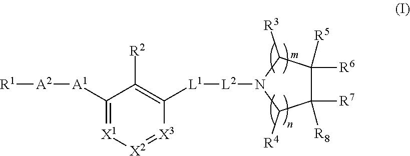
and/or stereoisomers, stable isotopes, or pharmaceutically acceptable salts or solvates thereof, wherein:
R¹ is an optionally substituted group selected from C1-C12 alkyl, C3-C12 cycloalkyl, 4-12 membered heterocyclyl containing 1-3 heteroatoms selected from N, O, and S as ring members, aryl, and heteroaryl containing 1-4 heteroatoms selected from N, O, and S as ring members; and wherein R¹ is optionally substituted with 1-4 substituents independently selected from R⁹, wherein R⁹ is selected from halo, —OH, NH₂, ═O, —CN, —OC(O)R¹⁰, —CO₂R¹⁰, —C(O)N(R^{11a}R^{11b}), —C(═NR¹²)N(R^{11a}R^{11b}), —C(O)R¹⁰, —OR¹⁰, —SR¹⁰, —S(O)₀₋₂R¹³, —S(O)(═NR¹²)R¹³, —S(O)₁₋₂N(R^{11a}R^{11b}), —N(R^{11a}R^{11b}) —N(R^{11a})C(O)R¹⁰, —N(R^{11a})C(═NR¹²)R¹³, —N(R^{11a})S(O)₁₋₂R¹³, —N(R^{11c})C(O)N(R^{11a}R^{11b}), —N(R^{11c})C(═NR¹²)N(R^{11a}R^{11b}), —N(R^{11c})S(O)₁₋₂N(R^{11a}R^{11b}) —N(R^{11a})CO₂R¹³, and an optionally substituted group selected from C1-C6 alkyl, C1-C6 alkoxy, C1-C6 haloalkyl, C1-C6 haloalkoxy, C3-C6 cycloalkyl, C3-C6 cycloalkylidenyl, C3-C6 cycloalkoxy, saturated or unsaturated 4-7 membered heterocyclyl containing 1-2 heteroatoms selected from N, O, and S as ring members, aryl, and heteroaryl containing 1-4 heteroatoms selected from N, O, and S as ring members; wherein the optional substituents are 1-4 substituents independently selected from -halo, —OH, NH₂, ═O, —CN, —SO₂NH₂, C1-C6 alkyl, C1-C6 haloalkyl, C1-C6 alkoxy, C1-C6 haloalkoxy, C3-C6 cycloalkyl, C3-C6 cycloalkoxy, C1-C6 alkylsulfonyl, C3-C6 cycloalkylsulfonyl, C1-C6 alkylsulfonylamino, C3-C6 cycloalkylsulfonylamino, C1-C6 alkylaminosulfonyl, and C3-C6 cycloalkylaminosulfonyl; wherein R¹⁰, R^{11a}, R^{11b} and R^{11c} are independently selected from H and an optionally substituted group selected from C1-C6 alkyl, C3-C6 cycloalkyl, C1-C6 haloalkyl, saturated or unsaturated 4-7 membered heterocyclyl containing 1-2 heteroatoms selected from N, O, and S as ring members, aryl, heteroaryl containing 1-4 heteroatoms selected from N, O, and S as ring members; R¹² is selected from H, —CN, —OH, and an optionally substituted group selected from C1-C4 alkyl and C1-C4 alkoxy; R¹³ is an optionally substituted group selected from C1-C6 alkyl, C3-C6 cycloalkyl C1-C6 haloalkyl, saturated or unsaturated 4-7 membered heterocyclyl containing 1-2 heteroatoms selected from N, O, and S as ring members, aryl, heteroaryl containing 1-4 heteroatoms selected from N, O, and S as ring members; wherein each of R¹⁰, R^{11a}, R^{11b}, R^{11c}, R¹² and R¹³ is optionally substituted with 1-3 groups independently selected from halo, —OH, NH₂, ═O, —CN, —SO₂NH₂, C1-C6 alkyl, C1-C6 haloalkyl, C1-C6 alkoxy, C1-C6 haloalkoxy, C3-C6 cycloalkyl, C3-C6 cycloalkoxy, C1-C6 alkylsulfonyl, C3-C6 cycloalkylsulfonyl, C1-C6 alkylsulfonylamino, C3-C6 cycloalkylsulfonylamino, C1-C6 alkylaminosulfonyl and C3-C6 cycloalkylaminosulfonyl; wherein two substituents on the same or adjacent carbon atoms of R¹ can optionally be taken together to form a 4-7 membered ring that can be saturated or unsaturated and optionally contains 1-2 heteroatoms selected from N, O and S as ring members and can optionally be substituted with 1-2 groups independently selected from R⁹;
R² is selected from H, —CN, ethynyl, halo, —CF₃, C1-C3 alkyl, and C1-C3alkoxy;
R³ and R⁴ are independently selected from H, ═O, OH, CN, halo, —CO₂R¹⁰, —C(O)N(R^{11a}R^{11b}), —N(R^{11a}R^{11b}), and an optionally substituted group selected from C1-C6 alkyl, C1-C6 alkyloxy, and C3-C6 cycloalkylidenyl optionally contains 1-2 heteroatoms as ring members selected from N, O and S, wherein the optional substituents are one or more groups independently selected from R⁹; wherein R³ and R⁴ can optionally be taken together to form a 4-7 membered cycloalkyl or 4-7 membered heterocyclyl optionally contains 1-2 heteroatoms as ring members selected from N, O and S and can optionally be substituted with at least one group selected from R⁹;
R⁵, R⁶, R⁷ and R⁸ are independently selected from H, OH, CN, halo, —CO₂R¹⁰, —C(O)N(R^{11a}R^{11b}), —N(R^{11a}R^{11b}), and an optionally substituted group selected from C1-C6 alkyl and C1-C6 alkyloxy, wherein the optional substituents are one or more groups independently selected from R⁹; or
wherein R⁵ and R⁶, or R⁷ and R⁸, can optionally be taken together to form a group selected from 3-7 membered cycloalkyl and 4-7 membered heterocyclyl containing 1-2 heteroatoms selected from N, O and S as ring members, wherein said 3-7 membered cycloalkyl or 4-7 membered heterocyclyl can optionally be fused with one or more groups selected from C3-C7 cycloalkyl, 4-10 membered heterocyclyl containing 1-3 heteroatoms selected from N, O, and S as ring members, aryl, and heteroaryl containing 1-4 heteroatoms selected from N, O, and S as ring members, and wherein any of the foregoing cyclic groups is optionally substituted with at least one group selected from R⁹;
m is 0, 1, or 2;
n is 1 or 2;
A¹ is selected from —S(O)₂N(H)C(O)—, —S(O)₂N(H)C(O)N(R¹⁴)—, —C(O)N(H)S(O)₂—, —C(O)N(H)S(O)₂N(R¹⁴)—, —S(O)(═NR¹⁵)N(H)C(O)—, —S(O)(═NR¹⁵)N(H)C(O)N(R¹⁴)—, —C(O)N(H)S(O) (═NR¹⁵)—, and —C(O)N(H)S(O) (═NR¹⁵)N(R¹⁴)—; wherein R¹⁴ is selected from H and an optionally substituted group selected from C1-C6 alkyl, C3-C6 cycloalkyl, C1-C6 haloalkyl, and 4-7 membered heterocyclyl containing 1-2 heteroatoms selected from N, O, and S as ring members; wherein R¹⁵ is selected from H, —CN, —OH, and an optionally substituted group selected from C1-C4 alkyl and C1-C4 alkoxy; and wherein R¹⁴ and R¹⁵ are optionally substituted with at least one group selected from R⁹;
A² is selected from a bond, —N(R¹⁴)—, and —O—;
X¹, X², and X³ are independently selected from —N— and —CH—,
L¹ is selected from a bond, —S—, —O—, and —C(O)—,
L² is selected from: 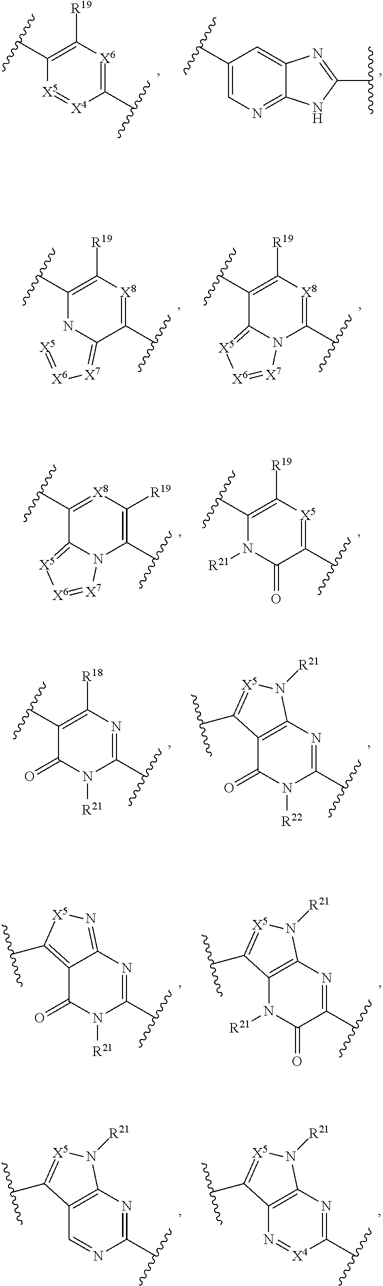 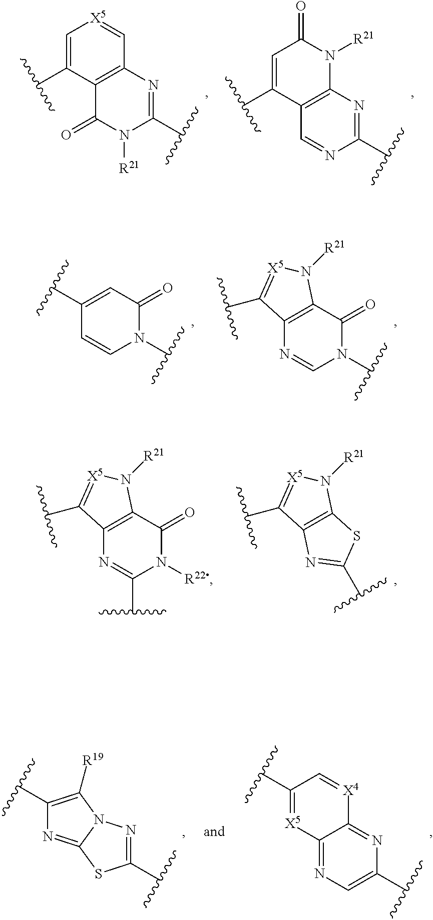
wherein R¹⁹ is selected from H, OH, CN, NH₂, halo, and an optionally substituted group selected from C1-C4 alkyl, C1-C6 haloalkyl, C3-C6 cycloalkyl, and aryl, wherein the optionally substituted groups can be substituted with at least one group selected from R⁹;
X⁴, X⁵, X⁶, X⁷, and X⁸ are independently selected from —N— and —C(R²⁰)—, wherein R²⁰ is selected from H, —CONH₂, OH, NH₂, and CN, and —CH₂H; and
R²¹ and R²² are independently selected from H, CH₃, and —CH₂H.

2. The compound of claim 1, wherein R¹ is selected from C1-C12 alkyl, C3-C12 cycloalkyl, 4-12 membered heterocyclyl containing 1-3 heteroatoms selected from N, O, and S as ring members, aryl, and heteroaryl containing 1-4 heteroatoms selected from N, O, and S as ring members; and wherein R¹ is optionally substituted with 1-4 substituents independently selected from R⁹.

3. The compound of claim 1 or claim 2, wherein R¹ is selected from C1-C6 alkyl, C3-C10 cycloalkyl, 4-10 membered heterocyclyl containing 1-3 heteroatoms selected from N, O, and S as ring members, C6-C10 aryl, and 4-10 membered heteroaryl containing 1-4 heteroatoms selected from N, O, and S as ring members; and wherein R¹ is optionally substituted with 1-4 substituents independently selected from R⁹.

4. The compound of any one of claims 1-3, wherein R¹ is selected from phenyl, pyridyl, pyrimidinyl, pyrazinyl, pyridazinyl, cyclohexyl, cyclopentyl, cyclobutyl, cyclopropyl, methyl, and ethyl; and wherein R¹ is optionally substituted with 1-2 substituents independently selected from R⁹.

5. The compound of any one of claims 1-4, wherein R² is selected from H and halo.

6. The compound of any one of claims 1-5, wherein R² is selected from H and Cl.

7. The compound of any one of claims 1-6, wherein R³ is H.

8. The compound of any one of claims 1-7, wherein R⁴ is H.

9. The compound of any one of claims 1-8, wherein R⁵ and R⁶ are independently selected from H, OH, CN, halo, —N(R^{11a}R^{11b}), and C1-C6 alkyl optionally substituted with 1-3 groups independently selected from R⁹.

10. The compound of any one of claims 1-9, wherein R⁵ and R⁶ are independently selected from H, NH₂, and C1-C6 alkyl optionally substituted with 1-3 groups independently selected from R⁹.

11. The compound of any one of claims 1-10, wherein R⁵ and R⁶ are independently selected from H, NH₂, and —CH₂NH₂.

12. The compound of any one of claims 1-8, wherein R⁵ and R⁶ are taken together to form a group selected from 3-7 membered cycloalkyl or 4-7 membered heterocyclyl containing 1-2 heteroatoms selected from N, O and S as ring members, wherein said 3-7 membered cycloalkyl or 4-7 membered heterocyclyl are optionally substituted with 1-3 groups independently selected from R⁹.

13. The compound of any one of claims 1-8 and 12, wherein R⁵ and R⁶ are taken together to form a tetrahydrofuranyl optionally substituted with 1-3 groups independently selected from R⁹.

14. The compound of any one of claims 1-13, wherein m is 1.

15. The compound of any one of claims 1-13, wherein m is 2.

16. The compound of any one of claims 1-15, wherein n is 1.

17. The compound of any one of claims 1-15, wherein n is 2.

18. The compound of any one of claims 1-17, wherein A¹ is selected from —S(O)₂N(H)C(O)—, —S(O)₂N(H)C(O)N(R¹⁴)—, and —C(O)N(H)S(O)₂—; wherein R¹⁴ is selected from H and an optionally substituted group selected from C1-C6 alkyl, C3-C6 cycloalkyl, C1-C6 haloalkyl, 4-7 membered heterocyclyl containing 1-2 heteroatoms selected from N, O, and S as ring members; and wherein R¹⁴ is optionally substituted with one to more groups independently selected from R⁹;

19. The compound of any one of claims 1-18, wherein A¹ is selected from —S(O)₂N(H)C(O)—, —S(O)₂N(H)C(O)N(H)—, and —C(O)N(H)S(O)₂—.

20. The compound of any one of claims 1-19, wherein X¹ is —CH—, X² is —CH—, and X³ is —CH—.

21. The compound of any one of claims 1-19, wherein X¹ is N, X² is —CH—, and X³ is —CH—.

22. The compound of any one of claims 1-21, wherein L¹ is selected from a bond, —S—, —S(O)—, —S(O)₂—, and —O—.

23. The compound of any one of claims 1-22, wherein L¹ is —S—.

24. The compound of any one of claims 1-23, wherein L² is selected from

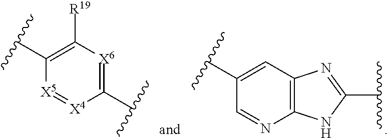

25. The compound of any one of claims 1-24, wherein L² is selected from

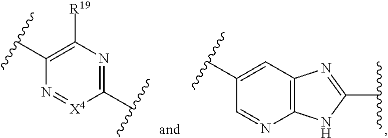

26. The compound of any one of claims 1-25, wherein R¹⁹ is selected from H, NH₂, and C1-C4 alkyl optionally substituted with 1-3 groups independently selected from R⁹.

27. The compound of any one of claims 1-26, wherein R¹⁹ is selected from H, NH₂, and methyl.

28. The compound of any one of claims 1-27, wherein X⁴ is selected from —CH— and —C(CH₂OH)—.

29. The compound of any one of claims 1-28, wherein X⁵ is —N—.

30. The compound of any one of claims 1-29, wherein X⁶ is —N—.

31. The compound of any one of claims 1-30, wherein R⁹ is selected from halo, NH₂, and C1-C6 alkyl.

32. The compound of claim 1, wherein the compound is of Formula IA, 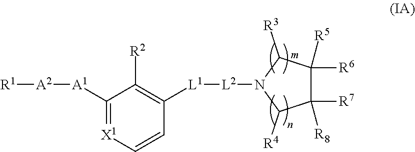
and/or a stereoisomer, a stable isotope, or a pharmaceutically acceptable salt or solvate thereof, wherein R¹, R², R³, R⁴, R⁵, R⁶, R⁷, R⁸, A¹, A², L¹, L², X¹, n, and m are as defined in claim 1.

33. The compound of claim 1, wherein the compound is of Formula IB, 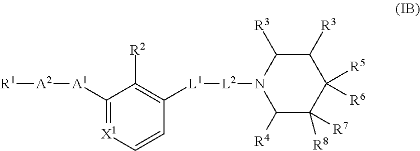
and/or a stereoisomer, a stable isotope, or a pharmaceutically acceptable salt or solvate thereof, wherein R¹, R², R³, R⁴, R⁵, R⁶, R⁷, R⁸, A¹, A², L¹, L², and X¹ are as defined in claim 1.

34. The compound of claim 1, wherein the compound is of Formula IC, 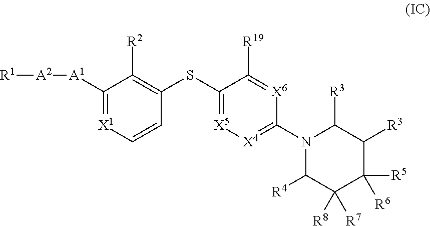
and/or a stereoisomer, a stable isotope, or a pharmaceutically acceptable salt or solvate thereof, wherein R¹, R², R³, R⁴, R⁵, R⁶, R⁷, R⁸, R¹⁹, A¹, A², X¹, X⁴, X⁵, and X⁶ are as defined in claim 1.

35. The compound of claim 1, wherein the compound is of Formula ID, 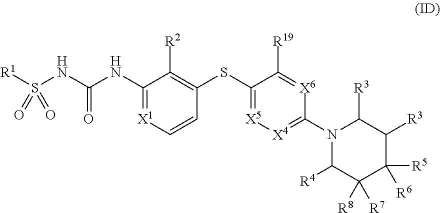
and/or a stereoisomer, a stable isotope, or a pharmaceutically acceptable salt or solvate thereof, wherein R¹, R², R³, R⁴, R⁵, R⁶, R⁷, R⁸, R¹⁹, X¹, X⁴, X⁵, and X⁶ are as defined in claim 1.

36. The compound of claim 1, wherein the compound is of Formula IE, 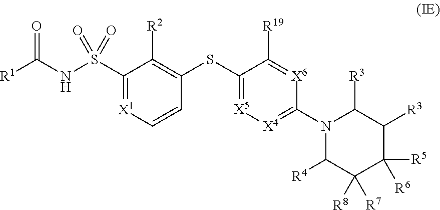
and/or a stereoisomer, a stable isotope, or a pharmaceutically acceptable salt or solvate thereof, wherein R¹, R², R³, R⁴, R⁵, R⁶, R⁷, R⁸, R¹⁹, X¹, X⁴, X⁵, and X⁶ are as defined in claim 1.

37. The compound of claim 1, wherein the compound is of Formula IF, 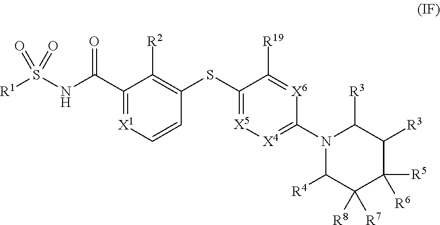
and/or a stereoisomer, a stable isotope, or a pharmaceutically acceptable salt or solvate thereof, wherein R¹, R², R³, R⁴, R⁵, R⁶, R⁷, R⁸, R¹⁹, X¹, X⁴, X⁵, and X⁶ are as defined in claim 1.

38. The compound of claim 1, which is selected from the following compounds, and/or a stereoisomer, a stable isotope, or a pharmaceutically acceptable salt or solvate thereof:
N-((3-((5-(4-amino-4-methylpiperidin-1-yl)pyrazin-2-yl)thio)-2-chlorophenyl)carbamoyl)benzenesulfonamide,
N-((3-((5-(4-(aminomethyl)-4-methylpiperidin-1-yl)pyrazin-2-yl)thio)-2-chlorophenyl)carbamoyl)benzenesulfonamide,
3-((5-(4-amino-4-methylpiperidin-1-yl)pyrazin-2-yl)thio)-2-chloro-N-(phenylsulfonyl)benzamide,
N-((3-((3-amino-5-(4-amino-4-methylpiperidin-1-yl)pyrazin-2-yl)thio)-2-chlorophenyl)carbamoyl)benzenesulfonamide,
3-((3-amino-5-(4-(aminomethyl)-4-methylpiperidin-1-yl)pyrazin-2-yl)thio)-2-chloro-N-(phenylsulfonyl)benzamide,
N-((4-((5-((3S,4S)-4-amino-3-methyl-2-oxa-8-azaspiro[4.5]decan-8-yl)pyrazin-2-yl)thio)-3-chloropyridin-2-yl)carbamoyl)benzenesulfonamide,
N-((3-((5-(4-(aminomethyl)-4-methylpiperidin-1-yl)pyrazin-2-yl)thio)-2-chlorophenyl)sulfonyl)benzamide,
N-((3-((3-amino-5-(4-amino-4-methylpiperidin-1-yl)pyrazin-2-yl)thio)-2-chlorophenyl)sulfonyl)benzamide,
N-((4-((5-(4-(aminomethyl)-4-methylpiperidin-1-yl)-6-(hydroxymethyl)-3-methylpyrazin-2-yl)thio)-3-chloropyridin-2-yl)carbamoyl)benzenesulfonamide,
N-((3-((5-((3S,4S)-4-amino-3-methyl-2-oxa-8-azaspiro[4.5]decan-8-yl)pyrazin-2-yl)thio)-2-chlorophenyl)carbamoyl)-2-fluorobenzenesulfonamide,
N-((3-((5-((3S,4S)-4-amino-3-methyl-2-oxa-8-azaspiro[4.5]decan-8-yl)-6-(hydroxymethyl)-3-methylpyrazin-2-yl)thio)-2-chlorophenyl)carbamoyl)benzenesulfonamide,
((3S,4S)-8-(5-((2-chloro-3-((N-(phenylcarbamoyl)sulfamoyl)amino)phenyl)thio)-pyrazin-2-yl)-3-methyl-2-oxa-8-azaspiro[4.5]decan-4-yl)amine,
N-((3-((5-((3S,4S)-4-amino-3-methyl-2-oxa-8-azaspiro[4.5]decan-8-yl)pyrazin-2-yl)thio)-2-chlorophenyl)carbamoyl)pyrrolidine-1-sulfonamide,
(S)—N-((4-((5-(7-amino-5,7-dihydrospiro[cyclopenta[b]pyridine-6,4′-piperidin]-1′-yl)pyrazin-2-yl)thio)-3-chloropyridin-2-yl)carbamoyl)benzenesulfonamide,
N-((4-((5-((3S,4S)-4-amino-3-methyl-2-oxa-8-azaspiro[4.5]decan-8-yl)-[1,2,4]triazolo[4,3-c]pyrimidin-8-yl)thio)-3-chloropyridin-2-yl)carbamoyl)benzenesulfonamide,
N—(N-(3-((5-((3S,4S)-4-amino-3-methyl-2-oxa-8-azaspiro[4.5]decan-8-yl)pyrazin-2-yl)thio)-2-chlorophenyl)sulfamoyl)pyrrolidine-1-carboxamide,
3-((5-((3S,4S)-4-amino-3-methyl-2-oxa-8-azaspiro[4.5]decan-8-yl)pyrazin-2-yl)thio)-2-chloro-N-(cyclopentylcarbamoyl)benzenesulfonamide,
3-((5-(4-amino-4-methylpiperidin-1-yl)pyrazin-2-yl)thio)-2-chloro-N-(cyclopropylsulfonyl)benzamide,
3-((5-(4-amino-4-methylpiperidin-1-yl)pyrazin-2-yl)thio)-2-chloro-N-(methylsulfonyl)benzamide,
3-((5-(4-amino-4-methylpiperidin-1-yl)pyrazin-2-yl)thio)-2-chloro-N-(cyclohexylsulfonyl)benzamide,
3-((5-(4-(aminomethyl)-4-methylpiperidin-1-yl)pyrazin-2-yl)thio)-2-chloro-N-(phenylsulfonyl)benzamide,
3-((5-(4-(aminomethyl)-4-methylpiperidin-1-yl)pyrazin-2-yl)thio)-2-chloro-N-(pyridin-2-ylsulfonyl)benzamide,
3-((5-(4-(aminomethyl)-4-methylpiperidin-1-yl)pyrazin-2-yl)thio)-2-chloro-N-((2-fluorophenyl)sulfonyl)benzamide,
3-((5-(4-(aminomethyl)-4-methylpiperidin-1-yl)pyrazin-2-yl)thio)-2-chloro-N-(pyridin-3-ylsulfonyl)benzamide,
3-((5-(4-(aminomethyl)-4-methylpiperidin-1-yl)pyrazin-2-yl)thio)-2-chloro-N-(cyclopropylsulfonyl)benzamide,
3-((5-(4-(aminomethyl)-4-methylpiperidin-1-yl)pyrazin-2-yl)thio)-2-chloro-N-(methylsulfonyl)benzamide,
3-((5-(4-(aminomethyl)-4-methylpiperidin-1-yl)pyrazin-2-yl)thio)-2-chloro-N-(cyclohexylsulfonyl)benzamide,
3-((5-(4-(aminomethyl)-4-methylpiperidin-1-yl)pyrazin-2-yl)thio)-2-chloro-N-(pyridin-4-ylsulfonyl)benzamide,
N-((3-((5-(4-(aminomethyl)-4-methylpiperidin-1-yl)pyrazin-2-yl)thio)-2-chlorophenyl)carbamoyl)methanesulfonamide,
3-((5-(4-(aminomethyl)-4-methylpiperidin-1-yl)pyrazin-2-yl)thio)-2-chloro-N-(cyclopentylsulfonyl)benzamide,
(S)-3-((3-amino-5-(4-amino-2-oxa-8-azaspiro[4.5]decan-8-yl)pyrazin-2-yl)thio)-2-chloro-N-(phenylsulfonyl)benzamide,
N-((3-((5-(4-(aminomethyl)-4-methylpiperidin-1-yl)pyrazin-2-yl)thio)-2-chlorophenyl)carbamoyl)cyclopropanesulfonamide,
N-((3-((5-(4-amino-4-methylpiperidin-1-yl)pyrazin-2-yl)thio)-2-chlorophenyl)sulfonyl)benzamide,
3-((3-amino-5-(4-(aminomethyl)-4-methylpiperidin-1-yl)pyrazin-2-yl)thio)-2-chloro-N-((2-fluorophenyl)sulfonyl)benzamide,
3-((3-amino-5-(4-(aminomethyl)-4-methylpiperidin-1-yl)pyrazin-2-yl)thio)-2-chloro-N-(pyridin-2-ylsulfonyl)benzamide,
3-((3-amino-5-(4-(aminomethyl)-4-methylpiperidin-1-yl)pyrazin-2-yl)thio)-2-chloro-N-(cyclopropylsulfonyl)benzamide,
3-((3-amino-5-(4-(aminomethyl)-4-methylpiperidin-1-yl)pyrazin-2-yl)thio)-2-chloro-N-(methylsulfonyl)benzamide,
3-((3-amino-5-(4-(aminomethyl)-4-methylpiperidin-1-yl)pyrazin-2-yl)thio)-2-chloro-N-(pyridin-3-ylsulfonyl)benzamide,
3-((3-amino-5-(4-(aminomethyl)-4-methylpiperidin-1-yl)pyrazin-2-yl)thio)-2-chloro-N-(pyridin-4-ylsulfonyl)benzamide,
N-((3-((3-amino-5-(4-(aminomethyl)-4-methylpiperidin-1-yl)pyrazin-2-yl)thio)-2-chlorophenyl)carbamoyl)methanesulfonamide,
N-((3-((3-amino-5-(4-(aminomethyl)-4-methylpiperidin-1-yl)pyrazin-2-yl)thio)-2-chlorophenyl)carbamoyl)pyridine-4-sulfonamide,
N-((3-((3-amino-5-(4-(aminomethyl)-4-methylpiperidin-1-yl)pyrazin-2-yl)thio)-2-chlorophenyl)carbamoyl)cyclopentanesulfonamide,
N-((3-((3-amino-5-(4-(aminomethyl)-4-methylpiperidin-1-yl)pyrazin-2-yl)thio)-2-chlorophenyl)carbamoyl)cyclopropanesulfonamide,
3-((3-amino-5-(4-(aminomethyl)-4-methylpiperidin-1-yl)pyrazin-2-yl)thio)-2-chloro-N-(cyclopentylsulfonyl)benzamideN-((3-((3-amino-5-(4-(aminomethyl)-4-methylpiperidin-1-yl)pyrazin-2-yl)thio)-2-chlorophenyl)carbamoyl)pyridine-2-sulfonamide,
N-((3-((3-amino-5-(4-(aminomethyl)-4-methylpiperidin-1-yl)pyrazin-2-yl)thio)-2-chlorophenyl)carbamoyl)pyridine-3-sulfonamide,
N-((3-((3-amino-5-(4-(aminomethyl)-4-methylpiperidin-1-yl)pyrazin-2-yl)thio)-2-chlorophenyl)carbamoyl)-2-fluorobenzenesulfonamide,
N-((4-((5-(4-(aminomethyl)-4-methylpiperidin-1-yl)pyrazin-2-yl)thio)-3-chloropyridin-2-yl)carbamoyl)benzenesulfonamide,
N-((3-((5-((3S,4S)-4-amino-3-methyl-2-oxa-8-azaspiro[4.5]decan-8-yl)pyrazin-2-yl)thio)-2-chlorophenyl)carbamoyl)benzenesulfonamide,
N-((4-((5-((3S,4S)-4-amino-3-methyl-2-oxa-8-azaspiro[4.5]decan-8-yl)pyrazin-2-yl)thio)-3-chloropyridin-2-yl)carbamoyl)-3-fluorobenzenesulfonamide,
N-((4-((5-((3S,4S)-4-amino-3-methyl-2-oxa-8-azaspiro[4.5]decan-8-yl)pyrazin-2-yl)thio)-3-chloropyridin-2-yl)carbamoyl)pyridine-3-sulfonamide,
N-((4-((5-((3S,4S)-4-amino-3-methyl-2-oxa-8-azaspiro[4.5]decan-8-yl)pyrazin-2-yl)thio)-3-chloropyridin-2-yl)carbamoyl)-2-fluorobenzenesulfonamide,
N-((4-((5-((3S,4S)-4-amino-3-methyl-2-oxa-8-azaspiro[4.5]decan-8-yl)pyrazin-2-yl)thio)-3-chloropyridin-2-yl)carbamoyl)-4-fluorobenzenesulfonamide,
N-((4-((5-((3S,4S)-4-amino-3-methyl-2-oxa-8-azaspiro[4.5]decan-8-yl)pyrazin-2-yl)thio)-3-chloropyridin-2-yl)carbamoyl)-6-methoxypyridine-3-sulfonamide,
N-((4-((5-((3S,4S)-4-amino-3-methyl-2-oxa-8-azaspiro[4.5]decan-8-yl)pyrazin-2-yl)thio)-3-chloropyridin-2-yl)carbamoyl)-1-methyl-1H-pyrazole-4-sulfonamide,
N-((4-((5-((3S,4S)-4-amino-3-methyl-2-oxa-8-azaspiro[4.5]decan-8-yl)pyrazin-2-yl)thio)-3-fluoropyridin-2-yl)carbamoyl)benzenesulfonamide,
N-((4-((5-((3S,4S)-4-amino-3-methyl-2-oxa-8-azaspiro[4.5]decan-8-yl)pyrazin-2-yl)thio)-3-chloropyridin-2-yl)carbamoyl)cyclopropanesulfonamide,
N-((4-((5-((3S,4S)-4-amino-3-methyl-2-oxa-8-azaspiro[4.5]decan-8-yl)pyrazin-2-yl)thio)-3-chloropyridin-2-yl)carbamoyl)-5-fluoropyridine-3-sulfonamide,
N-((4-((5-((3S,4S)-4-amino-3-methyl-2-oxa-8-azaspiro[4.5]decan-8-yl)-6-(hydroxymethyl)-3-methylpyrazin-2-yl)thio)-3-chloropyridin-2-yl)carbamoyl)benzenesulfonamide,
N-((5-((5-((3S,4S)-4-amino-3-methyl-2-oxa-8-azaspiro[4.5]decan-8-yl)pyrazin-2-yl)thio)-4-chloropyridin-3-yl)carbamoyl)benzenesulfonamide,
N-((3-((5-((3S,4S)-4-amino-3-methyl-2-oxa-8-azaspiro[4.5]decan-8-yl)pyrazin-2-yl)thio)-2-chlorophenyl)carbamoyl)pyridine-3-sulfonamide,
N-((3-((5-((3S,4S)-4-amino-3-methyl-2-oxa-8-azaspiro[4.5]decan-8-yl)pyrazin-2-yl)thio)-2-chlorophenyl)carbamoyl)-3-fluorobenzenesulfonamide,
N-((3-((5-((3S,4S)-4-amino-3-methyl-2-oxa-8-azaspiro[4.5]decan-8-yl)pyrazin-2-yl)thio)-2-chlorophenyl)carbamoyl)pyridine-2-sulfonamide,
N-((3-((5-((3S,4S)-4-amino-3-methyl-2-oxa-8-azaspiro[4.5]decan-8-yl)pyrazin-2-yl)thio)-2-chlorophenyl)carbamoyl)-6-methoxypyridine-3-sulfonamide,
N-((3-((5-((3S,4S)-4-amino-3-methyl-2-oxa-8-azaspiro[4.5]decan-8-yl)pyrazin-2-yl)thio)-2-chlorophenyl)carbamoyl)-3,5-dimethylisoxazole-4-sulfonamide,
N-((3-((5-((3S,4S)-4-amino-3-methyl-2-oxa-8-azaspiro[4.5]decan-8-yl)pyrazin-2-yl)thio)-2-chlorophenyl)carbamoyl)cyclopropanesulfonamide,
(S)—N-((3-((5-(7-amino-5,7-dihydrospiro[cyclopenta[c]pyridine-6,4′-piperidin]-1′-yl)pyrazin-2-yl)thio)-2-chlorophenyl)carbamoyl)benzenesulfonamide,
N-((3-((5-((3S,4S)-4-amino-3-methyl-2-oxa-8-azaspiro[4.5]decan-8-yl)pyrazin-2-yl)thio)-2-chlorophenyl)carbamoyl)tetrahydro-2H-pyran-4-sulfonamide,
N-((3-((5-((3S,4S)-4-amino-3-methyl-2-oxa-8-azaspiro[4.5]decan-8-yl)pyrazin-2-yl)thio)-2-chlorophenyl)carbamoyl)cyclohexanesulfonamide,
N-((3-((5-((3S,4S)-4-amino-3-methyl-2-oxa-8-azaspiro[4.5]decan-8-yl)pyrazin-2-yl)thio)-2-chlorophenyl)carbamoyl)-1-methylpiperidine-4-sulfonamide,
4-((5-((3S,4S)-4-amino-3-methyl-2-oxa-8-azaspiro[4.5]decan-8-yl)pyrazin-2-yl)thio)-3-chloro-N-(phenylsulfonyl)picolinamide,
N-((3-((5-((3S,4S)-4-amino-3-methyl-2-oxa-8-azaspiro[4.5]decan-8-yl)pyrazin-2-yl)thio)-2-chlorophenyl)carbamoyl)pyridine-4-sulfonamide,
N-((3-((5-((3S,4S)-4-amino-3-methyl-2-oxa-8-azaspiro[4.5]decan-8-yl)pyrazin-2-yl)thio)-2-chlorophenyl)carbamoyl)cyclopentanesulfonamide,
N-((3-((5-((3S,4S)-4-amino-3-methyl-2-oxa-8-azaspiro[4.5]decan-8-yl)pyrazin-2-yl)thio)-2-chlorophenyl)carbamoyl)morpholine-4-sulfonamide,
N-((3-((5-((3S,4S)-4-amino-3-methyl-2-oxa-8-azaspiro[4.5]decan-8-yl)pyrazin-2-yl)thio)-2-chlorophenyl)carbamoyl)piperidine-1-sulfonamide,
N-((4-((5-((3S,4S)-4-amino-3-methyl-2-oxa-8-azaspiro[4.5]decan-8-yl)pyrazin-2-yl)thio)-3-chloropyridin-2-yl)carbamoyl)-3,5-dimethylisoxazole-4-sulfonamide,
N-((4-((5-((3S,4S)-4-amino-3-methyl-2-oxa-8-azaspiro[4.5]decan-8-yl)pyrazin-2-yl)thio)-3-chloropyridin-2-yl)carbamoyl)methanesulfonamide,
N-((3-((5-((3S,4S)-4-amino-3-methyl-2-oxa-8-azaspiro[4.5]decan-8-yl)pyrazin-2-yl)thio)-2-chlorophenyl)carbamoyl)-4-fluorobenzenesulfonamide,
N-((3-((5-((3S,4S)-4-amino-3-methyl-2-oxa-8-azaspiro[4.5]decan-8-yl)pyrazin-2-yl)thio)-2-chlorophenyl)carbamoyl)-1-methyl-1H-pyrazole-4-sulfonamide,
N—(N-(3-((5-((3S,4S)-4-amino-3-methyl-2-oxa-8-azaspiro[4.5]decan-8-yl)pyrazin-2-yl)thio)-2-chlorophenyl)sulfamoyl)benzamide,
N-((3-((5-((3S,4S)-4-amino-3-methyl-2-oxa-8-azaspiro[4.5]decan-8-yl)pyrazin-2-yl)thio)-2-chlorophenyl)carbamoyl)-3-hydroxypyrrolidine-1-sulfonamide,
N-((3-((5-((3S,4S)-4-amino-3-methyl-2-oxa-8-azaspiro[4.5]decan-8-yl)pyrazin-2-yl)thio)-2-chlorophenyl)carbamoyl)cyclobutanesulfonamide,
N-((3-((5-((3S,4S)-4-amino-3-methyl-2-oxa-8-azaspiro[4.5]decan-8-yl)pyrazin-2-yl)thio)-2-chlorophenyl)carbamoyl)tetrahydrofuran-3-sulfonamide, and
3-amino-N-((3-((5-((3S,4S)-4-amino-3-methyl-2-oxa-8-azaspiro[4.5]decan-8-yl)pyrazin-2-yl)thio)-2-chlorophenyl)carbamoyl)pyrrolidine-1-sulfonamide.

39. A pharmaceutical composition comprising a compound of any one of claims 1-38, and/or a stereoisomer, a stable isotope, or a pharmaceutically acceptable salt or solvate thereof, admixed with at least one pharmaceutically acceptable carrier.

40. A method to treat a disease in a patient in need thereof whose disease is a SHP2-associated disease, comprising administering to the subject in need of such treatment a therapeutically effective amount of a compound of any one of claims 1-38 and/or a stereoisomer, a stable isotope, or a pharmaceutically acceptable salt or solvate thereof, or a pharmaceutical composition of claim 39.

41. The method of claim 40, wherein the method comprises determining if the disease in the patient is a SHP2-associated disease, and administering to a subject in need of such treatment a therapeutically effective SHP2 inhibiting amount of a compound of any one of Embodiments 1-38, and/or a stereoisomer, a stable isotope, or a pharmaceutically acceptable salt or solvate thereof, or a pharmaceutical composition or a pharmaceutical composition of claim 39.

42. The method of claim 40 or claim 41, wherein the SHP2-associated disease or disorder is mediated by the activity of SHP2.

43. The method of claim 42, wherein the SHP2-associated disease or disorder is selected from Noonan Syndrome, Leopard Syndrome, juvenile myelomonocytic leukemias, neuroblastoma, melanoma, acute myeloid leukemia, breast cancer, esophageal cancer, lung cancer, colon cancer, head cancer, neuroblastoma, squamous-cell carcinoma of the head and neck, gastric carcinoma, anaplastic large-cell lymphoma and glioblastoma.

44. The method of any of one of claim 40-43, wherein the compound of any one of claims 1-38, and/or a stereoisomer, a stable isotope, or a pharmaceutically acceptable salt or solvate thereof, or a pharmaceutical composition of claim 39, is orally administered.

45. A use of a compound of any one of claims 1-38, and/or a stereoisomer, a stable isotope, or a pharmaceutically acceptable salt or solvate thereof, or a pharmaceutical composition according to claim 39 as a medicament, in the manufacture of a medicament, or in medicine for treatment of a SHP2-associated disease or disorder.

46. The use of claim 45 wherein the SHP2-associated disease or disorder is mediated by the activity of SHP2.

47. The use of claim 46, wherein the SHP2-associated disease or disorder is selected from Noonan Syndrome, Leopard Syndrome, juvenile myelomonocytic leukemias, neuroblastoma, melanoma, acute myeloid leukemia, breast cancer, esophageal cancer, lung cancer, colon cancer, head cancer, neuroblastoma, squamous-cell carcinoma of the head and neck, gastric carcinoma, anaplastic large-cell lymphoma and glioblastoma.

48. The use of any of one of claims 45-37, wherein the medicament is formulated for oral administration.

49. A compound of any one of claims 1-38, and/or a stereoisomer, a stable isotope, or a pharmaceutically acceptable salt or solvate thereof, or a pharmaceutical composition of claim 39 for use in treating a SHP2-associated disease or disorder.

50. The compound or pharmaceutical composition of claim 49, wherein the SHP2-associated disease is a SHP2-associated cancer.

51. The compound or pharmaceutical composition of claim 49, wherein the SHP2-associated disease or disorder is a SHP2-associated cancer, and the use comprises determining if the cancer in a patient is a SHP2-associated cancer, and administering to the patient in need of such treatment a therapeutically effective amount of the compound or pharmaceutical composition.

52. The compound or pharmaceutical composition of any of one of claims 49-51, wherein the SHP2-associated cancer is selected from juvenile myelomonocytic leukemias, neuroblastoma, melanoma, acute myeloid leukemia, breast cancer, esophageal cancer, lung cancer, colon cancer, head cancer, neuroblastoma, squamous-cell carcinoma of the head and neck, gastric carcinoma, anaplastic large-cell lymphoma and glioblastoma.

53. A method of inhibiting SHP2 activity in vitro or in vivo for a SHP2-associated cancer cell with a compound of any one of claims 1-38, and/or a stereoisomer, a stable isotope, or a pharmaceutically acceptable salt or solvate thereof.
